(19) United States
(12) Patent Application Publication
(10) Pub. No.: US 2011/0065676 A1
(43) Pub. Date: Mar. 17, 2011

(54) COMBINATION THERAPIES COMPRISING PAR1 ANTAGONISTS WITH NAR AGONISTS

(75) Inventors: Michael Sidney Perelman, Summit, NJ (US); Madhu Chintala, Colts Neck, NJ (US)

(73) Assignee: Schering Corporation, Kenilworth, NJ (US)

(21) Appl. No.: 12/820,456

(22) Filed: Jun. 22, 2010

Related U.S. Application Data
(60) Provisional application No. 61/219,957, filed on Jun. 24, 2009.

(51) Int. Cl. A61K 31/60; A61K 31/519; A61K 31/496; A61K 31/5377; A61K 31/551; A61K 31/443; A61K 31/4365; A61K 31/444; A61P 9/00; A61P 25/00; A61P 35/00

(52) U.S. Cl. 514/161; 514/262.1; 514/260.1; 514/267; 514/264.1; 514/252.16; 514/234.2; 514/218; 514/337; 514/301; 514/235.2; 514/333

(57) ABSTRACT

The present invention is directed to a pharmaceutical composition comprising an effective amount of at least one PAR1 antagonist, at least one NAR agonist, optionally, an effective amount of at least one cardiovascular agent, and, optionally, a pharmaceutically acceptable carrier. The present invention also provides for the use of theses pharmaceutical compositions to treat various diseases associated with thrombosis.

This application claims the benefit of U.S. provisional patent application No. 61/219,957, filed Jun. 24, 2009; which is herein incorporated by reference in its entirety.

### BACKGROUND OF THE INVENTION

Thrombin is known to have a variety of activities in different cell types. One role of thrombin is to activate thrombin receptors which are known to be present in such cell types as human platelets, vascular smooth muscle cells, endothelial cells and fibroblasts. PAR1 antagonists have been identified based on structure-activity studies involving substitutions of amino acids on thrombin receptors. In Bernatowicz et al, J. Med Chem., 1996, 39, pp. 4879-4887 tetra- and pentapeptides are disclosed as being potent PAR1 antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide PAR1 antagonists are also disclosed in WO 94/03479, published Feb. 17, 1994.

PAR1 antagonists have been suggested in the literature as being potentially useful in treating a variety of cardiovascular diseases or conditions including, for example, thrombosis, vascular restenosis, deep venous thrombosis, lung embolism, cerebral infarction, heart disease, disseminated intravascular coagulation syndrome, hypertension (Suzuki, Shuichi, PCT Int. Appls. WO 0288092 (2002), WO 0285850 (2002) and WO 0285855 (2002)), arrhythmia, inflammation, angina, stroke, atherosclerosis, ischemic conditions (Zhang, Han-cheng, PCT Int. Appl. WO 0100659 (2001), WO 0100657(2001) and WO 0100656 (2001)).

Thrombin activates human platelets through a proteolytic activation protease-activated receptor (PAR): protease-activated receptor-1 or PAR1 . PAR1 (M. L. Kahn et al, Nature, 1998, 394, 690-694). PAR1 is a high-affinity receptor for platelet activation at low concentrations of thrombin.

PAR1 antagonists are disclosed in U.S. Pat. Nos. 6,063,847; 6,326,380; 6,645,987; 6,894,065 and 6,645,987; in U.S. Publication Nos. 2003/0203927A1; 2004/0152736A1; 2003/0216437; 2004/192753A1 and 2002/049350A1; and WO99/26943 WO03/089428 and WO03/033501 all assigned to Schering-Plough and incorporated by reference. The use of a small subset of PAR1 antagonists to treat a variety of conditions and diseases is disclosed in U.S. Publication No. 04/0192753. A bisulfate salt of a particular PAR1 antagonist is disclosed in U.S. Publication No. 2004/0176418A1. The last two documents are also assigned to Schering-Plough and incorporated by reference. Other PAR1 antagonists are disclosed in WO02/088092; WO02/085855; WO02/085850, all assigned to Eisai Co., LTD. and incorporated by reference.

Nicotinic acid, commonly known as niacin, plays an important role in the production of several sex and stress-related hormones, particularly those made by the adrenal gland. It also plays a role in removing toxic and harmful chemicals from the body.

When taken in large doses, nicotinic acid increases the level of high density lipoprotein (HDL) in blood, and is sometimes prescribed for patients with low HDL, and at high risk of heart attack. Nicotinic acid is also used in the treatment of hyperlipidemia because it reduces very low density lipoprotein (VLDL), a precursor of low density lipoprotein (LDL) secretion from the liver, and inhibits cholesterol synthesis. Nicotinic acid has also been used to treat metabolic syndrome, but there are problems with the clinical use of nicotinic acid, including skin flushing and diarrhea, even with moderate doses.

The use of heterocyclic compounds as nicotinic acid receptor agonists is known in the art and such compounds are disclosed, for example, in M. Ridi, Gazzetta Chim. Ital. (1950) vol. 80, p. 121 and M. Ridi, Gazzetta Chim. Ital. (1952) vol. 82, p. 23, which disclose syntheses of barbituric acid derivatives useful as nicotinic acid receptor agonists. FR 2563223 discloses nucleoside analogs. T. Paterson et al., J. Chem. Soc., Perkins Trans. I (1972), vol. 8, pp. 1041-1050 discloses the synthesis of 8—Substituted pyrido[2,3-d]pyrimidines. S. Rao, Indian J. Chem. (1974), 12(10), pp. 1028-1030 discloses the synthesis of pyrano[2,3-d]pyrimidines. M. Skof, Heterocycles, (1999), 51(5), pp. 1051-1058 discloses one step transformations of (S)-1-benzoyl-3-[(E)-dimethylaminomethylidene]-5-methoxycarbonyl-pyrrolidin-2—One into quinolizinyl- and 2H-2-pyranonyl-substituted alanine derivatives. R. Toplak J. Heterocyclic Chem. (1999), 36(1), pp. 225-235 discloses the synthesis of pyran-2—Ones.

International Publication No. WO 05/077950 describes xanthine derivatives which are agonists of the nicotinic acid receptor HM74A.

International Publication No. WO 04/110368 describes combination therapies for the treatment of hypertension comprising the combination of an anti-obesity agent and an anti-hypertensive agent.

International Publication No. WO 05/000217 describes combination therapies for the treatment of dyslipidemia comprising the administration of a combination of an anti-obesity agent and an anti-dyslipidemic agent.

International Publication No. WO 04/110375 describes combination therapies for the treatment of diabetes comprising the administration of a combination of an anti-obesity agent and an anti-diabetic agent.

U.S. Patent Publication No. 2004/0122033 describes combination therapies for the treatment of obesity comprising the administration of a combination of an appetite suppressant and/or metabolic rate enhancers and/or nutrient absorption inhibitors. U.S. Patent Publication No. 2004/0229844 describes combination therapies for treating atherosclerosis comprising the administration of a combination of nicotinic acid or another nicotinic acid receptor agonist and a DP receptor antagonist.

In the search for enhanced efficacy and safety, researchers have explored various therapeutic combinations of two or more distinct active pharmaceutical agents. Such agents may act by very different biochemical pathways to provide particularly beneficial therapeutic results. The two or more active agents may be delivered as either co-administered monotherapy formulations, or as a single co-formulation. Co-formulations have the patient-compliance advantages of reducing the number of distinct doses and fixing the ratio of the two active agents being administered. An example of such a combination is CADUET®, which is a single dosage form comprising amlodipine (marketed in the U.S. as NORVASC®) and atorvastatin (marketed in the U.S. as LIPITOR®).

NAR agonists by their mechanism of action, lower plasma triglycerides and LDL cholesterol, and thereby inhibit the development of atherosclerosis. PAR1 antagonists on the other hand, inhibit intimal hyperplasia and restenosis. Thus the combination of NAR agonist and PAR1 antagonist markedly inhibits the development of dyslipidemia, atherosclerosis and restenosis. An additional benefit of the combination in man is that this combination inhibits thrombosis which is widely associated with atherosclerosis and plaque rupture in coronary artery disease.

### SUMMARY OF THE INVENTION

The present invention is directed to a pharmaceutical composition comprising an effective amount of at least one PAR1 antagonist, at least one NAR agonist, optionally, an effective amount of at least one cardiovascular agent, and, optionally, a pharmaceutically acceptable carrier.

More specifically, this invention is directed to a pharmaceutical composition comprising

i) an effective amount of at least one PAR1 antagonist which is selected from the group consisting of:
a) Formula I-A:

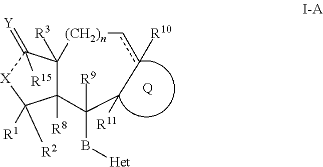

or a pharmaceutically acceptable salt or solvate thereof, wherein:

the single dotted line represents an optional single bond;

[symbol] represents an optional double bond;

n is 0-2;

Q is

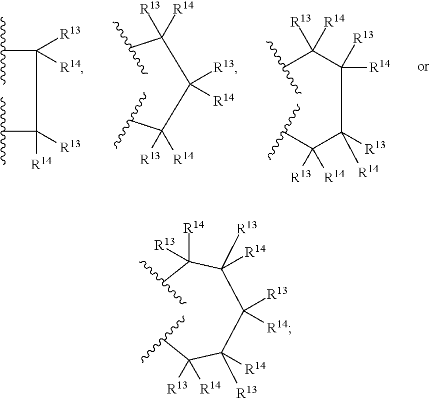

R¹ is independently selected from the group consisting of H, (C₁-C₆)alkyl, fluoro(C₁-C₆)alkyl-, trifluoro-(C₁-C₆)alkyl-, (C₃-C₆)cycloalkyl, (C₂-C₆)alkenyl, hydroxy-(C₁-C₆)alkyl-, and amino(C₁-C₆)alkyl-;

R² is independently selected from the group consisting of H, (C₁-C₆)alkyl, fluoro(C₁-C₆)alkyl-, difluoro(C₁-C₆)alkyl-, trifluoro-(C₁-C₆)alkyl-, (C₃-C₁₀)cycloalkyl, (C₂-C₆)alkenyl, hydroxy-(C₁-C₆)alkyl-, and amino(C₁-C₆)alkyl-;

R³ is H, hydroxy, (C₁-C₆)alkoxy, —SOR¹⁶, —SO₂R¹⁷, —C(O)OR¹⁷, —C(O)NR¹⁸R¹⁹, —(C₁-C₆)alkyl-C(O)NR¹⁸R^{1 9}, (C₁-C₆)alkyl, halogen, fluoro(C₁-C₆)alkyl-, difluoro(C₁-C₆)alkyl-, trifluoro(C₁-C₆)alkyl-, (C₃-C₁₀)cycloalkyl, (C₃-C₁₀)-cycloalkyl-(C₁-C₆)alkyl-, (C₂-C₆)alkenyl, aryl(C₁-C₆)alkyl-, aryl(C₂-C₆)alkenyl-, heteroaryl(C₁-C₆)alkyl-, heteroaryl(C₂-C₆)alkenyl-, hydroxy(C₁-C₆)-alkyl-, —NR²²R²³, NR²²R²³—(C₁-C₆)alkyl-, aryl, thio(C₁-C₆)alkyl-, (C₁-C₆)alkyl-thio(C₁-C₆)alkyl-, (C₁-C₆)alkoxy(C₁-C₆)alkyl-, NR¹⁸R¹⁹—C(O)—(C₁-C₆)alkyl- or (C₃-C₁₀)cycloalkyl-(C₁-C₆)alkyl-;

Het is a mono- or bi-cyclic heteroaryl group of 5 to 10 atoms comprised of 1 to 9 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by W;

W is 1 to 4 substituents independently selected from the group consisting of H, (C₁-C₆)alkyl, fluoro(C₁-C₆)alkyl-, difluoro(C₁-C₆)alkyl-, trifluoro(C₁-C₆)alkyl-, (C₃-C₁₀)cycloalkyl, hydroxy(C₁-C₆)alkyl-, dihydroxy(C₁-C₆)alkyl-, NR²⁵R²⁶(C₁-C₆)alkyl-, thio(C₁-C₆)alkyl-, —OH, (C₁-C₆)alkoxy, halogen, —NR⁴R⁵, —C(O)OR¹⁷, —C(O)R¹⁶, (C₁-C₆)alkylthio-, R²¹-aryl, R²¹-aryl(C₁-C₆)alkyl-, aryl wherein adjacent carbons form a ring comprising a methylenedioxy group, and R²¹-heteroaryl;

R⁴ and R⁵ are independently selected from the group consisting of H, (C₁-C₆)alkyl, phenyl, benzyl and (C₃-C₁₀)cycloalkyl, or R⁴ and R⁵ taken together are —(CH₂)₄—, —(CH₂)₅— or —(CH₂)₂NR⁷—(CH₂)₂— and form a ring with the nitrogen to which they are attached;

R⁶ is H, (C₁-C₆)alkyl or phenyl;

R⁷ is H, (C₁-C₆)alkyl, —C(O)—R¹⁶, —C(O)OR¹⁷ or —SO₂R¹⁷;

R⁸, R¹⁰ and R¹¹ are independently selected from the group consisting of R¹ and —OR¹, provided that when the optional double bond is present, R¹⁰ is absent;

R⁹ is H, OH or (C₁-C₆)alkoxy;

B is —(CH₂)ₙ₃-, cis or trans —(CH₂)ₙ₄CR¹²═CR^{12a}(CH₂)ₙ₅ or —(CH₂)ₙ₄C≡C(CH₂)ₙ₅—, wherein n₃ is 0-5, n₄ and n₅ are independently 0-2, and R¹² and R^{12a} are independently selected from the group consisting of H, (C₁-C₆)alkyl and halogen;

X is —O— or —NR⁶— when the dotted line represents a single bond, or X is —OH or —NHR²⁰ when the bond is absent;

Y is oxo, thioxo, (H, H), (H, OH) or (H, (C₁-C₆)alkoxy) when the dotted line represents a single bond, or when the bond is absent, Y is oxo, (H, H), (H, OH), (H, SH) or (H, (C₁-C₆)alkoxy);

each R¹³ is independently selected from H, (C₁-C₆)alkyl, (C₃-C₁₀)cycloalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halo(C₁-C₆)alkyl, and halogen;

each R¹⁴ is independently selected from H, (C₁-C₆)alkyl, —OH, (C₁-C₆)alkoxy, R²⁷-aryl(C₁-C₆)alkyl, heteroaryl, heteroarylalkyl, heterocyclyl, heterocyclylalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halogen and halo(C₁-C₆)alkyl; or

R¹³ and R¹⁴ taken together form a spirocyclic or a heterospirocyclic ring of 3-6 atoms;

wherein at least one of R¹³ or R¹⁴ is selected from the group consisting of —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4;

R¹⁵ is absent when the dotted line represents a single bond and is H, (C₁-C₆)alkyl, —NR¹⁸R¹⁹, or —OR¹⁷ when the bond is absent;

R¹⁶ is independently selected from the group consisting of (C₁-C₆)alkyl, phenyl and benzyl;

R^{16b} is H, (C₁-C₆)alkyl, (C₁-C₆)alkoxy(C₁-C₆)alkyl-, R²²—O—C(O)—(C₁-C₆)alkyl-, (C₃-C₁₀)cycloalkyl, R²¹-aryl, R²¹-aryl(C₁-C₆)alkyl, halo(C₁-C₆)alkyl, (C₂-C₆)alkenyl, halosubstituted (C₂-C₆)alkenyl, (C₂-C₆)alkynyl, halosubstituted (C₂-C₆)alkynyl, R²¹-heteroaryl, R²¹—(C₁-C₆)alkyl heteroaryl, R²¹—(C₁-C₆)alkyl heterocycloalkyl, R²⁸R²⁹N—(C₁-C₆)alkyl, R²⁸R²⁹N—(CO)—(C₁-C₆)alkyl, R²⁸R²⁹N—(CO)O—(C₁-C₆)alkyl, R²⁸O(CO)N(R²⁹)—(C₁-C₆)alkyl, R²⁸S(O)₂N(R²⁹)—(C₁-C₆)alkyl, R²⁸R²⁹N—(CO)—N(R²⁹)—(C₁-C₆)alkyl, R²⁸R²⁹N—S(O)2N(R²⁹)—(C₁-C₆)alkyl, R²⁸—(CO)N(R²⁹)—(C₁-C₆)alkyl, R²⁸R²⁹N—S(O)₂—(C₁-C₆)alkyl, HOS(O)₂—(C₁-C₆)alkyl, (OH)₂P(O)₂—(C₁-C₆)alkyl, R²⁸—S—(C₁-C₆)alkyl, R²⁸—S(O)₂—(C₁-C₆)alkyl or hydroxy(C₁-C₆)alkyl);

R¹⁷, R¹⁸ and R¹⁹ are independently selected from the group consisting of H, (C₁-C₆)alkyl, phenyl, and benzyl;

R²⁰ is H, (C₁-C₆)alkyl, phenyl, benzyl, —C(O)R⁶ or —SO₂R⁶;

R²¹ is 1 to 3 substituents independently selected from the group consisting of H, —CN, —CF₃, —OCF₃, halogen, —NO₂, (C₁-C₆)alkyl, —OH, (C₁-C₆)alkoxy, (C₁-C₆)-alkylamino-, di-((C₁-C₆)alkyl)amino-, NR²⁵R²⁶⁻(C₁-C₆)alkyl-, hydroxy-(C₁-C₆)alkyl-, —C(O)OR¹⁷, —COR¹⁷, —NHCOR¹⁶, —NHSO₂R¹⁶, —NHSO₂CH₂CF₃, —C(O)NR²⁵R²⁶, —NR²⁵—C(O)—NR²⁵R²⁶, —S(O)R¹³, —S(O)₂R¹³ and —SR¹³;

R²² is H or (C₁-C₆)alkyl;

R²³ is H, (C₁-C₆)alkyl, —C(O)R²⁴, —SO₂R²⁴, —CONHR²⁴ or —SO₂NHR²⁴;

R²⁴ is (C₁-C₆)alkyl, hydroxy (C₁-C₆)alkyl or NR²⁵R²⁶-((C₁-C₆)alkyl)-;

R²⁵ and R²⁶ are independently selected from the group consisting of H and (C₁-C₆)alkyl;

R²⁷ is 1, 2 or 3 substituents selected from the group consisting of H, (C₁-C₆)alkyl, (C₃-C₆)cycloalkyl, (C₁-C₆)alkoxy, halogen and —OH; and

R²⁸ and R²⁹ are independently selected from the group consisting of H, (C₁-C₆)alkyl, (C₁-C₆)alkoxy, R²⁷-aryl(C₁-C₆)alkyl, heteroaryl, heteroaryl(C₁-C₆)alkyl, hydroxy(C₁-C₆)alkyl, (C₁-C₆)alkoxy(C₁-C₆)alkyl, heterocyclyl, heterocyclyl(C₁-C₆)alkyl, and halo(C₁-C₆)alkyl; or

R²⁸ and R²⁹ taken together form a spirocyclic or a heterospirocyclic ring of 3-6 atoms;

b) Formula I-B

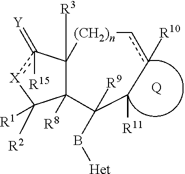

or a pharmaceutically acceptable salt thereof, wherein:

the single dotted line represents an optional double bond;

the double dotted line represents an optional single bond;

n is 0-2;

Q is

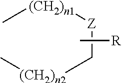

wherein n₁ and n₂ are independently 0-2; or when the double bond is not present, Q is also fused R-substituted aryl or R-substituted heteroaryl;

R is 1 to 3 substituents independently selected from the group consisting of H, (C₁-C₆)alkyl, halogen, hydroxy, amino, (C₁-C₆)alkyl-amino, (C₁-C₆)dialkylamino, (C₁-C₆)alkoxy, —COR¹⁶, —COOR¹⁷, —SOR¹⁶, —SO₂R¹⁶, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)-alkyl, heteroaryl(C₂-C₆)alkenyl, hydroxy(C₁-C₆)alkyl, amino(C₁-C₆)-alkyl, aryl and thio(C₁-C₆)alkyl;

R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy-(C₁-C₆)alkyl, amino(C₁-C₆)alkyl, aryl and thio(C₁-C₆)alkyl; or R¹ and R² together form an oxo group;

R³ is H, hydroxy, (C₁-C₆)alkoxy, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl or thio(C₁-C₆)alkyl;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl, (C₃-C₁₀)cycloalkyl; heterocycloalkyl; heterocycloalkyl substituted by (C₁-C₆)alkyl or (C₂-C₆)alkenyl; (C₂-C₆)alkenyl; R²¹-aryl(C₁-C₆)alkyl; R²¹-aryl-(C₂-C₆)-alkenyl; heteroaryl(C₁-C₆)alkyl; heteroaryl(C₂-C₆)-alkenyl; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; (C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —C(R⁶)₂C(O)NR⁴R⁵; (C₁-C₆)alkoxy; (C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, aryl wherein adjacent carbons form a ring with a methylenedioxy group, —C(O)NR⁴R⁵ and heteroaryl; R²¹-aryl; aryl wherein adjacent carbons form a ring with a methylenedioxy group; heteroaryl; heteroaryl substituted by 1 to 4 substituents selected from the group consisting of halogen, (C₁-C₆)alkoxy, (C₁-C₆)alkyl, (C₁-C₆)alkylamino, di-((C₁-C₆)alkyl)amino, —OCF₃, —NO₂, hydroxy(C₁-C₆)alkyl, —CHO and phenyl; and heteroaryl wherein adjacent carbon atoms form a ring with a C₃-C₅ alkylene group or a methylenedioxy group;

R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₄—, —(CH₂)₅— or —(CH₂)₂NR⁷—(CH₂)₂— and form a ring with the nitrogen to which they are attached;

R⁶ is independently selected from the group consisting of H, (C₁-C₆)alkyl or phenyl;

R⁷ is H or (C₁-C₆)alkyl;

R⁸, R¹⁰ and R¹¹ are independently selected from the group consisting of R¹ and —OR¹, provided that when the optional double bond is present, R¹⁰ is absent, and when ring Q is aromatic, R¹⁰ and R¹¹ are absent;

R⁹ is H, OH, (C₁-C₆)alkoxy, halogen or halo(C₁-C₆)alkyl;

B is —(CH₂)ₙ₃—, —CH₂—O—, —CH₂S—, —CH₂—NR⁶—, —C(O)NR⁶—. —NR⁶C(O)—,

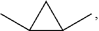

cis or trans —(CH₂)ₙ₄CR¹²═CR^{12a}(CH₂)ₙ₅ or, —(CH₂)ₙ₄C≡CR(CH₂)ₙ₅—, wherein n₃ is 0-5, n₄ and n₅ are independently 0-2, and R¹² and R^{12a} are independently selected from the group consisting of H, (C₁-C₆)alkyl and halogen;

X is —O— or —NR⁶— when the double dotted line represents a single bond, or X is —OH or —NHR²⁰ when the bond is absent;

Y is oxo, thioxo, (H, H), (H, OH) or (H, (C₁-C₆)alkoxy) when the double dotted line represents a single bond, or when the bond is absent, Y is oxo, (H, H), (H, OH), (H, SH) or (H, (C₁-C₆)alkoxy);

R¹⁵ is absent when the double dotted line represents a single bond and is H, (C₁-C₆)alkyl, —NR¹⁸R¹⁹, or —OR¹⁷ when the bond is absent; or Y is

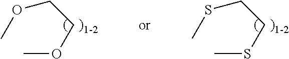

and R¹⁵ is H or (C₁-C₆)alkyl;

R¹⁶ and R^{16a} are independently selected from the group consisting of (C₁-C₆)lower alkyl, phenyl or benzyl;

R¹⁷, R¹⁸ and R¹⁹ are independently selected from the group consisting of H, (C₁-C₆)alkyl, phenyl, benzyl;

R²⁰ is H, (C₁-C₆)alkyl, phenyl, benzyl, —C(O)R⁶ or —SO₂R⁶;

R²¹ is 1 to 3 substituents independently selected from the group consisting of —CF₃, —OCF₃, halogen, —NO₂, (C₁-C₆)alkyl, (C₁-C₆)alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶ and —NHSO₂CH₂CF₃;

Z is —CH₂—, —O—, —S(O)₀₋₂—, —NR²²—, —C(O)—, —C(═NOR¹⁷)— or —C(R¹³R¹⁴)—, wherein R¹³ and R¹⁴, together with the carbon to which they are attached, form a spirocycloalkyl group of 3 to 6 carbons, or a spiroheterocycloalkyl group of 3 to 6 members, comprised of 2 to 5 carbon atoms and 1 or 2 heteroatoms selected from the group consisting of O, S and N; and

R²² is H, (C₁-C₆)alkyl, phenyl, benzyl, —COR¹⁶ or —CONR¹⁸R¹⁹;
c) Formula I-C

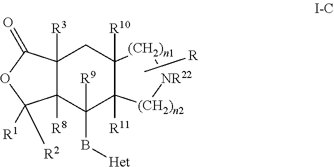

or a pharmaceutically acceptable salt thereof, wherein:

R is 1 to 3 substituents independently selected from the group consisting of H, (C₁-C₆)alkyl, halogen, hydroxy, amino, (C₁-C₆)alkyl-amino, (C₁-C₆)-dialkylamino, (C₁-C₆)alkoxy, —COR¹⁶, —COOR¹⁷, —SOR¹⁶, —SO₂R¹⁶, —SO₂NR¹⁷R¹⁸, —NR¹⁷SO₂R¹⁸, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, aryl(C₁-C₆)alkyl, hydroxy(C₁-C₆)alkyl, amino-(C₁-C₆)-alkyl, aryl and thio(C₁-C₆)alkyl;

R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, hydroxy-(C₁-C₆)alkyl, amino(C₁-C₆)alkyl, aryl and thio(C₁-C₆)alkyl; or R¹ and R² together form an oxo group;

R³ is H, hydroxy, (C₁-C₆)alkoxy, aryloxy, aryl(C₁-C₆)alkyloxy, heteroaryloxy, heteroaryl(C₁-C₆)alkyloxy, (C₃-C₁₀)cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², ——NR³³CONR³³R³⁴, —NR³³S(O)₂NR³³R³⁴, —(CR³³R³⁴)ₙNR⁴R⁵, —(CR³³R³⁴)ₙNR³³COOR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR³³S(O)₂NR³³R³⁴, (C₁-C₆)alkyl, halogen, (C₃-C₁₀)cycloalkyl, C₂-C₆)alkenyl, —CN, aryl, heteroaryl, heterocycloalkyl, —P(O)(OR⁷)₂ or (C₁-C₆)alkyl substituted by 1 to 3 substituents independently selected from the group consisting of halogen, —OH, —NH₂, aryl, —COOH, —SO₃H, thio and (C₁-C₆)alkylthio;

n is 1, 2, 3 or 4;

n1 and n2 are independently 0-3, provided both are not 0;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of (C₁-C₆)alkyl; —NR⁴R⁵; —NHCOR²⁶; —NHSO₂R¹⁶; R²¹-aryl; aryl wherein adjacent carbons form a ring with a methylenedioxy group; and
R²¹-heteroaryl;

R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;

R⁷ is H or (C₁-C₆)alkyl;

R⁸, R¹⁰ and R¹¹ independently independently selected from the group consisting of R¹ and —OR¹;

R⁹ is H, OH, —NR⁴R⁵, (C₁-C₆)alkoxy, halogen or halo(C₁-C₆)alkyl;

B is —(CH₂)ₙ₃— or cis or trans —(CH₂)ₙ₄CR¹²═CR^{12a}(CH₂)n₅, wherein n₃ is 0-5, n₄ and n₅ are independently 0-2, and R¹² and R^{12a} are independently selected from the group consisting of H, (C₁-C₆)alkyl and halogen;

R¹⁶ and R^{16a} are independently selected from the group consisting of (C₁-C₆), phenyl and benzyl;

R¹⁷, R¹⁸ and R¹⁹ are independently selected from the group consisting of H, (C₁-C₆)alkyl, phenyl and benzyl;

R²¹ is 1 to 3 substituents independently selected from the group consisting of H, —CF₃, —OCF₃, halogen, —NO₂, —CN, (C₁-C₆)alkyl, (C₁-C₆)alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O)NR²⁴R²⁵, —SO₂R³⁰, —P(O)(OR²⁹)₂, aryl, aryl(C₁-C₆)alkyl, heteroaryl, heterocycloalkyl, and —CR²⁹(═NOR²⁸);

R²² is —COR²³, —S(O)R³¹, —S(O)₂R³¹, —SO₂NR²⁴R²⁵ or —COOR²⁷;

R²³ is halo(C₁-C₆)alkyl; (C₂-C₆)alkenyl; halo(C₂-C₆)alkenyl; (C₂-C₆)alkynyl; (C₃-C₁₀)-cycloalkyl; (C₃-C₁₀)cycloalkyl(C₁-C₆)alkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₁-C₆)alkyl; heteroaryl; heterocycloalkyl; (C₁-C₆)alkyl substituted by 1-3 substituents independently selected from —COOH and —SO₃H; or

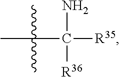

wherein R³⁵ and R³⁶ are independently selected from the group consisting of H, (C₁-C₆)alkyl, or R³⁷-substituted (C₁-C₆)alkyl, wherein R³⁷ is selected from the group consisting of HO—, HS—, CH₂S—, —NH₂, phenyl, p-hydroxyphenyl and indolyl;

R²⁴ and R²⁵ are independently selected form the group consisting of H, (C₁-C₆)alkyl, halo(C₁-C₆)alkyl, (C₂-C₆)alkenyl, halo(C₁-C₆)alkyl, (C₂-C₆)alkynyl, aryl, aryl-(C₁-C₆)alkyl, (C₃-C₁₀)-cycloalkyl, halo(C₃-C₁₀)cycloalkyl, (C₁-C₃)alkoxy(C₁-C₃)-hydroxy and (C₁-C₆)alkoxy;

R²⁶ is (C₃-C₁₀)-cycloalkyl, aryl, aryl-(C₁-C₆)alkyl, heteroaryl, heteroaryl-(C₁-C₆)alkyl or (C₁-C₆)alkylamino;

R²⁷ is (C₁-C₆)alkyl, phenyl, benzyl, (C₁-C₃)alkoxy(C₁-C₃)-alkyl, (C₃-C₁₀)cycloalkyl, carboxy(C₁-C₆)alkyl, sulfo(C₁-C₆)alkyl, or (C₁-C₆)alkyl substituted by NR¹⁸R¹⁹ and carboxy;

R²⁸ is H, (C₁-C₆)alkyl, phenyl, benzyl or (C₁-C₃)alkoxy(C₁-C₃)alkyl;

R²⁹ and R³⁰ are independently selected from the group consisting of H and C₁-C₆ alkyl;

R³¹ is (C₁-C₆)alkyl; halo(C₁-C₆)alkyl; (C₂-C₆)alkenyl; halo(C₂-C₆)alkyl; (C₂-C₆)alkynyl; (C₃-C₁₀)cycloalkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₁-C₆)alkyl; heteroaryl; heterocycloalkyl; (C₁-C₆)alkyl substituted by 1-3 substituents independently selected from —COOH and —SO₃H; or (C₁-C₆)alkoxy;

R³² is R³⁵—(C₁-C₆)alkyl, R³⁵—(C₃-C₁₀)cycloalkyl, R³⁵—(C₂-C₆)alkenyl, R³⁵—(C₂-C₆)-alkynyl or R³⁵-aryl, wherein R³⁵ is 1 or 2 substituents independently selected from the group consisting of H, —COOH, —NH₂, —SO₃H, oxo and ═NOR²⁸; and

R³³ and R³⁴ are independently selected from the group consisting of H, (C₁-C₆)alkyl and (C₃-C₁₀)cycloalkyl;
d) Formula I-D

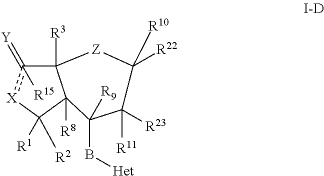

or a pharmaceutically acceptable salt thereof, wherein:

Z is —(CH₂)ₙ—;

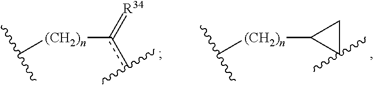

wherein R¹⁰ is absent; or

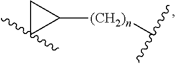

wherein R³ is absent;

the single dotted line represents an optional double bond;

the double dotted line represents an optional single bond;

n is 0-2;

R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy-(C₁-C₆)alkyl, (C₁-C₆)alkoxy(C₁-C₆)alkyl, amino-(C₁-C₆)alkyl, aryl and thio(C₁-C₆)alkyl; or R¹ and R² together form an oxo group;

R³ is H, hydroxy, (C₁-C₆)alkoxy, —NR¹⁸R¹⁹, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl;

R³⁴ is (H, R³), (H, R⁴³), oxo or ═NOR¹⁷ when the optional double bond is absent; R³⁴ is R⁴⁴ when the double bond is present;

Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl; (C₃-C₁₀)cycloalkyl; heterocycloalkyl; heterocycloalkyl substituted by (C₁-C₆)alkyl, (C₂-C₆)alkenyl, OH—(C₁-C₆)alkyl, or oxo; (C₂-C₆)alkenyl; R²¹-aryl(C₁-C₆)alkyl; R²¹-aryl-(C₂-C₆)-alkenyl;

R²¹-aryloxy; R²¹-aryl-NH—; heteroaryl(C₁-C₆)alkyl; heteroaryl(C₂-C₆)-alkenyl; heteroaryloxy; heteroaryl-NH—; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; —(C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; —C(═NOR¹⁷)R¹⁸; —(C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, aryl wherein adjacent carbons form a ring with a methylenedioxy group, —C(O)NR⁴R⁵ or heteroaryl; R²¹-aryl; aryl wherein adjacent carbons form a ring with a methylenedioxy group; R⁴¹-heteroaryl; and heteroaryl wherein adjacent carbon atoms form a ring with a C₃-C₅ alkylene group or a methylenedioxy group;

R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₄—(CH₂)₅— or —(CH₂)₂NR⁷—(CH₂)₂— and form a ring with the nitrogen to which they are attached;

R⁶ is independently selected from the group consisting of H, —(C₁-C₆)alkyl, phenyl, (C₃-C₁₀)cycloalkyl, (C₃-C₁₀)cycloalkyl(C₁-C₆)alkyl, (C₁-C₆)alkoxy(C₁-C₆)alkyl, hydroxy(C₁-C₆)alkyl and amino(C₁-C₆)alkyl;

R⁷ is H or (C₁-C₆)alkyl;

R⁸, R¹⁰ and R¹¹ are independently selected from the group consisting of R¹ and —OR¹, provided that when the optional double bond is present, R¹⁰ is absent;

R⁹ is H, OH, —(C₁-C₆)alkoxy, halogen or halo(C₁-C₆)alkyl;

B is —(CH₂)ₙ₃—, —CH₂—O—, —CH₂S—, —CH₂—NR⁶—, —C(O)NR⁶—, —NR⁶C(O)—,

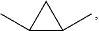

cis or trans —(CH₂)ₙ₄CR¹²═CR^{12a}(CH₂)ₙ₅ or —(CH₂)ₙ₄C≡C(CH₂)ₙ₅—, wherein n₃ is 0-5, n₄ and n₅ are independently 0-2, and R¹² and R^{12a} are independently selected from the group consisting of H, (C₁-C₆)alkyl and halogen;

X is —O— or —NR⁶— when the double dotted line represents a single bond, or X is H, —OH or —NHR²⁰ when the bond is absent;

Y is oxo, thioxo, (H, H), (H, OH) or (H, (C₁-C₆)alkoxy) when the double dotted line represents a single bond, or when the bond is absent, Y is oxo, ═NOR¹⁷, (H, H), (H, OH), (H, SH), (H, (C₁-C₆)alkoxy) or (H, —NHR⁴⁵);

R¹⁵ is absent when the double dotted line represents a single bond; R¹⁵ is H, (C₁-C₆)alkyl, —NR¹⁸R¹⁹ or —OR¹⁷ when the bond is absent; or Y is

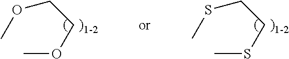

and R¹⁵ is H or (C₁-C₆)alkyl;

R¹⁶ is (C₁-C₆)lower alkyl, phenyl or benzyl;

R¹⁷, R¹⁸ and R¹⁹ are independently selected from the group consisting of H, (C₁-C₆)alkyl, phenyl, benzyl;

R²⁰ is H, (C₁-C₆)alkyl, phenyl, benzyl, —C(O)R⁶ or —SO₂R⁶;

R²¹ is 1 to 3 substituents independently selected from the group consisting of hydrogen, —CF₃, —OCF₃, halogen, —NO₂, —(C₁-C₆)alkyl, (C₁-C₆)alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶, —NHSO₂CH₂CF₃, heteroaryl or —C(═NOR¹⁷)R¹⁸;

R²² and R²³ are independently selected from the group consisting of hydrogen, R²⁴-(C₁-C₆)alkyl, R²⁴⁻(C₂-C₆)alkenyl, R²⁴—(C₂-C₆)alkynyl, R²⁷-hetero-cycloalkyl, R²⁵-aryl, R²⁵-aryl(C₁-C₆)alkyl, R²⁹—(C₃-C₁₀)cycloalkyl, R²⁹—(C₃-C₁₀)cycloalkenyl, —OH, —OC(O)R³⁰, —OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OR³⁰R³¹, R²⁴—(C₁-C₆)alkoxy, R²⁴—(C₂-C₆)-alkenyloxy, R²⁴—(C₂-C₆)alkynyloxy, R²⁷-heterocycloalkyloxy, R²⁹—(C₃-C₁₀)cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);

or R²² and R¹⁰ together with the carbon to which they are attached, or R²³ and R¹¹ together with the carbon to which they are attached, independently form a R⁴²-substituted carbocyclic ring of 3-10 atoms, or a R⁴²-substituted heterocyclic ring of 4-10 atoms wherein 1-3 ring members are independently selected from the group consisting of —O—, —NH— and —SO₀₋₂—, provided that when R²² and R¹⁰ form a ring, the optional double bond is absent;

R²⁴ is 1, 2 or 3 substituents independently selected from the group consisting of hydrogen, halogen, —OH, (C₁-C₆)alkoxy, R³⁵-aryl, (C₁-C₆)-alkyl-C(O)—, (C₂-C₆)-alkenyl-C(O)—, (C₂-C₆)alkynyl-C(O)—, heterocycloalkyl, R²⁶—(C₃-C₁₀)cycloalkyl, R²⁶—(C₃-C₁₀)cycloalkenyl, —OC(O)R³⁰, —C(O)OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₂-C₆)-alkenyloxy, R²⁴—(C₂-C₆)alkynyloxy, R²⁷-heterocycloalkyloxy, R²⁹—(C₃-C₁₀)-cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);

R²⁵ is 1, 2 or 3 substituents independently selected from the group consisting of hydrogen, heterocycloalkyl, halogen, —COOR³⁶, —CN, —C(O)NR³⁷R³⁸, —NR³⁹C(O)R⁴⁰, —OR³⁶, (C₃-C₁₀)cycloalkyl, (C₃-C₁₀)cycloalkyl-(C₁-C₆)alkyl, (C₁-C₆)alkyl(C₃-C₁₀)cycloalkyl-(C₁-C₆)alkyl, halo(C₁-C₆)alkyl(C₃-C₁₀)cycloalkyl(C₁-C₆)alkyl, hydroxy(C₁-C₆)alkyl, (C₁-C₆)alkoxy(C₁-C₆)alkyl, and R⁴¹-heteroaryl; or two R²⁵ groups on adjacent ring carbons form a fused methylenedioxy group

R²⁶ is 1, 2, or 3 substituents independently selected from the group consisting of hydrogen, halogen and (C₁-C₆)alkoxy;

R²⁷ is 1, 2 or 3 substituents independently selected from the group consisting of hydrogen, R²⁸—(C₁-C₆)alkyl, R²⁸—(C₂-C₆)alkenyl, R²⁸—(C₂-C₆)alkynyl,

R²⁸ is hydrogen, —OH or (C₁-C₆)alkoxy;

R²⁹ is 1, 2 or 3 substituents independently selected from the group consisting of hydrogen, (C₁-C₆)alkyl, —OH, (C₁-C₆)alkoxy and halogen;

R³⁰, R³¹ and R³² are independently selected from the group consisting of hydrogen, (C₁-C₆)-alkyl, (C₁-C₆)alkoxy(C₁-C₆)-alkyl, R²⁵-aryl(C₁-C₆)-alkyl, R³³—(C₃-C₁₀)cycloalkyl, R³⁴⁻(C₃-C₁₀)cycloalkyl(C₁-C₆)alkyl, R²⁵-aryl, heterocycloalkyl, heteroaryl, heterocycloalkyl(C₁-C₆)alkyl and heteroaryl(C₁-C₆)alkyl;

R³³ is hydrogen, (C₁-C₆)alkyl, OH—(C₁-C₆)alkyl or (C₁-C₆)alkoxy;

R³⁵ is 1 to 4 substituents independently selected from the group consisting of hydrogen, (C₁-C₆)alkyl, —OH, halogen, —CN, (C₁-C₆)alkoxy, trihalo(C₁-C₆)alkoxy, (C₁-C₆)alkylamino, di((C₁-C₆)alkyl)amino, —OCF₃, OH—(C₁-C₆)alkyl, —CHO, —C(O)(C₁-C₆)-alkylamino, —C(O)di((C₁-C₆)alkyl)amino, —NH₂, —NHC(O)(C₁-C₆)alkyl and —N((C₁-C₆)alkyl)C(O)(C₁-C₆)alkyl;

R³⁶ is hydrogen, (C₁-C₆)alkyl, halo(C₁-C₆)alkyl, dihalo(C₁-C₆)alkyl or trifluoro(C₁-C₆)alkyl,

R³⁷ and R³⁸ are independently selected from the group consisting of hydrogen, (C₁-C₆)alkyl, aryl(C₁-C₆)alkyl, phenyl and (C₃-C₁₅)cycloalkyl, or R³⁷ and R³⁸ together are —(CH₂)₄—, —(CH₂)₅— or —(CH₂)₂—NR³⁹—(CH₂)₂— and form a ring with the nitrogen to which they are attached;

R³⁹ and R⁴⁰ are independently selected from the group consisting of hydrogen, (C₁-C₆)alkyl, aryl(C₁-C₆)alkyl, phenyl and (C₃-C₁₅)-cycloalkyl, or R³⁹ and R⁴⁰ in the group —NR³⁹C(O)R⁴⁰, together with the carbon and nitrogen atoms to which they are attached, form a cyclic lactam having 5-8 ring members;

R⁴¹ is 1 to 4 substituents independently selected from the group consisting of hydrogen, (C₁-C₆)alkyl, (C₁-C₆)alkoxy, (C₁-C₆)alkylamino, di((C₁-C₆)alkyl)amino, —OCF₃, OH—(C₁-C₆)alkyl, —CHO and phenyl;

R⁴² is 1 to 3 substituents independently selected from the group consisting of hydrogen, —OH, (C₁-C₆)alkyl and (C₁-C₆)alkoxy;

R⁴³ is —NR³⁰R³¹, —NR³⁰C(O)R³¹, —NR³⁰C(O)NR³¹R³², —NHSO₂R³⁰ or —NHCOOR¹⁷;

R⁴⁴ is H, (C₁-C₆)alkoxy, —SOR¹⁶, —SO₂R¹⁷, —C(O)OR¹⁷, —C(O)NR¹⁸R¹⁹, —(C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl; and

R⁴⁵ is H, (C₁-C₆)alkyl, —COOR¹⁶ or —SO₂; and
e) Formula I-E-1, I-E-2 or I-E-3

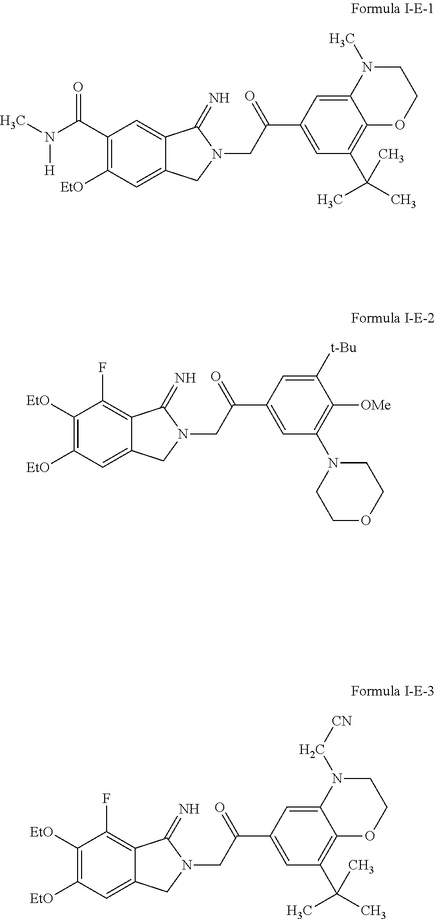

or a pharmaceutically acceptable salt thereof; and

ii) an effective amount of at least one NAR agonist which is selected from the group consisting of:

f) Formula II:

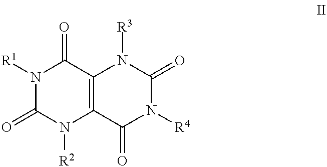

or a pharmaceutically acceptable salt, solvate, ester or prodrug thereof, wherein:

R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, heteroaryl, —OR⁵, —SR⁵, —N(R⁶)₂, —CN, —C(O)OR⁵ and —C(O)N(R⁶)₂;

R² is H, (C₁-C₆)alkyl, -(alkylene) aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵, —NHC(O)—R⁶ and —C(O)N(R⁶)₂;

R³ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁴ is H, (C₁-C₆)alkyl, -(alkylene) aryl, -(alkylene)ₙ-(C₃-C₁₀)cycloalkyl, -(alkylene)ₙ-cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)ₙ-aryl or (C₃-C₁₀)cycloalkyl; and

each occurrence of n is independently 0 or 1;

g) Formula III:

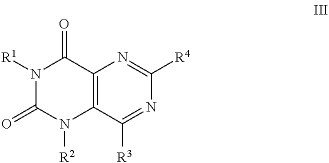

and pharmaceutically acceptable salts, solvates, esters and prodrugs thereof, wherein:

R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R² is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R³ is H, (C₁-C₆)alkyl, halo(C₁-C₆)alkyl, -(alkylene)ₙ-aryl, -(alkylene)ₙ-(C₃-C₁₀cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁴ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)ₙ-aryl or (C₃-C₁₀)cycloalkyl; and

each occurrence of n is independently 0 or 1;

h) Formula IV:

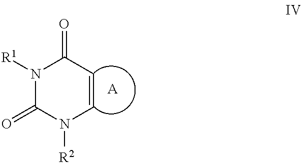

and pharmaceutically acceptable salts and solvates thereof, wherein:

A is selected from the group consisting of:

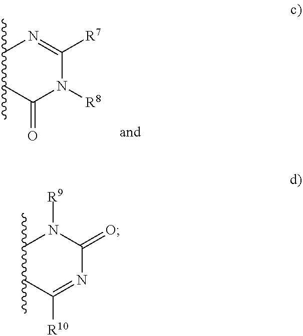

R¹ is H, (C₁-C₆)alkyl, -(alkylene)ₙ-aryl, -(alkylene)ₙ-(C₃-C₁₀)cycloalkyl, -(alkylene)ₙ—(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R² is H, (C₁-C₆)alkyl, -(alkylene)ₙ-aryl, -(alkylene)ₙ-(C₃-C₁₀)cycloalkyl, -(alkylene)ₙ(C₃-C₁₀)cycloalkenyl, -(alkylene)ₙ-heterocycloalkyl, -(alkylene)ₙ-heterocycloalkenyl or -(alkylene) heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)ₙ-aryl or (C₃-C₁₀)cycloalkyl;

R⁷ is H, alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁸ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR^{S}, —N(R⁶)₂, —CN, —C(O)OR⁵ and —C(O)N(R⁶)₂;

R⁹ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene) heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R¹⁰ is H, (C₁-C₆)alkyl, halo(C₁-C₆)alkyl, -(alkylene) aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene) heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂; and

each occurrence of n is independently 0 or 1;

i) Formula V:

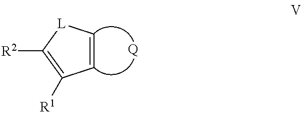

or a pharmaceutically acceptable salt, solvate, ester, or tautomer thereof, wherein:
Q is selected from the group consisting of:

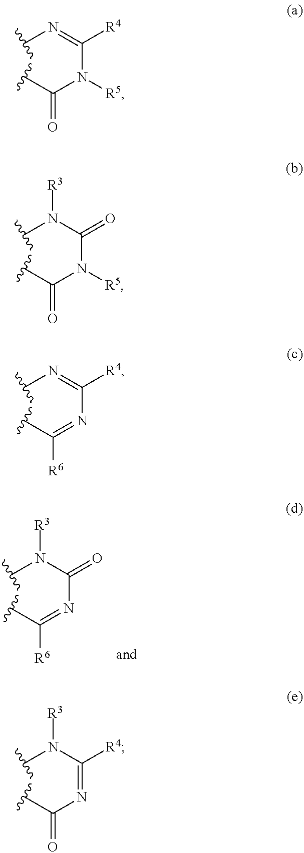

L is selected from the group consisting of:

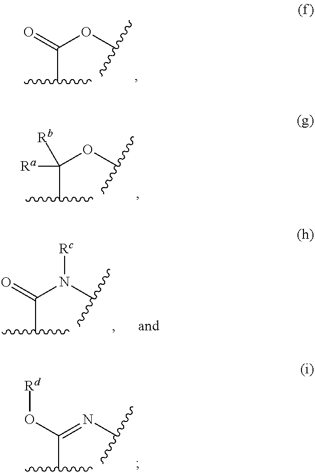

R¹ is selected from the group consisting of H, (C₁-C₆)alkyl, (C₂-C₆)alkenyl, (C₂-C₆)alkynyl, halo(C₁-C₆)alkyl, (C₁-C₆)alkyl substituted with one or more hydroxyl groups, (C₃-C₁₀)cycloalkyl, —C(O)—(C₁-C₆)alkyl, -alkylene-C(O)—O—(C₁-C₆)alkyl, —O—R¹⁰, -alkylene-O—(C₁-C₆)alkyl, aryl, -alkylene-aryl, heteroaryl, -alkylene-heteroaryl, halogen, —(CH₂)ₙ—N(R⁷)₂, -alkylene-(C₃-C₁₀)cycloalkyl, and -alkylene-(C₃-C₁₀)cycloalkenyl,
   wherein said (C₃-C₁₀)cycloalkyl or the (C₃-C₁₀)cycloalkyl portion of said -alkylene-cycloalkyl of R¹ is unsubstituted or substituted with one or more X groups, said aryl or the aryl portion of said -alkylene-aryl of R¹ is unsubstituted or substituted with one or more Y groups, and said heteroaryl or the heteroaryl portion of said -alkylene-heteroaryl of R¹ is unsubstituted or substituted with one or more Y groups;
R² is selected from the group consisting of H, halogen, (C₁-C₆)alkyl, halo(C₁-C₆)alkyl, (C₁-C₆)alkyl substituted with one or more —OH, —C(O)—(C₁-C₆)alkyl, —C(O)—O—(C₁-C₆)alkyl, —C(O)—OH, —O—R¹⁰, -alkylene-O—(C₁-C₆)alkyl, unsubstituted aryl, aryl substituted with one or more Y groups, unsubstituted heteroaryl, heteroaryl substituted with one or more Y groups, and halogen; or
R¹ and R² together with the ring carbon atoms to which they are shown attached, form a 5- or 6-membered cycloalkenyl ring or a 5- or 6-membered heterocyclic ring having 1 or 2 heteroatoms;
R³ is selected from the group consisting of H, (C₁-C₆)alkyl, (C₁-C₆)alkyl substituted with one or more hydroxyl groups, -alkylene-O—(C₁-C₆)alkyl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, —(C₂-C₆)alkenyl, aryl, and heteroaryl,
   wherein said (C₃-C₁₀)cycloalkyl or the (C₃-C₁₀)cycloalkyl portion of said -alkylene-(C₃-C₁₀)cycloalkyl of R³ is unsubstituted or substituted with one or more X groups, said aryl of R³ is unsubstituted or substituted with one or more Y groups, and said heteroaryl of R³ is unsubstituted or substituted with one or more Y groups;
R⁴ is selected from the group consisting of H, halogen, (C₁-C₆)alkyl, —O—R¹⁰, —C(O)—O—(C₁-C₆)alkyl, —S(O)ₘ—R⁹, —N(R⁷)₂, —N(R⁷)—NH—C(O)—(C₁-C₆)alkyl, —N(R⁷)—NH—C(O)—O—(C₁-C₆)alkyl, —O—N═C(R¹²)₂, —N(R⁷)—N═C(R¹²)₂, —C(O)—(C₁-C₆)alkyl, unsubstituted heterocyclyl, heterocyclyl substituted with one or more X groups, —O—N(R⁷)—C(O)—O—(C₁-C₆)alkyl, —C(O)—N(R⁷)₂, —CN, —N₃, and —O—C(O)—(C₁-C₆)alkyl;
R⁵ is selected from the group consisting of H, (C₁-C₆)alkyl, -alkylene-C(O)—R⁸, -alkylene-C(O)—N(R¹¹)₂, -alkylene-C(═N—O—(C₁-C₆)alkyl)-aryl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, -alkylene-C(O)-heterocyclyl, and (C₂-C₆)alkenyl,
   wherein said (C₃-C₁₀)cycloalkyl or the (C₃-C₁₀)cycloalkyl portion of said -alkylene-(C₃-C₁₀)cycloalkyl of R⁵ is unsubstituted or substituted with one or more X groups, and the aryl portion of said -alkylene-C(═N—O—(C₁-C₆)alkyl)-aryl of R⁵ is unsubstituted or substituted with one or more Y groups;
R⁶ is selected from the group consisting of H, (C₁-C₆)alkyl, (C₂-C₆)alkenyl, (C₁-C₆)alkyl substituted with one or more hydroxyl groups, -alkylene-O—(C₁-C₆)alkyl, —O—R¹⁰, halogen, aryl, heteroaryl, and —N(R⁷)₂,
   wherein said aryl of R⁶ is unsubstituted or substituted with one or more Y groups, and said heteroaryl of R⁶ is unsubstituted or substituted with one or more Z groups;
each R⁷ is independently selected from the group consisting of H, (C₁-C₆)alkyl, (C₃-C₁₀)cycloalkyl, aryl, —C(O)—(C₁-C₆)alkyl, and —C(O)-aryl,
   wherein said (C₃-C₁₀)cycloalkyl of R⁷ is unsubstituted or substituted with one or more X groups, and the aryl portion of said —C(O)-aryl or said aryl of R⁷ is unsubstituted or substituted with one or more Y groups; or
two R⁷ groups, together with the N atom to which they are bonded form a heterocyclyl;
R⁸ is selected from the group consisting of aryl, —OH, and heterocyclyl,
   wherein said heterocyclyl of R⁸ is unsubstituted or substituted with one or more X groups, and said aryl of R⁸ is unsubstituted or substituted with one or more Y groups;
R⁹ is selected from the group consisting of (C₁-C₆)alkyl, -alkylene-(C₃-C₁₀)cycloalkyl, (C₂-C₆)alkenyl, —N(R¹¹)₂, and -alkylene-aryl,
   wherein the (C₃-C₁₀)cycloalkyl portion of said -alkylene-(C₃-C₁₀)cycloalkyl of R⁹ is unsubstituted or substituted with one or more X groups, and the aryl portion of said -alkylene-aryl of R⁹ is unsubstituted or substituted with one or more Y groups, and
   with the proviso that when R⁹ is —N(R¹¹)₂, m is 1 or 2;
R¹⁰ is selected from the group consisting of H, (C₁-C₆)alkyl, -alkylene-aryl, -alkenylene-aryl, -alkylene-heteroaryl, (C₂-C₆)alkenyl, —C(O)—(C₁-C₆)alkyl, (C₂-C₆)alkynyl, and -alkylene-(C₃-C₁₀)cycloalkyl,
   wherein the (C₃-C₁₀)cycloalkyl portion of said -alkylene-(C₃-C₁₀)cycloalkyl of R¹⁰ is unsubstituted or substituted with one or more X groups, the aryl portion of said -alkylene-aryl or -alkenylene-aryl of R¹⁰ is unsubstituted or substituted with one or more Y groups, and the heteroaryl portion of said -alkylene-heteroaryl of R¹⁰ is unsubstituted or substituted with one or more Z groups;
each R¹¹ is independently selected from the group consisting of H, (C₁-C₆)alkyl, and aryl,
   wherein said aryl of R¹¹ is unsubstituted or substituted with one or more Y groups; or
two R¹¹ groups, together with the N atom to which they are attached, form a heterocyclyl;
each R¹² is independently selected from the group consisting of (C₁-C₆)alkyl, aryl, and heteroaryl,
   wherein said aryl of R¹² is unsubstituted or substituted with one or more Y groups and said heteroaryl of R¹² is unsubstituted or substituted with one or more Z groups;
R^{a} and R^{b} are each independently selected from the group consisting of H, (C₁-C₆)alkyl, aryl, and heteroaryl,
   wherein said aryl of R^{a} and R^{b} is unsubstituted or substituted with one or more Y groups, and said heteroaryl of R^{a} and R^{b} is unsubstituted or substituted with one or more Z groups;
R^{c} is selected from the group consisting of H, (C₁-C₆)alkyl, alkylene-aryl, and —C(O)—(C₁-C₆)alkyl,
   wherein the aryl portion of said alkylene-aryl of R^{c} is unsubstituted or substituted with one or more Y groups;
R^{d} is selected from the group consisting of H, (C₁-C₆)alkyl, and alkylene-aryl,
   wherein the aryl portion of said alkylene-aryl of R^{d} is unsubstituted or substituted with one or more Y groups;
each X is independently selected from the group consisting of halogen, (C₁-C₆)alkyl, halo(C₁-C₆)alkyl, —O—(C₁-C₆)alkyl, —O-halo(C₁-C₆)alkyl, and —OH;
each Y is independently selected from the group consisting of halogen, (C₁-C₆)alkyl, halo(C₁-C₆)alkyl, —O—(C₁-C₆)alkyl, —O-halo(C₁-C₆)alkyl, —CN, —NO₂, —OH, —S(O₂)—(C₁-C₆)alkyl, —S(O₂)-aryl, —S(O₂)—NH₂, —S(O₂)—NH—(C₁-C₆)alkyl, —S(O₂)—NH-aryl, —S(O₂)—N((C₁-C₆)alkyl)₂, —S(O₂)-N(aryl)₂, —S(O₂)—N((C₁-C₆)alkyl)(aryl), and aryl;
each Z is independently selected from the group consisting of (C₁-C₆)alkyl, halo(C₁-C₆)alkyl, halogen, —O—(C₁-C₆)alkyl, —O-halo(C₁-C₆)alkyl, —CN, —OH, aryl, and N-oxide;
n is 0, 1, 2, or 3;
m is 0, 1, or 2; and

with the proviso that when L is (f), and R², R³ and R⁵ are each H, then R¹ is not —CH₃; and

j) Formula VI:

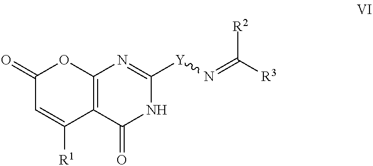

or a pharmaceutically acceptable salt, solvate, ester, or tautomer thereof, wherein:
R¹ is selected from the group consisting of H, R⁴, halo(C₁-C₆)alkyl, -alkylene-R⁴, -alkylene-R⁵, -alkylene-R⁶, (C₂-C₆)alkenyl, (C₂-C₆)alkynyl, and -alkylene-O—(C₁-C₆)alkyl;
R² is selected from the group consisting of (C₁-C₆)alkyl, halo(C₁-C₆)alkyl, -alkylene-R⁵, R⁴, R⁵, R⁶, R⁷ and -alkylene-O—R⁸;
R³ is selected from the group consisting of (C₁-C₆)alkyl, halo(C₁-C₆)alkyl, -alkylene-R⁵, R⁴, R⁵, R⁶, and R⁷; or
R² and R³ together with the carbon atom to which they are both attached form a (C₃-C₁₀)cycloalkyl or heterocycloalkyl ring, wherein said (C₃-C₁₀)cycloalkyl or heterocycloalkyl ring is unsubstituted or independently substituted with one or more X⁵ groups, and wherein said (C₃-C₁₀)cycloalkyl ring can form a spirocyclic compound with a second (C₃-C₁₀)cycloalkyl ring or with a heterocycloalkyl ring, wherein the second (C₃-C₁₀)cycloalkyl ring or the heterocycloalkyl ring is unsubstituted or independently substituted with one or more X⁵ groups;
R⁴ is unsubstituted (C₃-C₁₀)cycloalkyl or (C₃-C₁₀)cycloalkyl substituted with one or more X¹ groups;
R⁵ is unsubstituted aryl and aryl substituted with one or more X² groups;
R⁶ is selected from the group consisting of unsubstituted heteroaryl and heteroaryl substituted with one or more X³ groups;
R⁷ is unsubstituted heterocycloalkyl and heterocycloalkyl substituted with one or more X⁴ groups;
R⁸ is selected from the group consisting of H, (C₁-C₆)alkyl, R⁴, R⁵, R⁶, R⁷, —C(O)—(C₁-C₆)alkyl, —C(O)—R⁵;
each R⁹ is independently selected from the group consisting of H, (C₁-C₆)alkyl, R⁴, R⁵, R⁶, and R⁷;
R¹⁰ is selected from the group consisting of R⁹, —C(O)—(C₁-C₆)alkyl, and —C(O)—R⁵;
Y is —O— or —N(R¹⁰)—;
each X¹ is independently selected from the group consisting of halogen, (C₁-C₆)alkyl, —O—(C₁-C₆)alkyl, —OH, halo(C₁-C₆)alkyl, aryl, and (C₂-C₆)alkyne;
each X² is independently selected from the group consisting of halogen, (C₁-C₆)alkyl, —O—(C₁-C₆)alkyl, —OH, halo(C₁-C₆)alkyl, aryl, and (C₂-C₆)alkyne;
each X³ is independently selected from the group consisting of halogen, (C₁-C₆)alkyl, and N-oxide;
each X⁴ is independently selected form the group consisting of (C₁-C₆)alkyl, R⁵, —C(O)—(C₁-C₆)alkyl, —C(O)—R⁵, —C(O)—O—(C₁-C₆)alkyl, -alkylene-R⁵, R⁴, and —S(O₂)—(C₁-C₆)alkyl; and
each X⁵ is independently selected from the group consisting of (C₁-C₆)alkyl, a fused aryl ring, —C(O)—(C₁-C₆)alkyl, a fused heteroaryl ring, —C(O)—O—(C₁-C₆)alkyl, —C(O)—R⁵, —S(O₂)—(C₁-C₆)alkyl, —C(O)—N(R⁹)₂, R⁵, R⁶, —C(O)—R⁴, —S(O₂)—R⁴, —S(O₂)-alkylene-R⁴, —S(O₂)-alkylene-R⁵, —N(R⁹)—C(O)—O—(C₁-C₆)alkyl, —N(R⁹)—C(O)—O—R⁴, —N(R⁹)—C(O)—N(R⁹)₂ and —N(R⁹)₂;
   wherein said fused aryl ring of X⁵ is unsubstituted or independently substituted with one or more substituent selected from -alkylene-R⁷ or X², and said fused heteroaryl ring of X⁵ is unsubstituted or substituted with one or more X³ groups; and

k) Formula VII, VIII-A, VIII-B, IX or X:

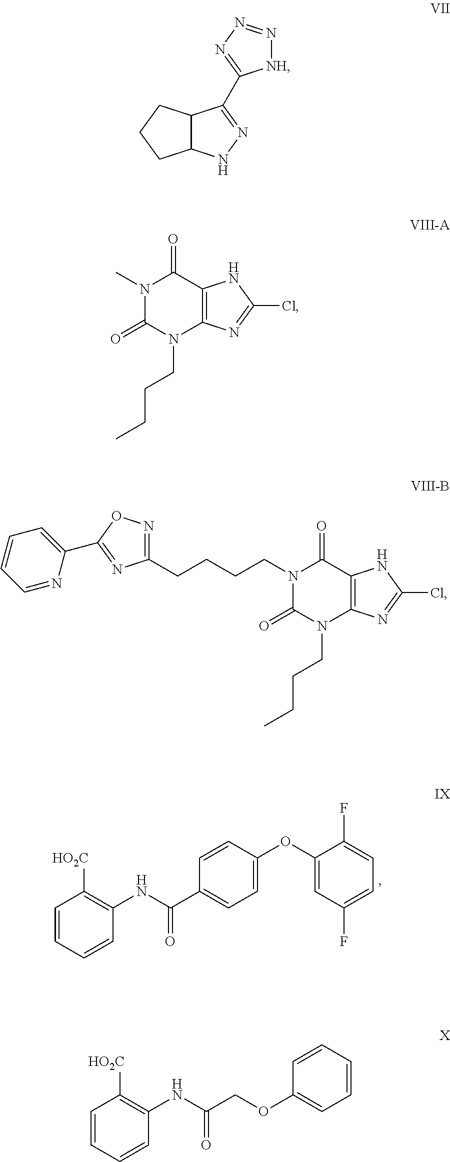

or a pharmaceutically acceptable salt, solvate, ester, or tautomer thereof;

iii) optionally, an effective amount of at least one cardiovascular agent and

iv) optionally a pharmaceutically acceptable carrier.

Cardiovascular agents include, for example, thromboxane A2 biosynthesis inhibitors (e.g. aspirin); thromboxane antagonists (e.g., seratrodast, picotamide and ramatroban); adenosine diphosphate (ADP) inhibitors (e.g., clopidogrel, clopidogrel bisulfate, prasugrel and elinogrel); cyclooxygenase inhibitors (aspirin, meloxicam, rofecoxib and celecoxib); angiotensin antagonists (e.g., valsartan, telmisartan, candesartan, irbesartan, losartan and eprosartan); endothelin antagonists (e.g., tersentan); phosphodiesterase inhibitors (e.g, milrinoone and enoximone); angiotensin converting enzyme (ACE) inhibitors (e.g., captopril, enalapril, enaliprilat, spirapril quinapril, perindopril, ramipril, fesoinopril, trandolapril, lisinopril, moexipril, and benazapril); neutral endopeptidase inhibitors (e.g., canodoxatril and exadotril); anticoagulants (e.g., ximelagatran, fondaparin and enoxaparin); diuretics (e.g., chlorothiazide, hydrochlorothiazide, ethacrynic acid, furosemide and amiloride); platelet aggregation inhibitors (e.g., abciximab and eptifibatide); GP Ilb/Illa antagonists; and GP1b antagonists (e.g., VCL).

This invention also relates to the use of the of following compounds for treating the following disease states by administering, either together or concomitantly, an effective amount of at least one PAR1 antagonist, at least one NAR agonist and, optionally, an effective amount of at least one cardiovascular agent to a mammal in need thereof: diseases associated with thrombosis (e.g., thrombosis, atherosclerosis, restenosis, hypertension, acute coronary syndrome, angina pectoris, arrhythmia, heart failure, cerebral ischemia, stroke, myocardial infarction, glomerulonephritis, thrombotic and thromboembolytic stroke, peripheral cardiovascular disease, deep vein thrombosis, cerebral ischemia, venous thromboembolism, and other cardiovascular diseases); inflammatory disorders (e.g., radiation and chemotherapy induced proliferative and inflammatory disorders of the gastrointestinal tract, lungs, and other organs, inflammatory disorders of the urinary bladder); fibrotic disorders of the liver, kidney, lung and intestinal tract; astrogliosis; cardiovascular disorders associated with hormone replacement therapy; disseminated intracascular coagulation syndrome, neurodegenerative diseases, and cancer.

### DETAILED DESCRIPTION

An embodiment of a compound of Formula I-A are compounds of the formula:

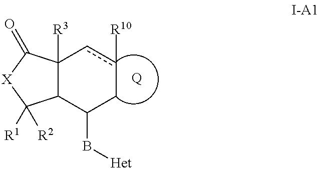

or a pharmaceutically acceptable isomer, salt or solvate thereof, wherein:

[symbol] represents an optional double bond;

Q is

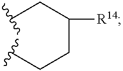

R¹ is methyl;

R² is H;

R³ is H;

Het is pyridyl, wherein a ring nitrogen can form an N-oxide group, wherein Het is attached to B by a carbon atom ring member of said Het, and wherein the Het group is substituted by W;

W is 1 to 4 moieties independently selected from the group consisting of phenyl or pyridyl, unsubstituted or substituted with R²¹;

R¹⁰ is H, provided that when the optional double bond shown in Formula I is present, R¹⁰ is absent;

B is —CH═CH—;

X is —O—;

Y is oxo;

R¹⁴ is NHC(O)OR^{16b}

R^{16b} is (C₁-C₆)alkoxy, (C₁-C₆)alkyl, (C₁-C₆)alkoxy(C₁-C₆)alkyl-, R²²—O—C(O)—(C₁-C₆)alkyl-, (C₃-C₆)cycloalkyl, R²⁸R²⁹N—(CO)—(C₁-C₆)alkyl, R²⁸R²⁹N—(CO)O—(C₁-C₆)alkyl, or hydroxy(C₁-C₆)alkyl);

R²¹ is 1 to 3 substituents independently selected from the group consisting of H, —CN, —CF₃, halogen and CONH₂;

R²² is H or (C₁-C₆)alkyl; and,

R²⁸ and R²⁹ are independently selected from the group consisting of H, (C₁-C₆)alkyl, (C₁-C₆)alkoxy, R²⁷-aryl(C₁-C₆)alkyl, heteroaryl, heteroaryl(C₁-C₆)alkyl hydroxy(C₁-C₆)alkyl, and (C₁-C₆)alkoxy(C₁-C₆)alkyl, heterocyclyl, heterocyclylalkyl, and halo(C₁-C₆)alkyl; or

R²⁸ and R²⁹ taken together form a spirocyclic or a heterospirocyclic ring of 3-6 atoms. These compounds are described in U.S. Pat. No. 7,304,078, herein incorporated by reference.

Another embodiment of the compounds of Formula 1-A1 are those wherein the optional double bond is absent.

Another embodiment of the compounds of Formula 1-A1 are those compounds selected from the group consisting of

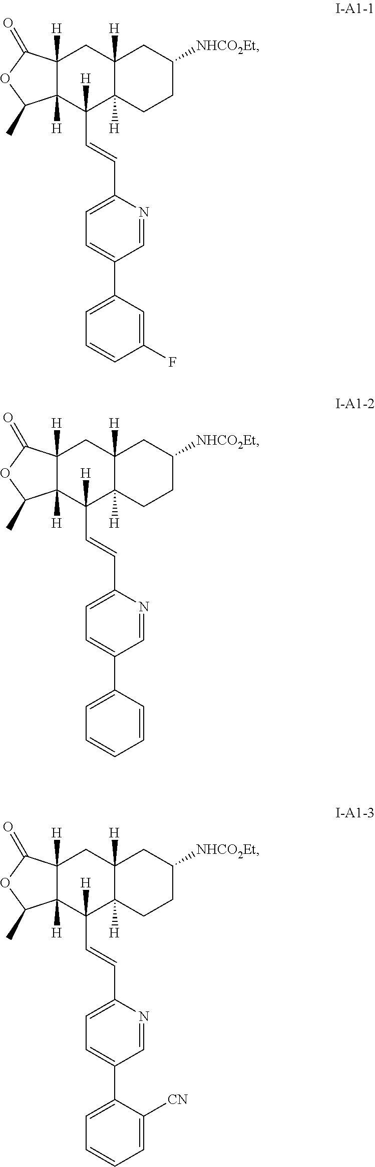

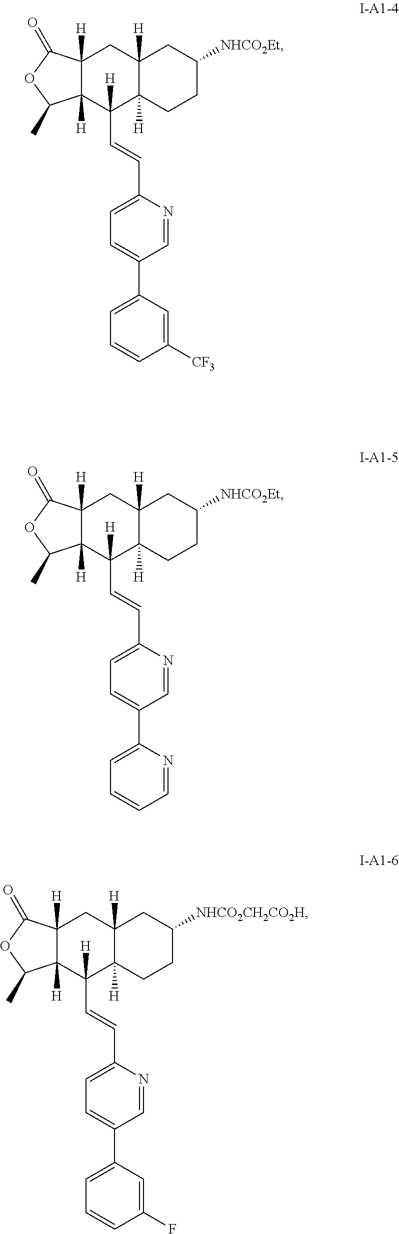

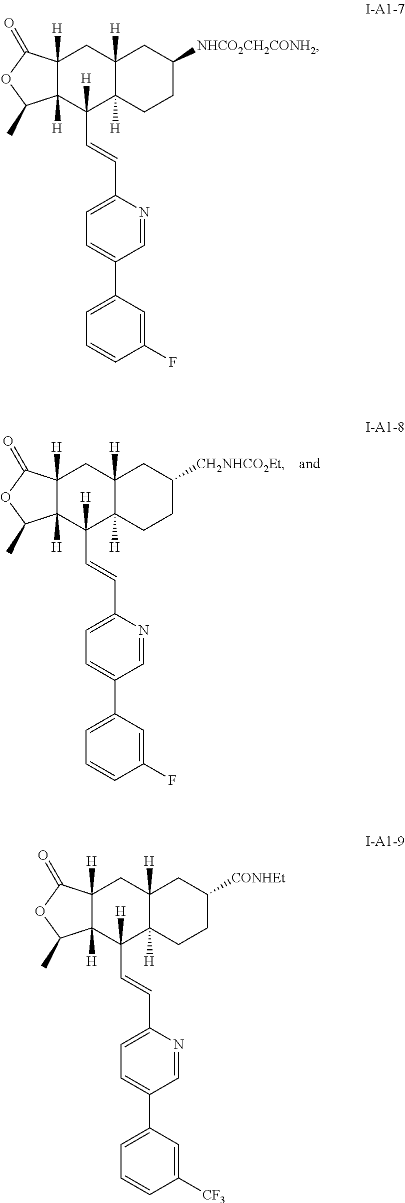

or a pharmaceutically acceptable isomer, salt or solvate thereof.

Another embodiment is the compound of the formula

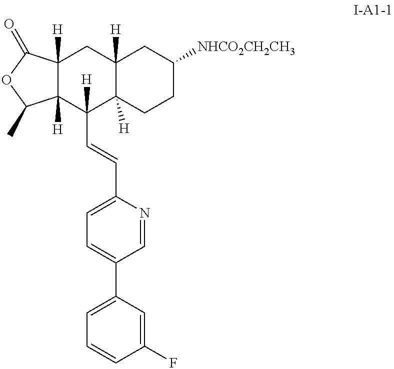

or a pharmaceutically acceptable isomer, salt or solvate thereof. A preferred salt is the bisulfate salt.

An embodiment of a compound of Formula 1-B are compounds of the structural formula

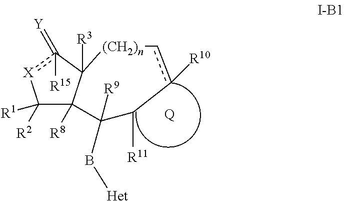

or a pharmaceutically acceptable salt thereof, wherein:

or a pharmaceutically acceptable salt thereof, wherein:

the single dotted line represents an optional double bond;

the double dotted line represents a single bond;

n is 0-2;

Q is

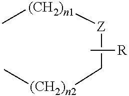

wherein n1 and n2 are independently 0-2; or when the double bond is not present, Q is also fused R-substituted aryl;

R is 1 to 3 substituents independently selected from the group consisting of H, (C₁-C₆)alkyl, halogen, hydroxy, amino, (C₁-C₆)alkyl-amino, (C₁-C₆)di-alkylamino, (C₁-C₆)alkoxy, —COR¹⁶, —COOR¹⁷, —SOR¹⁶, —SOR₂R¹⁶, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl-(C₂-C₆)alkenyl, heteroaryl(C₁-C₆)-alkyl, heteroaryl(C₂-C₆)alkenyl, hydroxy-(C₁-C₆)alkyl, amino(C₁-C₆)alkyl, aryl and thio(C₁-C₆)alkyl;

R¹ and R² are independently selected from the group consisting of H, C₁-C₆ alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)-alkyl, heteroaryl(C₂-C₆)alkenyl, hydroxy-(C₁-C₆)alkyl, amino(C₁-C₆)alkyl, aryl and thio(C₁-C₆)alkyl; or R¹ and R² together form an oxo group;

R³ is H, hydroxy, (C₁-C₆)alkoxy, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl-(C₂-C₆)alkenyl, heteroaryl(C₁-C₆)alkyl, heteroaryl(C₂-C₆)alkenyl, hydroxy-(C₁-C₆)alkyl, amino(C₁-C₆)alkyl, aryl or thio(C₁-C₆)alkyl;

Het is pyridyl, quinolyl or benzoquinolyl, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl; (C₃-C₁₀)cycloalkyl; heterocycloalkyl; heterocycloalkyl substituted by (C₁-C₆)alkyl or (C₂-C₆)alkenyl; (C₂-C₆)alkenyl; R²¹-aryl(C₁-C₆)alkyl; R²¹-aryl-(C₂-C₆)-alkenyl; heteroaryl(C₁-C₆)alkyl; heteroaryl(C₂-C₆)-alkenyl; hydroxy-(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; (C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; (C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, aryl wherein adjacent carbons form a ring with a methylenedioxy group, —C(O)NR⁴R⁵ and heteroaryl; R²¹-aryl; aryl wherein adjacent carbons form a ring with a methylenedioxy group; heteroaryl; heteroaryl substituted by 1 to 4 substituents selected from the group consisting of halogen, (C₁-C₆)alkoxy, (C₁-C₆)alkyl, (C₁-C₆)alkyl-amino, di-((C₁-C₆)alkyl)amino, —OCF₃, —NO₂, hydroxy(C₁-C₆)alkyl, —CHO and phenyl; and heteroaryl wherein adjacent carbon atoms form a ring with a C₃-C₅ alkylene group or a methylenedioxy group;

R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₄—, —(CH₂)₅— or —(CH₂)₂NR⁷—(CH₂)₂— and form a ring with the nitrogen to which they are attached;

R⁶ is independently selected from the group consisting of H, C₁-C₆ alkyl or phenyl;

R⁷ is H or (C₁-C₆)alkyl;

R⁸, R¹⁰ and R¹¹ are independently selected from the group consisting of R¹ and —OR¹, provided that when the optional double bond is present, R¹⁰ is absent, and when ring Q is aromatic, R¹⁰ and R¹¹ are absent;

R⁹ is H, OH, (C₁-C₆)alkoxy, halogen or halo(C₁-C₆)alkyl;

B is —(CH₂)ₙ₃—,

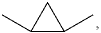

cis or trans —(CH₂)ₙ₄CR¹²═CR^{12a}(CH₂)ₙ₅₋ or —(CH₂)ₙ₄C≡CR^{12a}(CH₂)ₙ₅₋, wherein n₃ is 0-5, n₄ and n₅ are independently 0-2, and R¹² and R^{12a} are independently selected from the group consisting of H, (C₁-C₆)alkyl and halogen;

X is —O—;

Y is oxo, thioxo, (H, H), (H, OH) or (H, (C₁-C₆)alkoxy);

R¹⁵ is absent;

R¹⁶ and R^{16a} are independently selected from the group consisting of (C₁-C₆)lower alkyl, phenyl or benzyl;

R¹⁷, R¹⁸ and R¹⁹ are independently selected from the group consisting of H, (C₁-C₆)alkyl, phenyl, benzyl;

R²⁰ is H, (C₁-C₆)alkyl, phenyl, benzyl, —C(O)R⁶ or —SO₂R⁶;

R²¹ is 1 to 3 substituents independently selected from the group consisting of —CF₃, —OCF₃, halogen, —NO₂, (C₁-C₆)alkyl, (C₁-C₆)alkoxy, (C₁-C₆)alkylamino-, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶ and —NHSO₂CH₂CF₃; and

Z is —CH₂—, —C(O)—, —C(=NOR¹⁷)— or —C(R¹³R¹⁴)—, wherein R¹³ and R¹⁴, together with the carbon to which they are attached, form a spirocycloalkyl group of 3 to 6 carbons, or a spiroheterocycloalkyl group of 3 to 6 members, comprised of 2 to 5 carbon atoms and 1 or 2 heteroatoms selected from the group consisting of O, S and N. These compounds are described in U.S. Pat. Nos. 6,063,847, and 6,326,380; U.S. Publication Nos. 2004/0192753, and 2003/0216437; and International Publication Nos. WO99/26943, and WO03/089428, herein incorporated by reference.

An alternative embodiment of compounds of Formula I-B1 are those where n is zero.

An alternative embodiment of compound of Formula I-B1 are those wherein R² and R⁸ are each hydrogen and R³ is hydrogen or (C₁-C₆)alkyl.

Another embodiment of compounds of Formula I-B1 wherein R⁹ is H, OH or (C₁-C₆)alkoxy.

Another embodiment of compounds of Formula I-B1 wherein X is —O— and Y is oxo.

Another embodiment of compounds of Formula I-B1 wherein R¹ is (C₁-C₆)alkyl.

Another embodiment of compounds of Formula I-B1 wherein Q is R-substituted cyclohexyl or R-substituted phenyl.

Another embodiment of compounds of Formula I-B1 wherein R is H, fluorine, OH, (C₁-C₆)alkyl, (C₁-C₆)alkoxy or —COOR¹⁷.

Another embodiment of compounds of Formula I-B1 wherein B is —CH═CH—.

Another embodiment of compounds of Formula I-B1 wherein Het is pyridyl, W-substituted pyridyl, quinolyl or W-substituted quinolyl.

Another embodiment of compounds of Formula I-B1 wherein W is (C₁-C₆)alkyl, aryl, R²¹-aryl or heteroaryl.

Another embodiment of compounds of Formula I-B1 wherein n is zero; the double dotted line represents a single bond; R² and R⁸ are each hydrogen; R³ is hydrogen or (C₁-C₆)alkyl; R⁹ is H, OH or (C₁-C₆)alkoxy; X is —O—; Y is oxo; R¹ is (C₁-C₆)alkyl; Q is R-substituted cyclohexyl and R¹⁰ and R¹¹ are each hydrogen, or Q is R-substituted phenyl and R¹⁰ and R¹¹ are absent; B is —CH═CH—; and Het is pyridyl, W-substituted pyridyl, quinolyl or W-substituted quinolyl.

Another embodiment of compounds of Formula I-B1 wherein B is trans —CH═CH—.

Another embodiment of compound of Formula 1-B1 are compounds of the following formula:

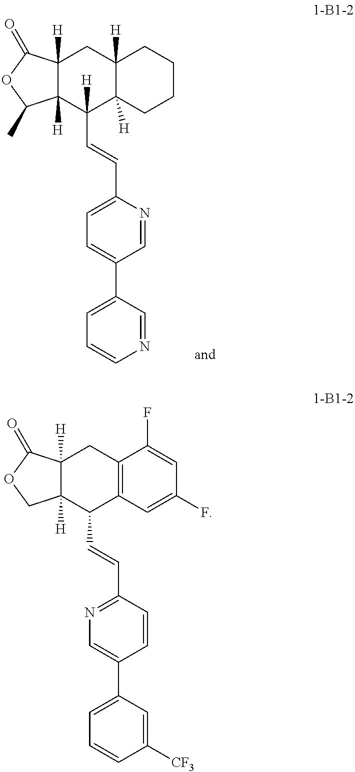

or a pharmaceutically acceptable salt thereof.

An embodiment of a compound of Formula I-C are compounds of the formula

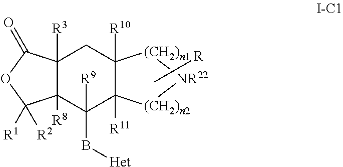

or a pharmaceutically acceptable salt thereof, wherein:

R is 1 to 3 substituents independently selected from the group consisting of H, (C₁-C₆)alkyl, halogen, hydroxy, amino, (C₁-C₆)alkyl-amino, (C₁-C₆)-dialkylamino, (C₁-C₆)alkoxy, —COR¹⁶ , —COOR¹⁷, —SOR¹⁶, —SO₂R¹⁶, —SO₂NR¹⁷R¹⁸, —NR¹⁷SO₂R¹⁸, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, aryl(C₁-C₆)alkyl, hydroxy(C₁-C₆)alkyl, amino-(C₁-C₆)-alkyl, aryl and thio(C₁-C₆)alkyl;

R¹ and R² are independently selected from the group consisting of H, (C₁-C₆)alkyl, fluoro(C₁-C₆)alkyl, difluoro(C₁-C₆)alkyl, trifluorO—(C₁-C₆)alkyl, (C₃-C₁₀)cycloalkyl, (C₂-C₆)alkenyl, aryl(C₁-C₆)alkyl, hydroxy-(C₁-C₆)alkyl, amino(C₁-C₆)alkyl, aryl and thio(C₁-C₆)alkyl; R³ is H, hydroxy, (C₁-C₆)alkoxy, aryloxy, aryl(C₁-C₆)alkyloxy, (C₃-C₁₀))cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², —NR³³CONR³³R³⁴, —NR³³S(O)₂NR³³R³⁴, —(CR³³R³⁴)ₙNR⁴R⁵, —(CR³³R³⁴)ₙNR³³COOR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR³³S(O)₂NR³³R³⁴, (C₁-C₆)alkyl, halogen, (C₃-C₁₀)cycloalkyl, (C₂-C₆alkenyl, —CN, aryl, —P(O)(OR⁷)₂ or (C₁-C₆)alkyl substituted by 1 to 3 substituents independently selected from the group consisting of halogen, —OH, —NH₂, aryl, —COOH, —SO₃H, thio and (C₁-C₆)alkylthio;

n is 1, 2, 3 or 4;

n1 and n2 are independently 0-3, provided that the sum of n1 and n2 is 3;

Het is a pyridine, wherein Het is attached to B by a carbon atom ring member, and wherein the Het group is substituted by 1 to 4 substituents, W, independently selected from the group consisting of
C₁-C₆ alkyl; —NR⁴R⁵; —NHCOR²⁶; —NHSO₂R¹⁶; R²¹-aryl; aryl wherein adjacent carbons form a ring with a methylenedioxy group; and
R²¹-heteroaryl;

R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;

R⁷ is H or (C₁-C₆)alkyl;

R⁸, R¹⁰ and R¹¹ are independently selected from the group consisting of R¹ and —OR¹;

R⁹ is H, OH, —NR⁴R⁵, (C₁-C₆)alkoxy, halogen or halo(C₁-C₆)alkyl;

B is cis or trans —(CH₂)ₙ₄CR¹²═CR^{12a}(CH₂)ₙ₅, wherein n₄ is 0-2, n₅ is 0-2, and R¹² and R^{12a} are independently selected from the group consisting of H, (C₁-C₆)alkyl and halogen;

R¹⁶ and R^{16a} are independently selected from the group consisting of (C₁-C₆)alkyl, phenyl and benzyl;

R¹⁷, R¹⁸ and R¹⁹ are independently selected from the group consisting of H, (C₁-C₆)alkyl, phenyl and benzyl;

R²¹ is 1 to 3 substituents independently selected from the group consisting of H, —CF₃, —OCF₃, halogen, —NO₂, —CN, (C₁-C₆)alkyl, (C₁-C₆)alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O)NR²⁴R²⁵, —SO₂R³⁰, —P(O)(OR²⁹)₂, aryl, aryl(C₁-C₆)alkyl, and —CR²⁹(═NOR²⁸);

R²² is —COR²³, —S(O)R³¹, —S(O)₂R³¹, —SO₂NR²⁴R²⁵ or —COOR²⁷;

R²³ is halo(C₁-C₆)alkyl; (C₂-C₆)alkenyl; halo(C₂-C₆)alkenyl; (C₂-C₆)alkynyl; (C₃-C₁₀)cycloalkyl; (C₃-C₁₀)cycloalkyl(C₁-C₆)alkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₂-C₆)alkyl; heteroaryl; heterocycloalkyl; (C₁-C₆)alkyl substituted by 1-3 substituents independently selected from —COOH and —SO₃H; or

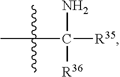

wherein R³⁵ and R³⁶ independently independently selected from the group consisting of H, (C₁-C₆)alkyl, or R³⁷-substituted (C₁-C₆)alkyl, wherein R³⁷ is selected from the group consisting of HO—, HS—, CH₂S—, —NH₂, phenyl, p-hydroxyphenyl and indolyl;

R²⁴ and R²⁵ are independently selected form the group consisting of H, C₁-C₆ alkyl, halo(C₁-C₆)alkyl, (C₂-C₆)alkenyl, halo(C₂-C₆)alkyl, (C₂-C₆)alkynyl, aryl, aryl-(C₁-C₆)alkyl, (C₃-C₁₀)cycloalkyl, halo(C₃-C₁₀)cycloalkyl, (C₁-C₃)alkoxy(C₁-C₃)-alkyl, hydroxy and (C₁-C₆)alkoxy;

R²⁶ is (C₃-C₁₀)-cycloalkyl, aryl, aryl-(C₁-C₆)alkyl, heteroaryl, heteroaryl-(C₁-C₆)alkyl or (C₁-C₆)alkylamino;

R²⁷ is (C₁-C₆)alkyl, phenyl, benzyl, (C₁-C₃)alkoxy(C₁-C₃)-alkyl, (C₃-C₁₀)-cycloalkyl, carboxy(C₁-C₆)alkyl, sulfo(C₁-C₆)alkyl, or (C₁-C₆)alkyl substituted by NR¹⁸R¹⁹ and carboxy;

R²⁸ is H, (C₁-C₆)alkyl, phenyl, benzyl or (C₁-C₃)alkoxy(C₁-C₃)alkyl;

R²⁹ and R³⁰ are independently selected from the group consisting of H and C₁-C₆ alkyl;

R³¹ is (C₁-C₆)alkyl; halo(C₁-C₆)alkyl; (C₂-C₆)alkenyl; halo(C₂-C₆)alkyl; (C₂-C₆)alkynyl; (C₃-C₁₀)cycloalkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₁-C₆)alkyl; heteroaryl; heterocycloalkyl; (C₁-C₆)alkyl substituted by 1-3 substituents independently selected from —COOH and —SO₃H; or (C₁-C₆)alkoxy;

R³² is R³⁵—(C₁-C₆)alkyl, R³⁵—(C₃-C₁₀)cycloalkyl, R³⁵—(C₂-C₆)alkenyl, R³⁵—(C₂-C₆)-alkynyl or R³⁵-aryl, wherein R³⁵ is 1 or 2 substituents independently selected from the group consisting of H, —COOH, —NH₂, —SO₃H, oxo and ═NOR²⁸; and

R³³ and R³⁴ are independently selected from the group consisting of H, (C₁-C₆)alkyl and (C₃-C₁₀)cycloalkyl. These compounds are described in U.S. Pat. No. 7,037,920; U.S. Publication No. 2003/0203927; and International Publication No. WO 03/033501, herein incorporated by reference.

An embodiment of the compounds of Formula I-C1 are those wherein R¹, R¹⁰ and R¹¹ are independently selected from the group consisting of H and (C₁-C₆)alkyl; R² and R⁸ are each hydrogen; and R⁹ is H, OH or (C₁-C₆)alkoxy.

Another embodiment of the compounds of Formula I-C1 are those wherein R is H, halogen, OH, (C₁-C₆)alkyl, (C₁-C₆)alkoxy or amino.

A further embodiment of the compounds of Formula I-C1 are those wherein B is —CH═CH—.

An embodiment of the compounds of Formula I-C1 are those wherein W is —NR⁴R⁵, —NHCOR²⁶, —NHSO₂R¹⁶, R²¹-aryl or heteroaryl.

An embodiment of the compounds of Formula I-C1 are those wherein R²¹ is 1 to 3 substituents independently selected from the group consisting of H, —CF₃, —OCF₃, halogen, —CN, (C₁-C₆)alkyl, (C₁-C₆)alkoxy, —NH₂ and —CR²⁹(═NOR²⁸).

An embodiment of the compounds of Formula I-C1 are those wherein R³ is H, hydroxy, (C₁-C₆)alkoxy, halogen, (C₃-C₁₀)cycloalkyl, —CN, (C₁-C₆)alkyl, —COOR¹⁷ or —NR⁴R⁵.

An embodiment of the compounds of Formula I-C1 are those wherein R²² is —COR²³, —S(O)₂R³¹ or —COOR²⁷.

An embodiment of the compounds of Formula I-C1 are those wherein R²³ is (C₃-C₁₀)-cycloalkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; (C₃-C₁₀)cycloalkyl(C₁-C₆)alkyl; aryl; or aryl(C₁-C₆)alkyl.

An embodiment of the compounds of Formula I-C1 are those wherein R²³ is (C₃-C₁₀)cycloalkyl; (C₃-C₁₀)cycloalkyl(C₁-C₆)alkyl or aryl-(C₂-C₆)alkyl.

An embodiment of the compounds of Formula I-C1 are those wherein R³¹ is (C₁-C₆)alkyl, (C₃-C₁₀)cycloalkyl, aryl or aryl(C₁-C₆)alkyl.

A further embodiment of the compounds of Formula I-C1 are those wherein R²⁷ is (C₁-C₆)alkyl, phenyl, benzyl, (C₁-C₃)alkoxy(C₁-C₃)alkyl or (C₃-C₁₀)cycloalkyl.

An embodiment of the compounds of Formula I-C1 are those compounds selected from the group consisting of compounds of the formula

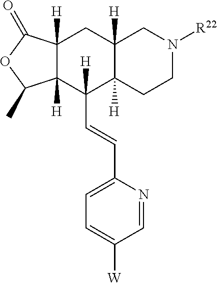

wherein W and R²² are as defined in the table:

| W | R²² |
|---|---|
| 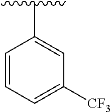 | —CO₂Et |
| 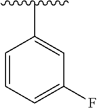 | —CO₂Et |
| 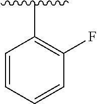 | —CO₂Et |
| 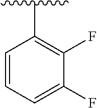 | —CO₂Et |
| 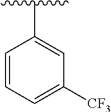 | 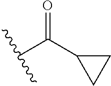 |
| 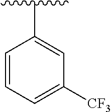 | 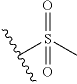 |
| 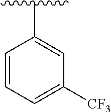 | 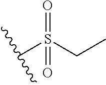 |
| 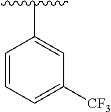 | 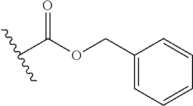 |
| 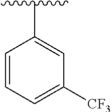 | 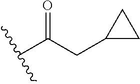 |
| 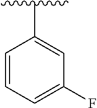 | 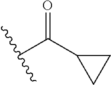 |
| 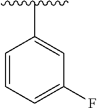 | 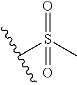 |
| 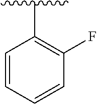 | 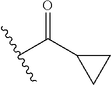 |
| 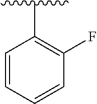 | 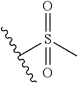 |
| 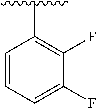 | 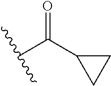 |
| 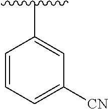 | —CO₂Et |
| 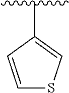 | —CO₂Et |
| 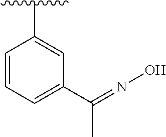 | —CO₂Et |
| 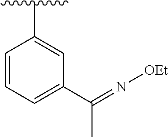 | —CO₂Et |
| 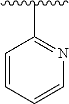 | —CO₂Et |
| 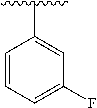 | —CO₂CH₂CH₂OMe |

and compounds of the formula

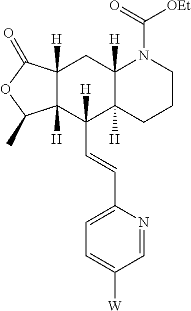

wherein W is as defined in the following table:

W

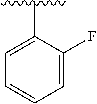

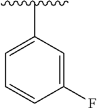

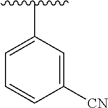

An embodiment of the compounds of Formula I-C1 are those compounds selected from the group consisting of compounds of the formula

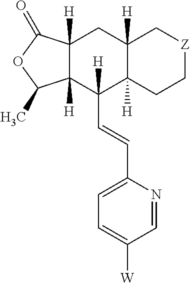

wherein W and Z are as defined in the following table:

| W | Z |
|---|---|
| 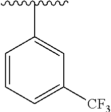 | 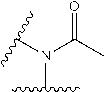 |
| 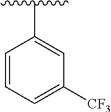 | 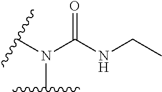 |
| 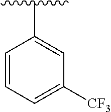 | 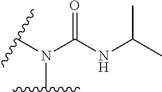 |
| 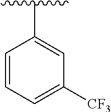 | 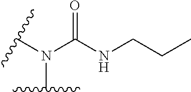 |
| 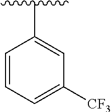 | 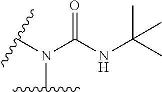 |
| 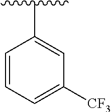 | —NH— |
| 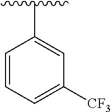 | 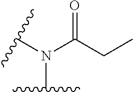 |
| 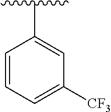 | 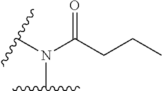 |
| 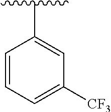 | 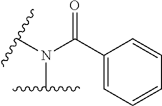 |
| 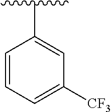 | 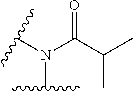 |
| 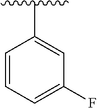 | 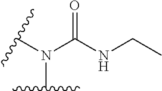 |
| 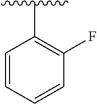 | 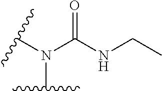 |
| 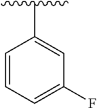 | 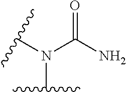 |
| 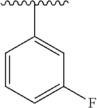 | 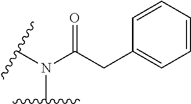 |
| 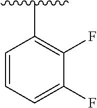 | 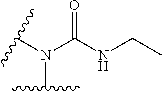 |
| 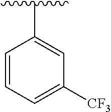 | —N(CH₃)— |

or a pharmaceutically acceptable salt thereof.

In another embodiment the PAR-1 antagonist is a compound of Formula-I D1

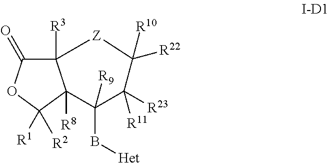

or a pharmaceutically acceptable salt thereof, wherein:

Z is —CH₂—;

R¹ is H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, hydroxy-(C₁-C₆)alkyl, (C₁-C₆)alkoxy(C₁-C₆)alkyl, amino-(C₁-C₆)alkyl, aryl or thio(C₁-C₆)alkyl;

R² is H;

R³ is H, hydroxy, (C₁-C₆)alkoxy, —NR¹⁸R¹⁹, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl;

Het is pyridyl, wherein the ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl; (C₃-C₁₀)cycloalkyl; (C₂-C₆)alkenyl; R²¹-aryl(C₁-C₆)alkyl; R²¹-aryl-(C₂-C₆)-alkenyl; R²¹-aryloxy; R²¹-aryl-NH—; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino-(C₁-C₆)alkyl; thio(C₁-C₆)alkyl; (C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; —C(═NOR¹⁷)R¹⁸; (C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, —C(O)NR⁴R⁵; and R²¹-aryl;

R⁴ and R⁵ are independently selected from the group consisting of H, (C₁-C₆)alkyl, phenyl, benzyl and (C₃-C₁₀)cycloalkyl;

R⁶ is independently selected from the group consisting of H, (C₁-C₆)alkyl, phenyl, (C₃-C₁₀)cycloalkyl, (C₃-C₁₀)cycloalkyl(C₁-C₆)alkyl, (C₁-C₆)alkoxy(C₁-C₆)alkyl, hydroxy(C₁-C₆)alkyl and amino(C₁-C₆)alkyl;

R⁷ is H or (C₁-C₆)alkyl;

R⁸, R¹⁰ and R¹¹ are each H;

R⁹ is H, OH, (C₁-C₆)alkoxy, halogen or halo(C₁-C₆)alkyl;

B is —CH═CH—;

R¹⁶ is (C₁-C₆)lower alkyl, phenyl or benzyl;

R¹⁷, R¹⁸ and R¹⁹ are independently selected from the group consisting of H, (C₁-C₆)alkyl, phenyl, benzyl;

R²¹ is 1 to 3 substituents independently selected from the group consisting of hydrogen, CN, —CF₃, —OCF₃, halogen, —NO₂, (C₁-C₆)alkyl, (C₁-C₆)alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶, —NHSO₂CH₂CF₃, heteroaryl or —C(═NOR¹⁷)R¹⁸;

R²² and R²³ are independently selected from the group consisting of hydrogen, R²⁴—(C₁-C₆)alkyl, R²⁴⁻(C₂-C₆)alkenyl, R²⁴—(C₂-C₆)alkynyl, R²⁵-aryl, R²⁵-aryl(C₁-C₆)alkyl, R²⁹—(C₃-C₁₀)cycloalkyl, R²⁹—(C₃-C₁₀)cyclo-alkenyl, —OH, —OC(O)R³⁰, —C(O)OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₁-C₆)alkoxy, R²⁴—(C₂-C₆)-alkenyloxy, R²⁴—(C₂-C₆)alkynyloxy, R²⁹—(C₃-C₁₀)cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);

or R²² and R¹⁰ together with the carbon to which they are attached, or R²³ and R¹¹ together with the carbon to which they are attached, independently form a R⁴²-substituted carbocyclic ring of 3-10 atoms;

R²⁴ is 1, 2 or 3 substituents independently selected from the group consisting of hydrogen, halogen, —OH, (C₁-C₆)alkoxy, R³⁵-aryl, (C₁-C₆)-alkyl-C(O)—, (C₂-C₆)-alkenyl-C(O)—, (C₂-C₆)alkynyl-C(O)—, R²⁶—(C₃-C₁₀)cycloalkyl, R₂₆—(C₃-C₁₀)cycloalkenyl, —OC(O)R³⁰, —C(O)OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OC(O)NR³⁰R³¹, R²⁹—(C₃-C₁₀)-cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);

R²⁵ is 1, 2 or 3 substituents independently selected from the group consisting of hydrogen, halogen, —COOR³⁶, —CN, —C(O)NR³⁷R³⁸, —NR³⁹C(O)R⁴⁰, —OR³⁶, (C₃-C₁₀)cycloalkyl, (C₃-C₁₀)cycloalkyl-C₁-C₆)alkyl, (C₁-C₆)alkyl(C₃-C₁₀)cycloalkyl-(C₁-C₆)alkyl, halo(C₁-C₆)alkyl(C₃-C₁₀)cycloalkyl(C₁-C₆)alkyl, hydroxy(C₁-C₆)alkyl, and (C₁-C₆)alkoxy(C₁-C₆)alkyl;

R²⁶ is 1, 2, or 3 substituents independently selected from the group consisting of hydrogen, halogen and (C₁-C₆)alkoxy;

R²⁹ is 1, 2 or 3 substituents independently selected from the group consisting of hydrogen, (C₁-C₆)alkyl, —OH, (C₁-C₆)alkoxy and halogen;

R³⁰, R³¹ and R³² are independently selected from the group consisting of hydrogen, (C₁-C₆)-alkyl, (C₁-C₆)alkoxy(C₁-C₆)-alkyl, R²⁵-aryl(C₁-C₆)-alkyl, R³³—(C₃-C₁₀)cycloalkyl, R³⁴₋(C₃-C₁₀)cycloalkyl(C₁-C₆)alkyl, and R²⁵-aryl;

R³³ is hydrogen, (C₁-C₆)alkyl, OH—(C₁-C₆)alkyl or (C₁-C₆)alkoxy;

R³⁵ is 1 to 4 substituents independently selected from the group consisting of hydrogen, (C₁-C₆)alkyl, —OH, halogen, —CN, (C₁-C₆)alkoxy, trihalo(C₁-C₆)alkoxy, (C₁-C₆)alkylamino, di((C₁-C₆)alkyl)amino, —OCF₃, OH—(C₁-C₆)alkyl, —CHO, —C(O)(C₁-C₆)-alkylamino, —C(O)di((C₁-C₆)alkyl)amino, —NH₂, —NHC(O)(C₁-C₆)alkyl and —N((C₁-C₆)alkyl)C(O)(C₁-C₆)alkyl;

R³⁶ is hydrogen, (C₁-C₆)alkyl, halo(C₁-C₆)alkyl, dihalo(C₁-C₆)alkyl or trifluoro(C₁-C₆)alkyl,

R³⁷ and R³⁸ are independently selected from the group consisting of hydrogen, (C₁-C₆)alkyl, aryl(C₁-C₆)alkyl, phenyl and (C₃-C₁₅)cycloalkyl;

R³⁹ and R⁴⁰ are independently selected from the group consisting of hydrogen, (C₁-C₆)alkyl, aryl(C₁-C₆)alkyl, phenyl and (C₃-C₁₅)-cycloalkyl; and

R⁴² is 1 to 3 substituents independently selected from the group consisting of hydrogen, —OH, (C₁-C₆)alkyl and (C₁-C₆)alkoxy. These compounds are described in U.S. Pat. No. 6,645,987, herein incorporated by reference

In another embodiment is a compound of Formula I-D1 wherein R⁸, R¹⁰ and R¹¹ are each hydrogen, and R³ is hydrogen, OH, (C₁-C₆)alkoxy, —NHR¹⁸ or (C₁-C₆)alkyl.

In another embodiment is a compound of Formula I-D1 wherein R⁹ is H, OH or (C₁-C₆)alkoxy.

In another embodiment is a compound of Formula I-D1 wherein R¹ is (C₁-C₆)alkyl.

In another embodiment is a compound of Formula I-D1 wherein W is (C₁-C₆)alkyl, or R²¹-aryl.

In another embodiment is a compound of Formula I-D1 wherein R²² and R²³ are independently selected from the group consisting of OH, (C₁-C₆)alkyl, (C₂-C₆)-alkenyl, (C₂-C₆)alkynyl, trifluoro(C₁-C₆)-alkyl, trifluoro(C₂-C₆)-alkenyl, trifluoro(C₂-C₆)alkynyl, (C₃-C₁₀)cycloalkyl, R²⁵-aryl, R²⁵-aryl(C₁-C₆)alkyl, R²⁵-arylhydroxy(C₁-C₆)alkyl, R²⁵-aryl(C₁-C₆)alkoxy(C₁-C₆)alkyl, (C₃-C₁₀)-cycloalkyl-(C₁-C₆)alkyl, (C₁-C₆)alkoxy, (C₃-C₁₀)cycloalkyloxy, (C₁-C₆)alkoxy(C₁-C₆)alkyl, OH—(C₁-C₆)alkyl, and trifluoro(C₁-C₆)alkoxy.

In another embodiment is a compound of Formula I-D wherein R²² and R²³ are independently selected from the group consisting of (C₁-C₆)alkyl and OH—(C₁-C₆)alkyl.

In another embodiment is a compound of Formula I-D1 wherein R² is hydrogen; R³ is hydrogen OH, (C₁-C₆)alkoxy, —NHR¹⁸ or (C₁-C₆)alkyl; R⁹ is H, OH or (C₁-C₆)alkoxy; and R¹ is (C₁-C₆)alkyl.

In another embodiment is a compound of Formula I-D1 wherein R²² and R²³ are independently selected from the group consisting of OH, (C₁-C₆)alkyl, (C₂-C₆)-alkenyl, (C₂-C₆)alkynyl, trifluoro(C₁-C₆)-alkyl, trifluoro(C₂-C₆)-alkenyl, trifluoro(C₂-C₆)alkynyl, (C₃-C₁₀)cycloalkyl, R²⁵-aryl, R²⁵-aryl(C₁-C₆)alkyl, R²⁵-arylhydroxy(C₁-C₆)alkyl, R²⁵-aryl(C₁-C₆)alkoxy(C₁-C₆)alkyl, (C₃-C₁₀)-cycloalkyl-(C₁-C₆)alkyl, (C₁-C₆)alkoxy, (C₃-C₁₀)cycloalkyloxy, (C₁-C₆)alkoxy(C₁-C₆)alkyl, OH—(C₁-C₆)alkyl, and trifluoro(C₁-C₆)alkoxy.

In another embodiment is a compound of Formula I-D wherein R²² and R²³ are independently selected from the group consisting of (C₁-C₆)alkyl and OH—(C₁-C₆)alkyl.

In another embodiment is a compound of Formula I-D wherein B is trans —CH═CH—.

In another embodiment is a compound of Formula I-D selected from the group consisting of compounds:

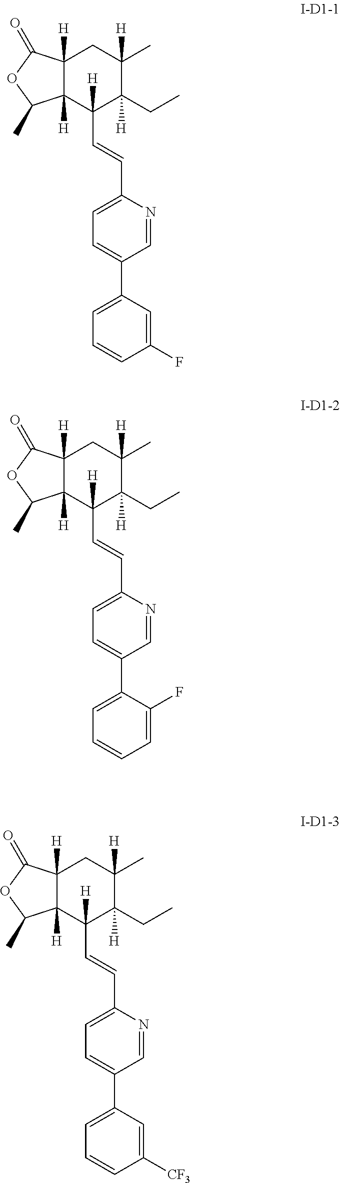

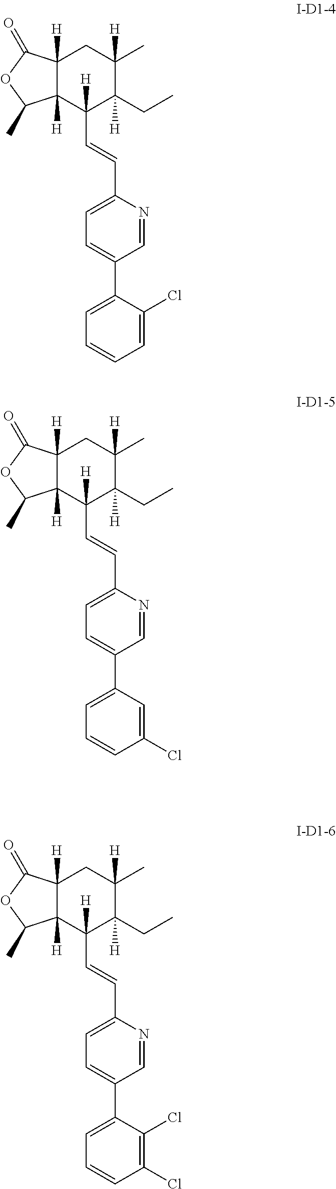

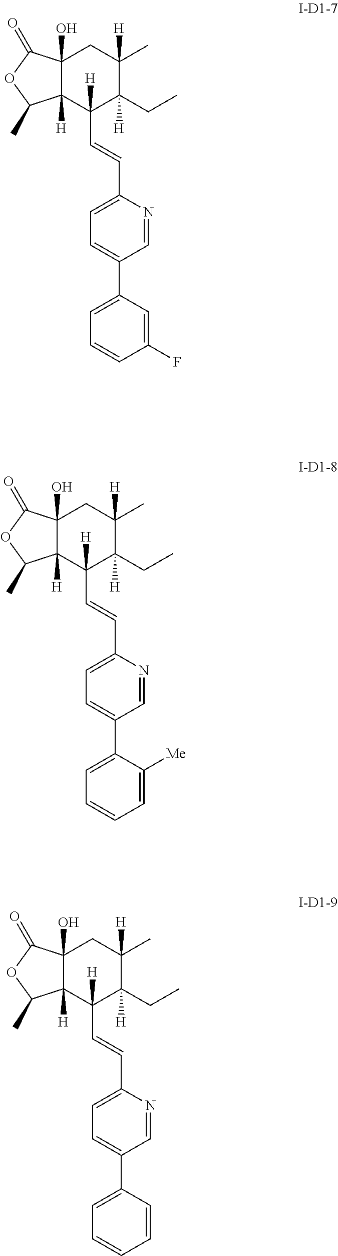

Another embodiment of the compounds of Formula 1-D includes the following:

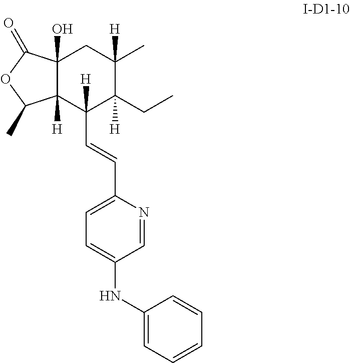

Other embodiments of compounds of formula 1-D include compounds of the formula

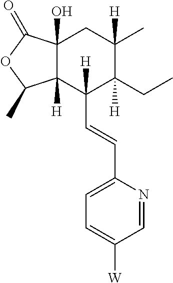

wherein W is as defined in the following table:

| | Analytical |
|---|---|
| W | Data |
| 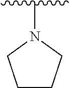 | HRMS (MH⁺) 385.2490 |
| 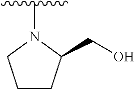 | HRMS (MH⁺) 415.2601 |
| 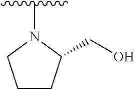 | HRMS (MH⁺) 414.2593 |
| 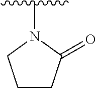 | HRMS (MH⁺) 399.2278 |

Another embodiment of the compounds of formula I-D1 is a compound of the formula

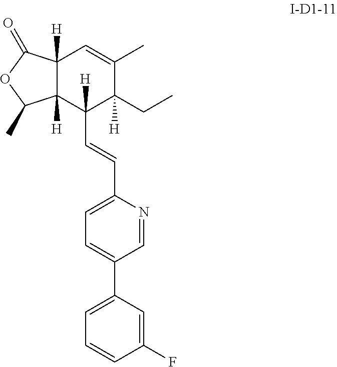

Further embodiments releaded to Formula I-D1-11 are as follows:

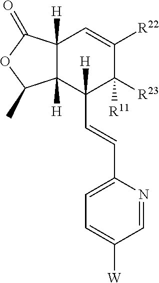

wherein R¹¹, R²², R²³ and W are as defined in the table (Me is methyl, Et is ethyl, Bn is benzyl):

| | | | | HRMS |
|---|---|---|---|---|
| R²² | R²³ | R¹¹ | W | (MH⁺) |
| H | H | H | 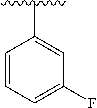 | 350.1565 |
| Me | —CH₂OBn | H | 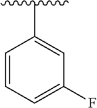 | 484.2299 |
| Me | H | —CH₂OBn | 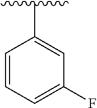 | 484.2294 |
| Me | H | Et | 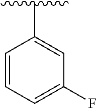 | 392.2021 |
| Me | Me | H | 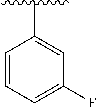 | 378.1870 |
| Me | H | Me | 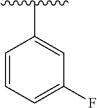 | 378.1870 |
| Me | H | H | 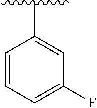 | 364.1714 |
| Me | —CH₂OH | H | 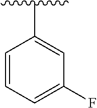 | 394.1821 |

Another embodiment of the compounds of Formula 1-D1 include is a compound of the formula

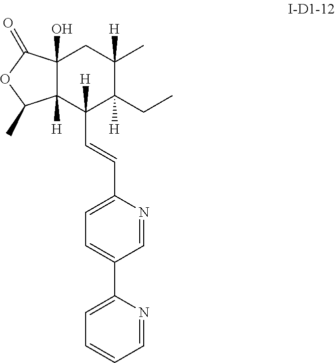

Additional embodiments related to the compound of Formula I-D1 include:

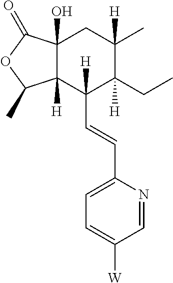

wherein W is as defined in the table:

| | HRMS |
|---|---|
| W | (MH⁺) |
| 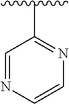 | 394.2127 |
| 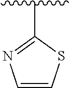 | 399.1750 |

Additional embodiments of Formula I-D include:

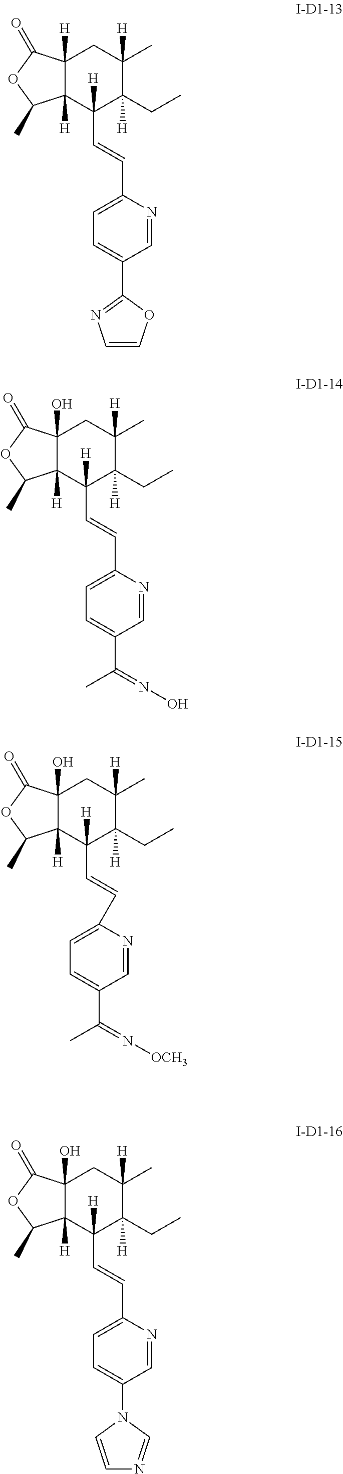

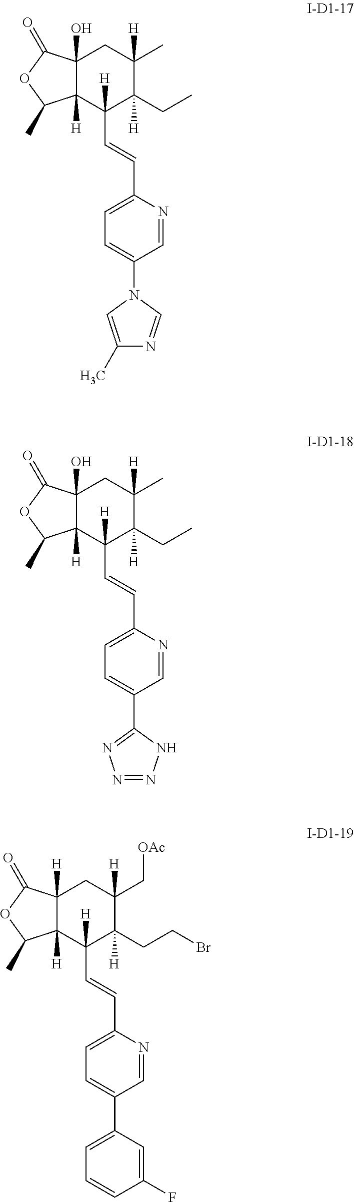

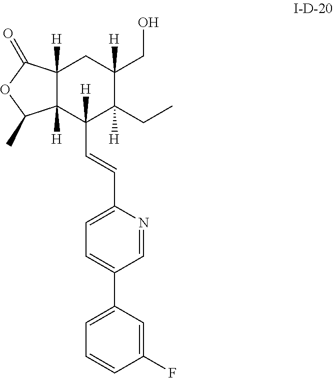

Another embodiment of the compound of Formula 1-D1 are as follows:

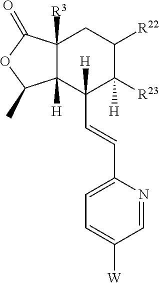

wherein R³, R²², R²³ and W are as defined in the table (Me is methyl, Et is ethyl):

| | | | | HRMS |
|---|---|---|---|---|
| R³ | R²² | R²³ | W | (MH⁺) |
| H | —CH₂OH | Et | 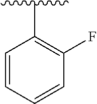 | 410.2138 |
| H | —CH═N—OH | Et | 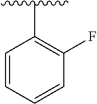 | 423.2090 |
| H | —CH═N—OMe | Et | 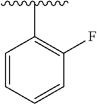 | 437.2235 |
| H | —CH═N—OEt | Et | 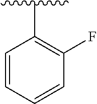 | 451.2396 |
| OH | —CH₂OH | Et | 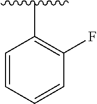 | 426.2075 |

In another embodiment the PAR-1 antagonist is a compound of the Formula I-E-1 or I-E-2

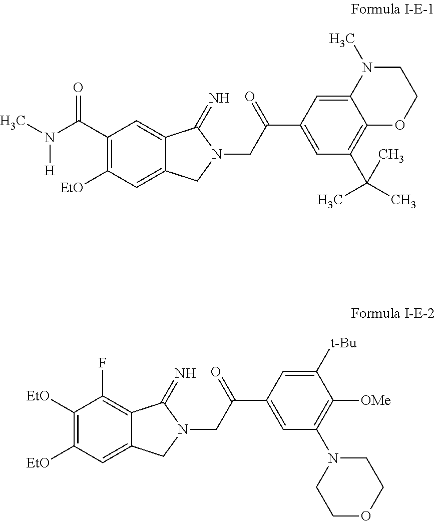

or a pharmaceutically acceptable salt thereof.

In another embodiment the PAR1 antagonist is a compound of the Formula I-E-3

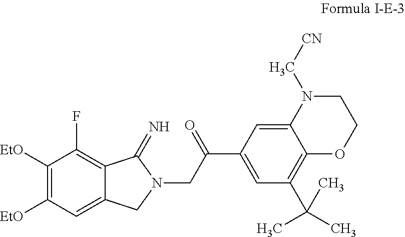

or a pharmaceutically acceptable salt thereof.

A preferred salt for the compound of Formula I-1E-1 is the hydrobromide salt.

The compounds of Formulae I-E-1 to I-E-3 are made by Eisai.

In another embodiment the NAR agonist is a compound of Formula II:

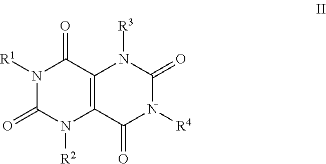

or a pharmaceutically acceptable salt, solvate, ester or prodrug thereof, wherein:

R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene) heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, heteroaryl, —OR⁵, —SR⁵, —N(R⁶)₂, —CN, —C(O)OR⁵ and —C(O)N(R⁶)₂;

R² is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵, —NHC(O)—R⁶ and —C(O)N(R⁶)₂;

R³ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁴ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)ₙ-aryl or (C₃-C₁₀)cycloalkyl; and

each occurrence of n is independently 0 or 1. These compounds are described in International Publication No. WO2008/127591, herein incorporated by reference.

In another embodiment, the NAR agonist is selected from the group of compounds consisting of Formula III:

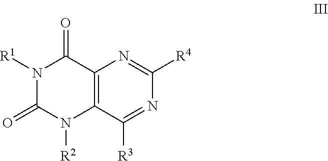

and pharmaceutically acceptable salts, solvates, esters and prodrugs thereof, wherein:

R¹ is H, (C₁-C₆)alkyl, -(alkylene)n-aryl, -(alkylene)n-(C₃-C₁₀)cycloalkyl, -(alkylene)ₙ-(C₃-C₁₀)cycloalkenyl, -(alkylene)n-heterocycloalkyl, -(alkylene)n-heterocycloalkenyl or -(alkylene)n-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R² is H, (C₁-C₆)alkyl, -(alkylene)n-aryl, -(alkylene)n-(C₃-C₁₀)cycloalkyl, -(alkylene)n-(C₃-C₁₀)cycloalkenyl, -(alkylene)n-heterocycloalkyl, -(alkylene)n-heterocycloalkenyl or -(alkylene)n-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R³ is H, (C₁-C₆)alkyl, halo(C₁-C₆)alkyl, -(alkylene)n-aryl, -(alkylene)n-(C₃-C₁₀)cycloalkyl, -(alkylene)n-(C₃-C₁₀)cycloalkenyl, -(alkylene)n-heterocycloalkyl, -(alkylene)n-heterocycloalkenyl, -(alkylene)n-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁴ is H, (C₁-C₆)alkyl, halo(C₁-C₆)alkyl, -(alkylene)n-aryl, -(alkylene)n-(C₃-C₁₀)cycloalkyl, -(alkylene)n-(C₃-C₁₀)cycloalkenyl, -(alkylene)ₙ-heterocycloalkyl, -(alkylene)n-heterocycloalkenyl, -(alkylene)n-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)n-aryl or (C₃-C₁₀)cycloalkyl; and

each occurrence of n is independently 0 or 1. These compounds are described in International Publication No. WO2008/127591, herein incorporated by reference.

In another embodiment, the NAR agonist is selected from the group of compounds consisting of Formula IV:

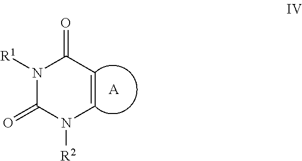

and pharmaceutically acceptable salts and solvates thereof, wherein:

A is selected from the group consisting of:

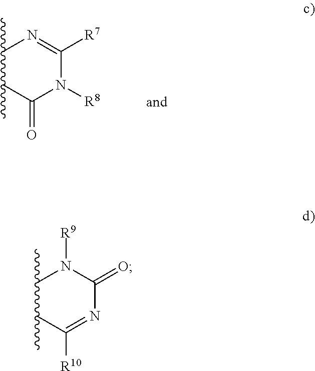

R¹ is H, (C₁-C₆)alkyl, -(alkylene)n-aryl, -(alkylene)n-(C₃-C₁₀)cycloalkyl, -(alkylene)n-(C₃-C₁₀)cycloalkenyl, -(alkylene)n-heterocycloalkyl, -(alkylene)ₙ-heterocycloalkenyl or -(alkylene)n-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R² is H, (C₁-C₆)alkyl, -(alkylene)ₙ-aryl, -(alkylene)n-(C₃-C₁₀)cycloalkyl, -(alkylene)ₙ—(C₃-C₁₀)cycloalkenyl, -(alkylene)ₙ-heterocycloalkyl, -(alkylene)ₙ-heterocycloalkenyl or -(alkylene)n-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

each occurrence of R⁵ is independently H, (C₁-C₆)alkyl, aryl or (C₃-C₁₀)cycloalkyl;

each occurrence of R⁶ is independently H, (C₁-C₆)alkyl, -(alkylene)ₙ-aryl or (C₃-C₁₀)cycloalkyl;

R⁷ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁸ is H, (C₁-C₆)alkyl, halo(C₁-C₆)alkyl, -(alkylene) aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R⁹ is H, (C₁-C₆)alkyl, halo(C₁-C₆)alkyl, -(alkylene) aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;

R¹⁰ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂; and

each occurrence of n is independently 0 or 1. These compounds are described in International Publication No. WO2008/127591, herein incorporated by reference.

In another embodiment the NAR agonist is a compound of Formula V:

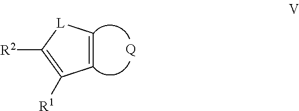

or a pharmaceutically acceptable salt, solvate, ester, or tautomer thereof, wherein:

Q is selected from the group consisting of:

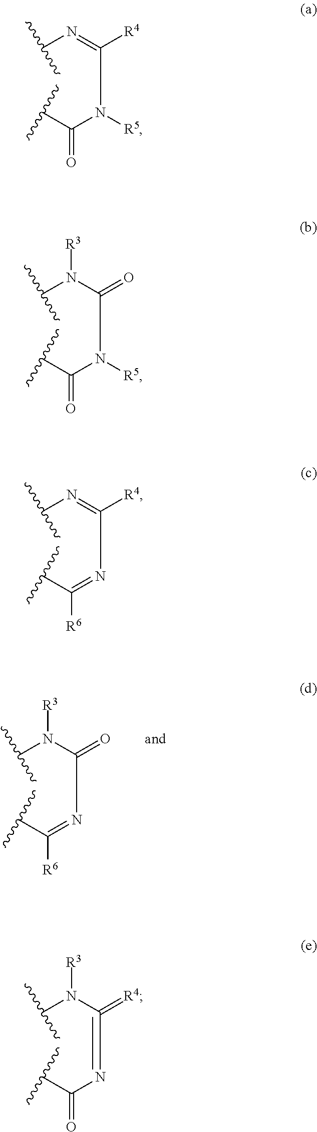

L is selected from the group consisting of:

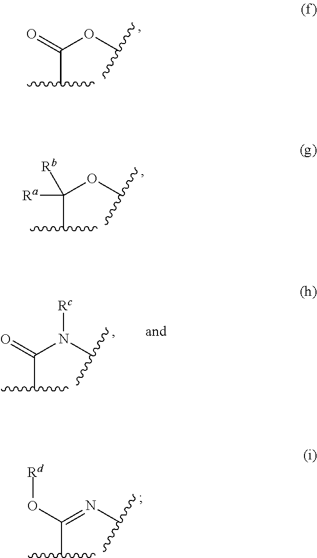

R¹ is selected from the group consisting of H, (C₁-C₆)alkyl, (C₂-C₆)alkenyl, (C₂-C₆)alkynyl, halo(C₁-C₆)alkyl, (C₁-C₆)alkyl substituted with one or more hydroxyl groups, (C₃-C₁₀)cycloalkyl, —C(O)—(C₁-C₆)alkyl, -alkylene-C(O)—O-alkyl, —O—R¹⁰, -alkylene-O—(C₁-C₆)alkyl, aryl, -alkylene-aryl, heteroaryl, -alkylene-heteroaryl, halogen, —(CH₂)ₙ—N(R⁷)₂, -alkylene-(C₃-C₁₀)cycloalkyl, and -alkylene-(C₃-C₁₀)cycloalkenyl,
   wherein said (C₃-C₁₀)cycloalkyl or the (C₃-C₁₀)cycloalkyl portion of said -alkylene-(C₃-C₁₀)cycloalkyl of R¹ is unsubstituted or substituted with one or more X groups, said aryl or the aryl portion of said -alkylene-aryl of R¹ is unsubstituted or substituted with one or more Y groups, and said heteroaryl or the heteroaryl portion of said -alkylene-heteroaryl of R¹ is unsubstituted or substituted with one or more Y groups;
R² is selected from the group consisting of H, halogen, (C₁-C₆)alkyl, halo(C₁-C₆)alkyl, (C₁-C₆)alkyl substituted with one or more —OH, —C(O)—(C₁-C₆)alkyl, —C(O)—O—(C₁-C₆)alkyl, —C(O)—OH, —O—R¹⁰, -alkylene-O—(C₁-C₆)alkyl, unsubstituted aryl, aryl substituted with one or more Y groups, unsubstituted heteroaryl, heteroaryl substituted with one or more Y groups, and halogen; or
R¹ and R² together with the ring carbon atoms to which they are shown attached, form a 5- or 6-membered cycloalkenyl ring or a 5- or 6-membered heterocyclic ring having 1 or 2 heteroatoms;
R³ is selected from the group consisting of H, (C₁-C₆)alkyl, (C₁-C₆)alkyl substituted with one or more hydroxyl groups, -alkylene-O—(C₁-C₆)alkyl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, (C₂-C₆)alkenyl, aryl, and heteroaryl,
   wherein said (C₃-C₁₀)cycloalkyl or the (C₃-C₁₀)cycloalkyl portion of said -alkylene-cycloalkyl of R³ is unsubstituted or substituted with one or more X groups, said aryl of R³ is unsubstituted or substituted with one or more Y groups, and said heteroaryl of R³ is unsubstituted or substituted with one or more Y groups;
R⁴ is selected from the group consisting of H, halogen, (C₁-C₆)alkyl, —O—R¹⁰, —C(O)—O—(C₁-C₆)alkyl, —S(O)ₘ—R⁹, —N(R⁷)₂, —N(R⁷)—NH—C(O)—(C₁-C₆)alkyl, —N(R⁷)—NH—C(O)—O—(C₁-C₆)alkyl, —O—N═C(R¹²)₂, —N(R⁷)—N═C(R¹²)₂, —C(O)—(C₁-C₆)alkyl, unsubstituted heterocyclyl, heterocyclyl substituted with one or more X groups, —O—N(R⁷)—C(O)—O—(C₁-C₆)alkyl, —C(O)—N(R⁷)₂, —CN, —N₃, and —O—C(O)—(C₁-C₆)alkyl;
R⁵ is selected from the group consisting of H, (C₁-C₆)alkyl, -alkylene-C(O)—R⁸, -alkylene-C(O)—N(R¹¹)₂, -alkylene-C(═N—O—(C₁-C₆)alkyl)-aryl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, -alkylene-C(O)-heterocyclyl, and (C₂-C₆)alkenyl,
   wherein said (C₃-C₁₀)cycloalkyl or the (C₃-C₁₀)cycloalkyl portion of said -alkylene-(C₃-C₁₀)cycloalkyl of R⁵ is unsubstituted or substituted with one or more X groups, and the aryl portion of said -alkylene-C(═N—O—(C₁-C₆)alkyl)-aryl of R⁵ is unsubstituted or substituted with one or more Y groups;
R⁶ is selected from the group consisting of H, (C₁-C₆)alkyl, (C₂-C₆)alkenyl, (C₁-C₆)alkyl substituted with one or more hydroxyl groups, -alkylene-O—(C₁-C₆)alkyl, —O—R¹⁰, halogen, aryl, heteroaryl, and —N(R⁷)₂,
   wherein said aryl of R⁶ is unsubstituted or substituted with one or more Y groups, and said heteroaryl of R⁶ is unsubstituted or substituted with one or more Z groups;
each R⁷ is independently selected from the group consisting of H, (C₁-C₆)alkyl, (C₃-C₁₀)cycloalkyl, aryl, —C(O)—(C₁-C₆)alkyl, and —C(O)-aryl,
   wherein said (C₃-C₁₀)cycloalkyl of R⁷ is unsubstituted or substituted with one or more X groups, and the aryl portion of said —C(O)-aryl or said aryl of R⁷ is unsubstituted or substituted with one or more Y groups; or
two R⁷ groups, together with the N atom to which they are bonded form a heterocyclyl; R⁸ is selected from the group consisting of aryl, —OH, and heterocyclyl, wherein said heterocyclyl of R⁸ is unsubstituted or substituted with one or more X groups, and said aryl of R⁸ is unsubstituted or substituted with one or more Y groups;
R⁹ is selected from the group consisting of (C₁-C₆)alkyl, -alkylene-(C₃-C₁₀)cycloalkyl, (C₂-C₆)alkenyl, —N(R¹¹)₂, and -alkylene-aryl,
   wherein the (C₃-C₁₀)cycloalkyl portion of said -alkylene-(C₃-C₁₀)cycloalkyl of R⁹ is unsubstituted or substituted with one or more X groups, and the aryl portion of said -alkylene-aryl of R⁹ is unsubstituted or substituted with one or more Y groups, and
   with the proviso that when R⁹ is —N(R¹¹)₂, m is 1 or 2;
R¹⁰ is selected from the group consisting of H, (C₁-C₆)alkyl, -alkylene-aryl, -alkenylene-aryl, -alkylene-heteroaryl, (C₂-C₆)alkenyl, —C(O)—(C₁-C₆)alkyl, (C₂-C₆)alkynyl, and -alkylene-(C₃-C₁₀)cycloalkyl,
   wherein the (C₃-C₁₀)cycloalkyl portion of said -alkylene-(C₃-C₁₀)cycloalkyl of R¹⁰ is unsubstituted or substituted with one or more X groups, the aryl portion of said -alkylene-aryl or -alkenylene-aryl of R¹⁰ is unsubstituted or substituted with one or more Y groups, and the heteroaryl portion of said -alkylene-heteroaryl of R¹⁰ is unsubstituted or substituted with one or more Z groups;
each R¹¹ is independently selected from the group consisting of H, (C₁-C₆)alkyl, and aryl,
   wherein said aryl of R¹¹ is unsubstituted or substituted with one or more Y groups; or
two R¹¹ groups, together with the N atom to which they are attached, form a heterocyclyl;
each R¹² is independently selected from the group consisting of (C₁-C₆)alkyl, aryl, and heteroaryl,
   wherein said aryl of R¹² is unsubstituted or substituted with one or more Y groups and said heteroaryl of R¹² is unsubstituted or substituted with one or more Z groups;
R^{a} and R^{b} are each independently selected from the group consisting of H, (C₁-C₆)alkyl, aryl, and heteroaryl,
   wherein said aryl of R^{a} and R^{b} is unsubstituted or substituted with one or more Y groups, and said heteroaryl of R^{a} and R^{b} is unsubstituted or substituted with one or more Z groups;
R^{c} is selected from the group consisting of H, (C₁-C₆)alkyl, alkylene-aryl, and —C(O)—(C₁-C₆)alkyl,
   wherein the aryl portion of said alkylene-aryl of R^{c} is unsubstituted or substituted with one or more Y groups;
R^{d} is selected from the group consisting of H, (C₁-C₆)alkyl, and alkylene-aryl,
   wherein the aryl portion of said alkylene-aryl of R^{d} is unsubstituted or substituted with one or more Y groups;
each X is independently selected from the group consisting of halogen, (C₁-C₆)alkyl, halo(C₁-C₆)alkyl, —O—(C₁-C₆)alkyl, —O-halo(C₁-C₆)alkyl, and —OH;
each Y is independently selected from the group consisting of halogen, (C₁-C₆)alkyl, halo(C₁-C₆)alkyl, —O—(C₁-C₆)alkyl, —O-halo(C₁-C₆)alkyl, —CN, —NO₂, —OH, —S(O₂)—(C₁-C₆)alkyl, —S(O₂)-aryl, —S(O₂)-NH₂, —S(O₂)—NH—(C₁-C₆)alkyl, —S(O₂)—NH-aryl, —S(O₂)-N(alkyl)₂, —S(O₂)-N(aryl)₂, —S(O₂)-N(alkyl)(aryl), and aryl;
each Z is independently selected from the group consisting of (C₁-C₆)alkyl, halo(C₁-C₆)alkyl, halogen, —O—(C₁-C₆)alkyl, —O-halo(C₁-C₆)alkyl, —CN, —OH, aryl, and N-oxide;
n is 0, 1, 2, or 3;
m is 0, 1, or 2; and

with the proviso that when L is (f), and R², R³ and R⁵ are each H, then R¹ is not —CH₃. These compounds are described in U.S. Publication Nos. 2007/0066630, and 2006/0264489, herein incorporated by reference.

In another embodiment the NAR agonist is a compound of Formula VI:

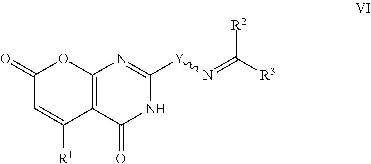

or a pharmaceutically acceptable salt, solvate, ester, or tautomer thereof, wherein:
R¹ is selected from the group consisting of H, R⁴, halo(C₁-C₆)alkyl, -alkylene-R⁴, -alkylene-R⁵, -alkylene-R⁶, (C₂-C₆)alkenyl, (C₂-C₆)alkynyl, and -alkylene-O—(C₁-C₆)alkyl;
R² is selected from the group consisting of (C₁-C₆)alkyl, halo(C₁-C₆)alkyl, -alkylene-R⁵, R⁴, R⁵, R⁶, R⁷ and -alkylene-O—R⁸;
R³ is selected from the group consisting of (C₁-C₆)alkyl, halo(C₁-C₆)alkyl, -alkylene-R⁵, R⁴, R⁵, R⁶, and R⁷; or
R² and R³ together with the carbon atom to which they are both attached form a (C₃-C₁₀)cycloalkyl or heterocycloalkyl ring, wherein said (C₃-C₁₀)cycloalkyl or heterocycloalkyl ring is unsubstituted or independently substituted with one or more X⁵ groups, and wherein said (C₃-C₁₀)cycloalkyl ring can form a spirocyclic compound with a second (C₃-C₁₀)cycloalkyl ring or with a heterocycloalkyl ring, wherein the second (C₃-C₁₀)cycloalkyl ring or the heterocycloalkyl ring is unsubstituted or independently substituted with one or more X⁵ groups;
R⁴ is unsubstituted (C₃-C₁₀)cycloalkyl or (C₃-C₁₀)cycloalkyl substituted with one or more X¹ groups;
R⁵ is unsubstituted aryl and aryl substituted with one or more X² groups;
R⁶ is selected from the group consisting of unsubstituted heteroaryl and heteroaryl substituted with one or more X³ groups;
R⁷ is unsubstituted heterocycloalkyl and heterocycloalkyl substituted with one or more X⁴ groups;
R⁸ is selected from the group consisting of H, (C₁-C₆)alkyl, R⁴, R⁵, R⁶, R⁷, —C(O)—(C₁-C₆)alkyl, —C(O)—R⁵;
each R⁹ is independently selected from the group consisting of H, (C₁-C₆)alkyl, R⁴, R⁵, R⁶, and R⁷;
R¹⁰ is selected from the group consisting of R⁹, —C(O)—(C₁-C₆)alkyl, and —C(O)—R⁵;
Y is —O— or —N(R¹⁰)—;
each X¹ is independently selected from the group consisting of halogen, (C₁-C₆)alkyl, —O—(C₁-C₆)alkyl, —OH, halo(C₁-C₆)alkyl, aryl, and (C₂-C₆)alkyne;
each X² is independently selected from the group consisting of halogen, (C₁-C₆)alkyl, —O—(C₁-C₆)alkyl, —OH, halo(C₁-C₆)alkyl, aryl, and (C₂-C₆)alkyne;
each X³ is independently selected from the group consisting of halogen, (C₁-C₆)alkyl, and N-oxide;
each X⁴ is independently selected form the group consisting of (C₁-C₆)alkyl, R⁵, —C(O)—(C₁-C₆)alkyl, —C(O)—R⁵, —C(O)—O—(C₁-C₆)alkyl, —(C₂-C₆)alkylene-R⁵, R⁴, and —S(O₂)—(C₁-C₆)alkyl; and
each X⁵ is independently selected from the group consisting of (C₁-C₆)alkyl, a fused aryl ring, —C(O)—(C₁-C₆)alkyl, a fused heteroaryl ring, —C(O)—O—(C₁-C₆)alkyl, —C(O)—R⁵, —S(O₂)—(C₁-C₆)alkyl, —C(O)—N(R⁹)₂, R⁵, R⁶, —C(O)—R⁴, —C(O)—O—R⁴, —S(O₂)—R⁴, —S(O₂)-alkylene-R⁴, —S(O₂)-alkylene-R⁵, —N(R⁹)—C(O)—O—(C₁-C₆)alkyl, —N(R⁹)—C(O)—O—R⁴, —N(R⁹)—C(O)—N(R⁹)₂ and —N(R⁹)₂;
   wherein said fused aryl ring of X⁵ is unsubstituted or independently substituted with one or more substituent selected from -alkylene-R⁷ or X², and said fused heteroaryl ring of X⁵ is unsubstituted or substituted with one or more X³ groups.
   These compounds are described in U.S. Publication No. 2007/0173495, herein incorporated by reference.

In another embodiment of the compounds of formula II-B, or a pharmaceutically acceptable salt, solvate, ester, or tautomer thereof, R′ is (C₁-C₆)alkyl; and R² and R³ are each independently selected from the group consisting of (C₁-C₆)alkyl, halo(C₁-C₆)alkyl, -alkylene-R⁵, R⁴, R⁵, R⁶, and R⁷.

In another embodiment of the compounds of formula II-B, or a pharmaceutically acceptable salt, solvate, ester, or tautomer thereof, R¹ is —CH₂CH₃, —CH₂CH₂CH₂CH₃, —CH₂CH₂CH₂CH₂CH₃, —CH₂CH₂CH(CH₃)₂, or —CH₂CH₂CH₂-cyclopropyl; R² and R³ are each independently selected from the group consisting of —CH₃, —CH₂CH₃, —CH₂CH₂CH₃, —CH₂CH₂CH₂CH₃, —CH₂C(CH₃)₃, —CH₂CH₂CF₃, —CH₂CH₂—R⁵, cyclopropyl, piperazinyl, piperidinyl, morpholinyl, phenyl, thiophenyl, pyridyl, and thiazolyl; and R⁵ is phenyl.

In another embodiment the NAR agonist is a compound of the Formula VII, VIII-A, VIII-B, IX or X:

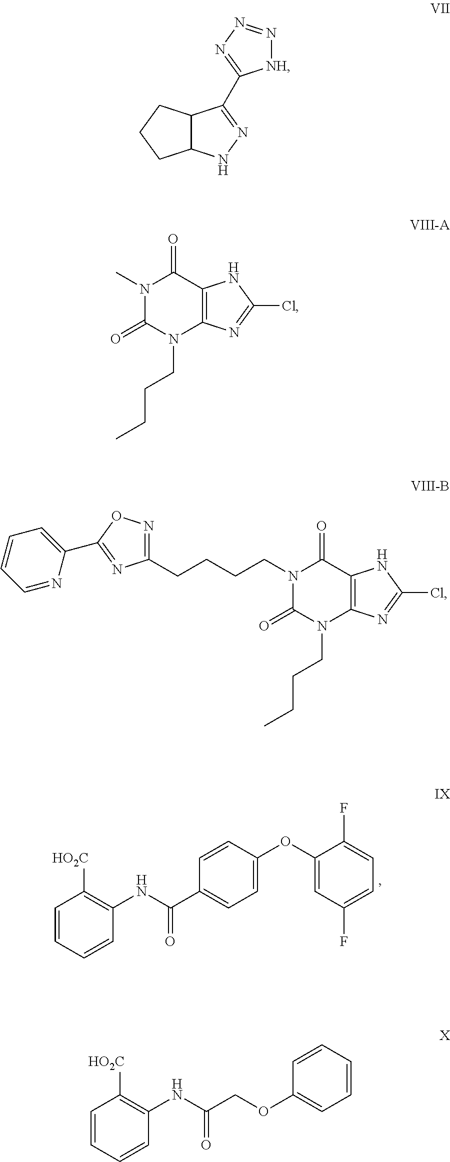

or a pharmaceutically acceptable salt, solvate, ester, or tautomer thereof.

The compound of Formula VII is made by Merck and Arena.

The compounds of Formula VIII-A or VIII-B are made by GSK.

The compound of Formula IX is made by Roche.

The compound of Formula X is made by GSK.

In another embodiment the NAR agonist is selected from the group consisting of:

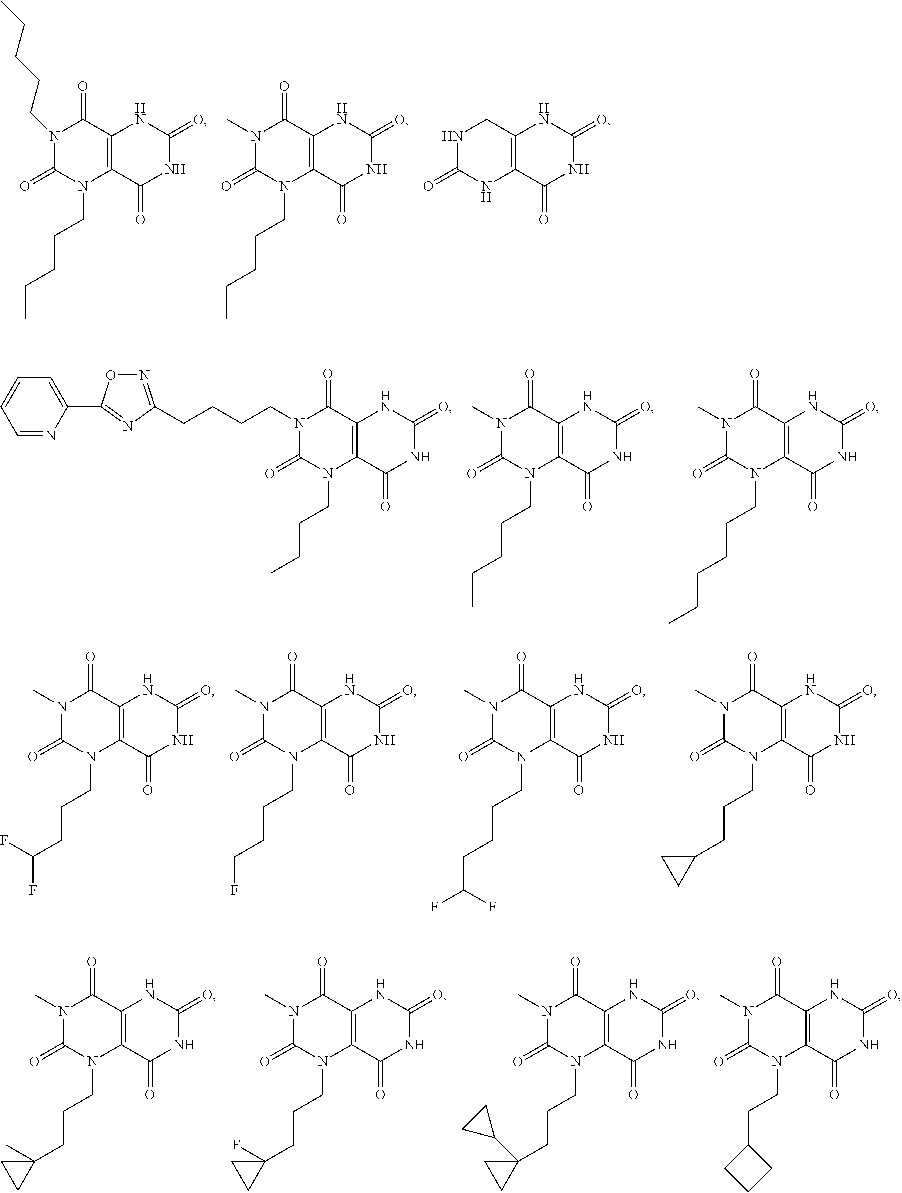

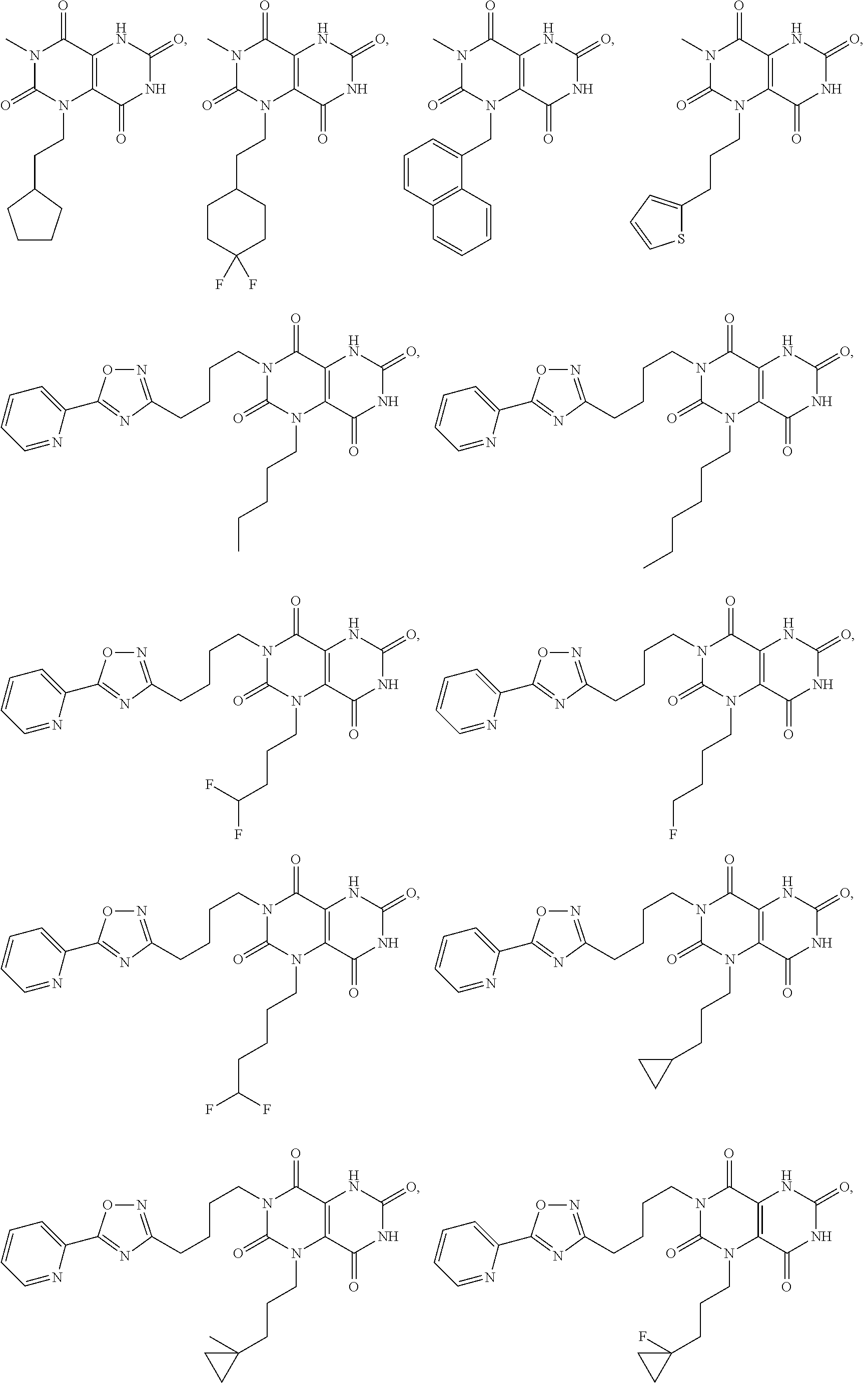

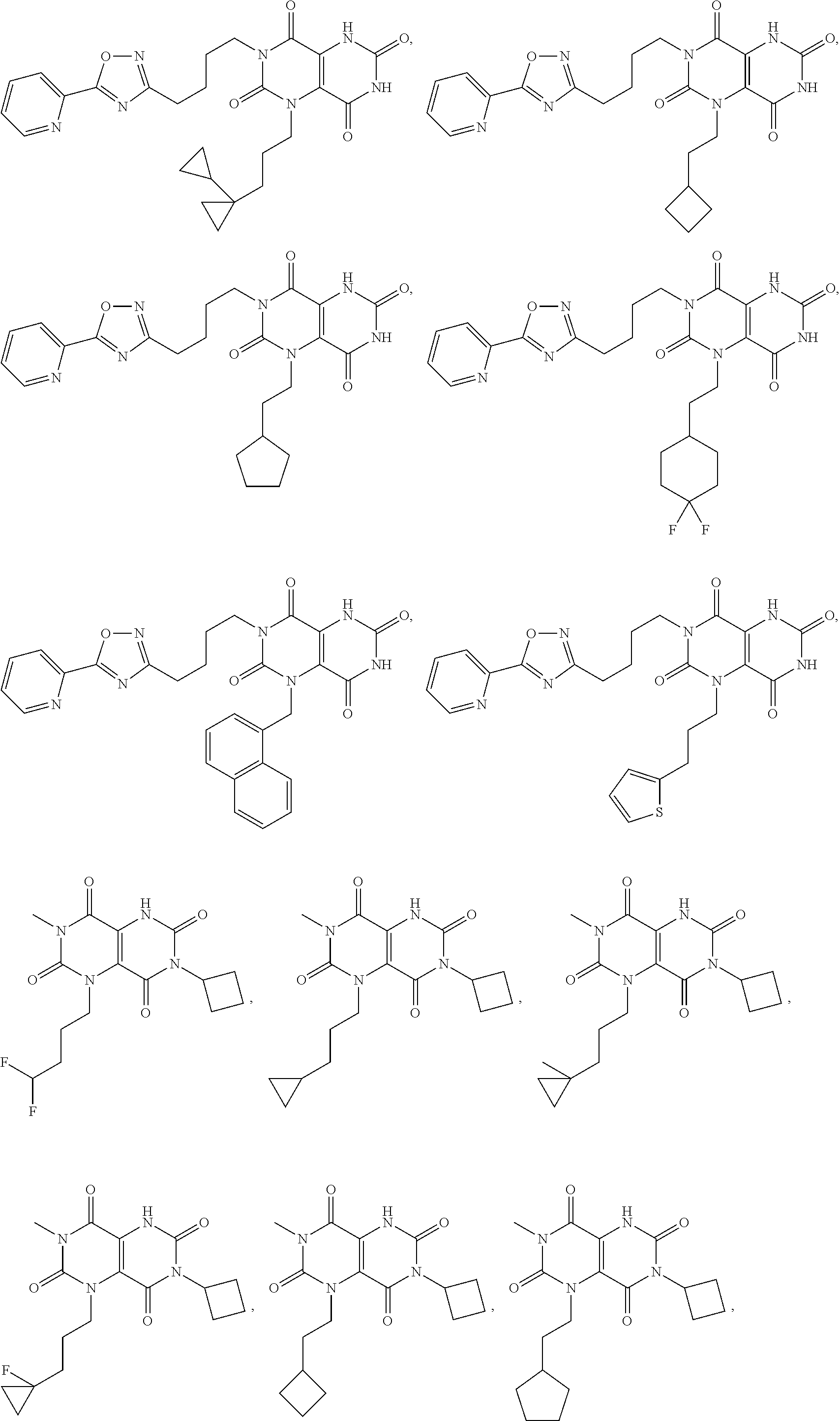

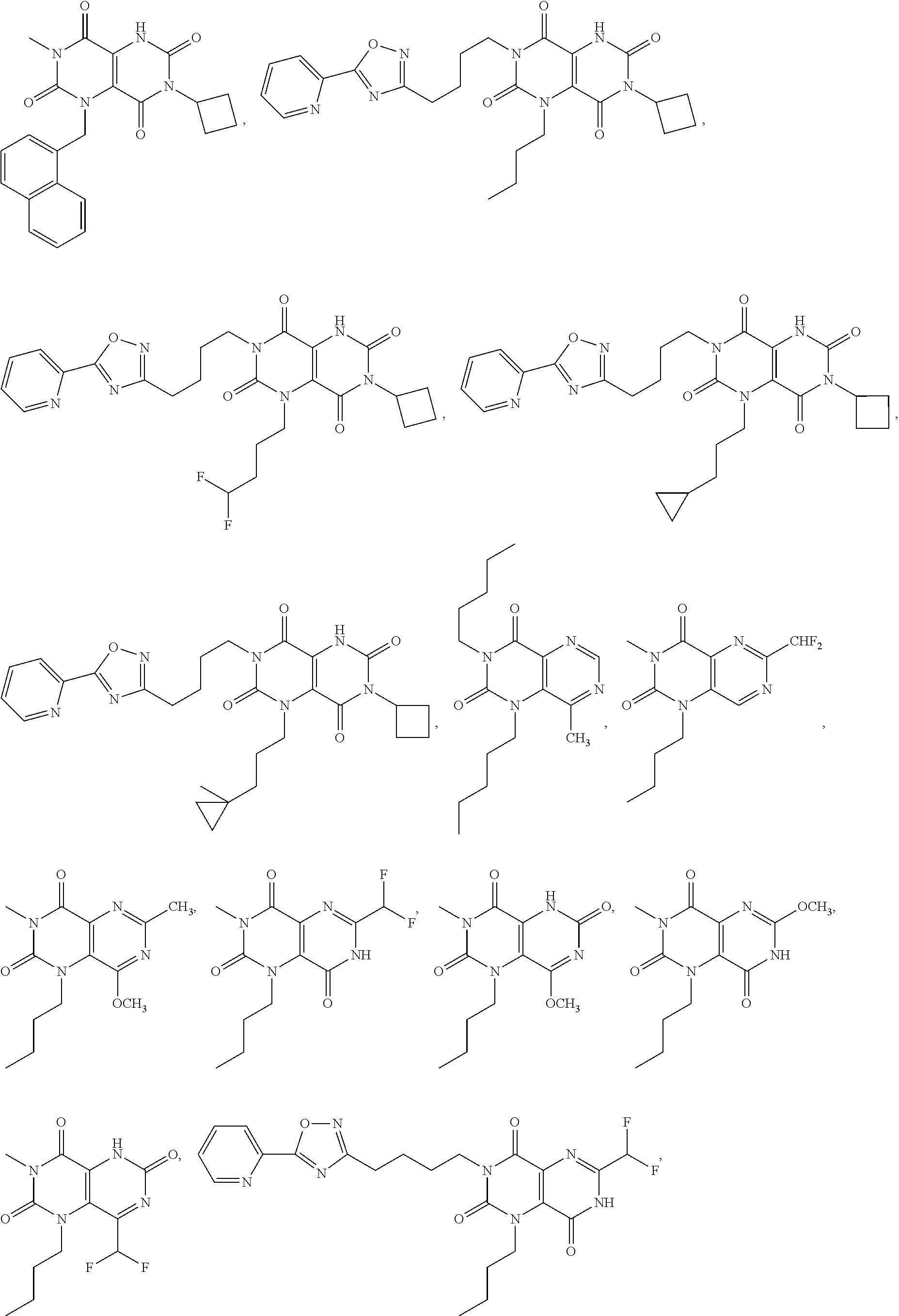

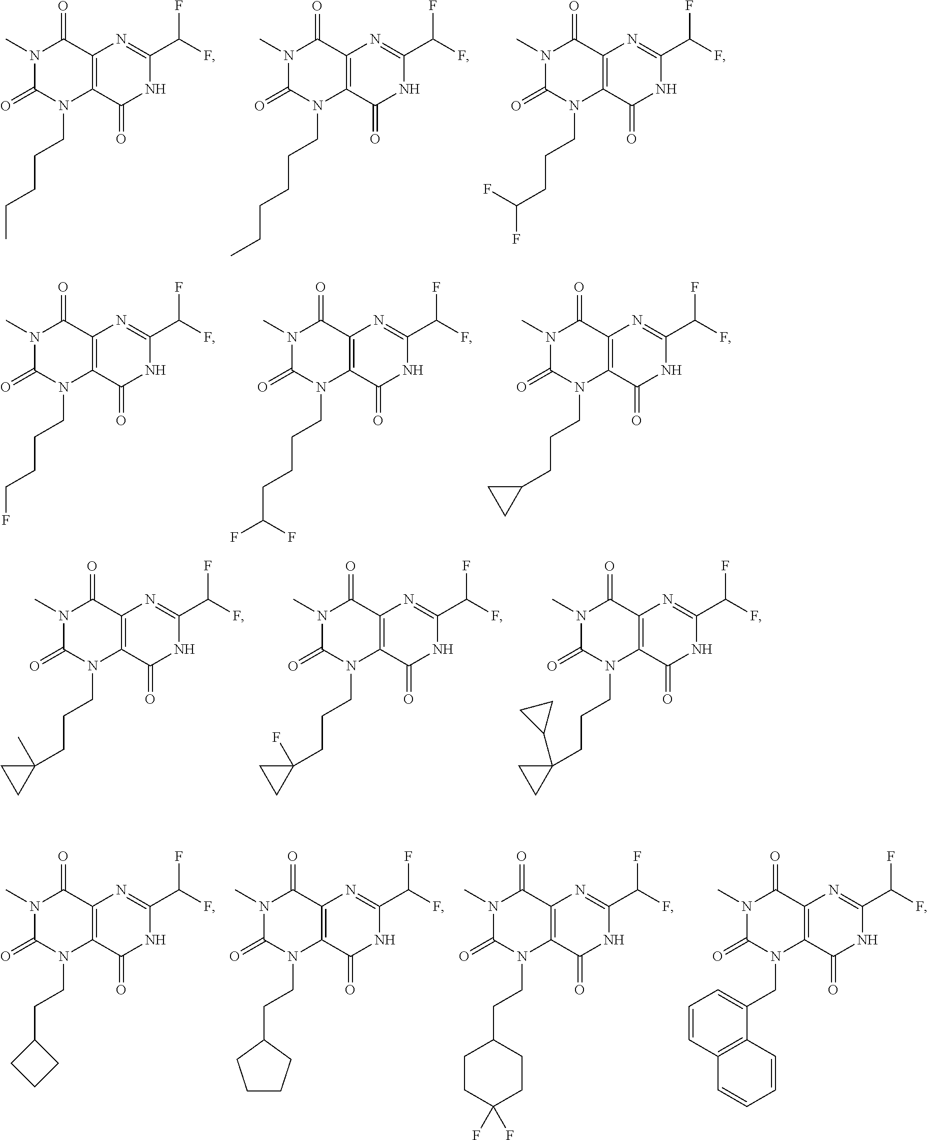

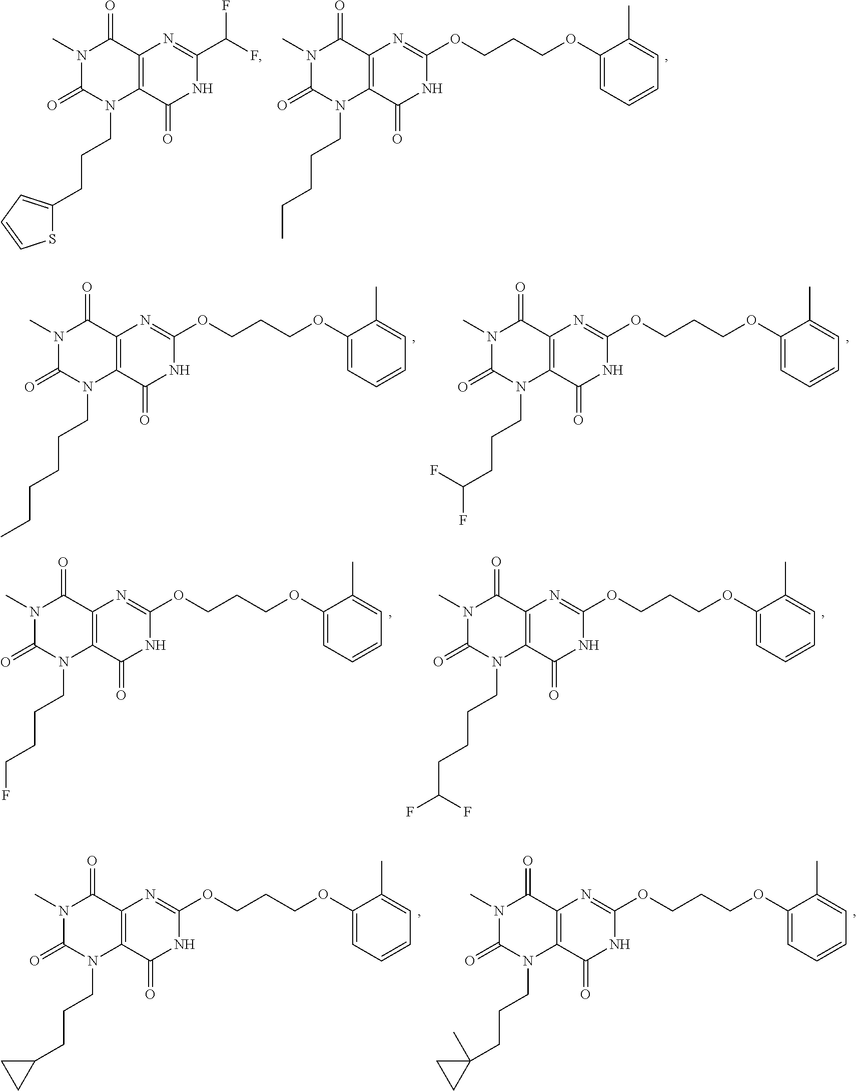

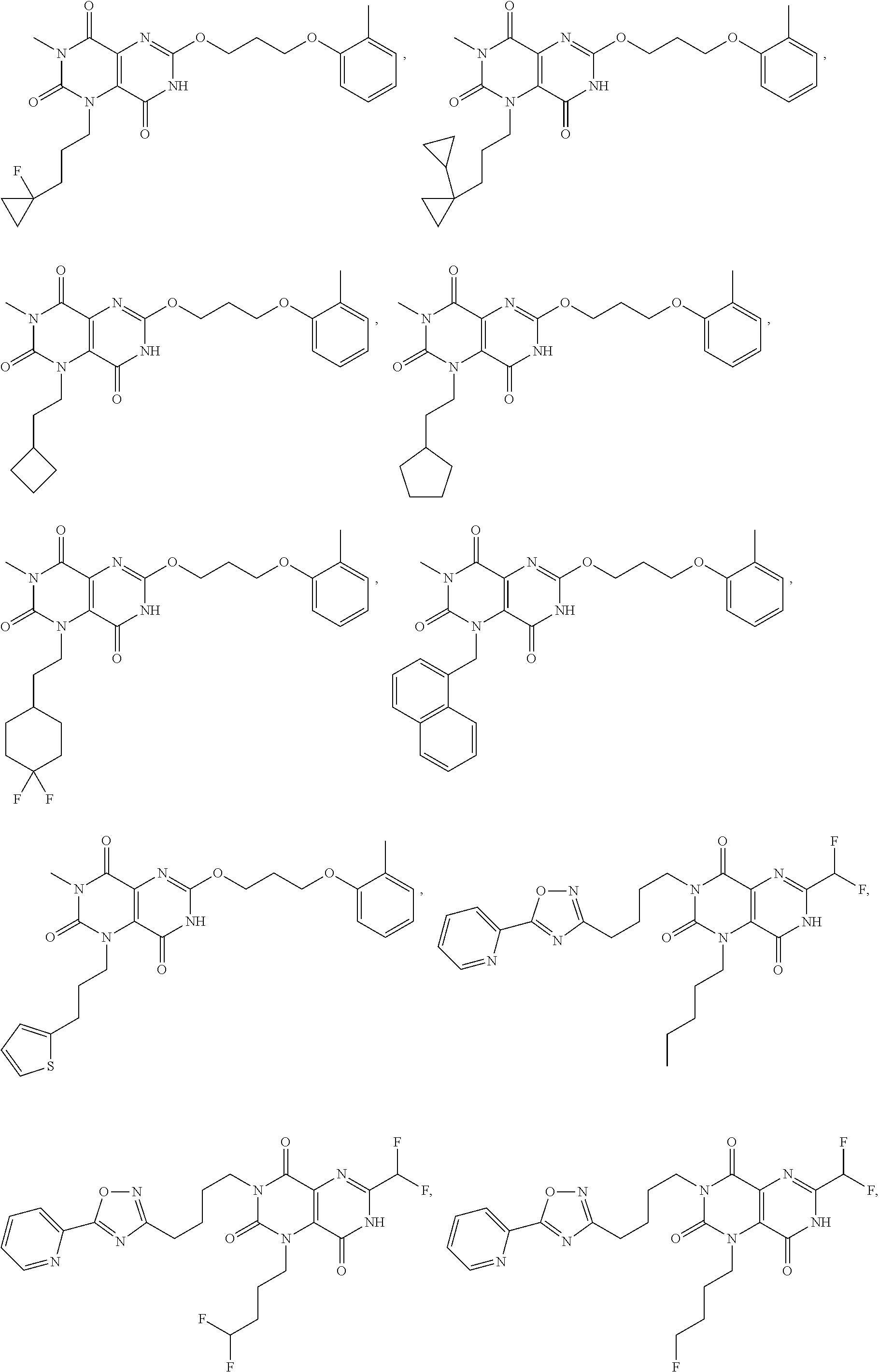

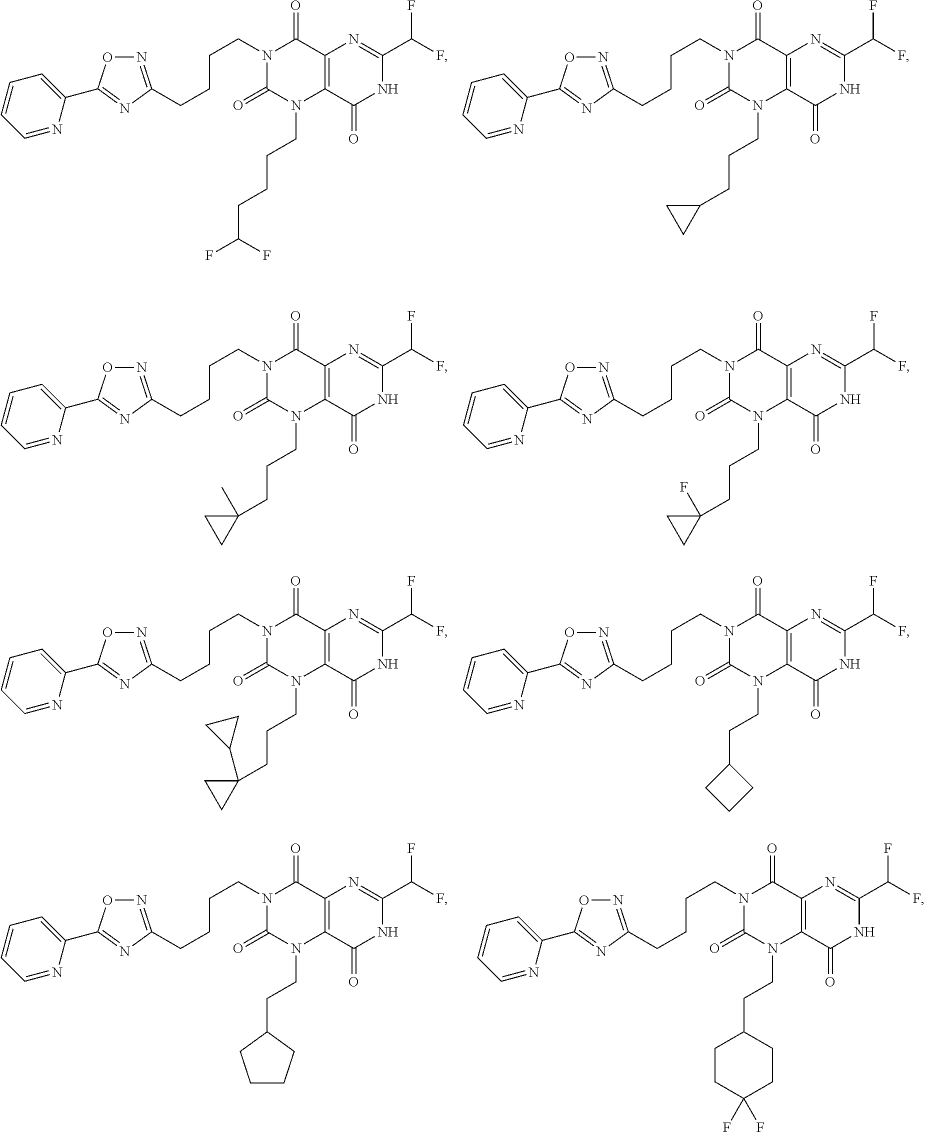

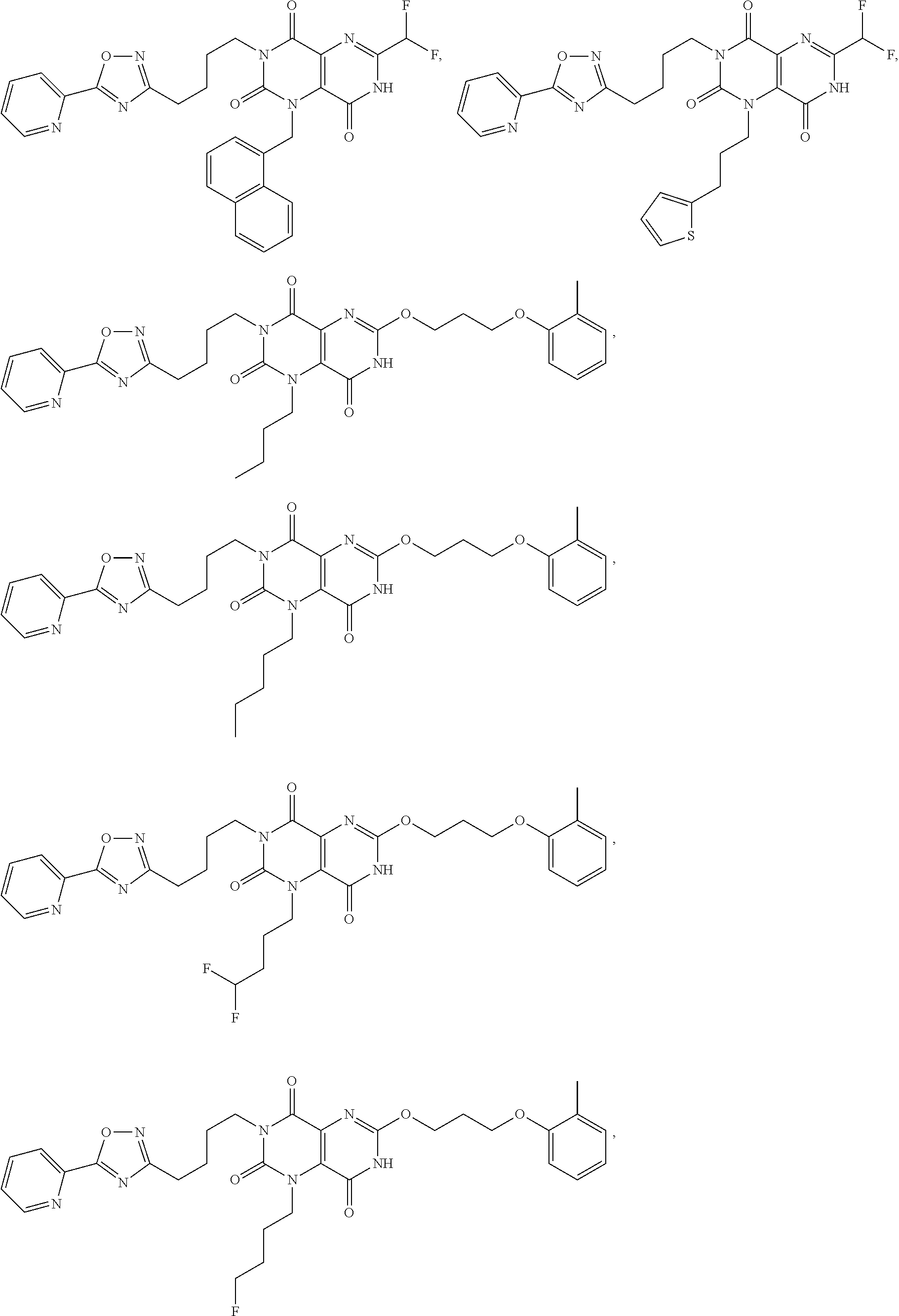

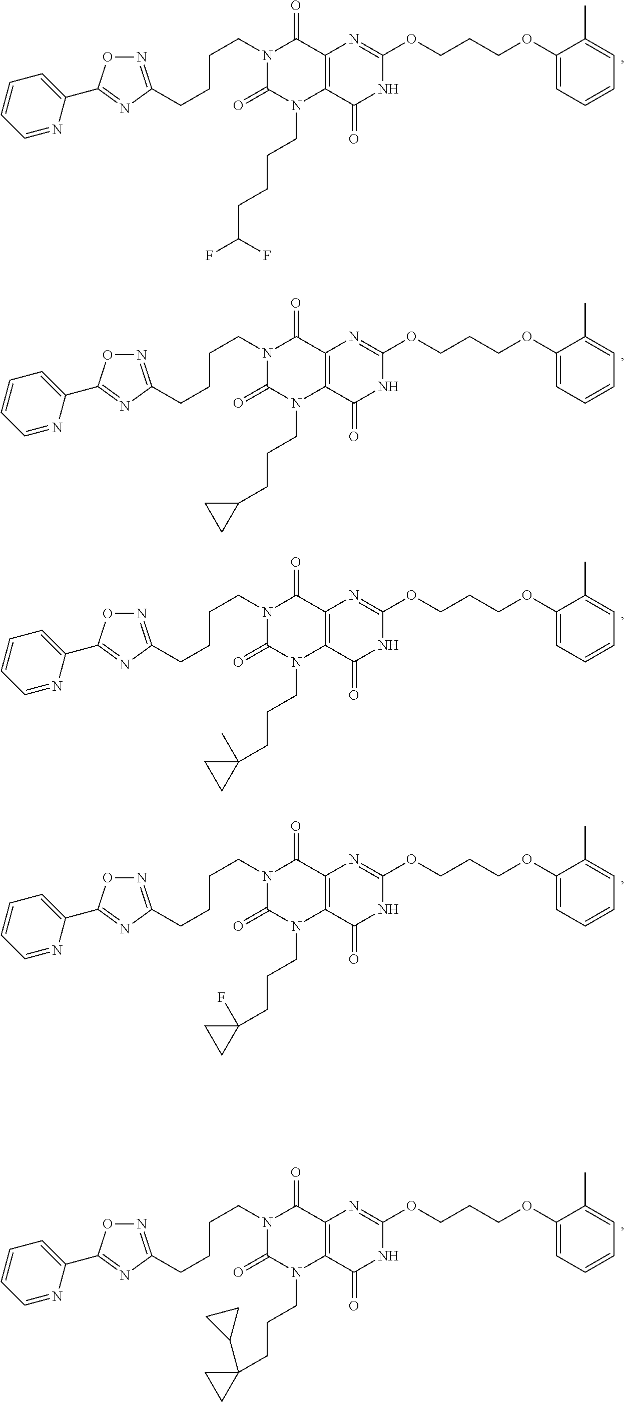

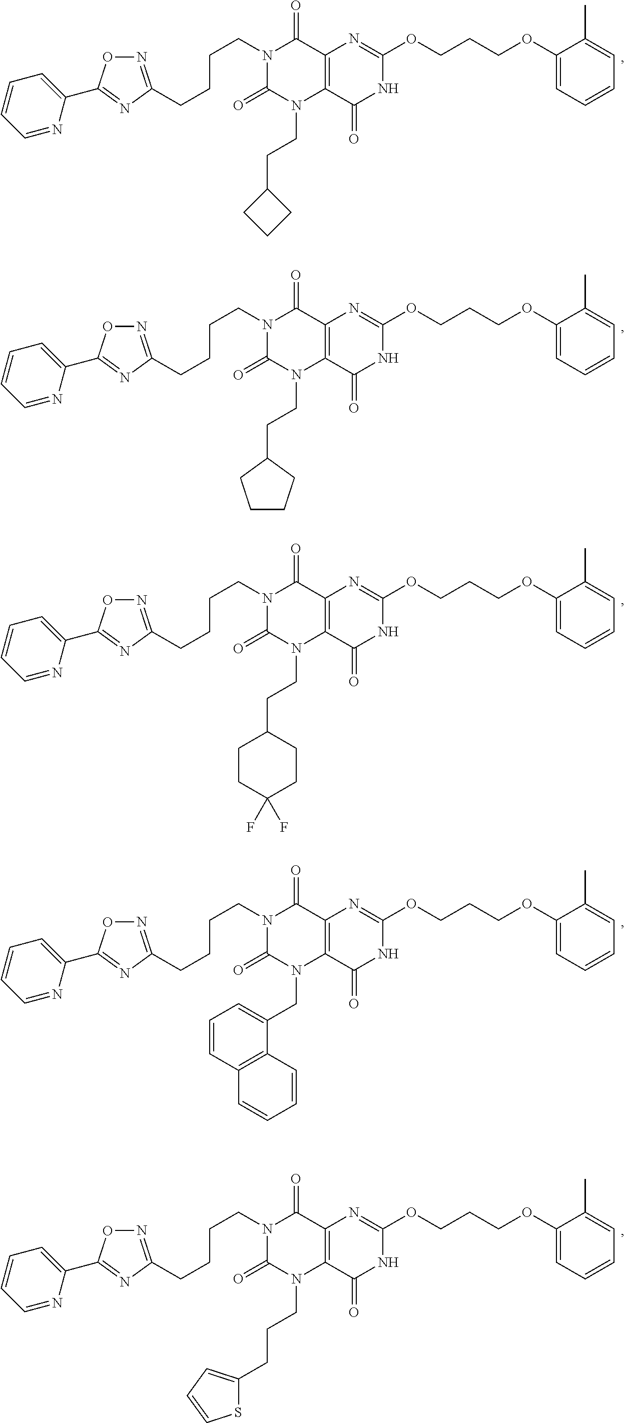

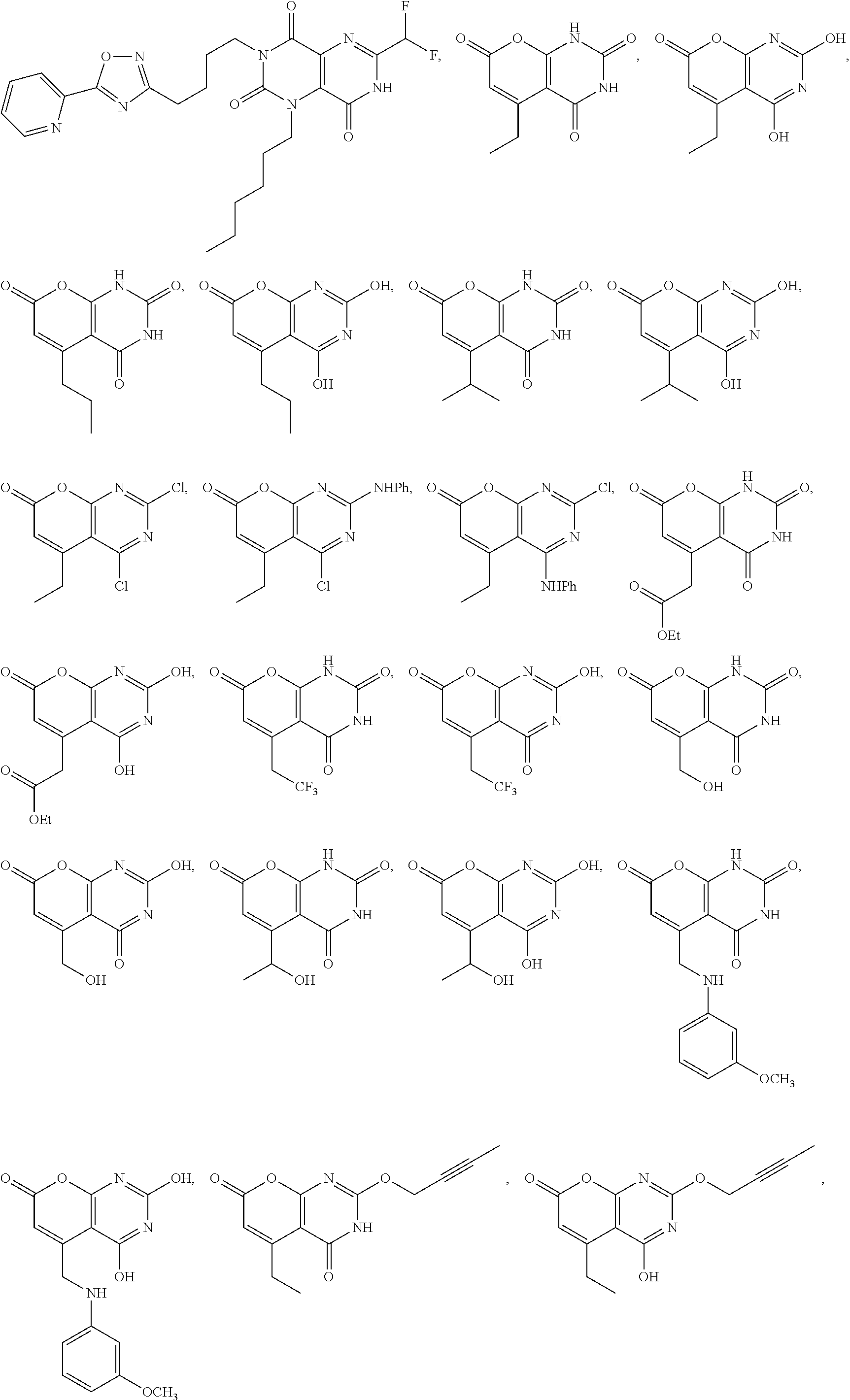

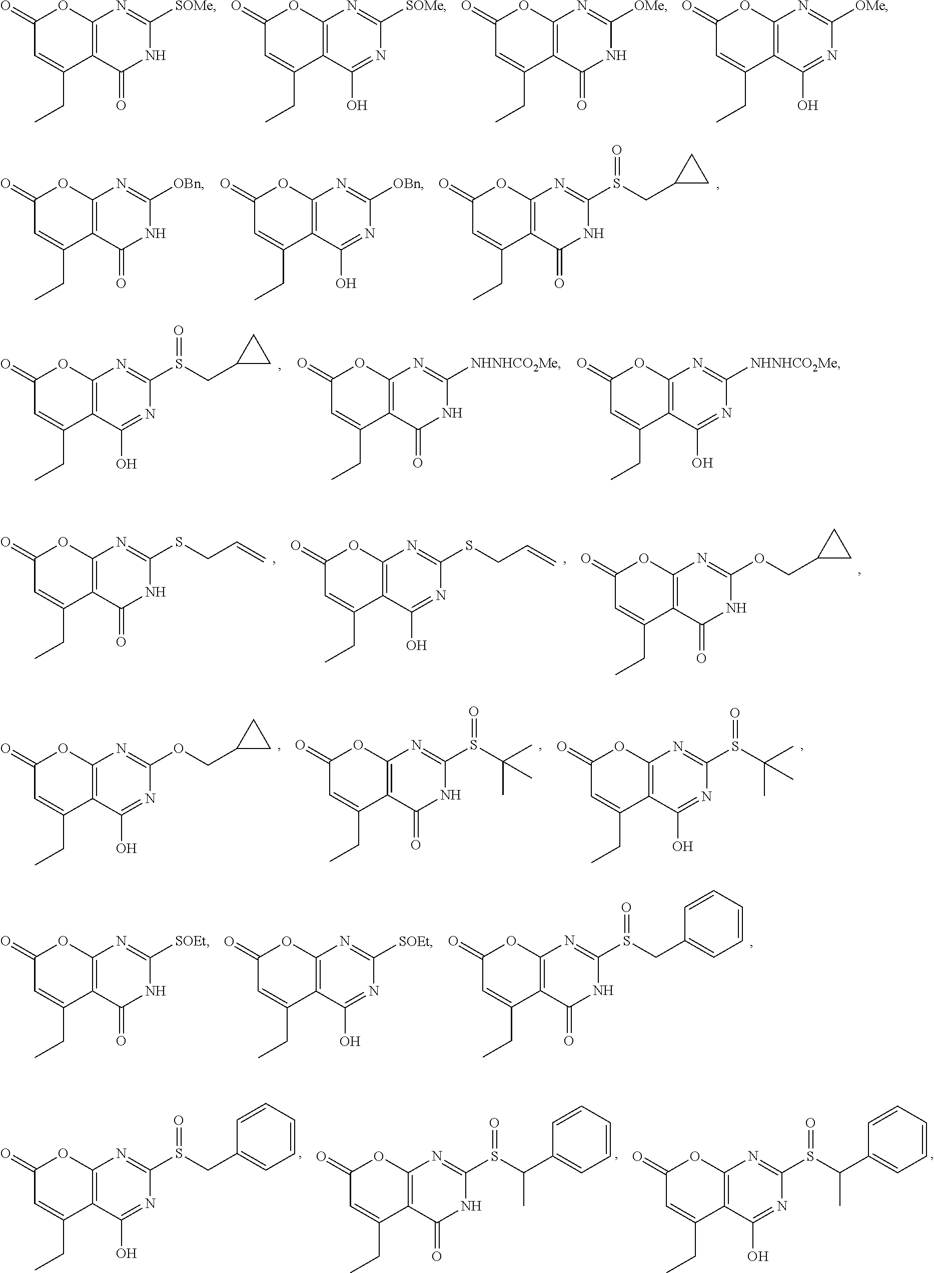

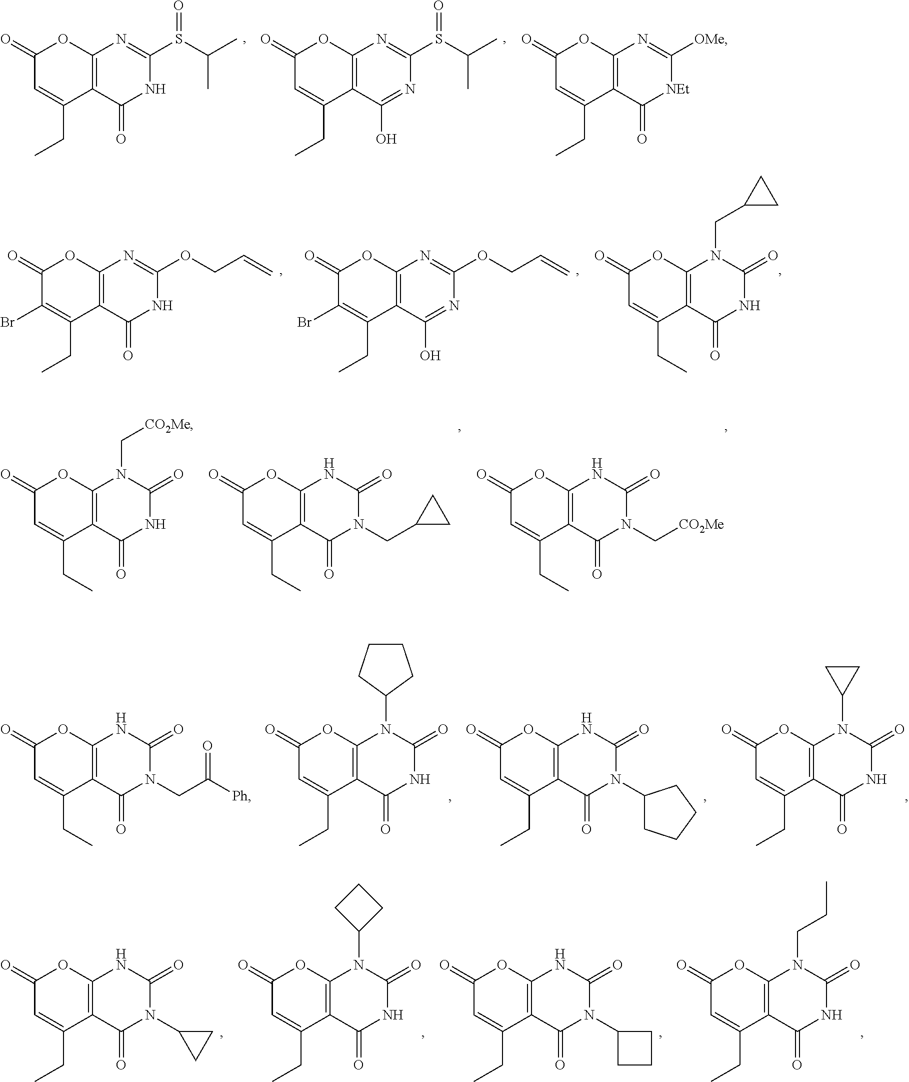

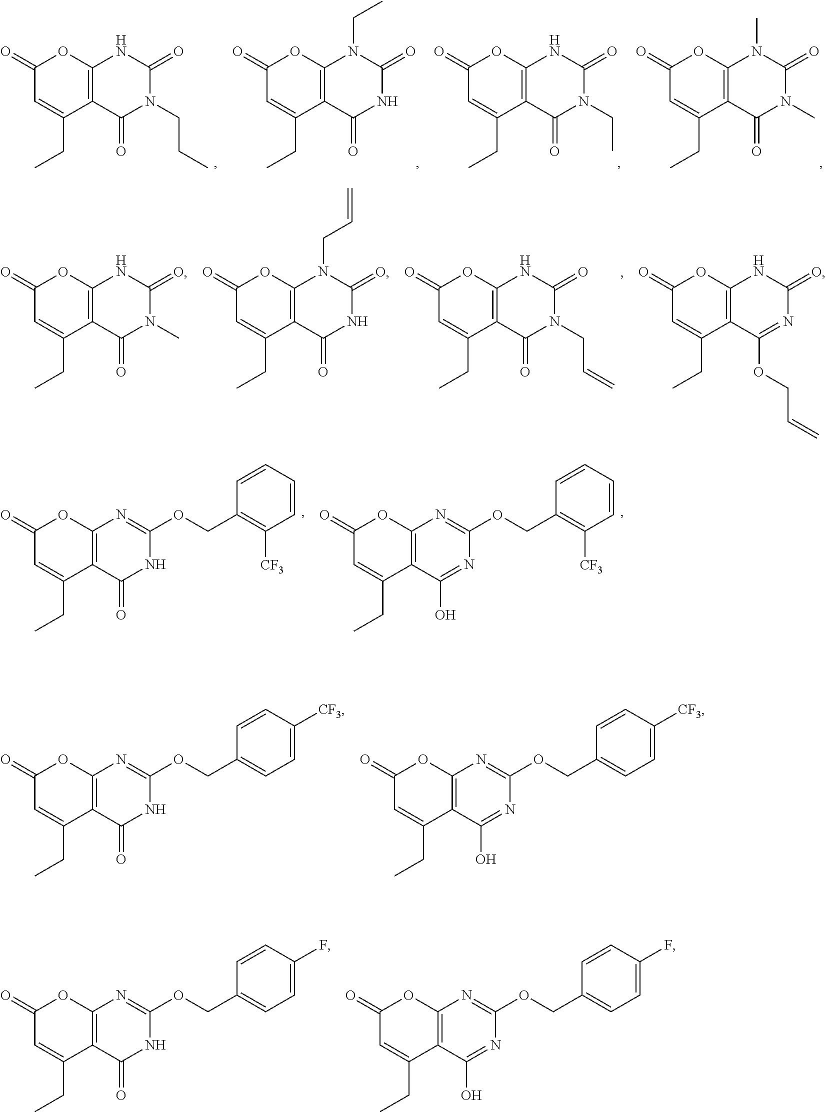

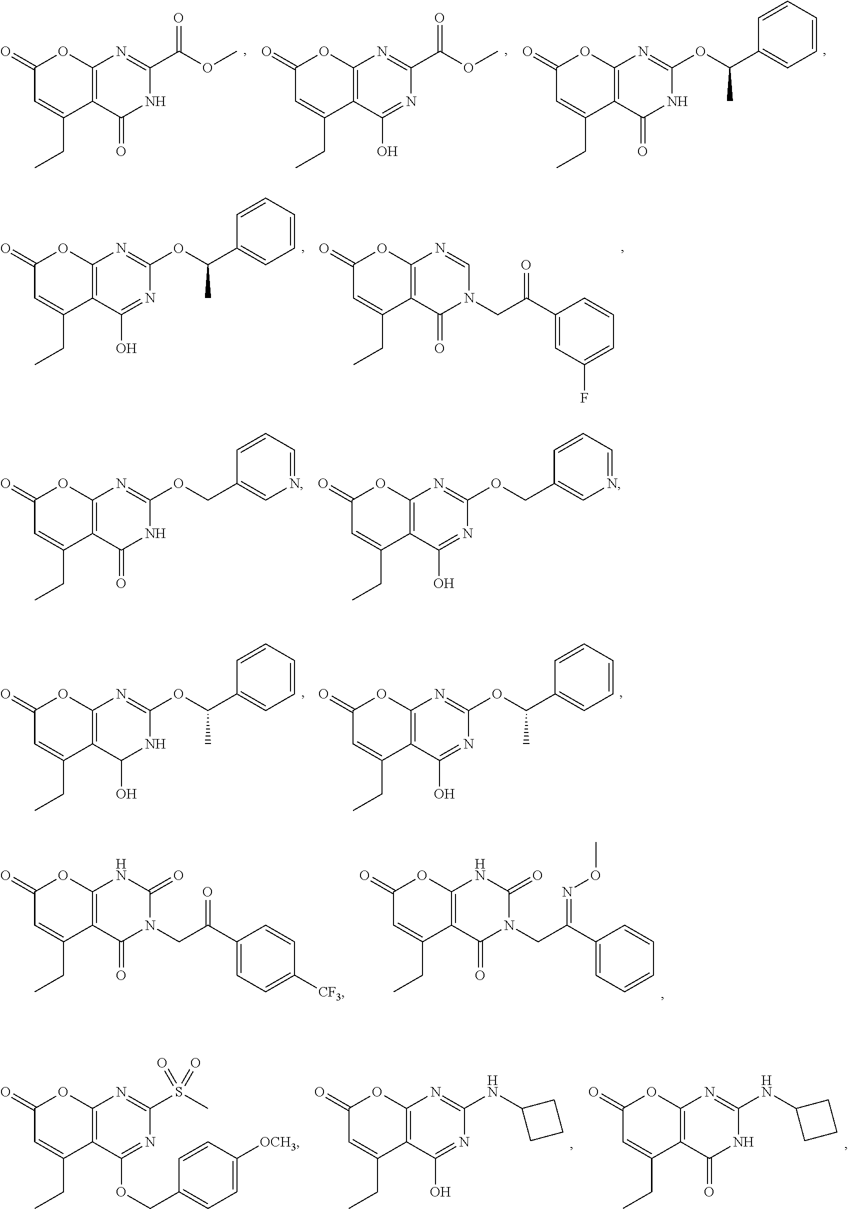

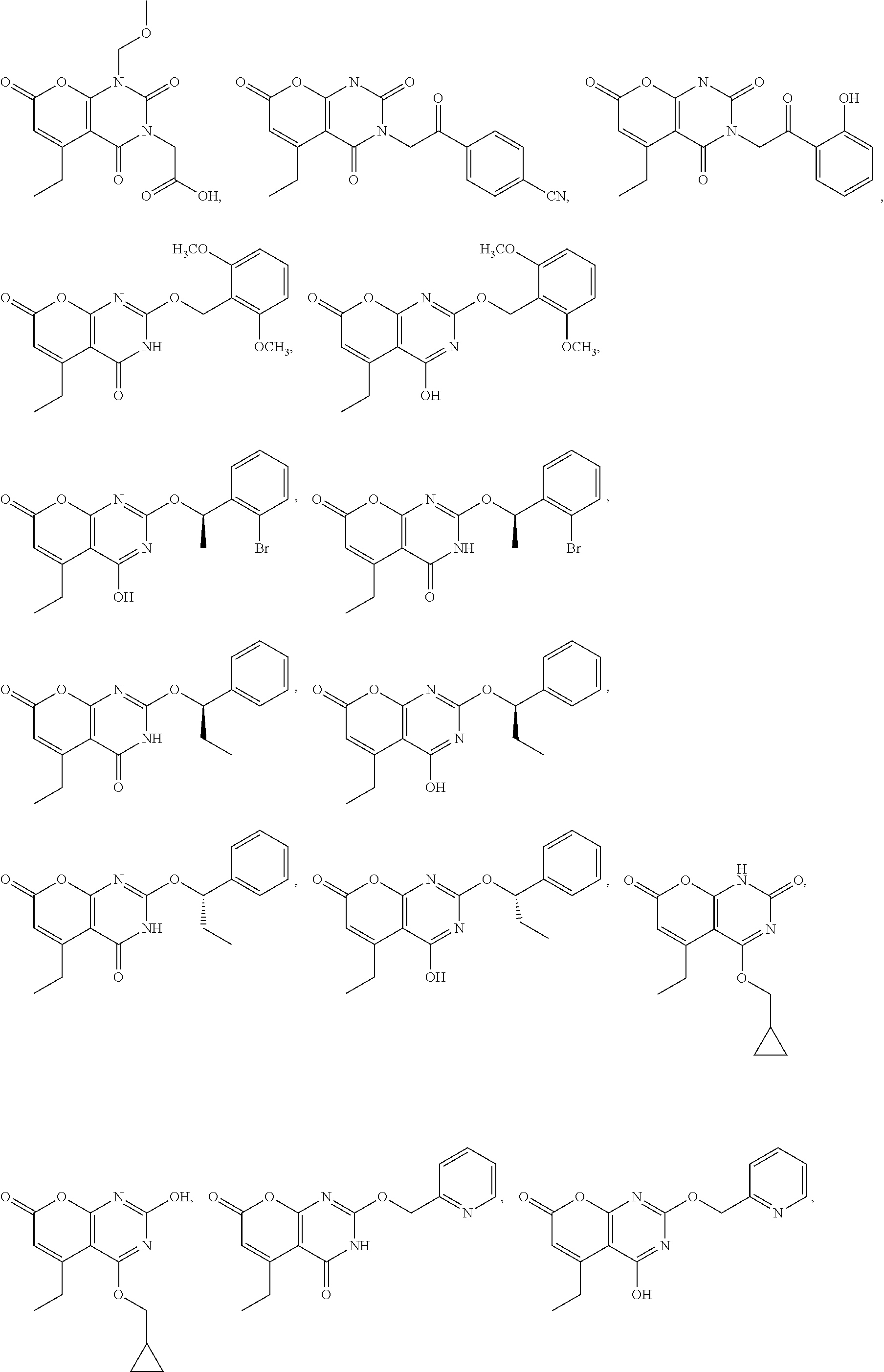

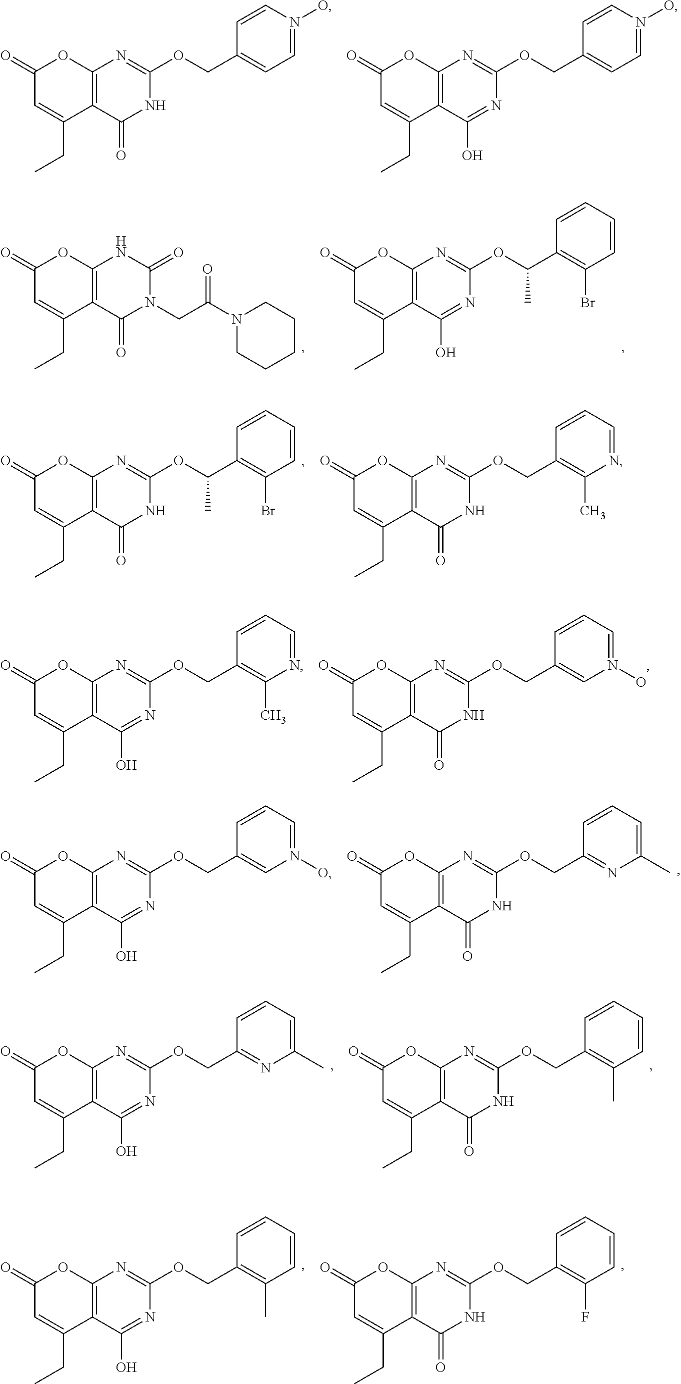

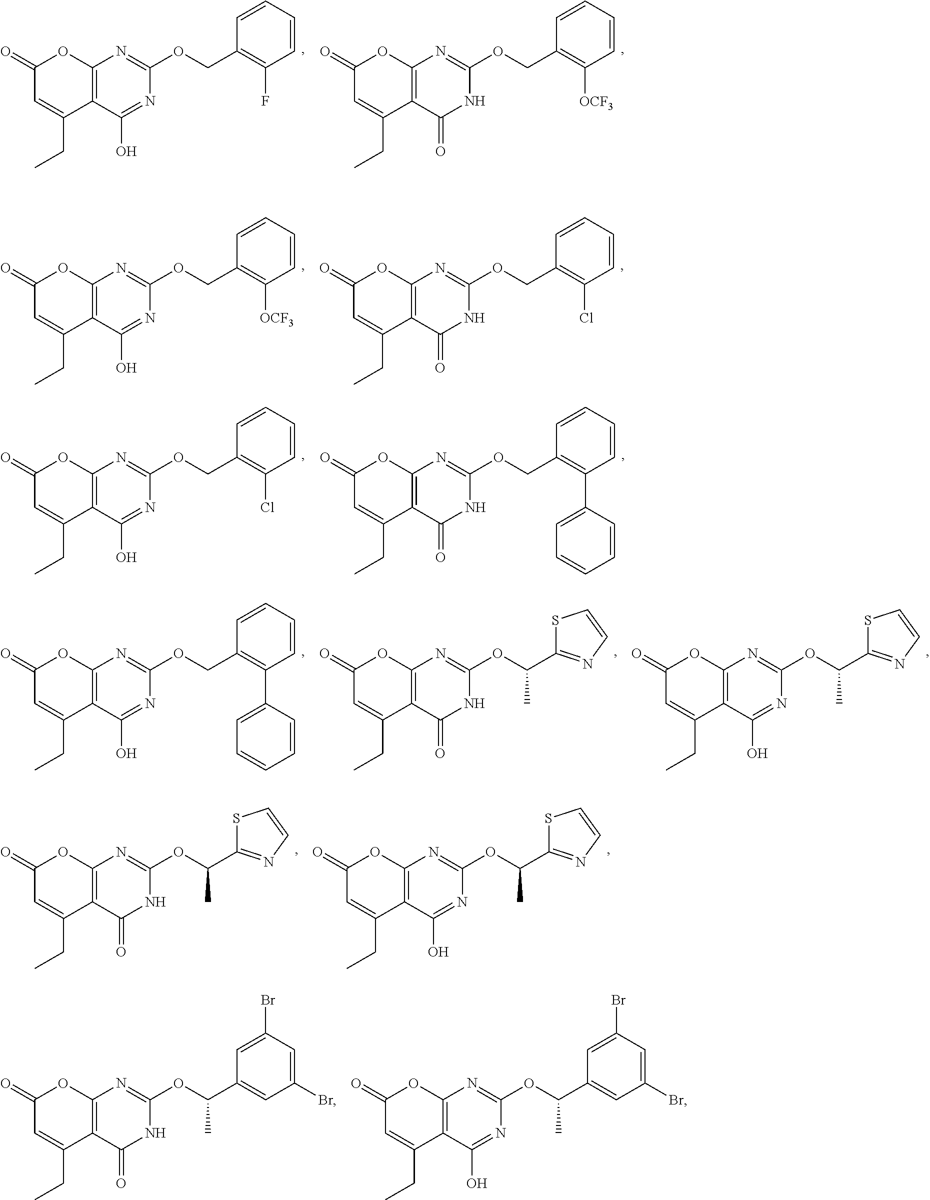

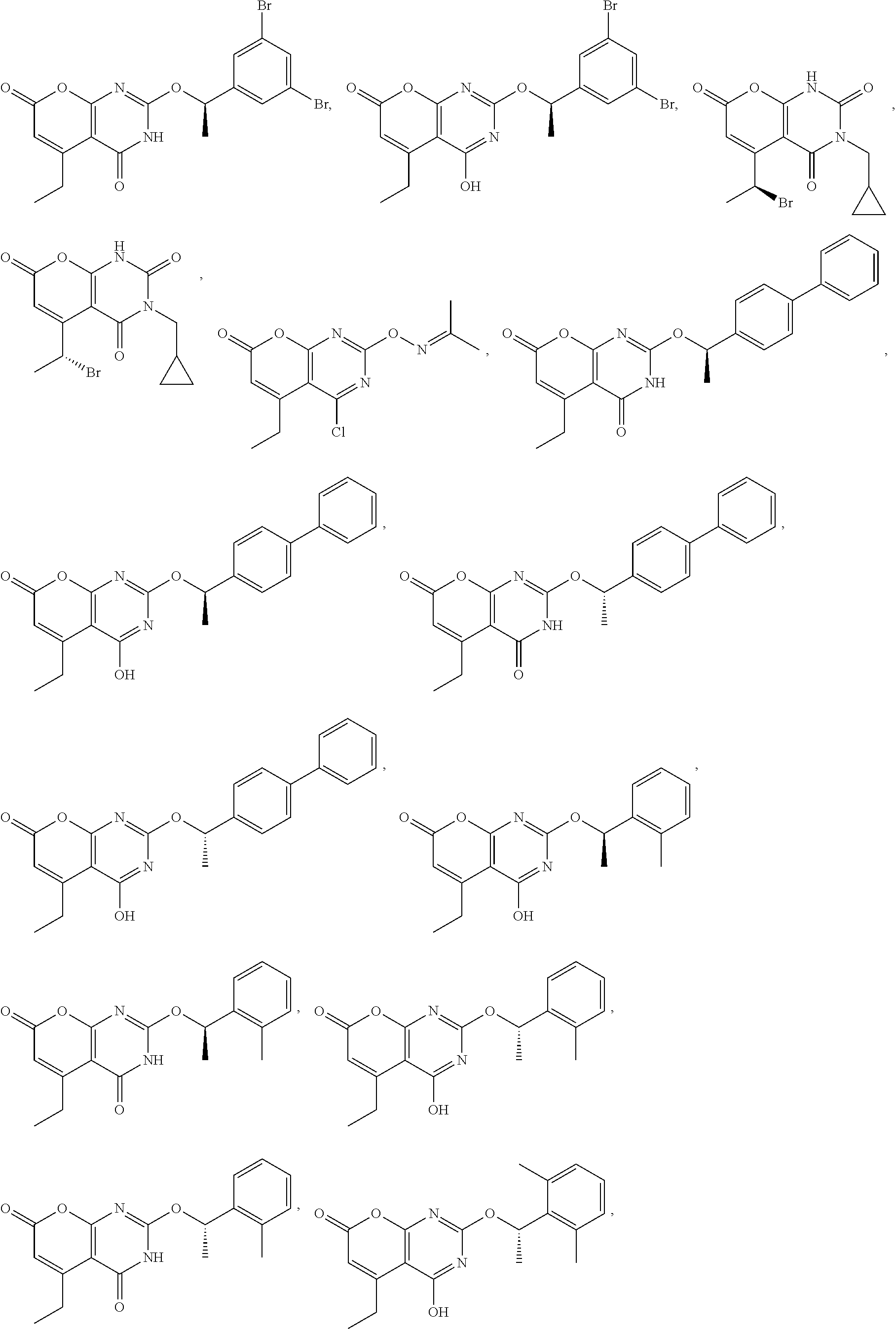

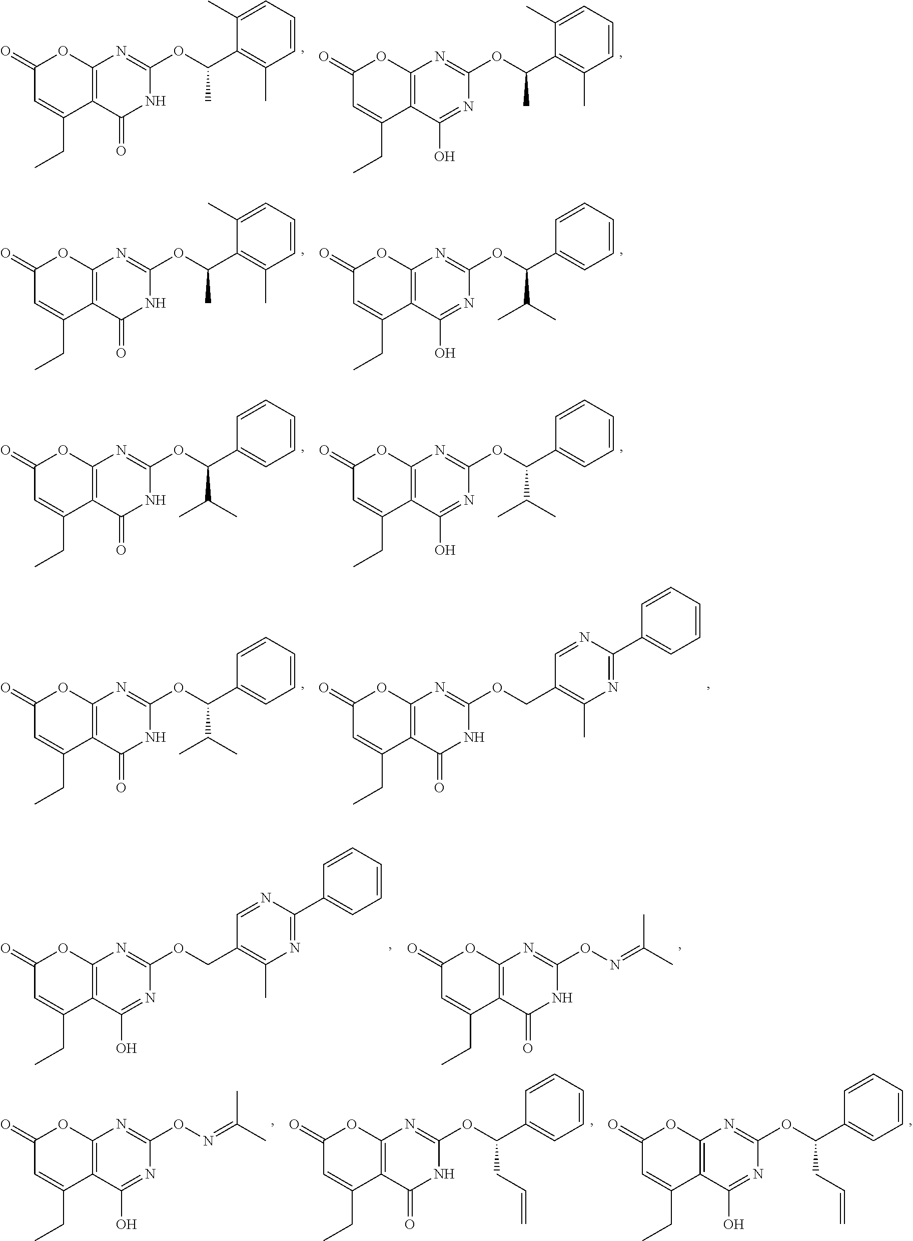

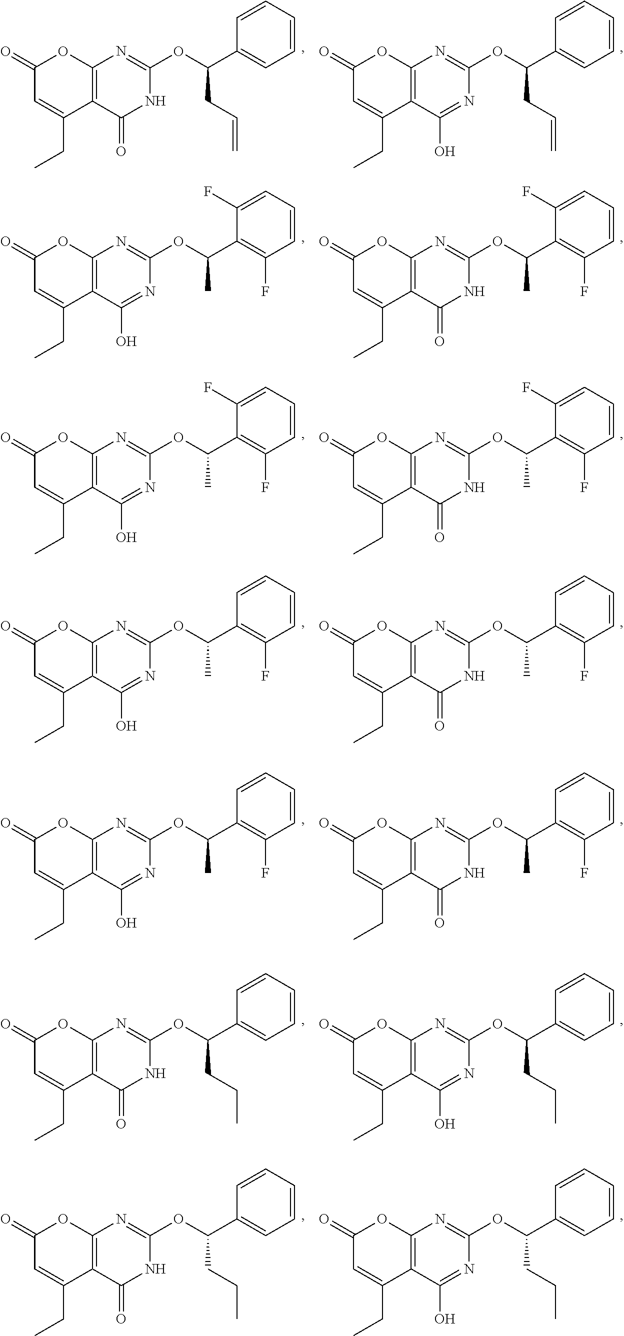

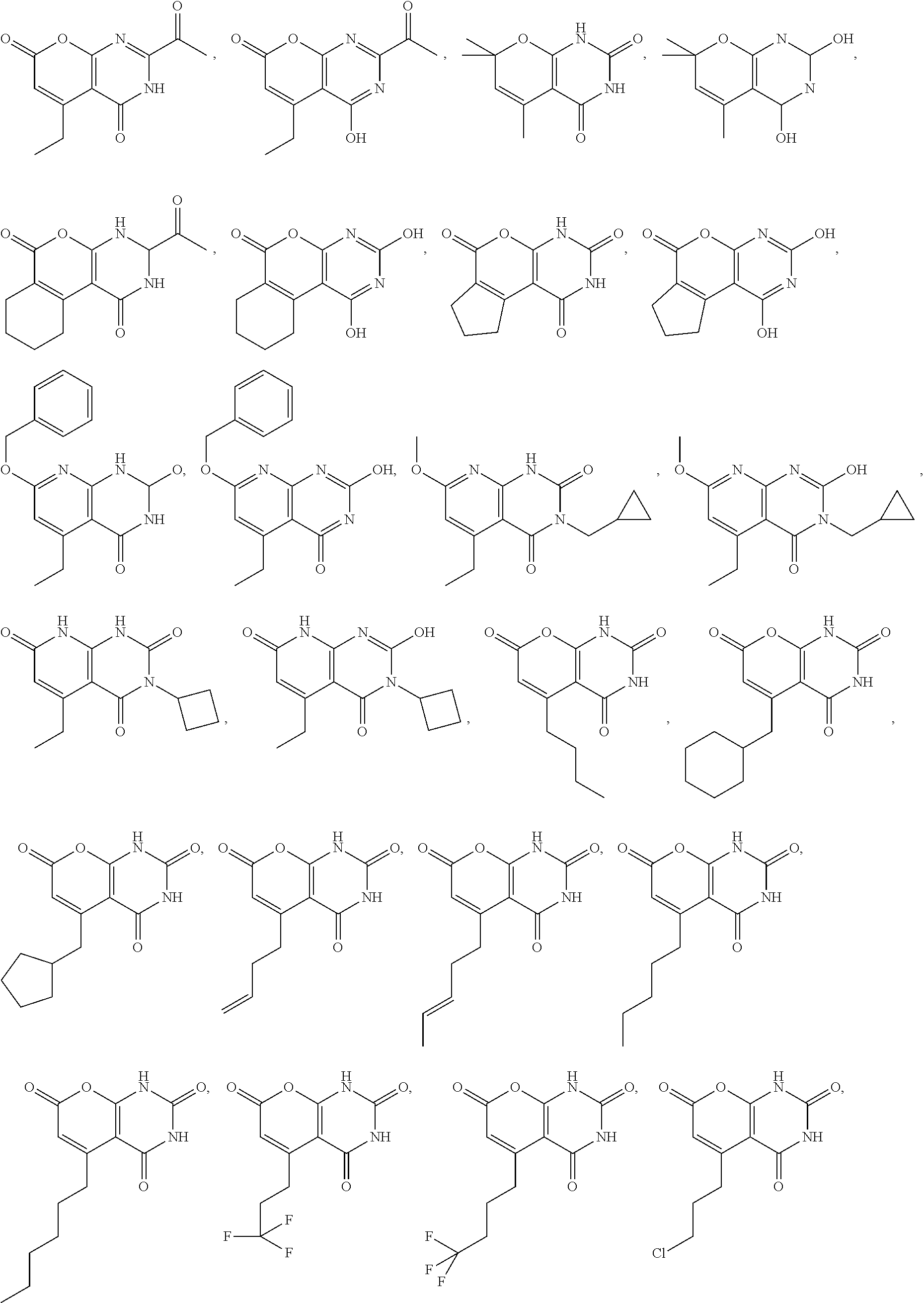

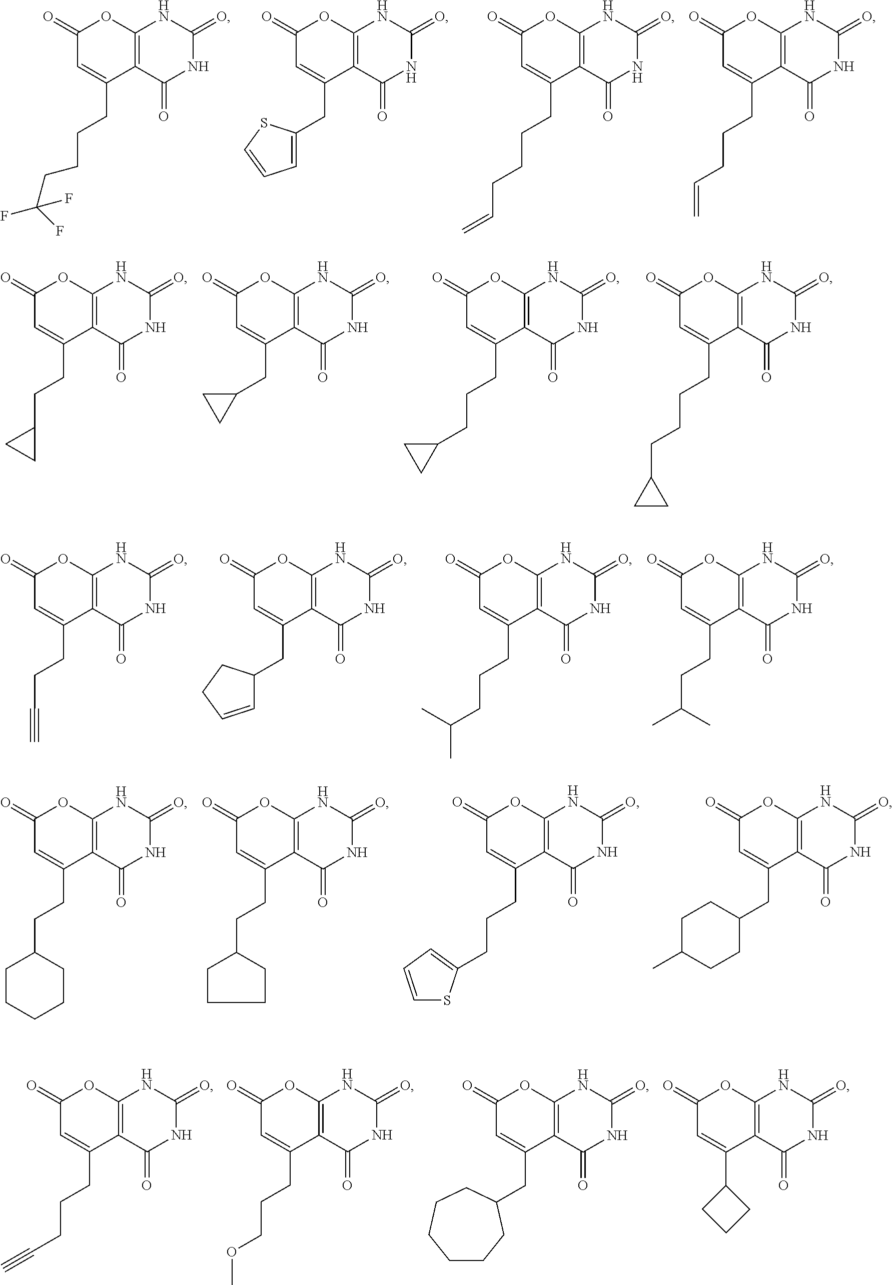

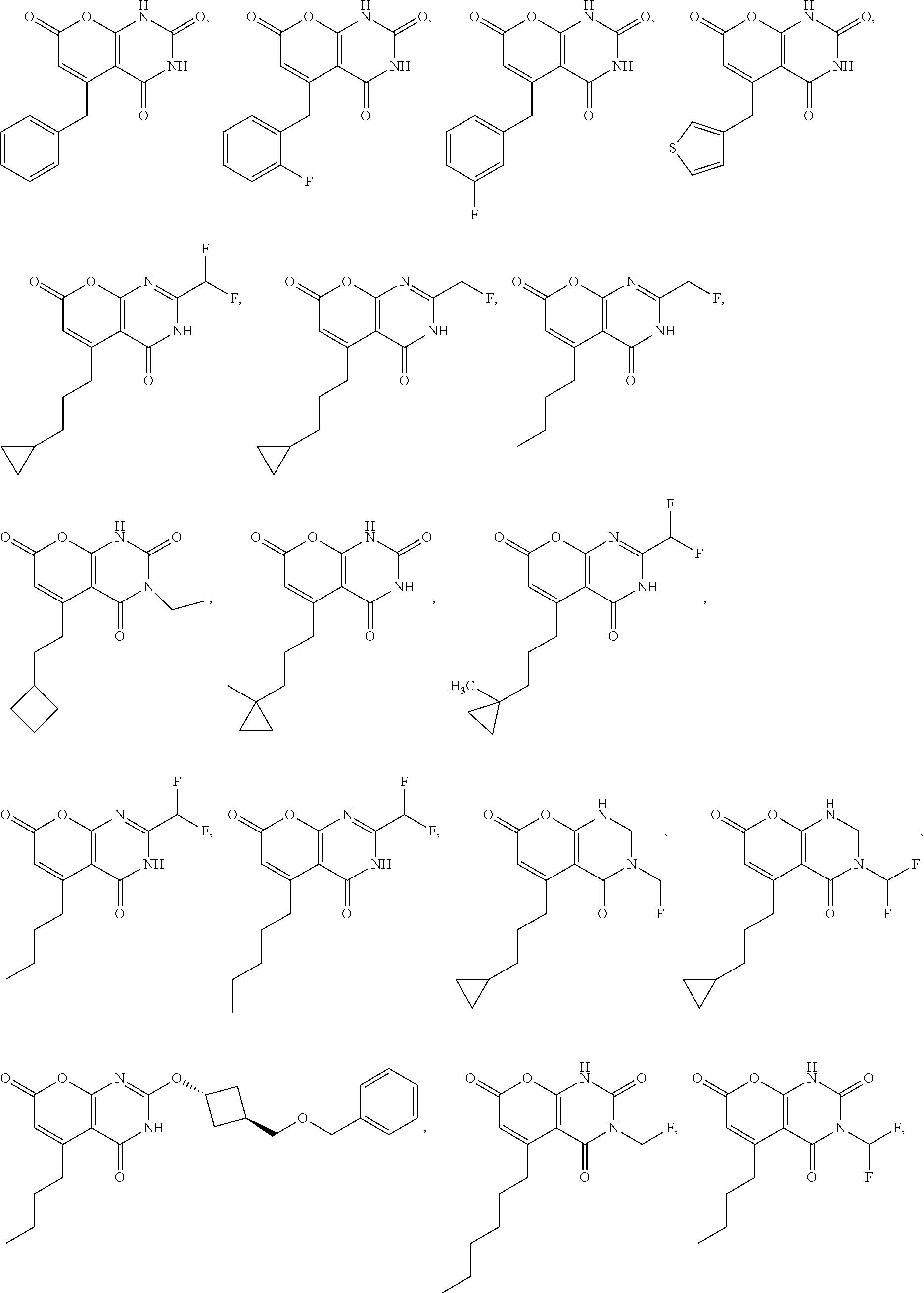

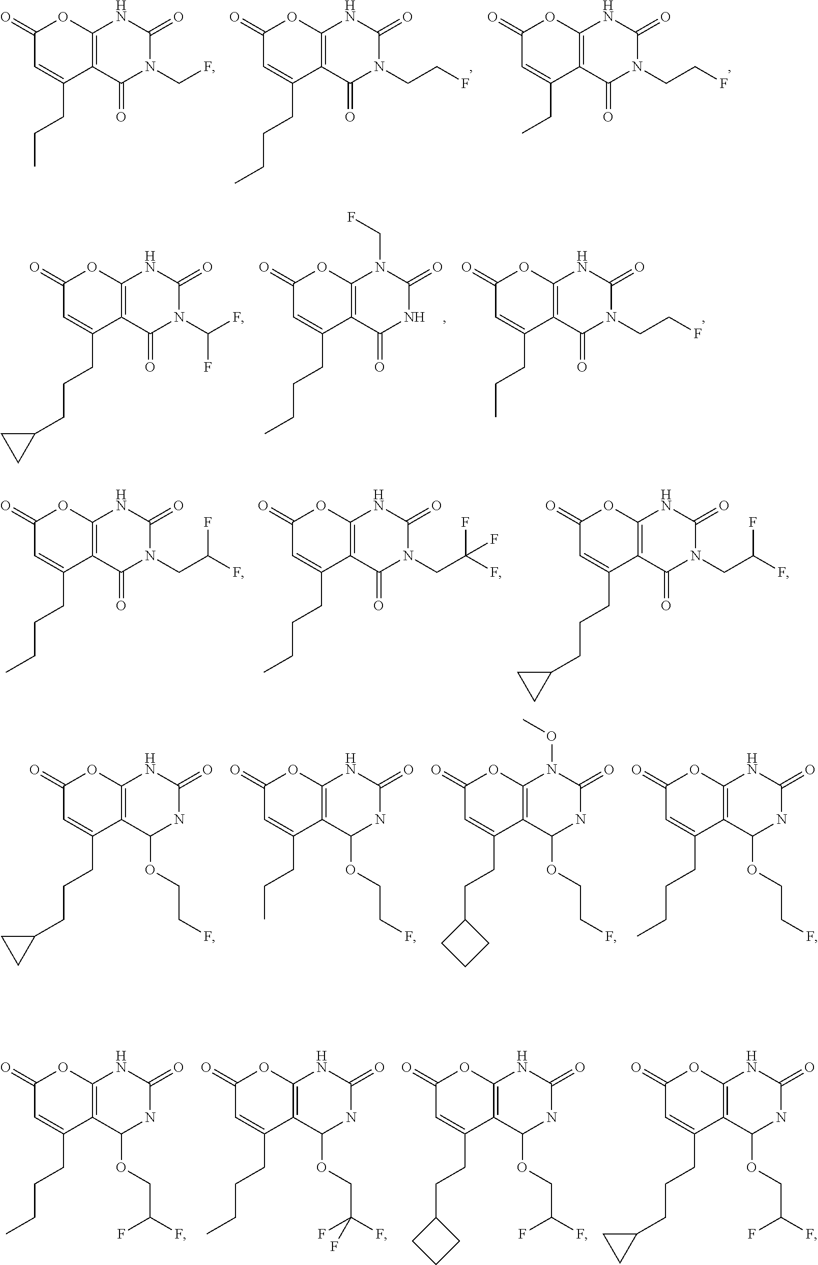

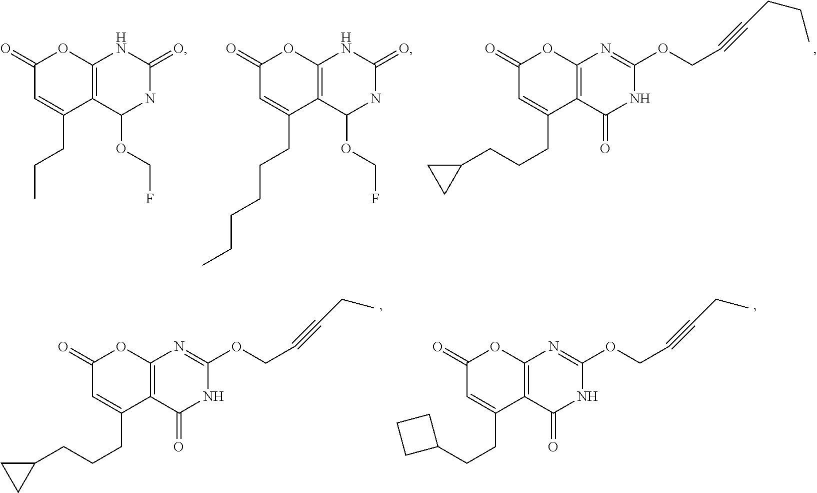

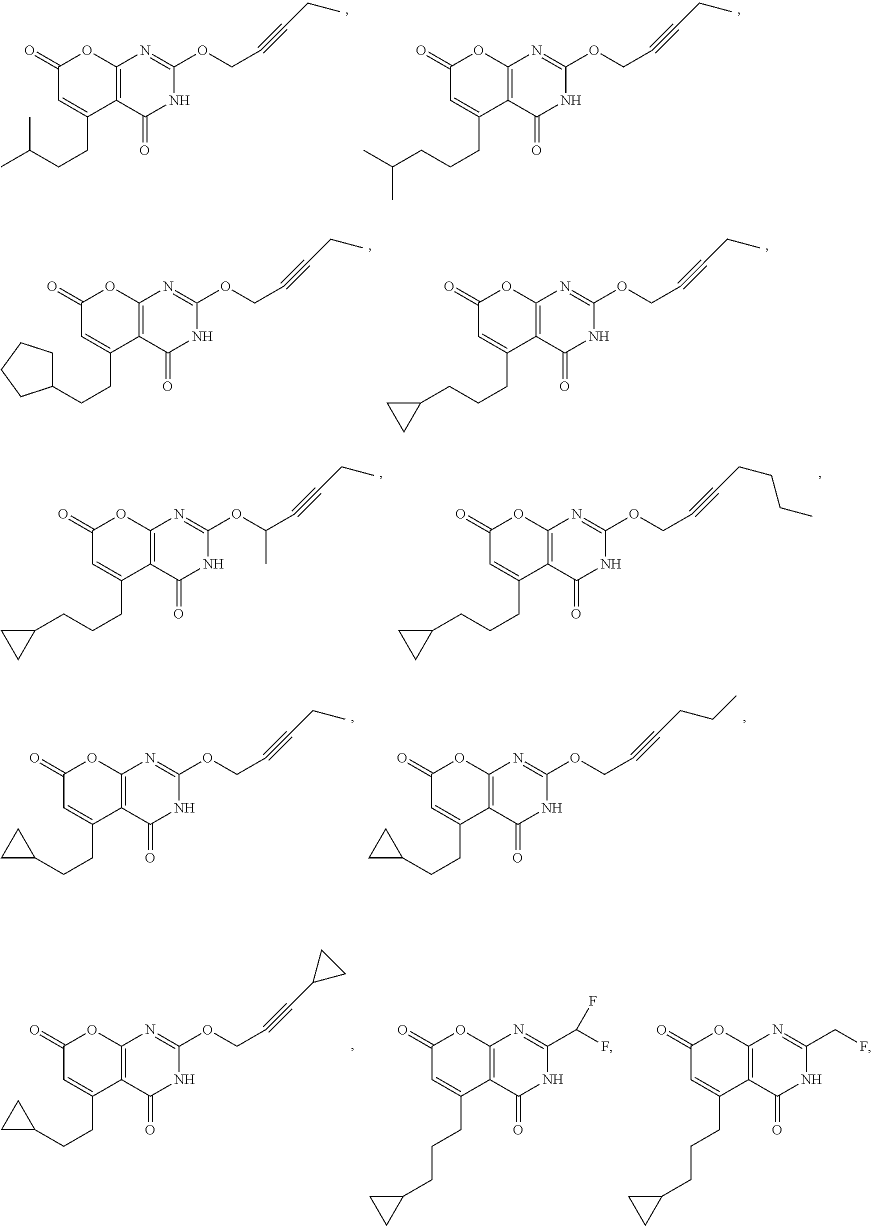

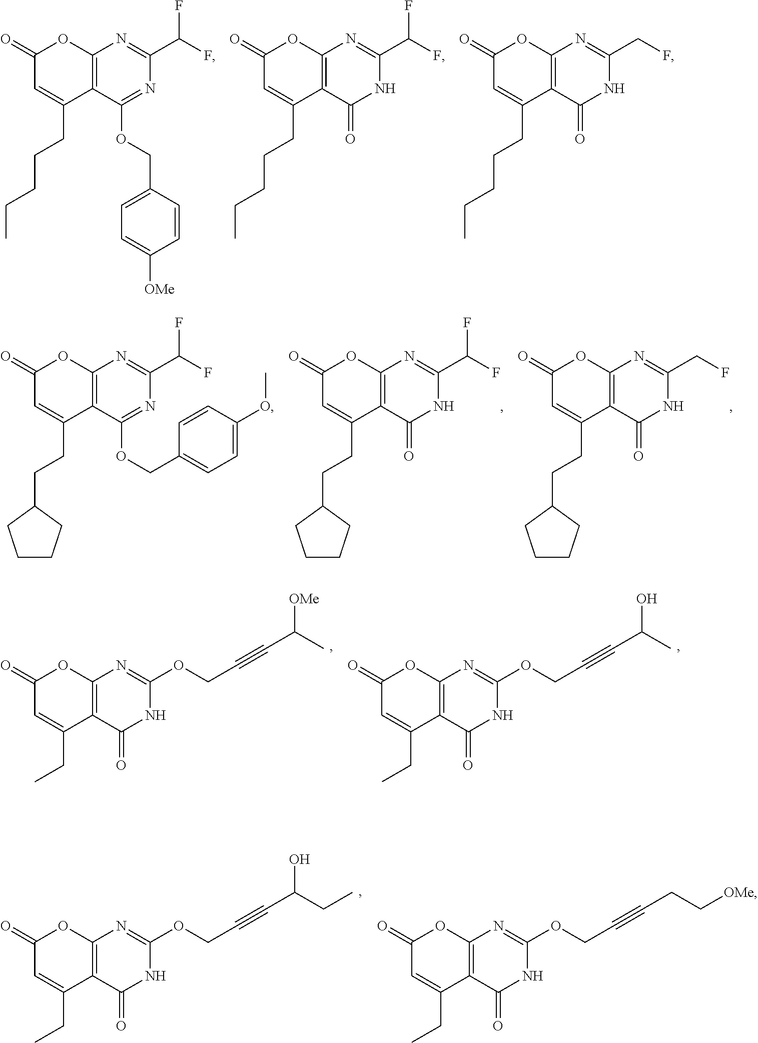

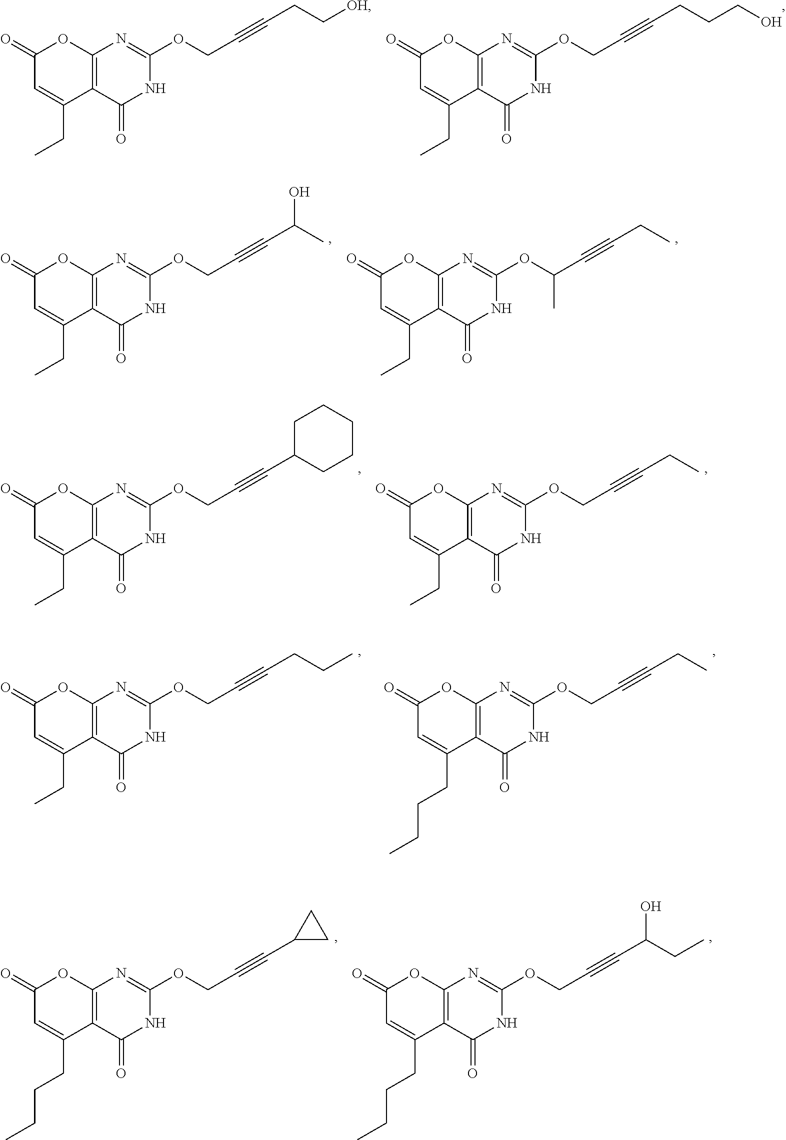

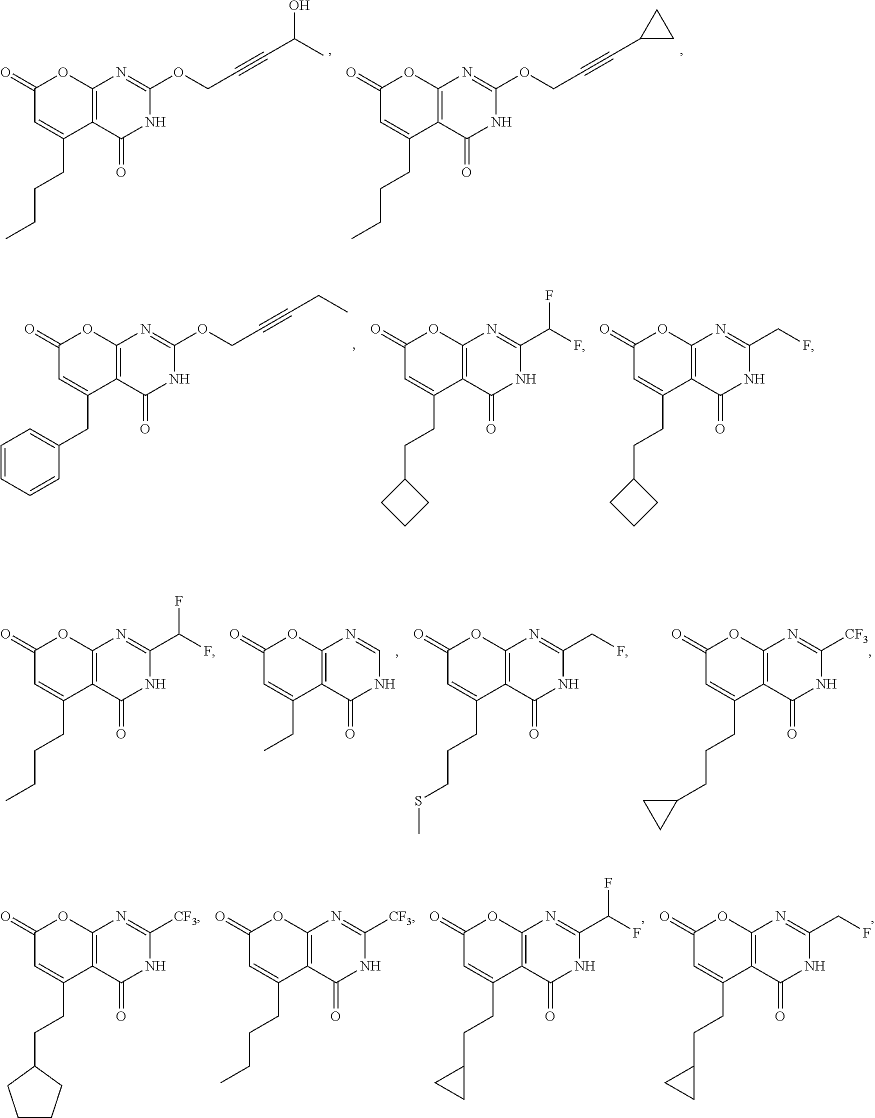

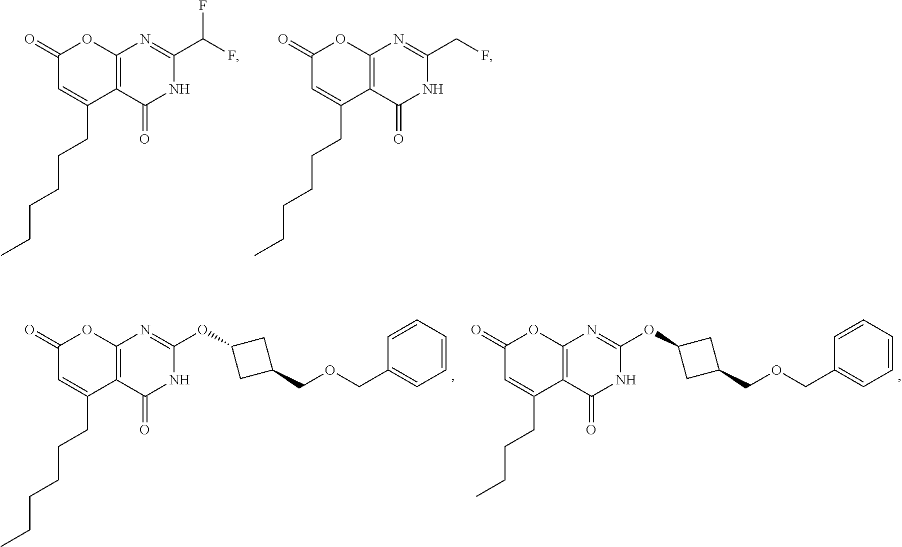

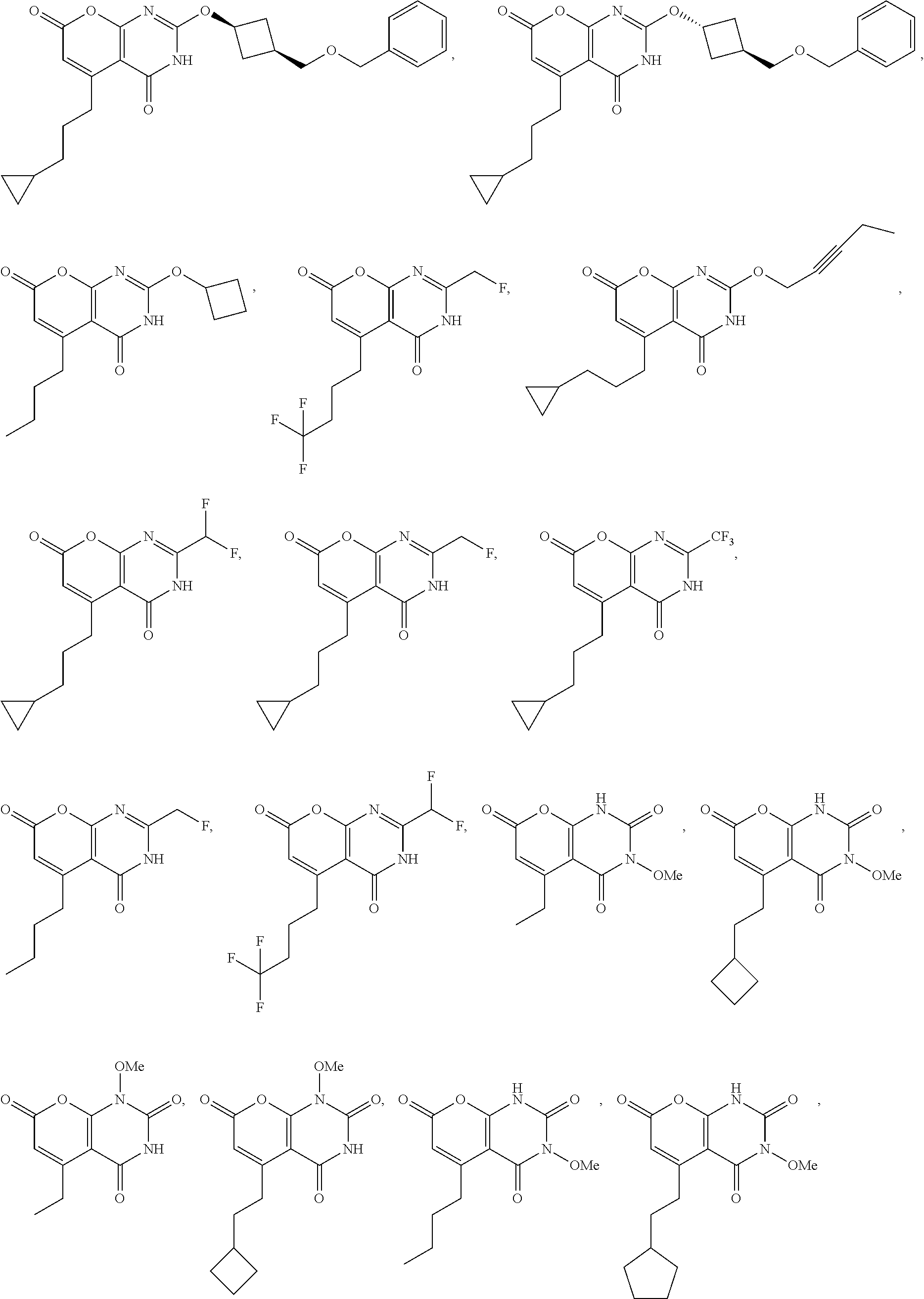

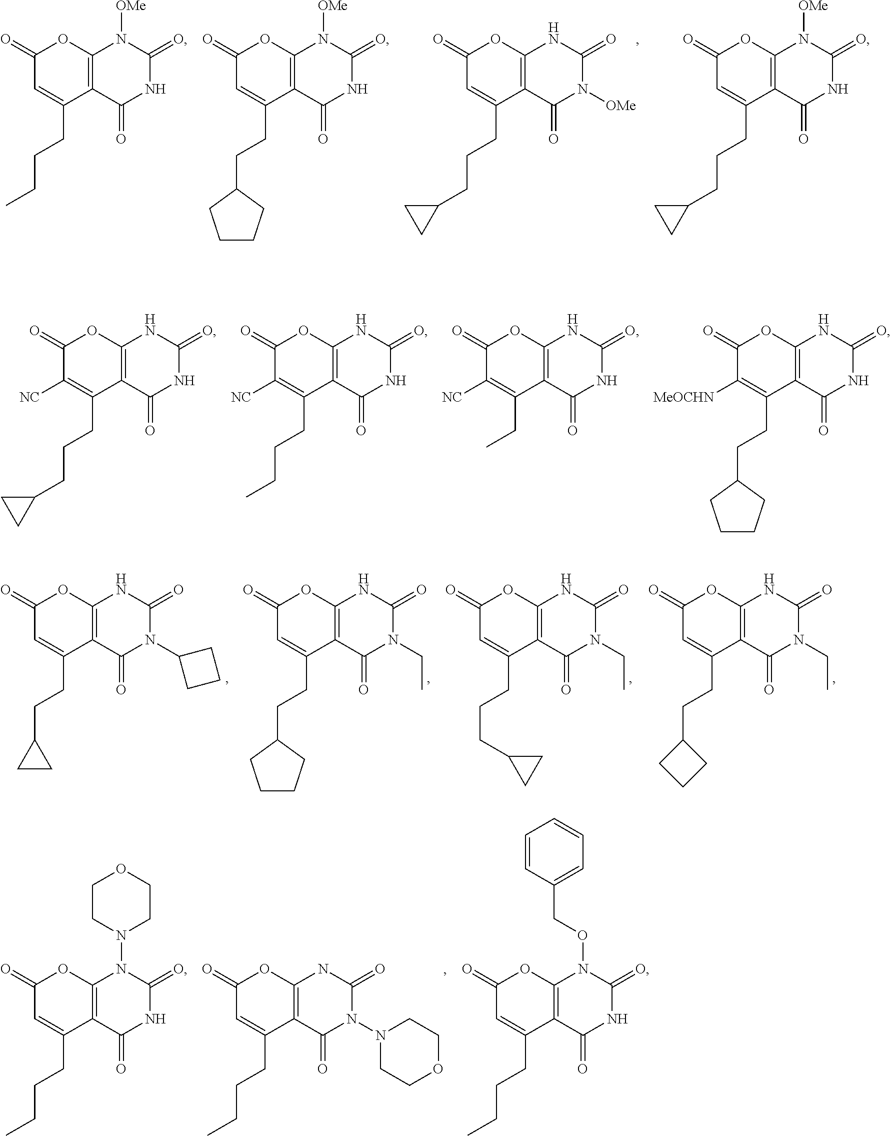

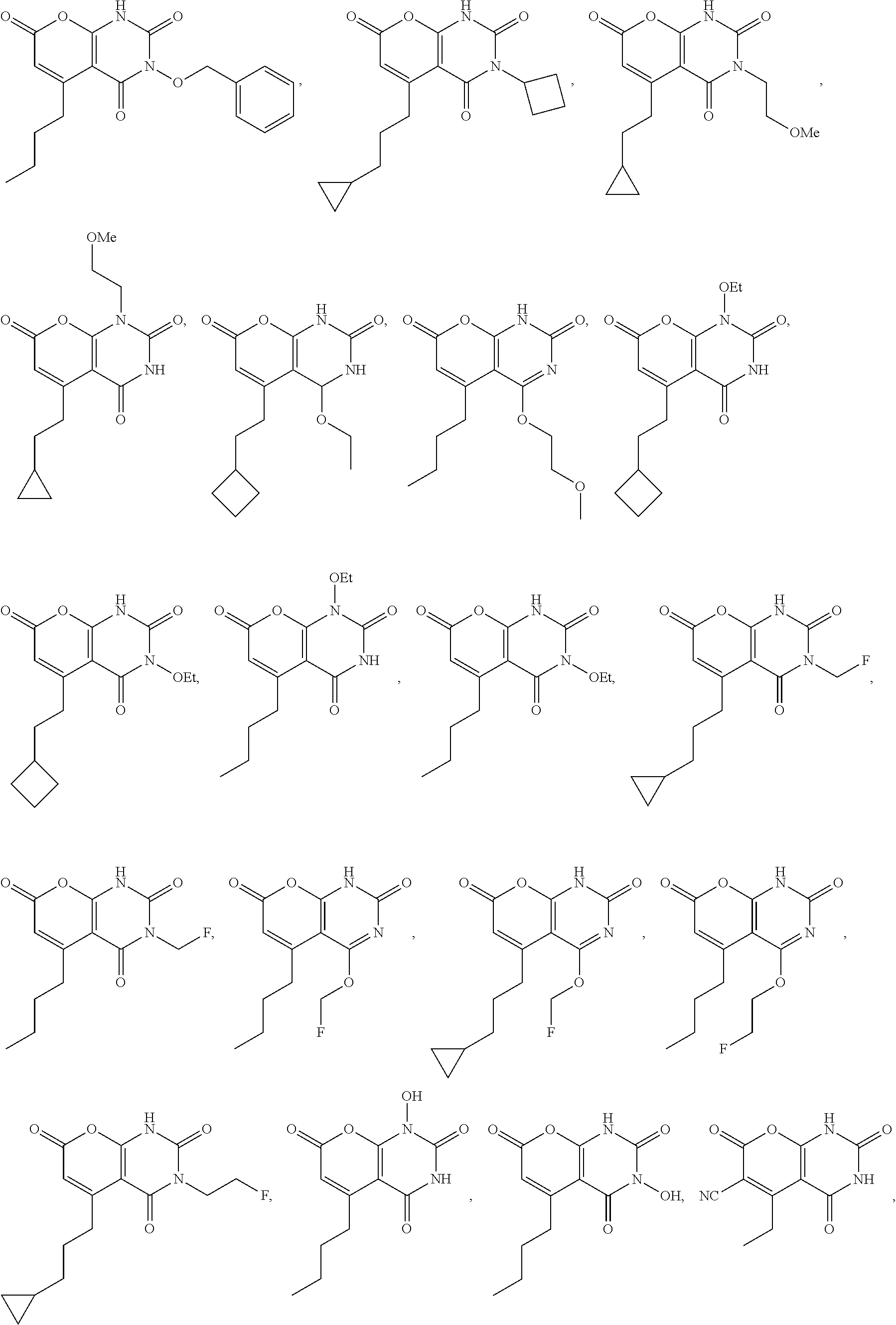

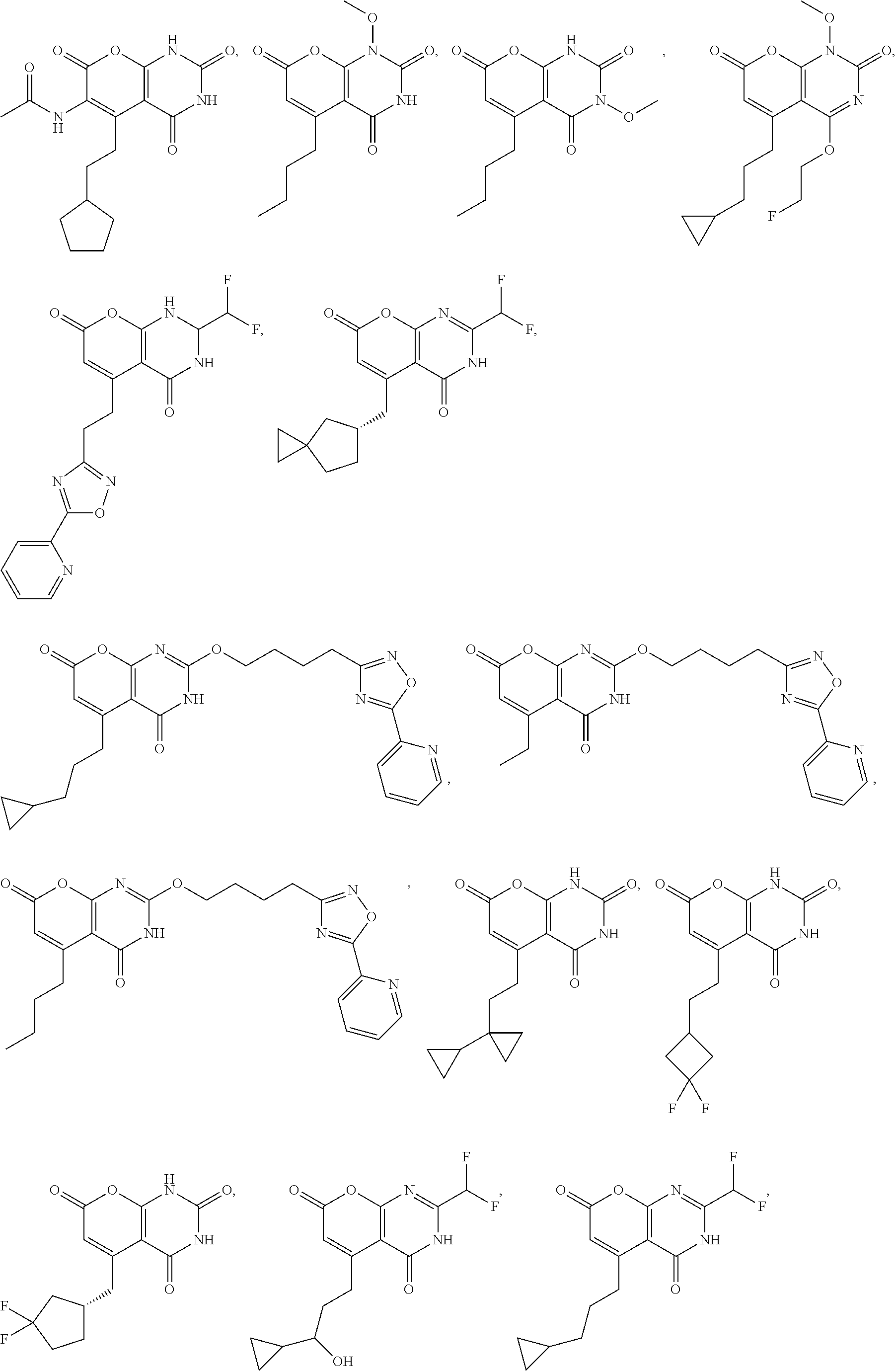

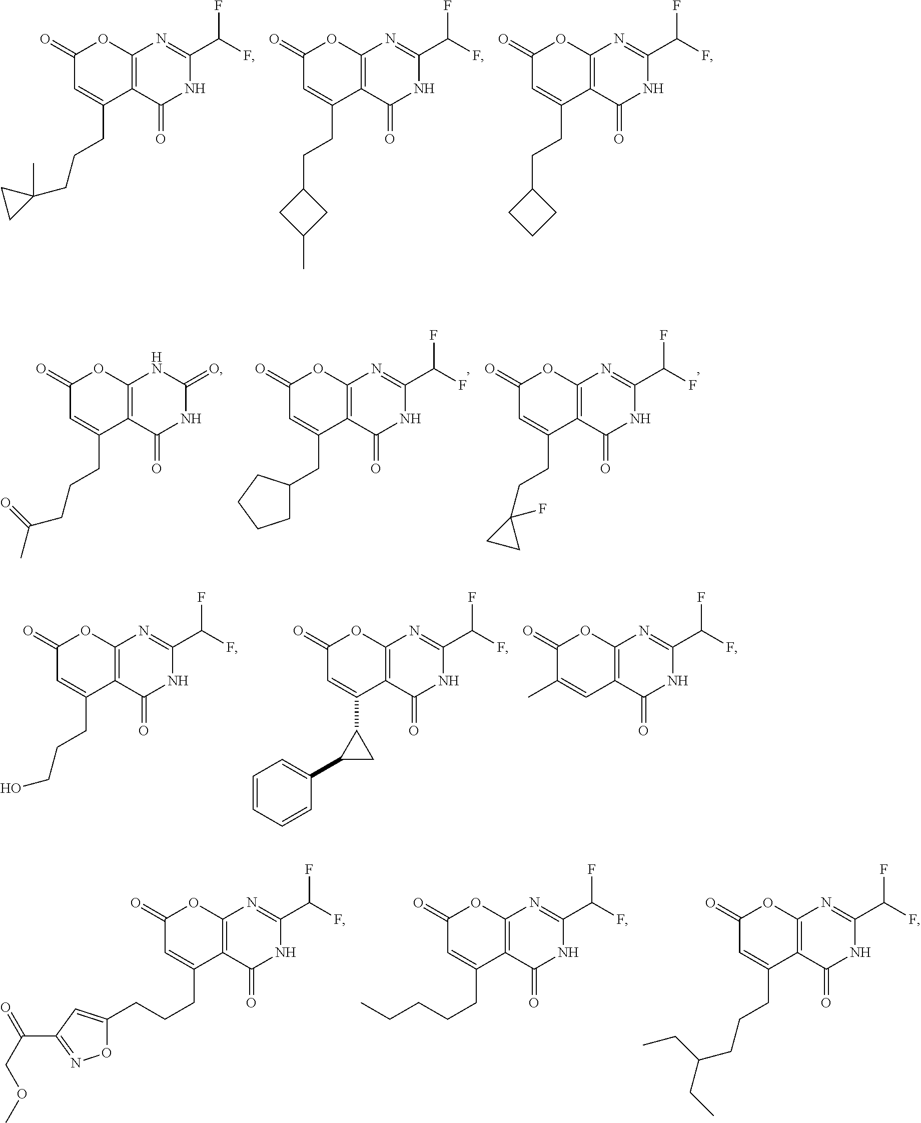

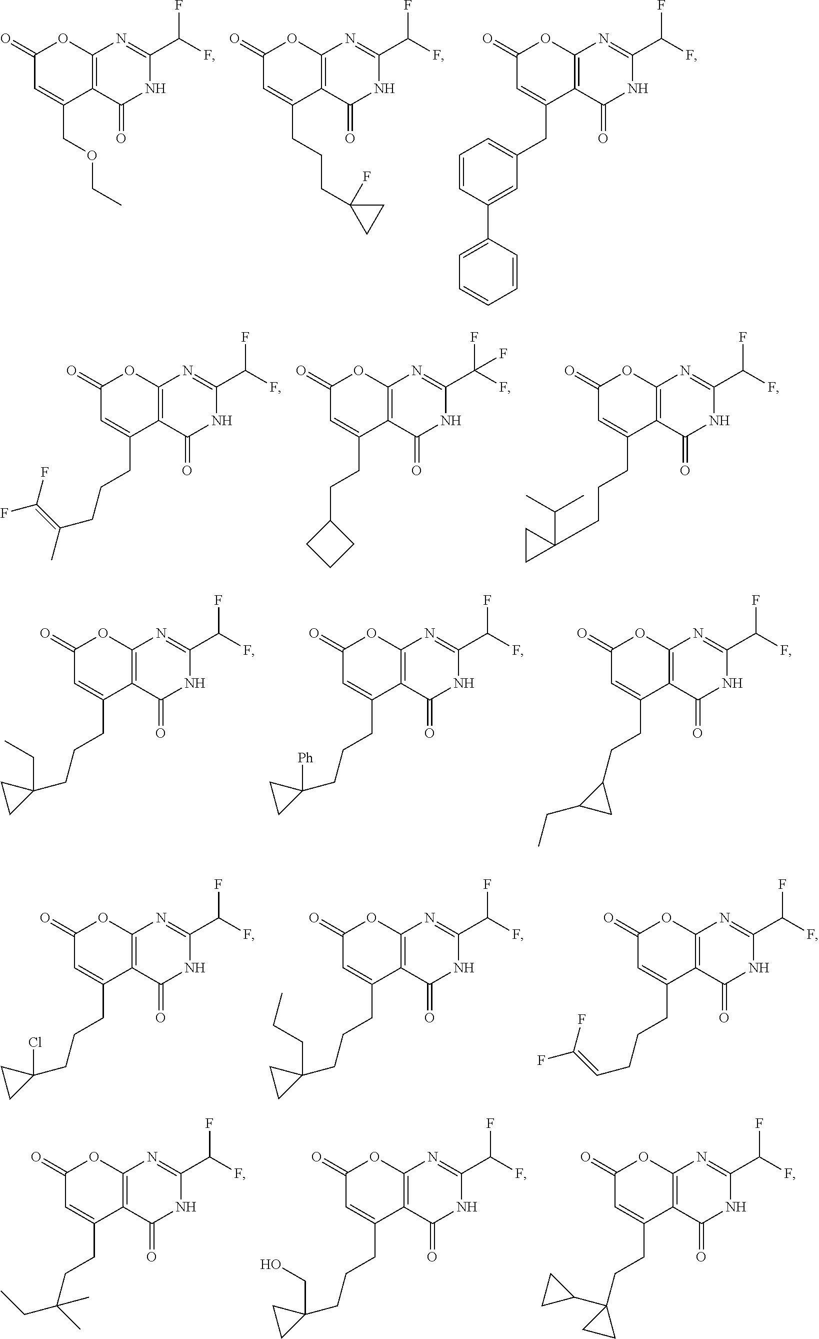

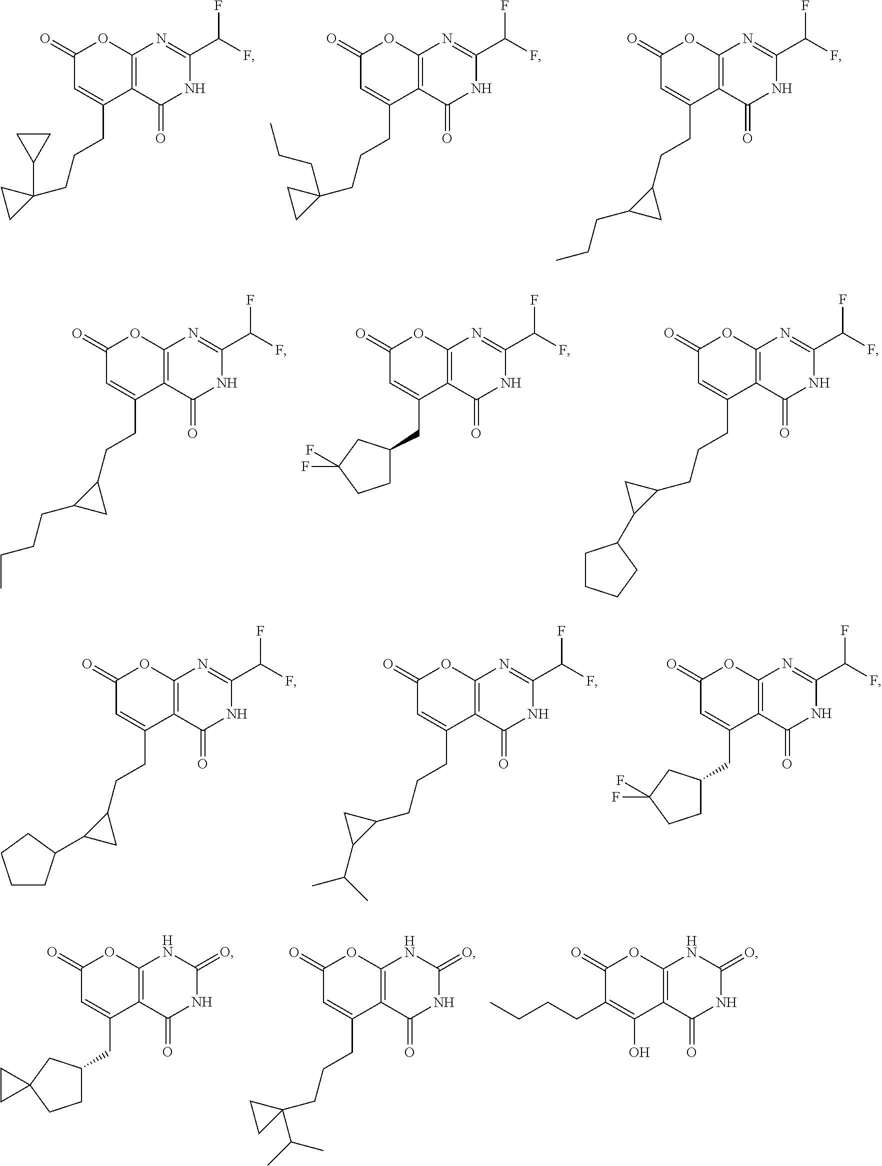

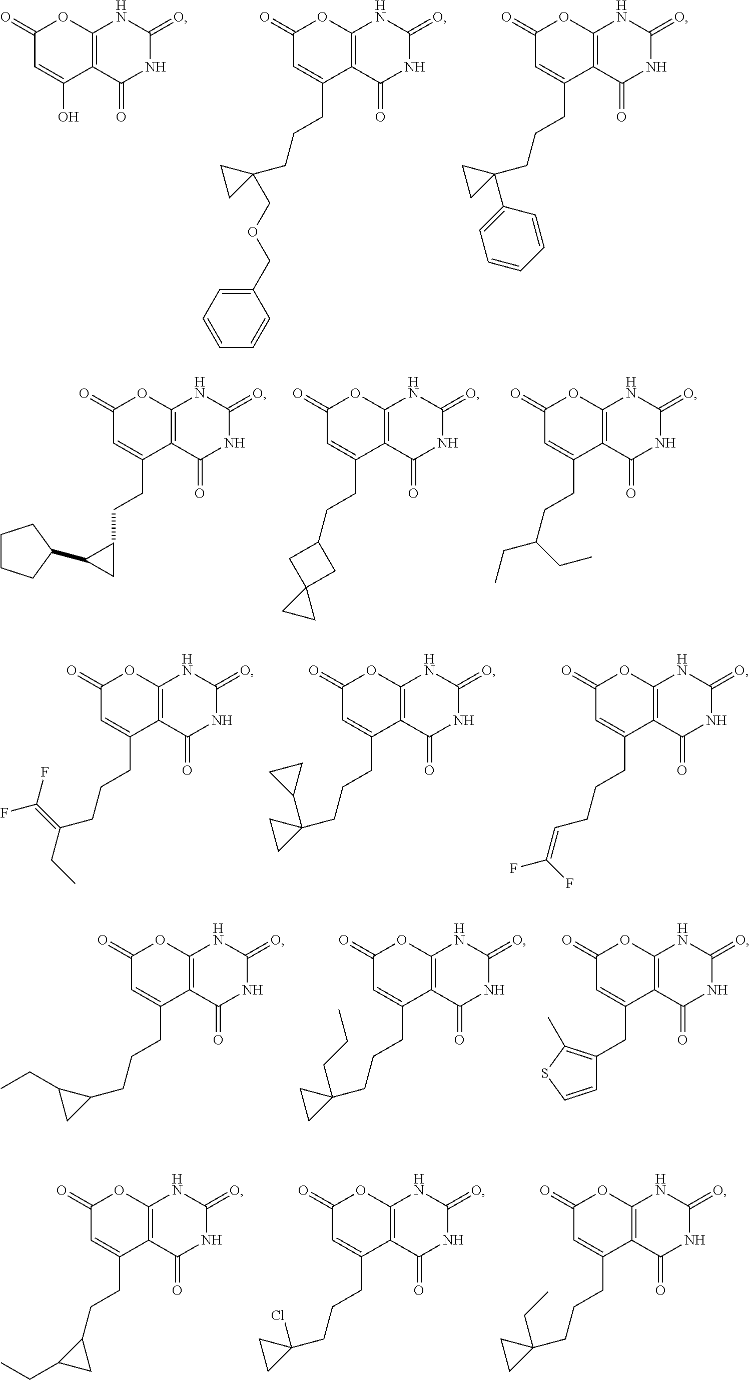

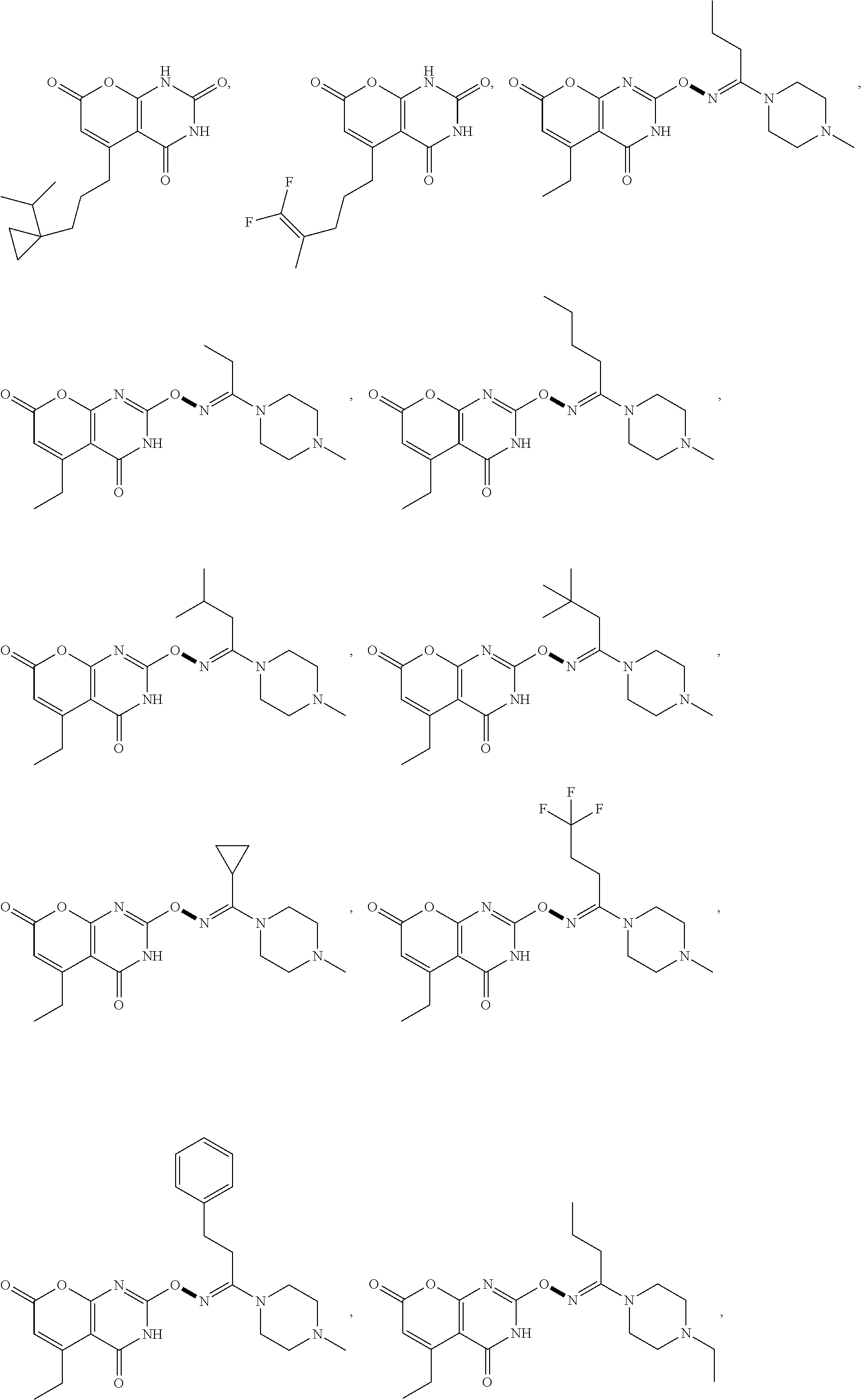

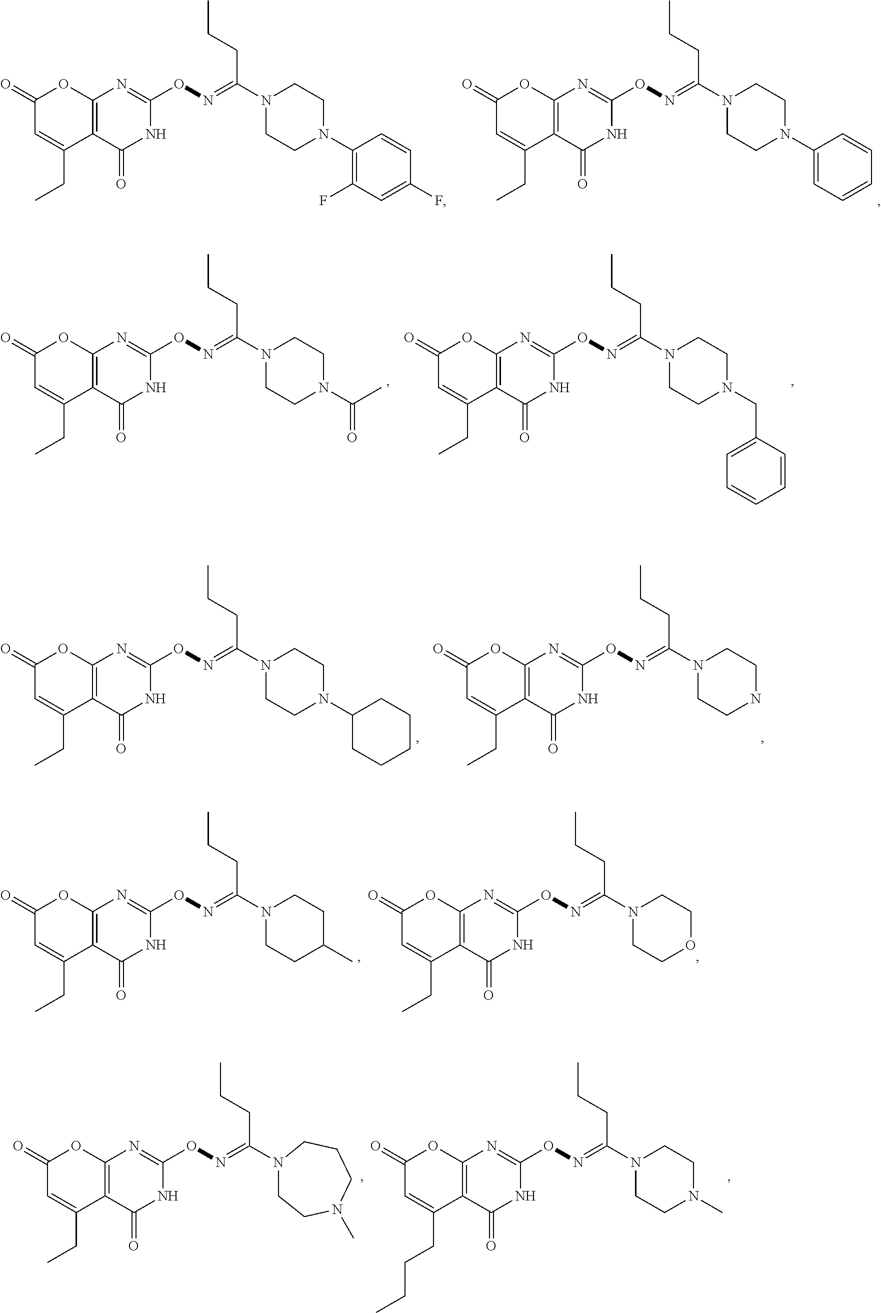

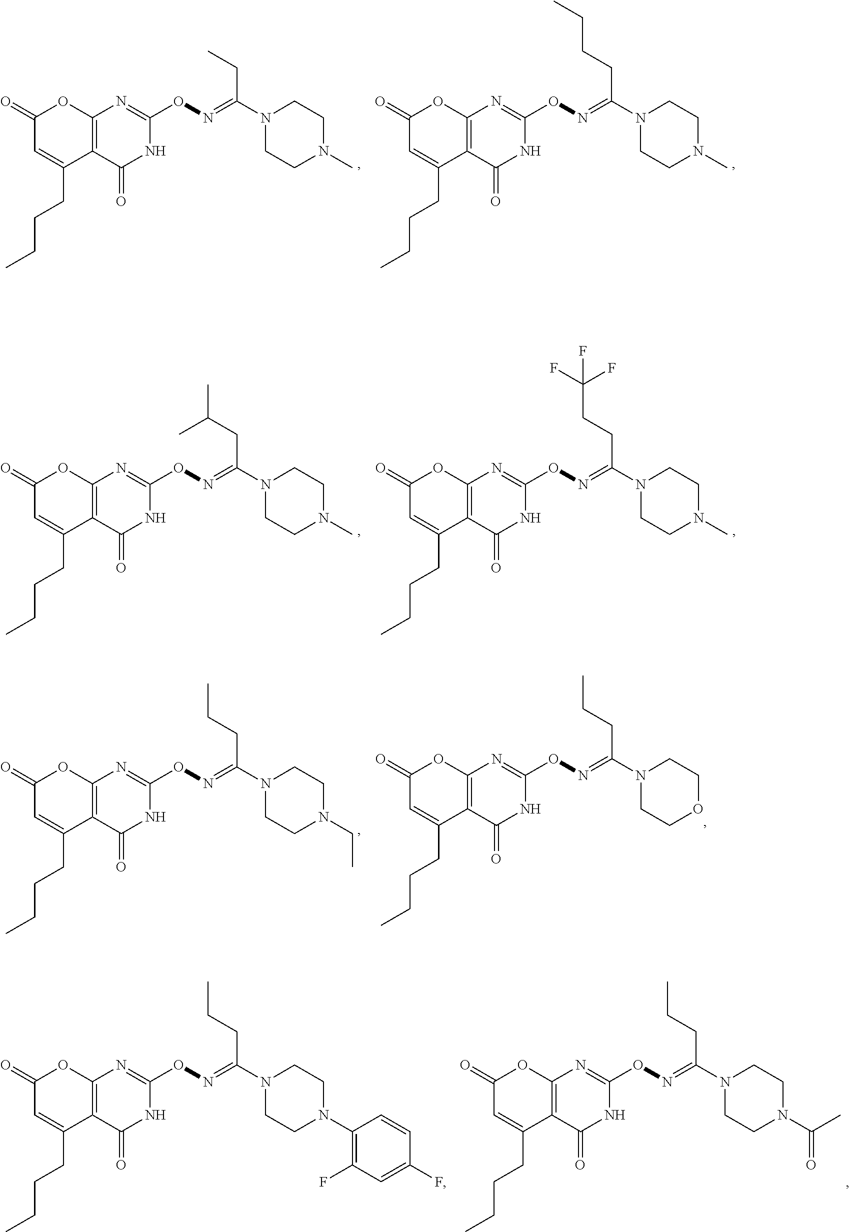

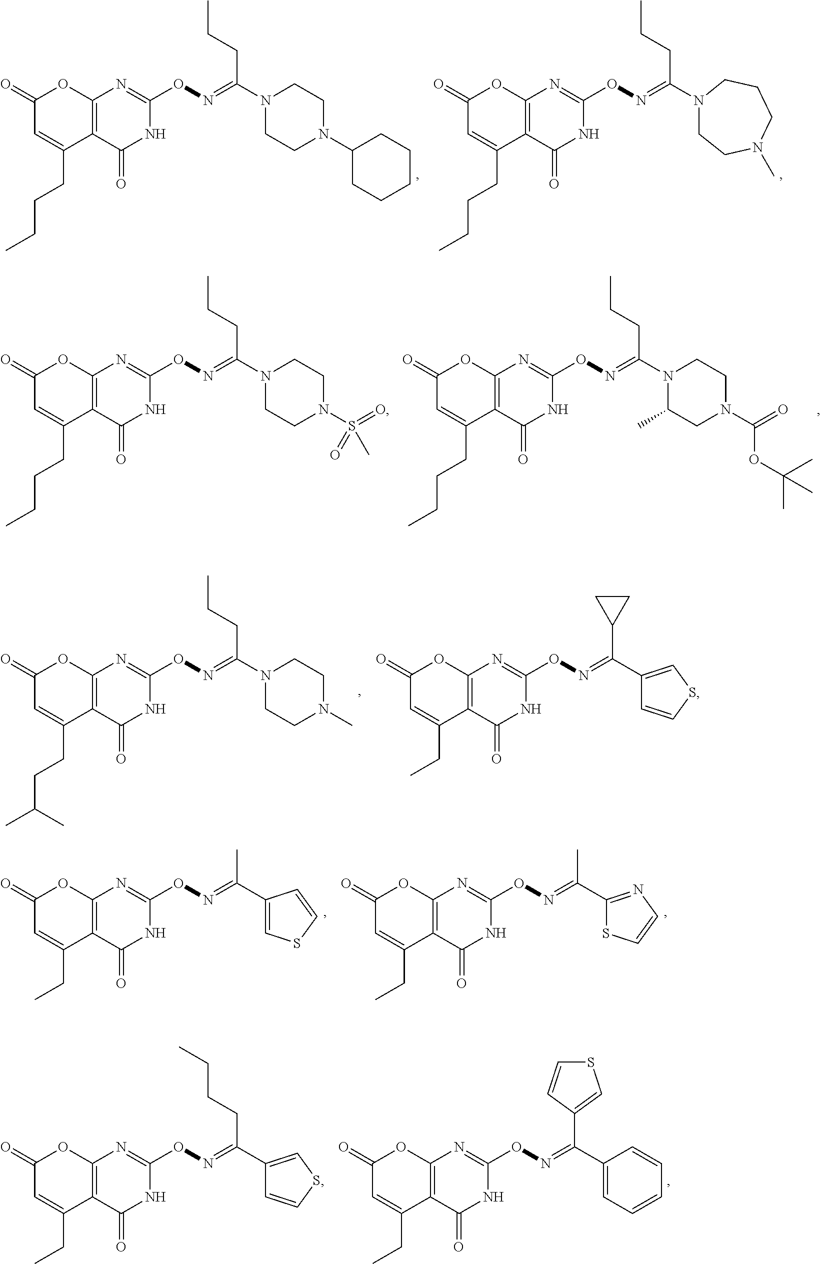

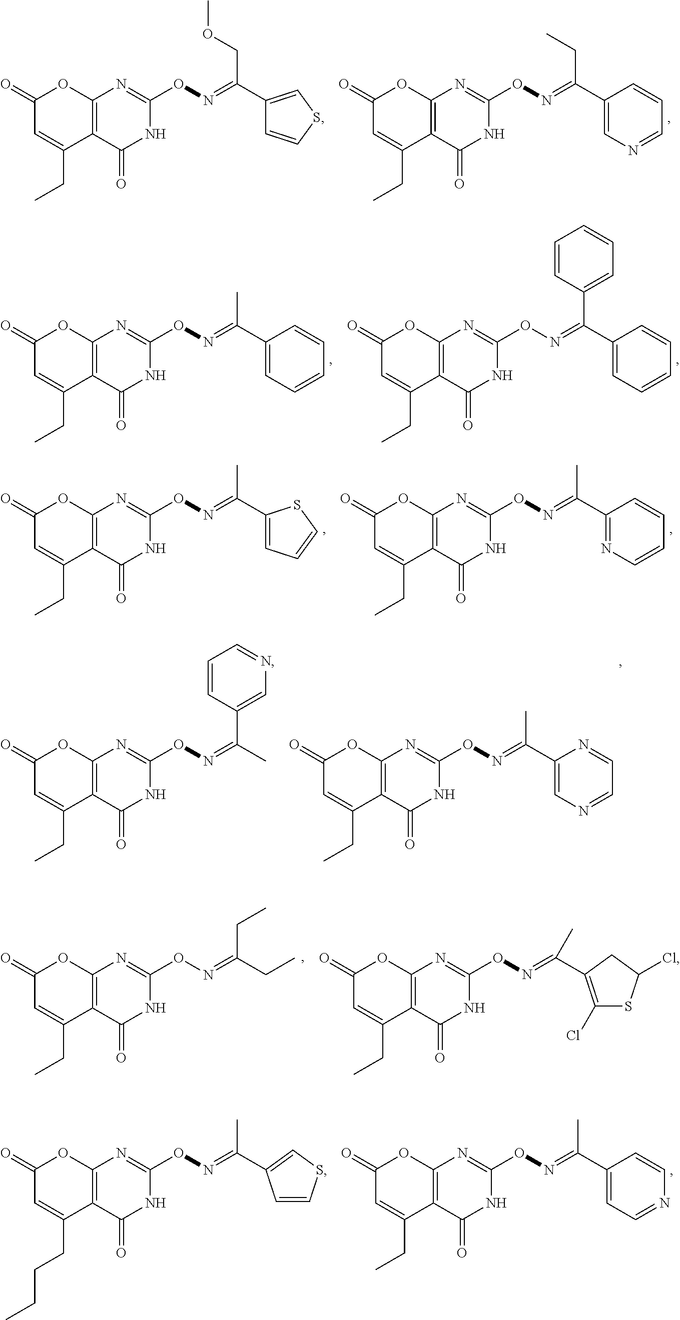

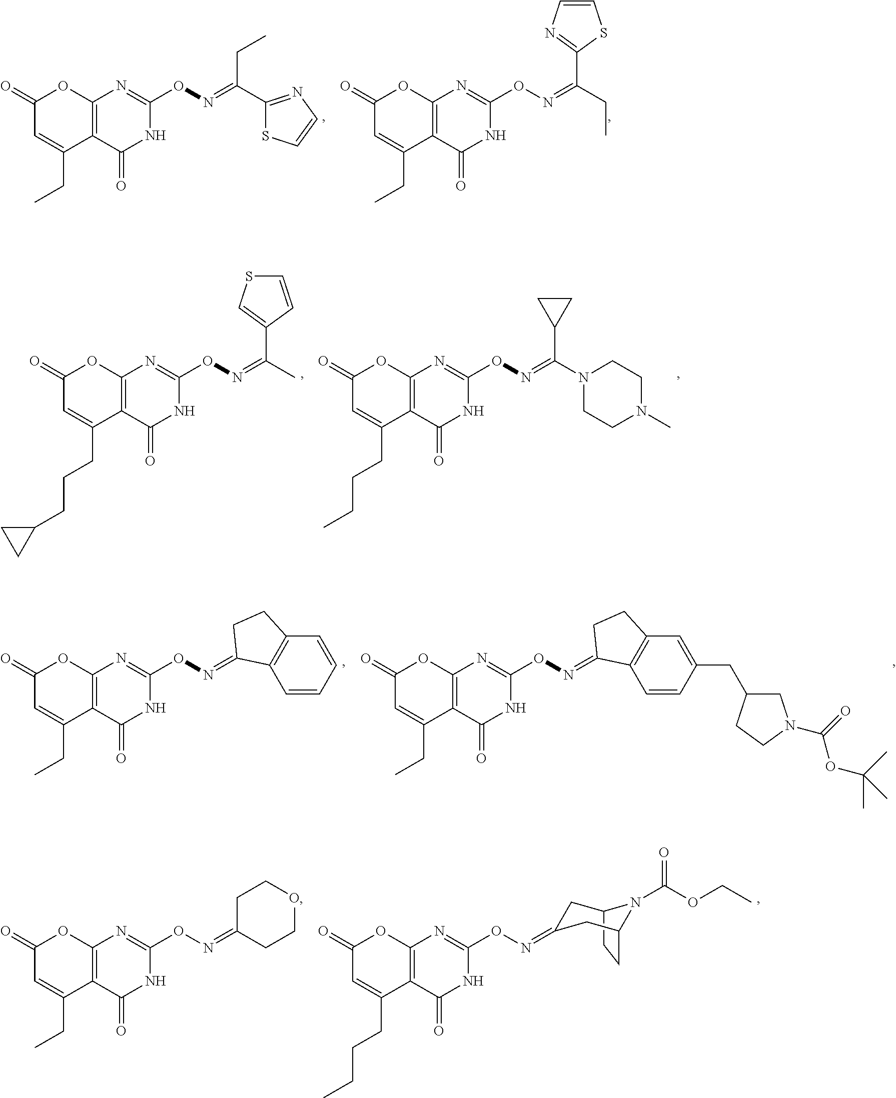

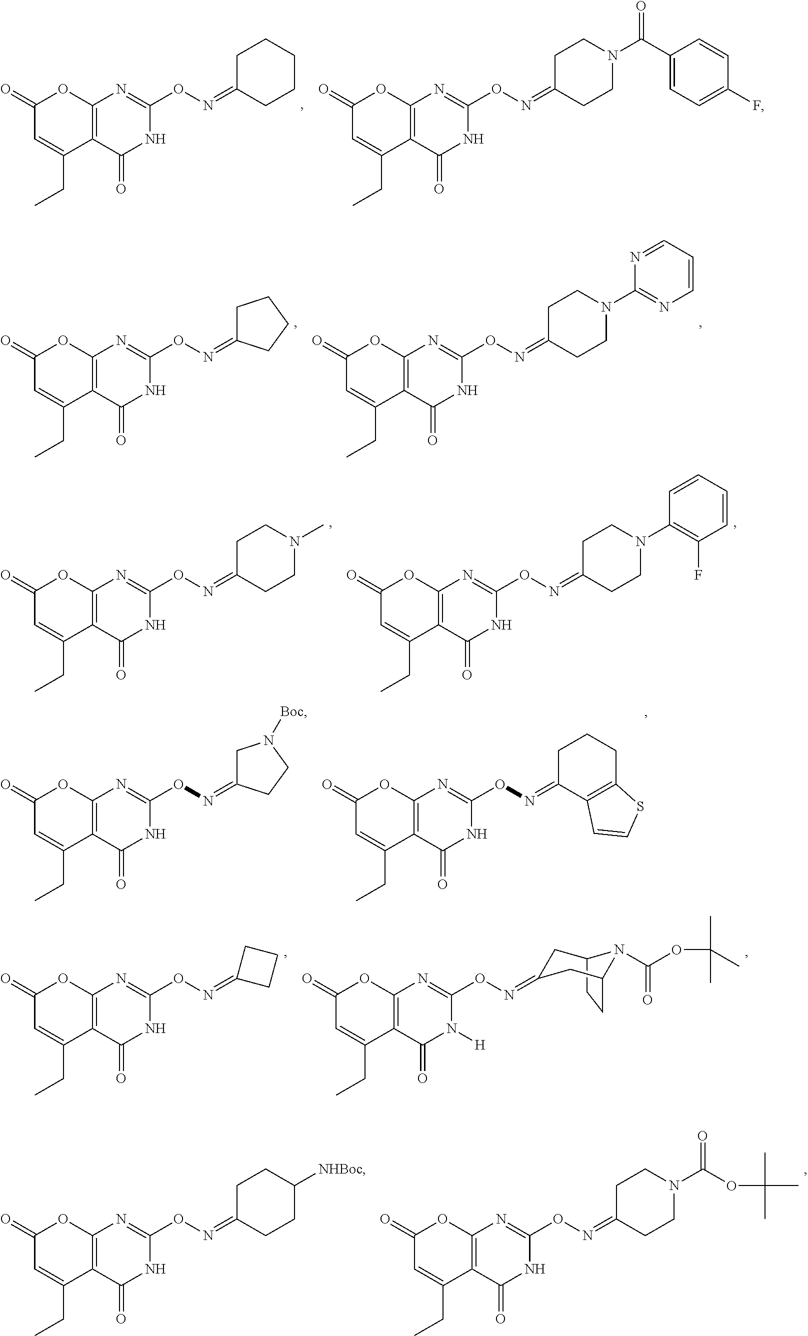

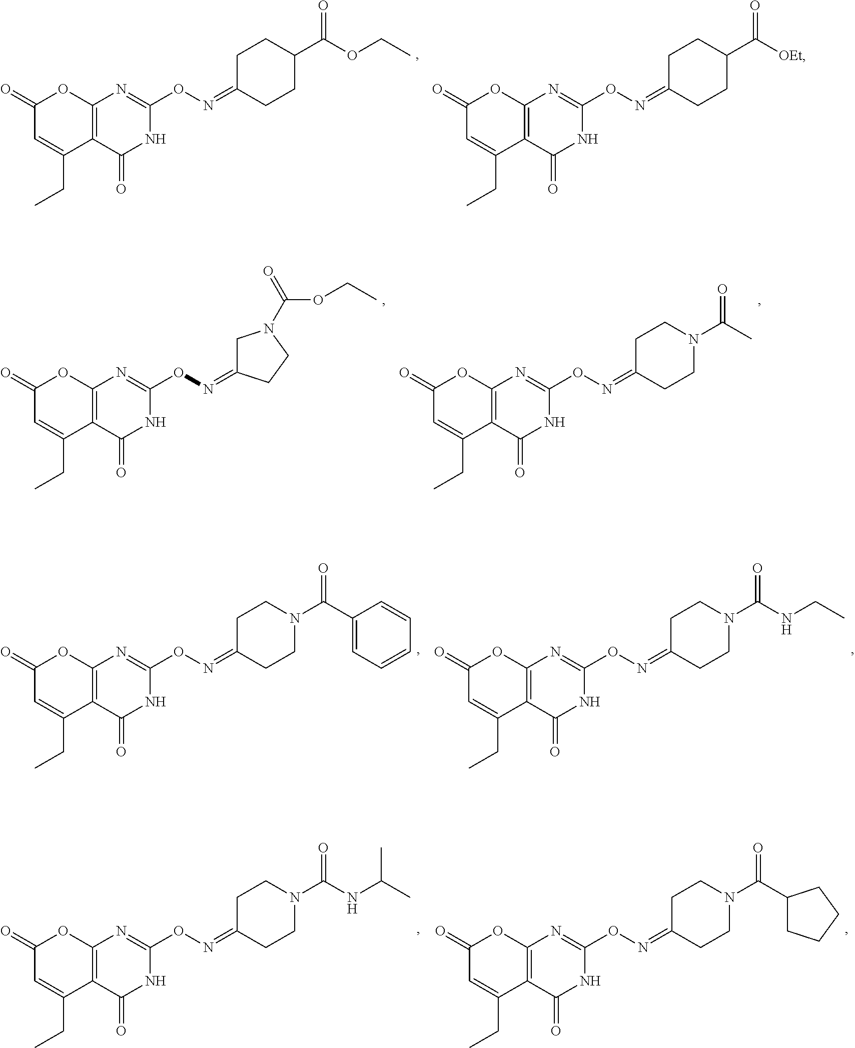

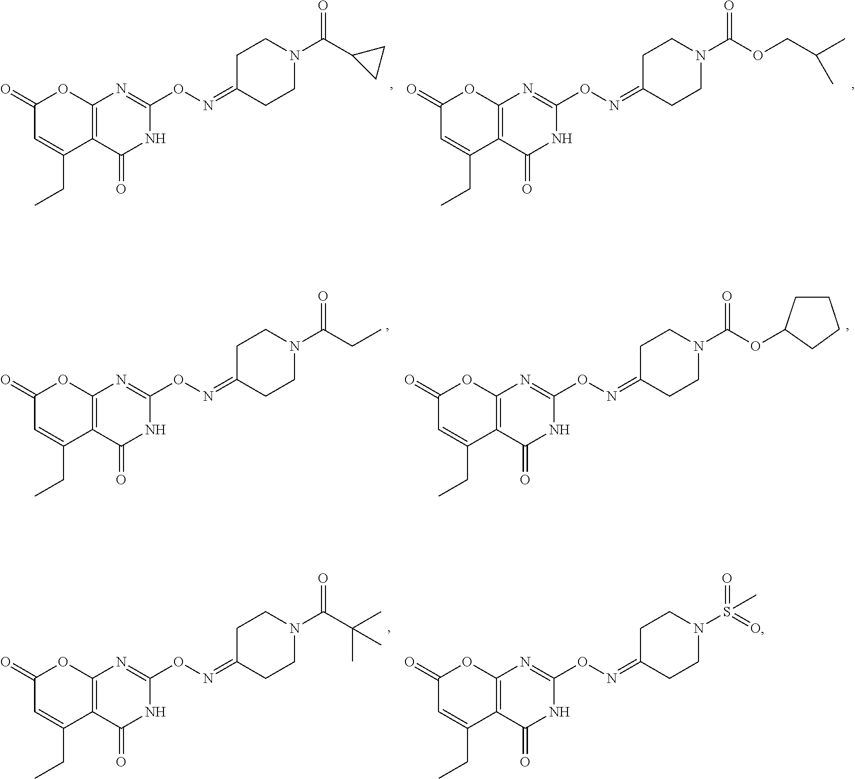

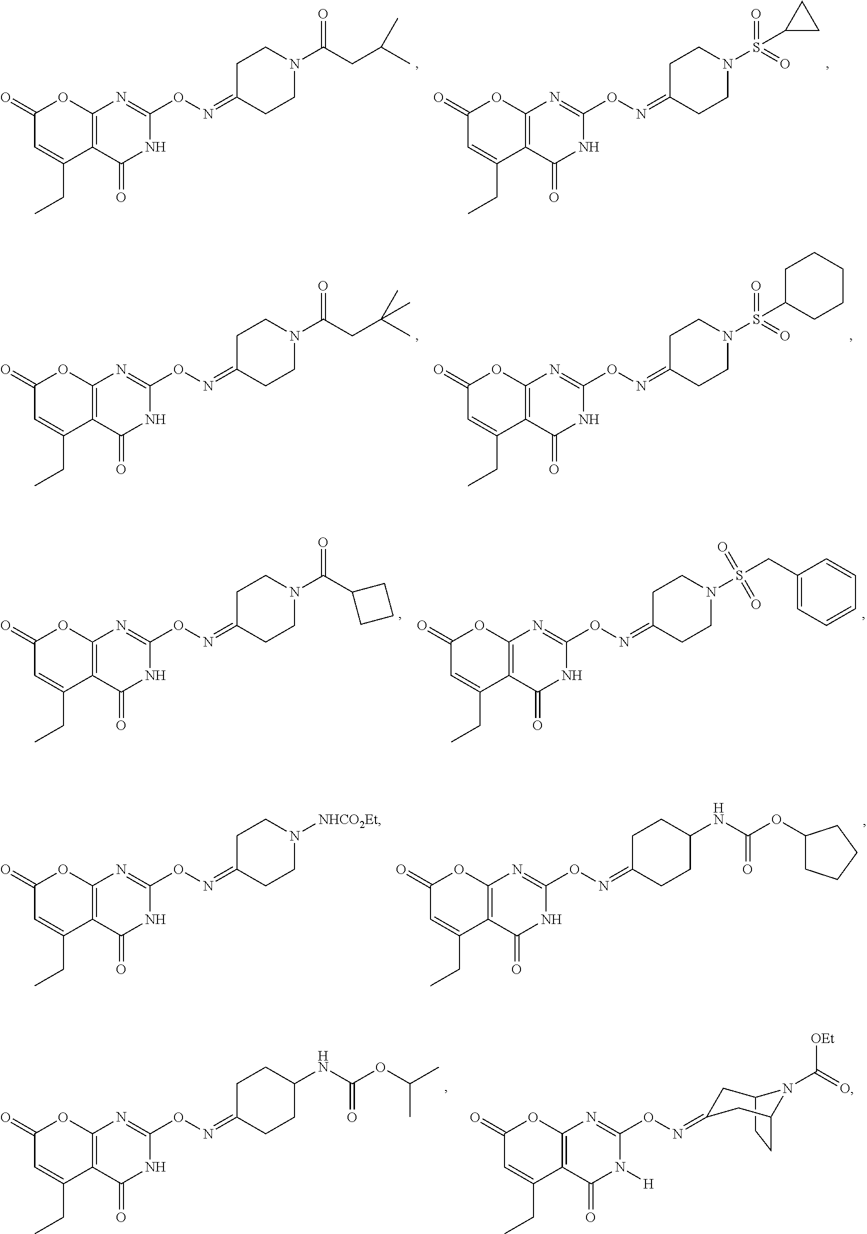

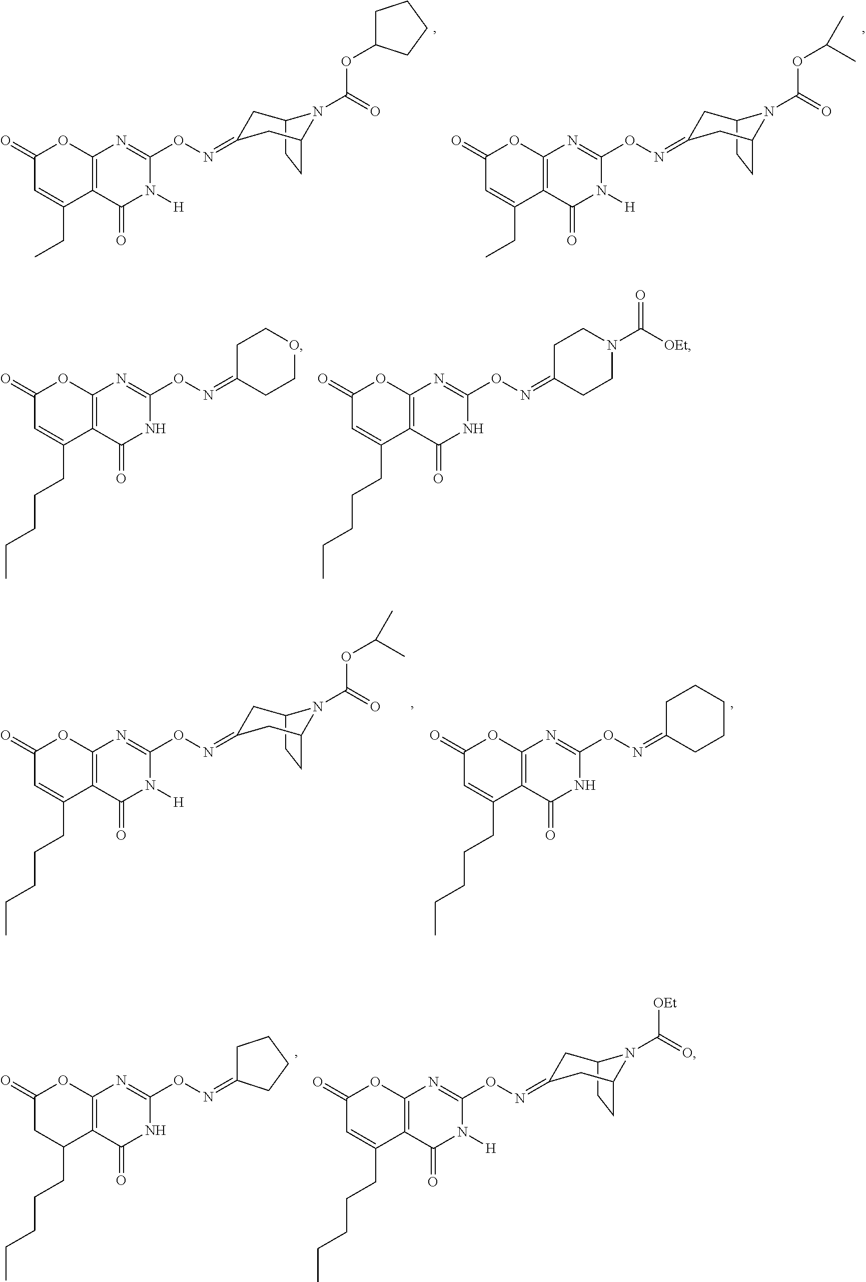

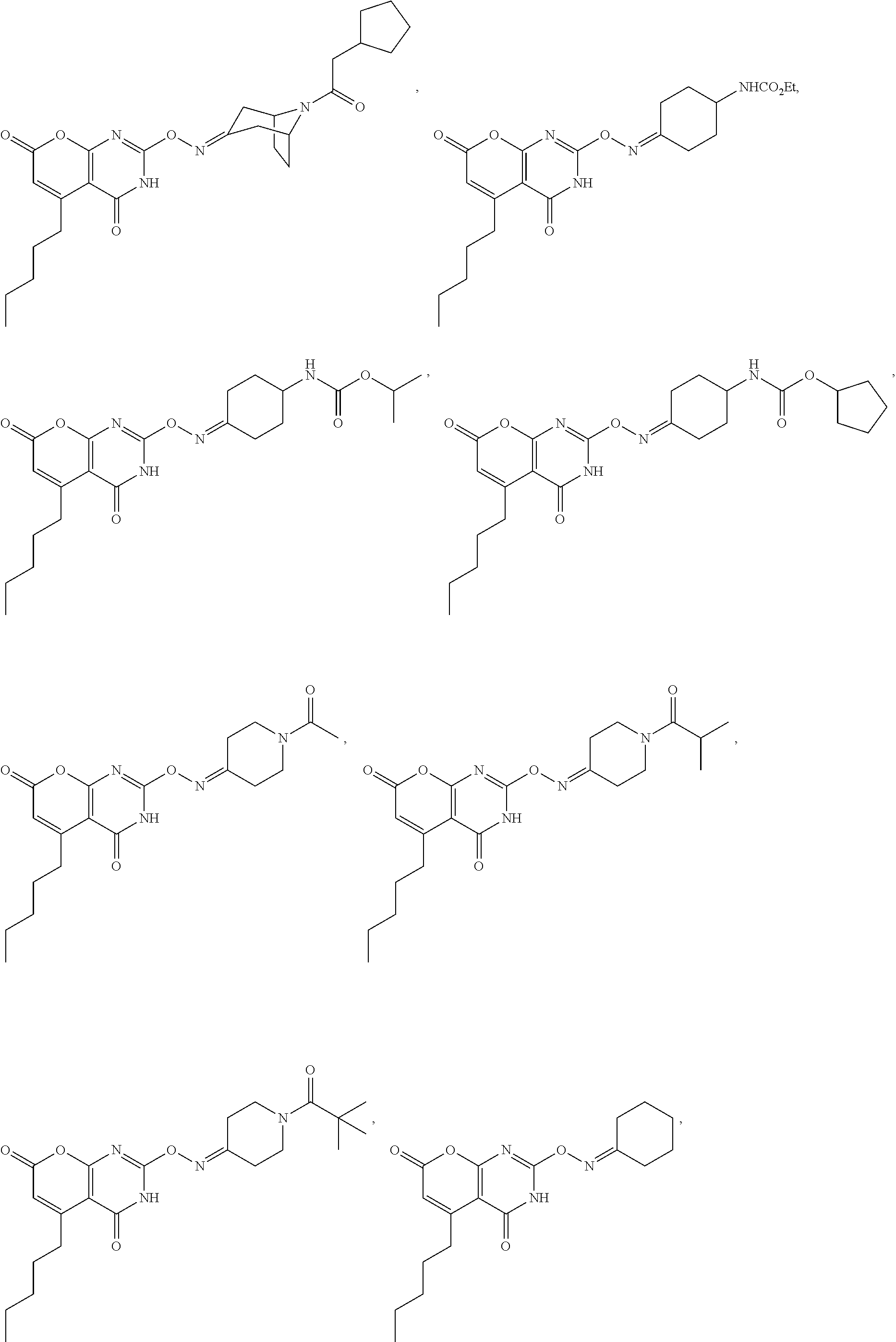

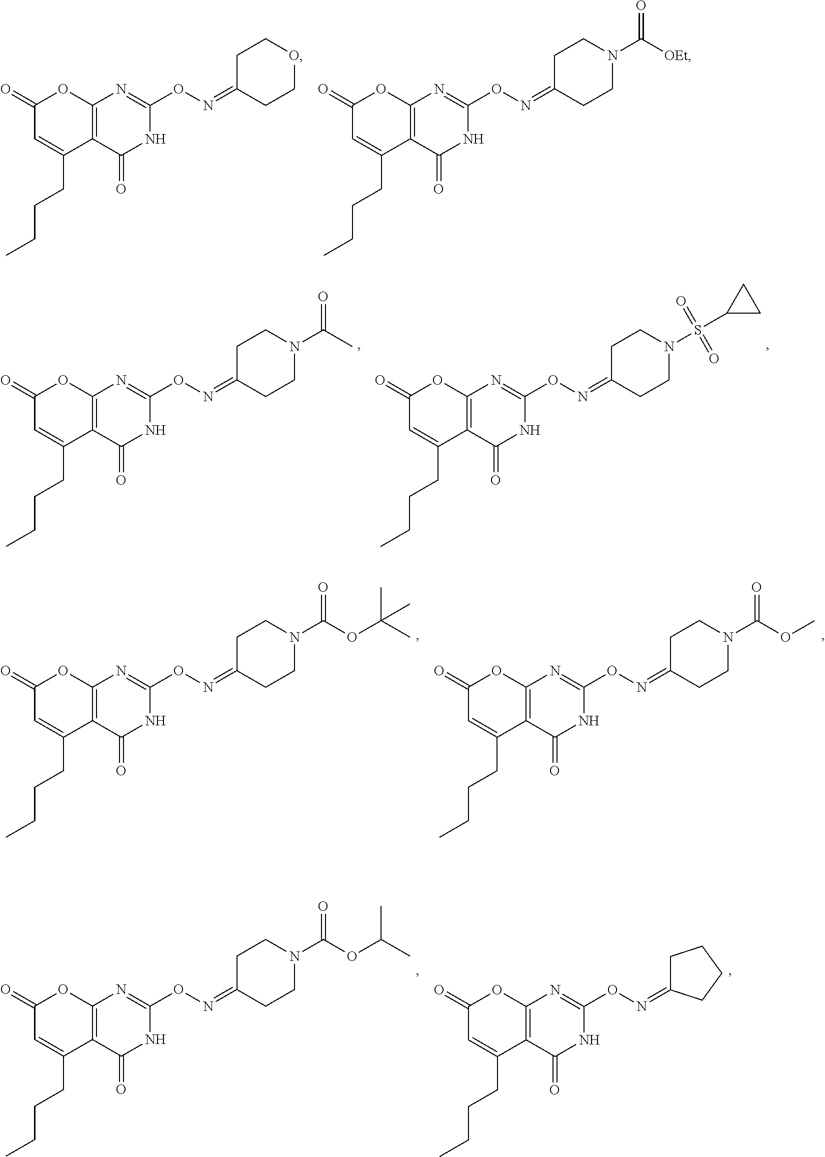

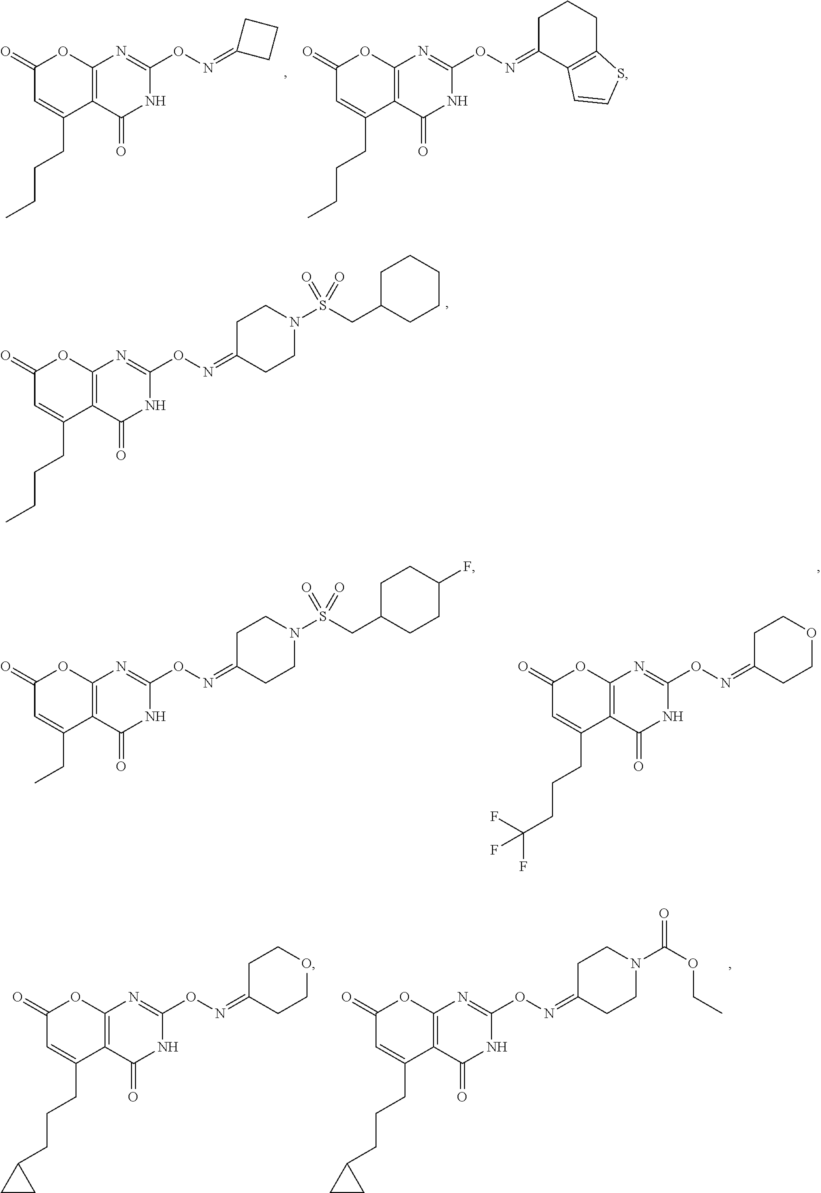

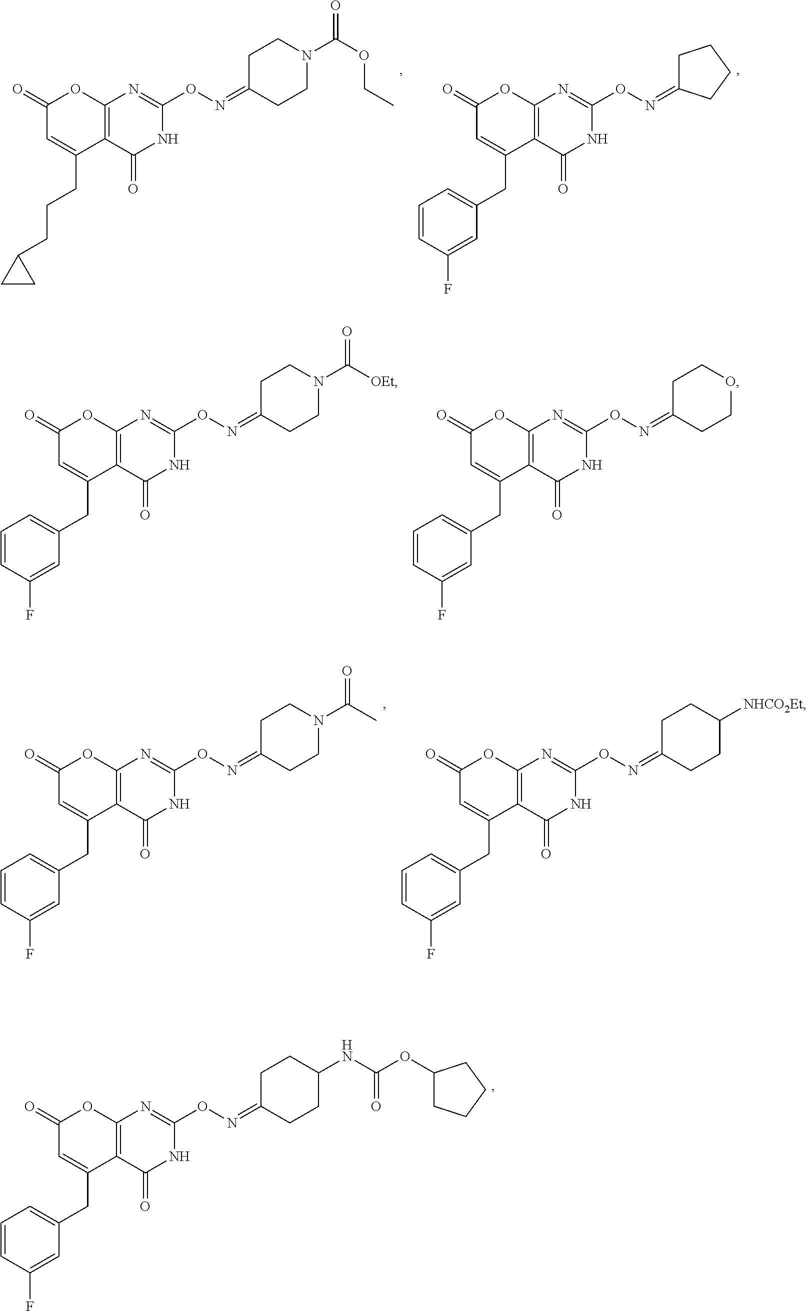

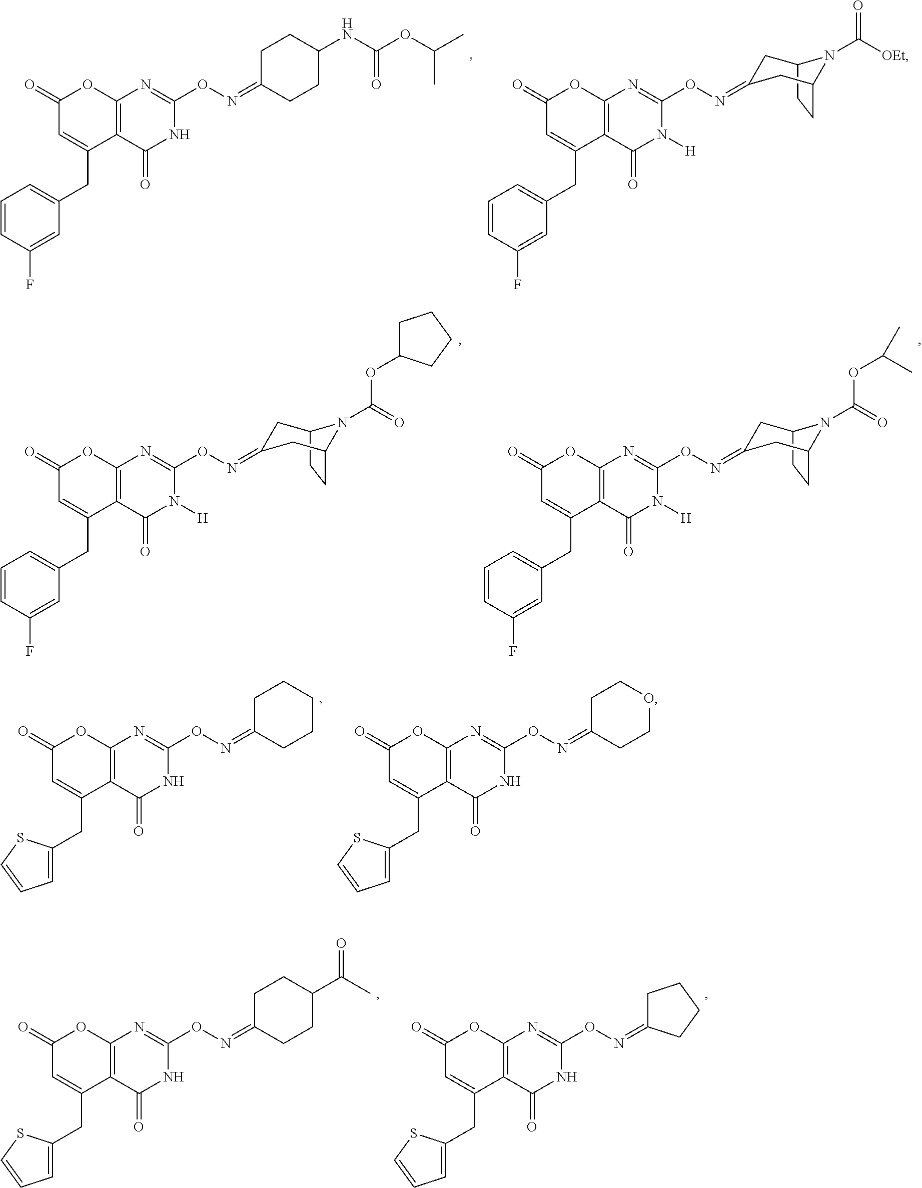

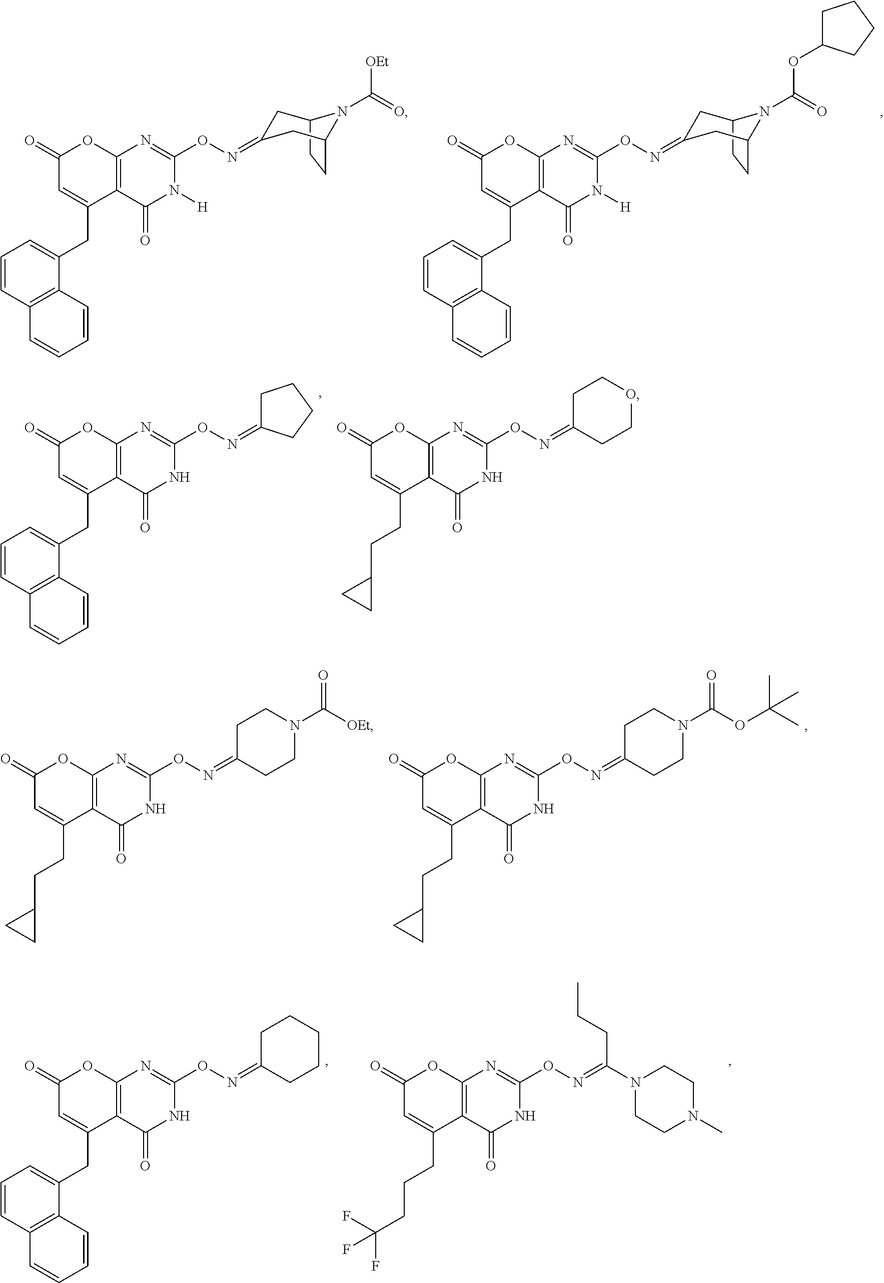

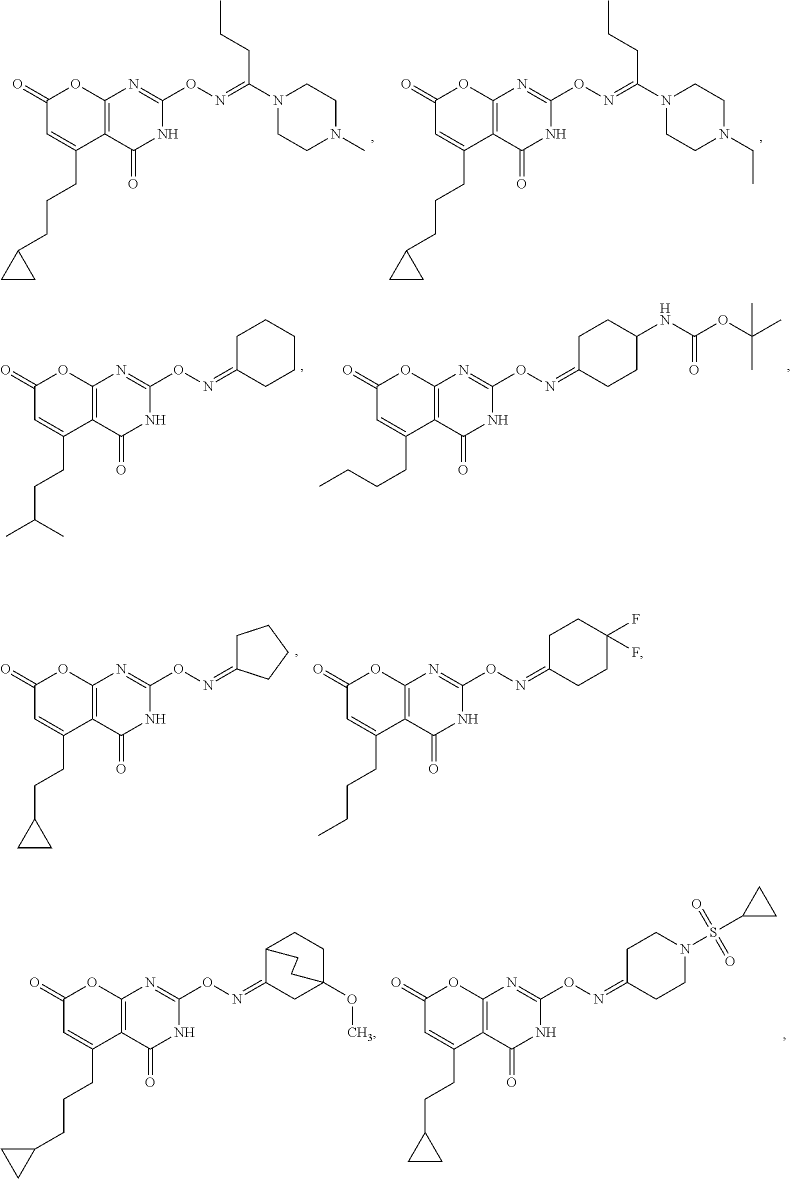

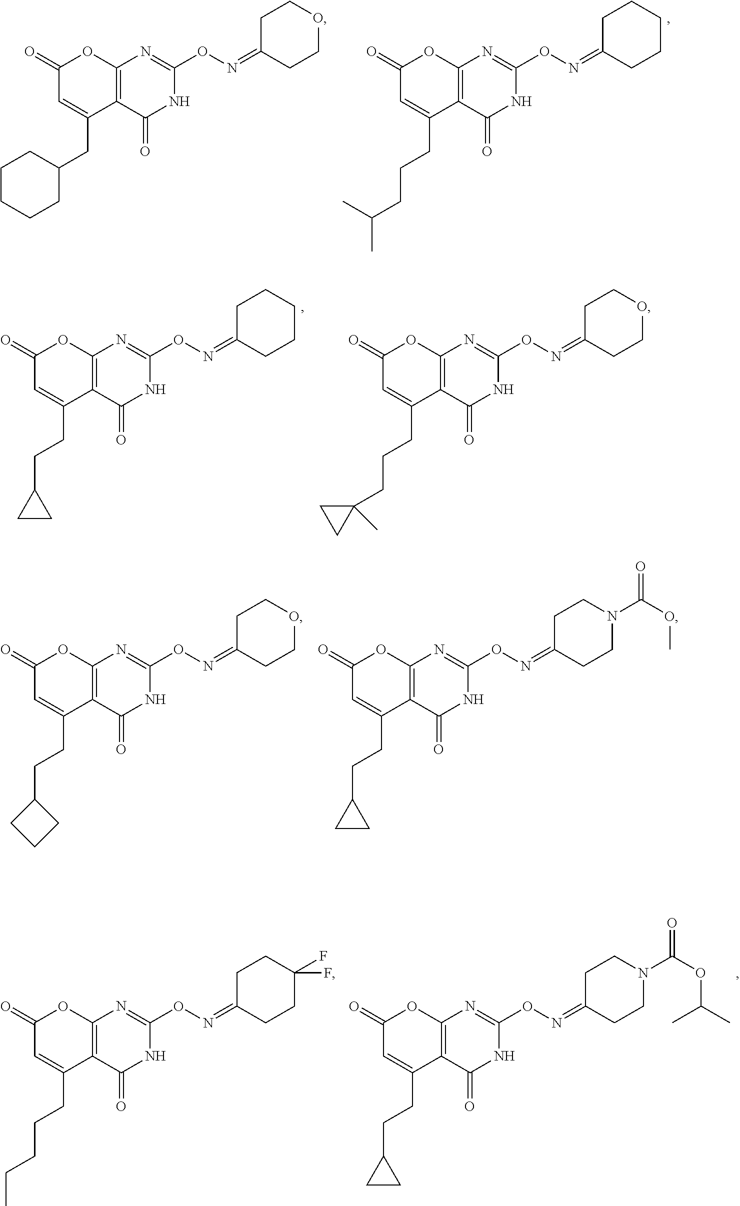

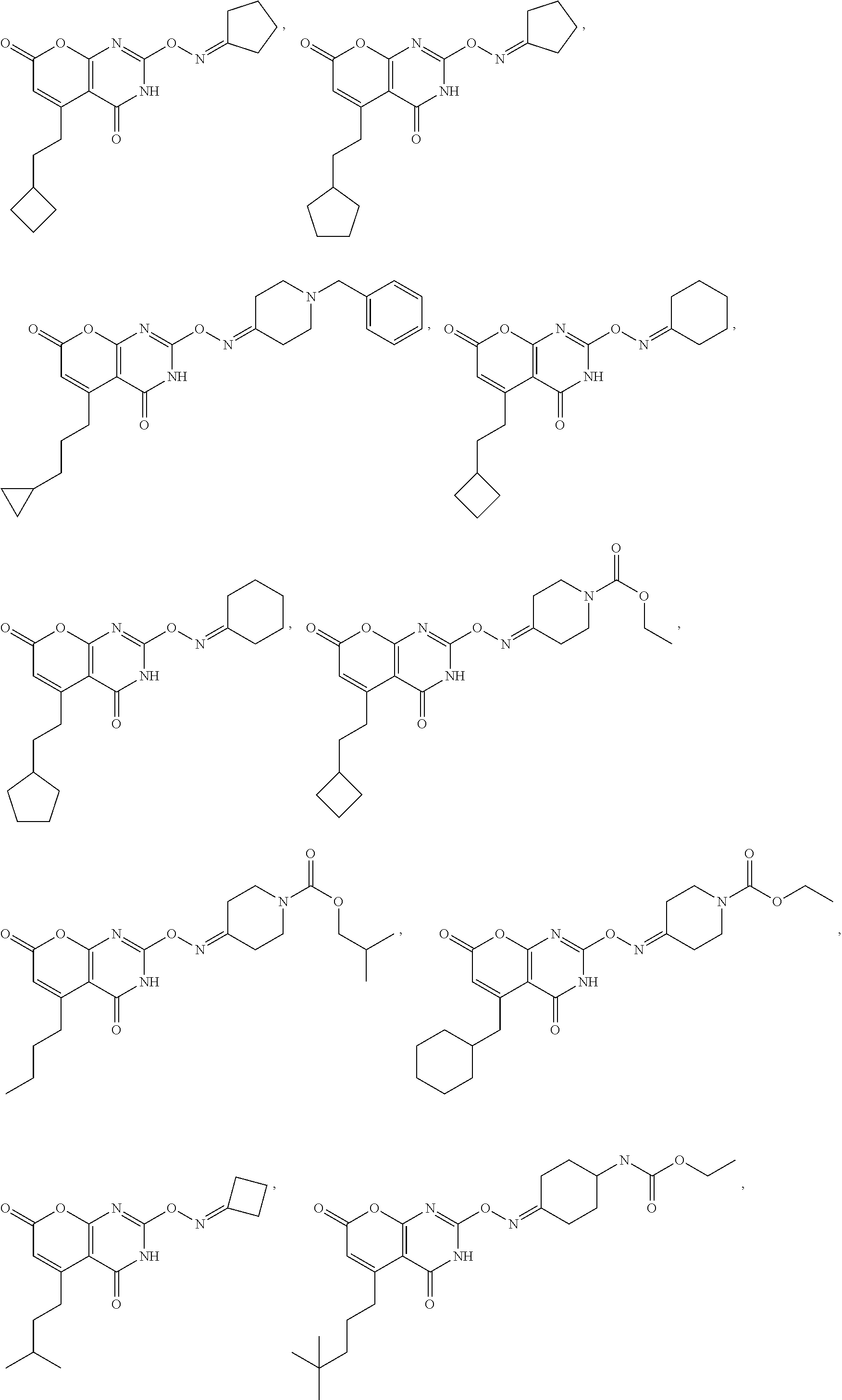

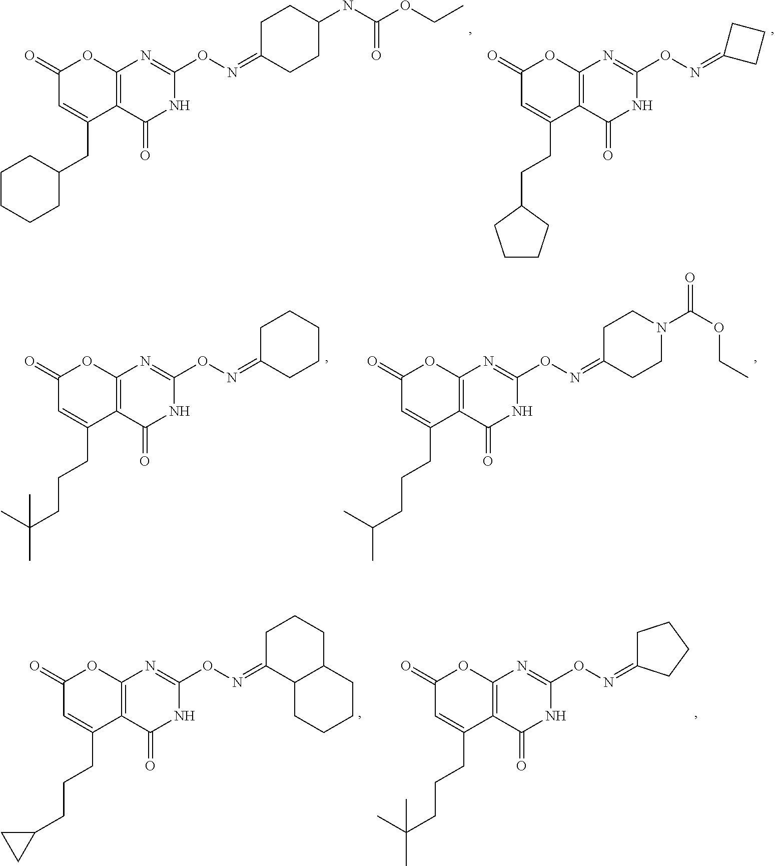

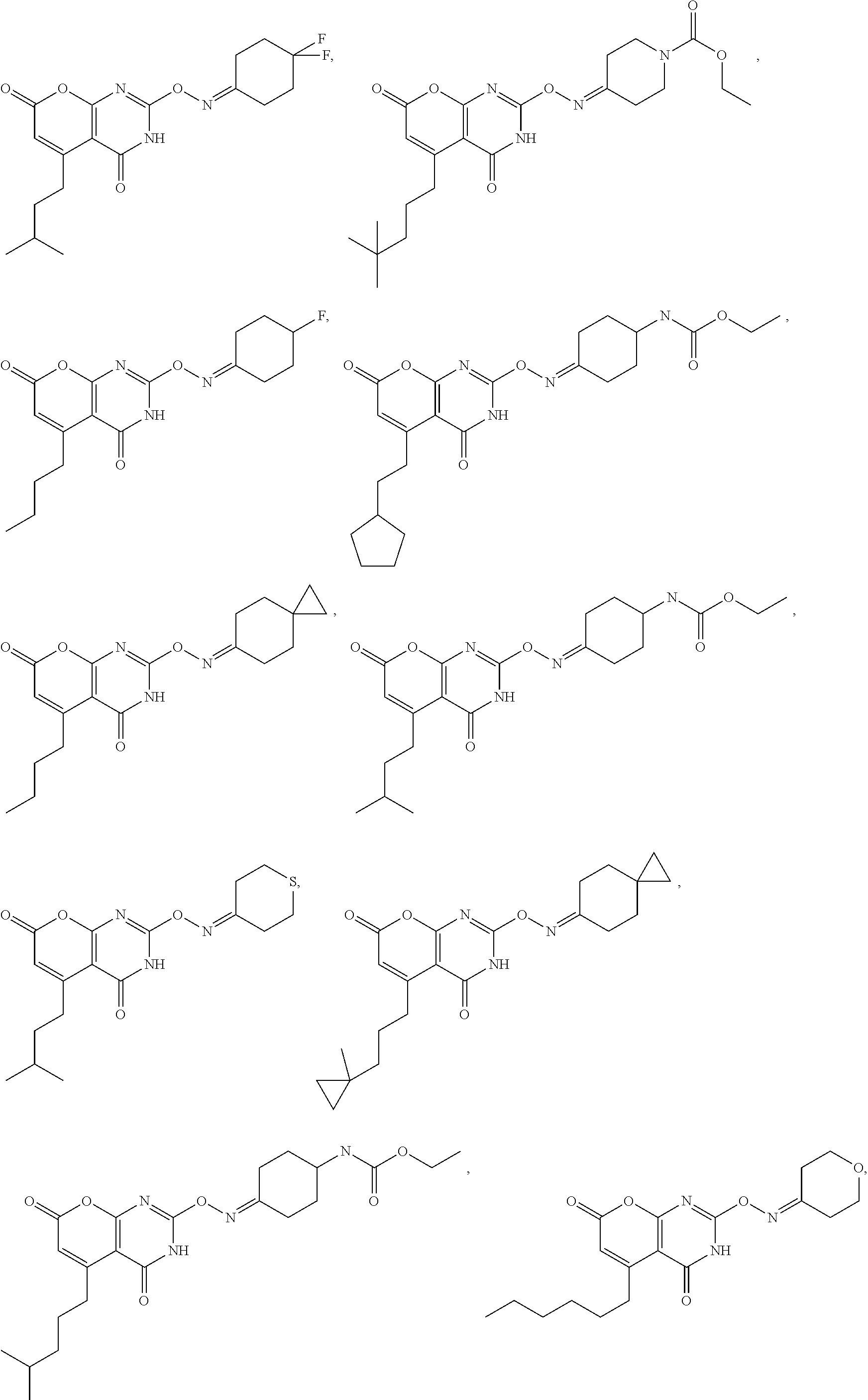

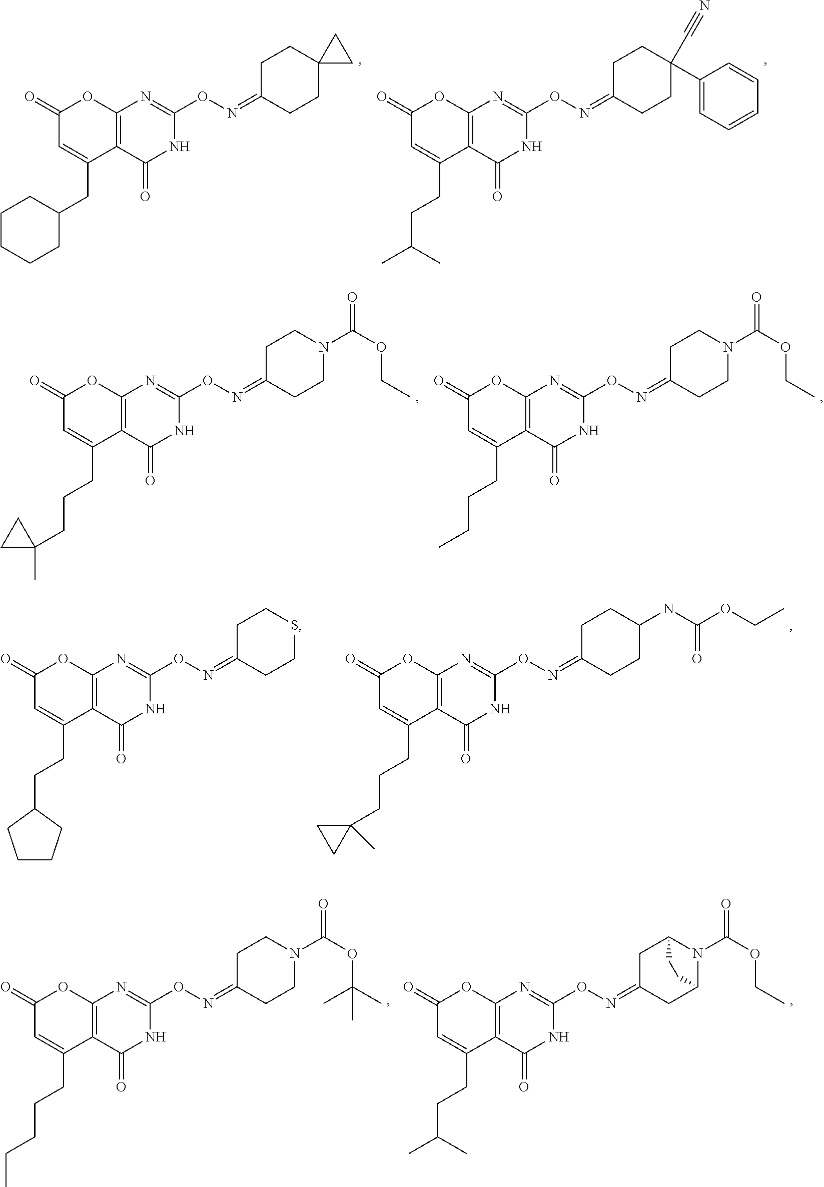

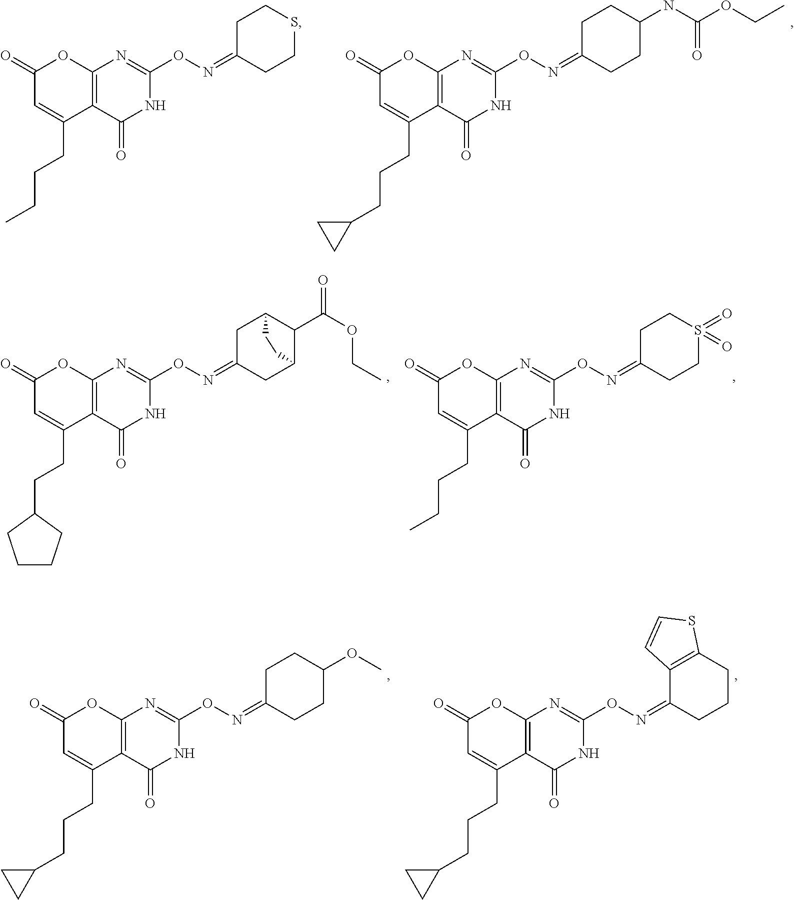

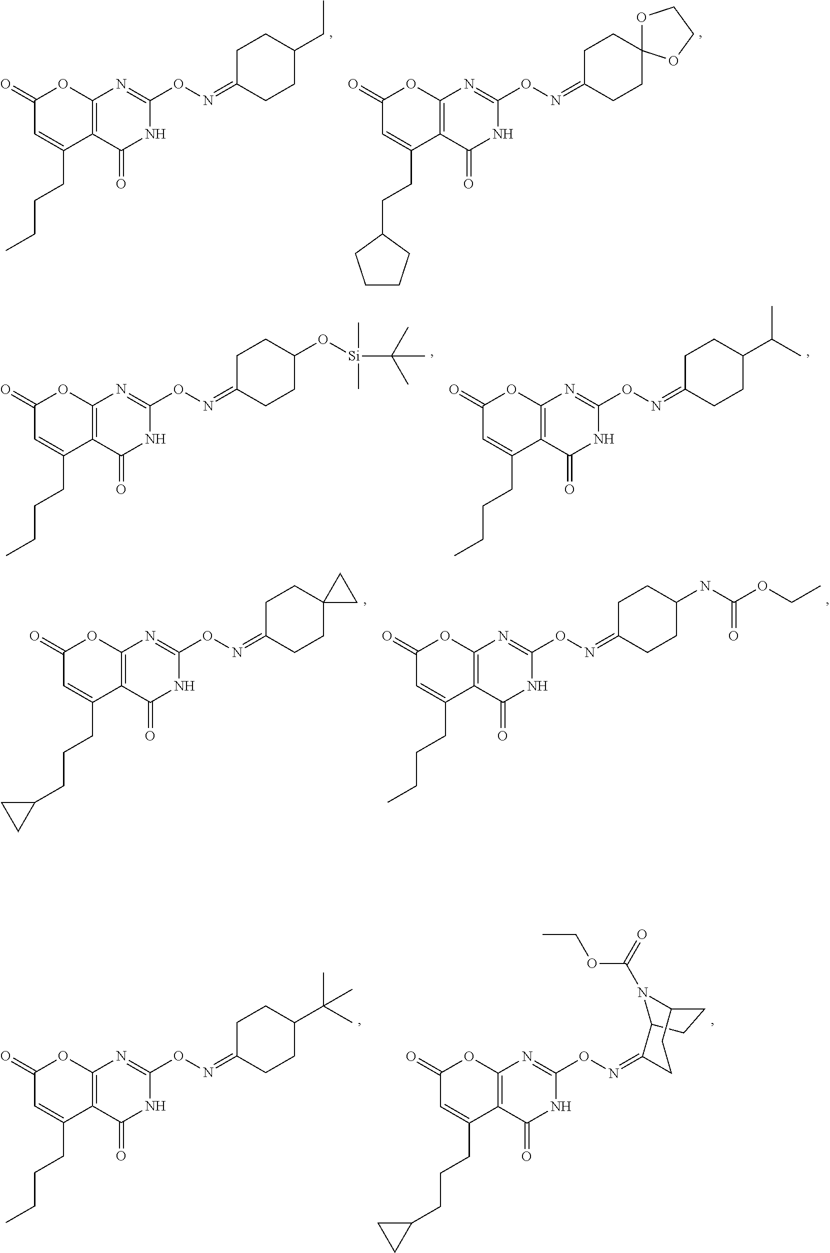

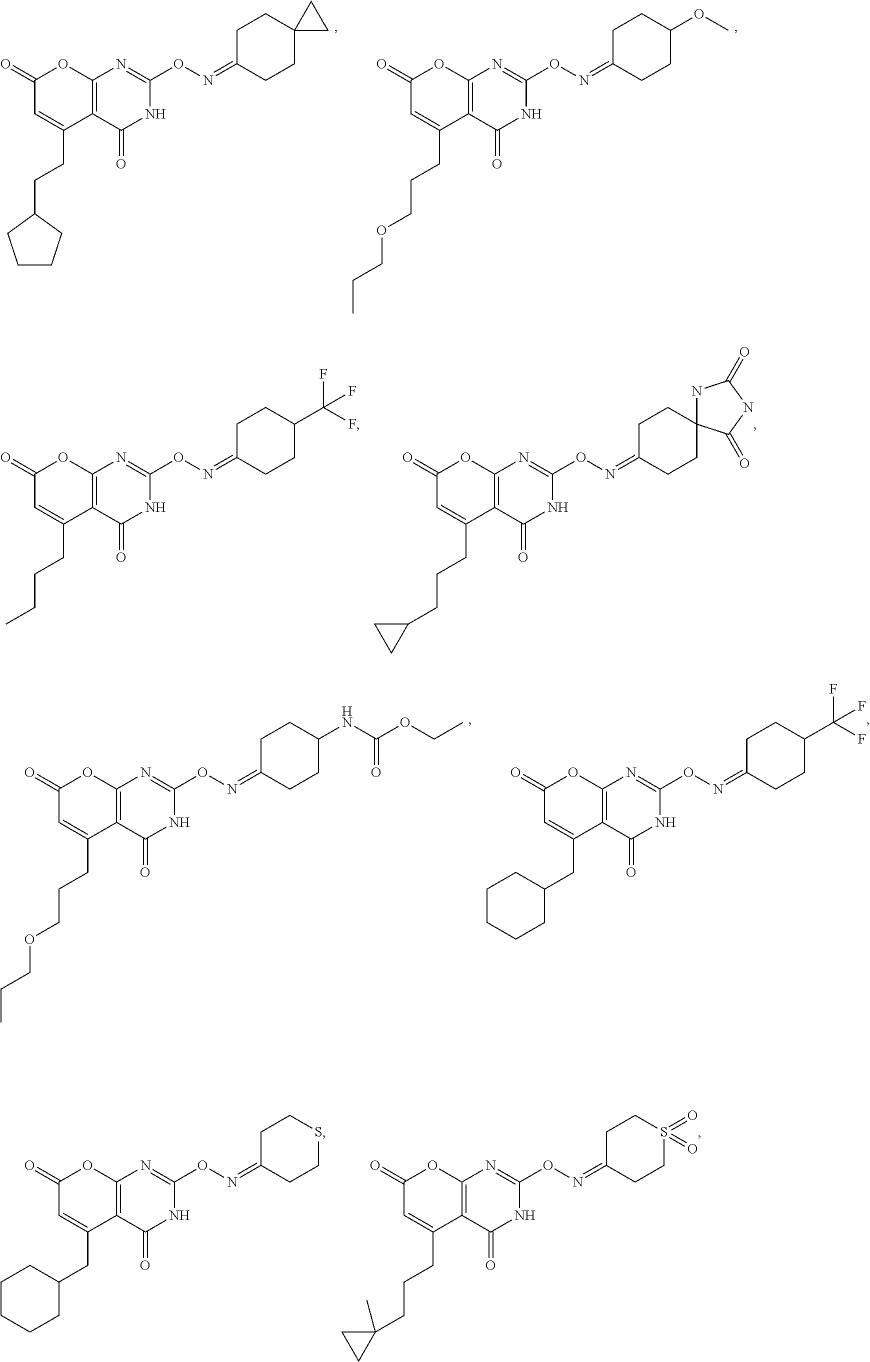

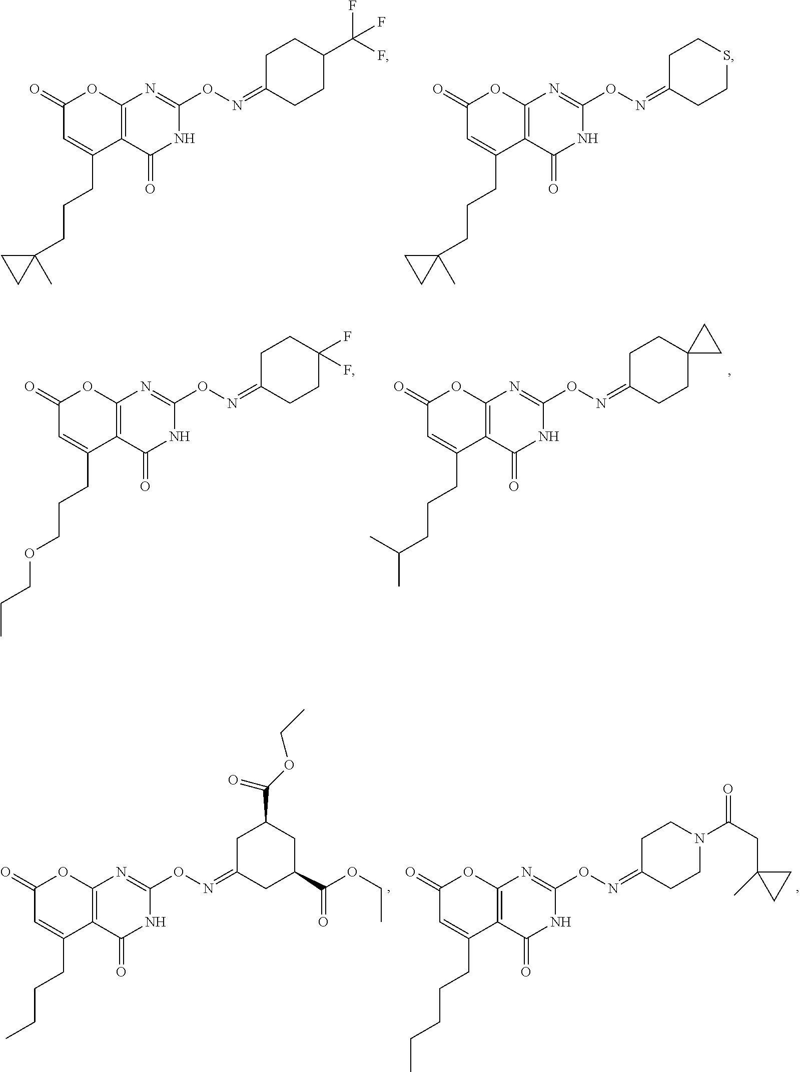

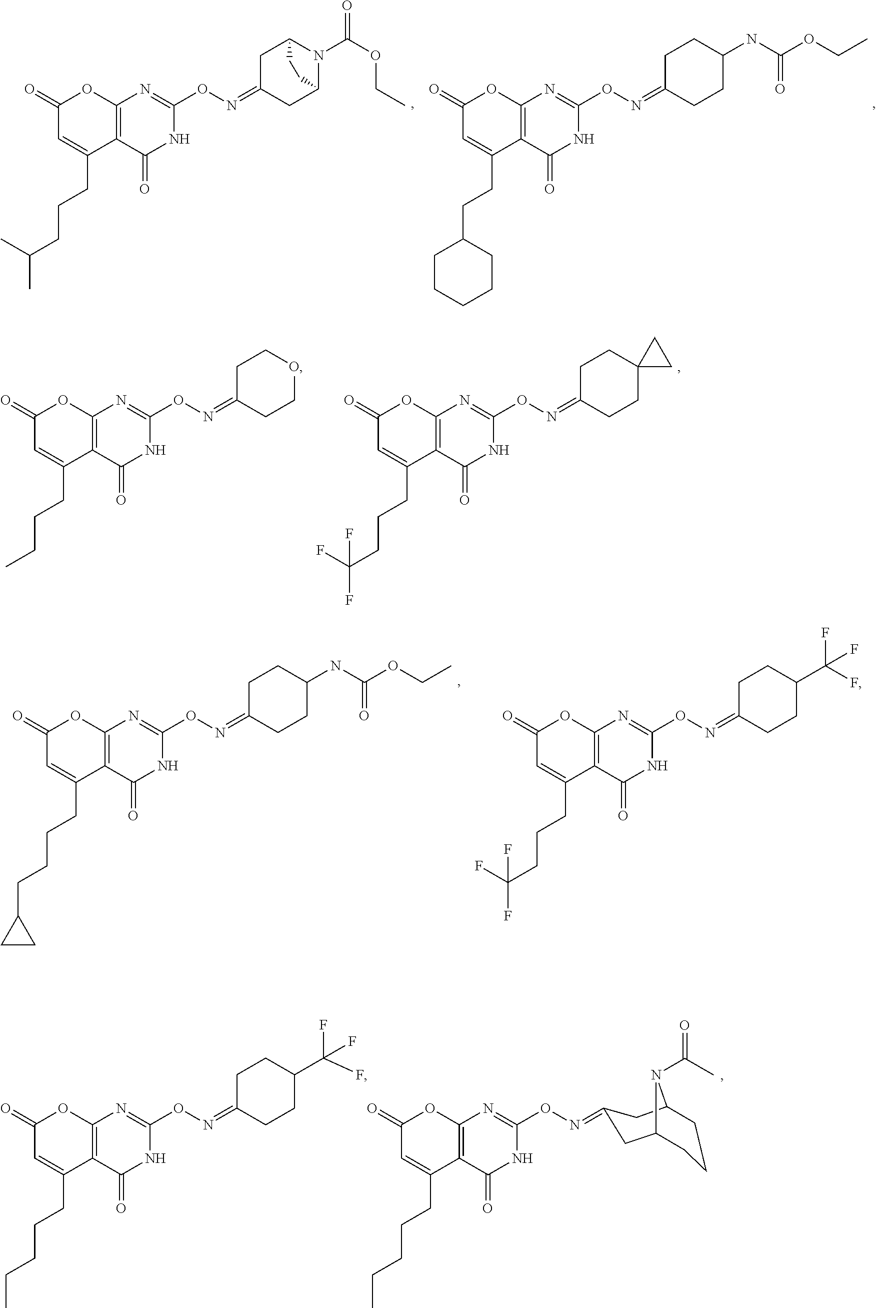

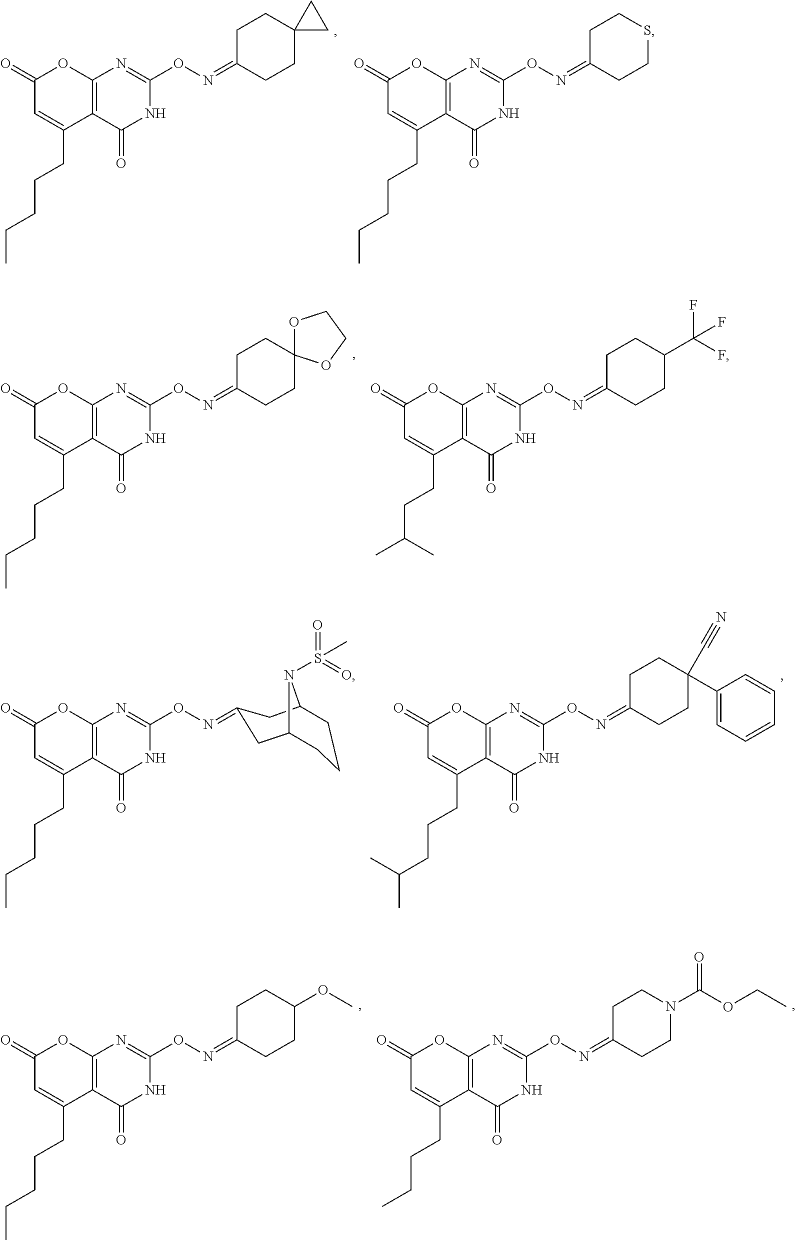

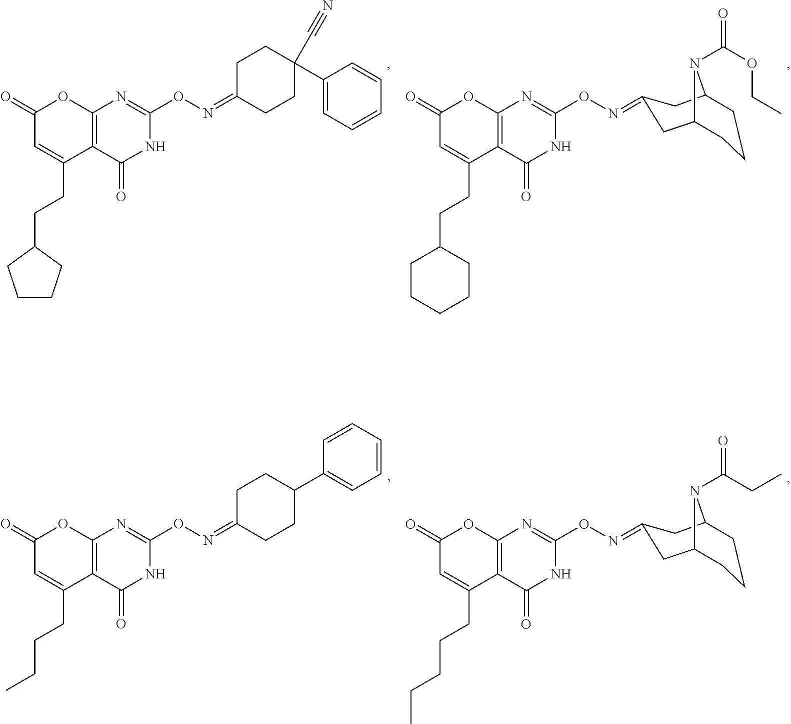

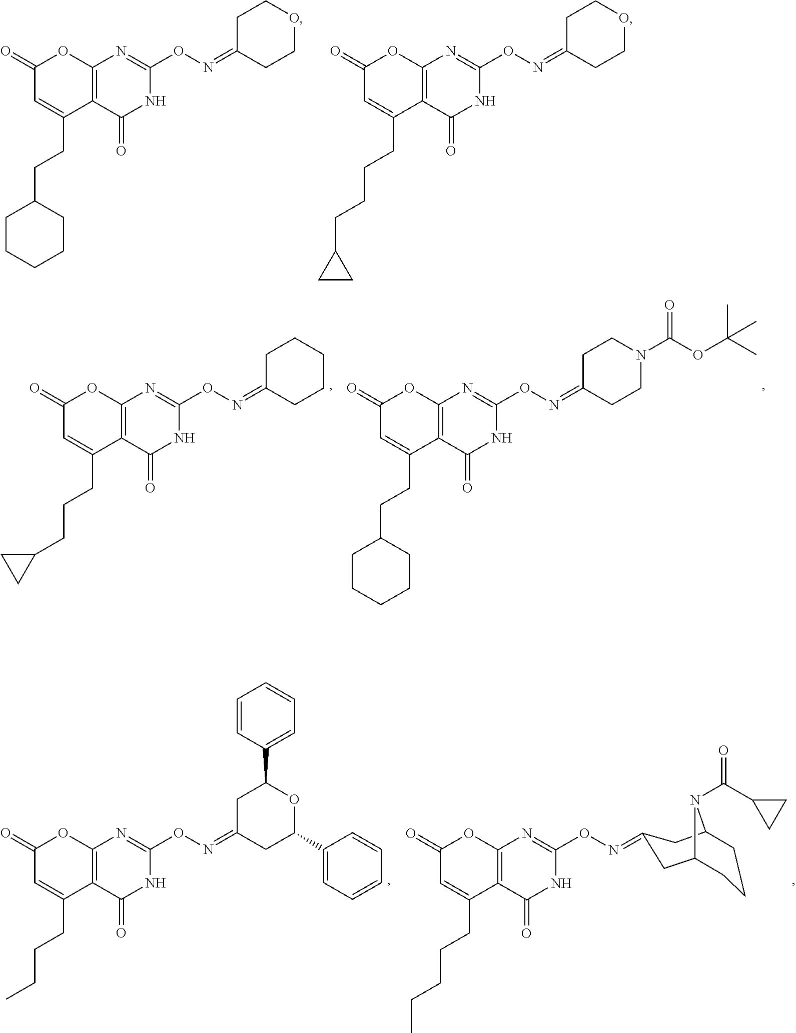

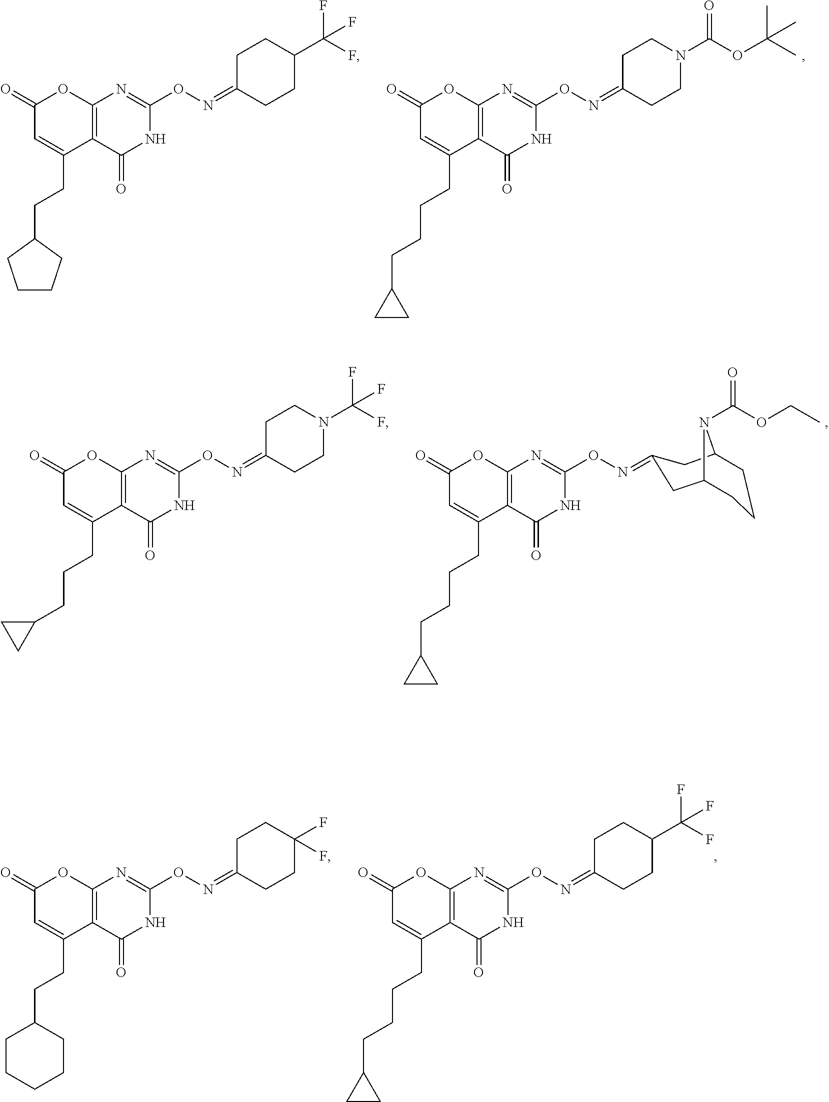

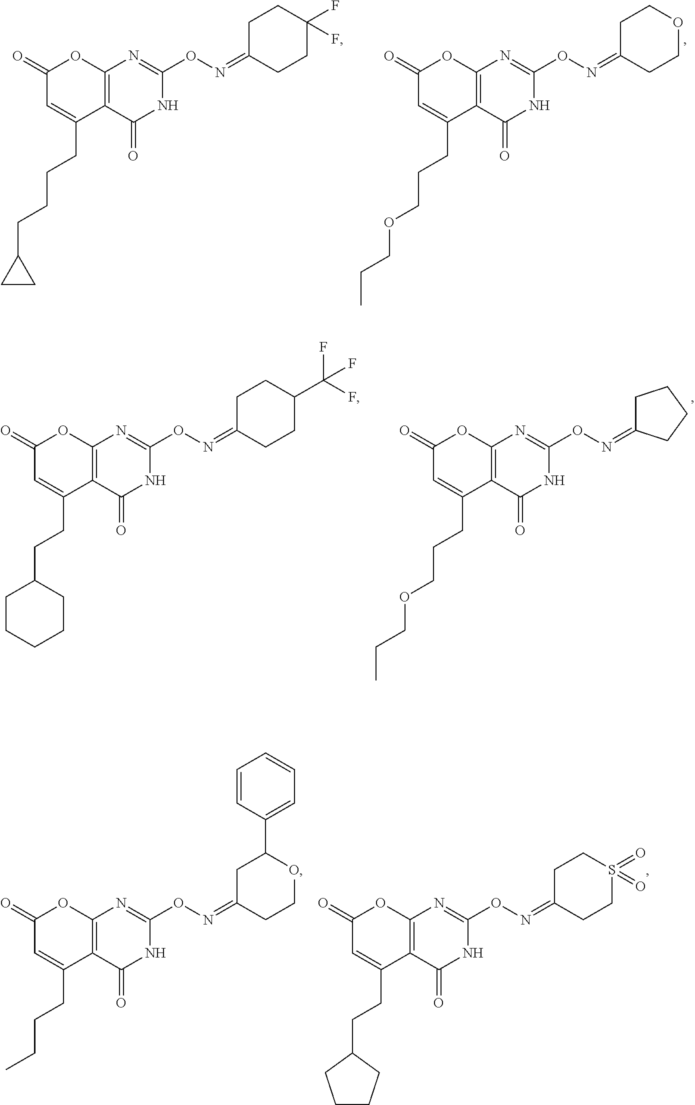

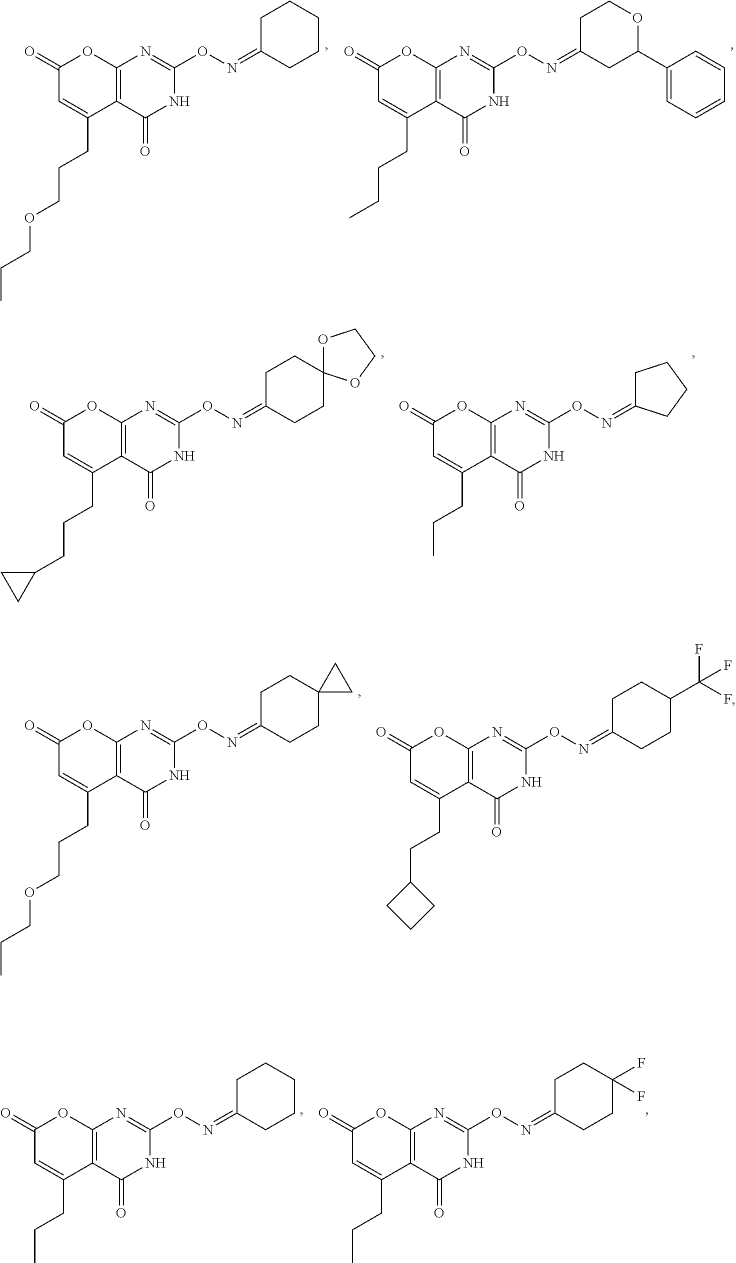

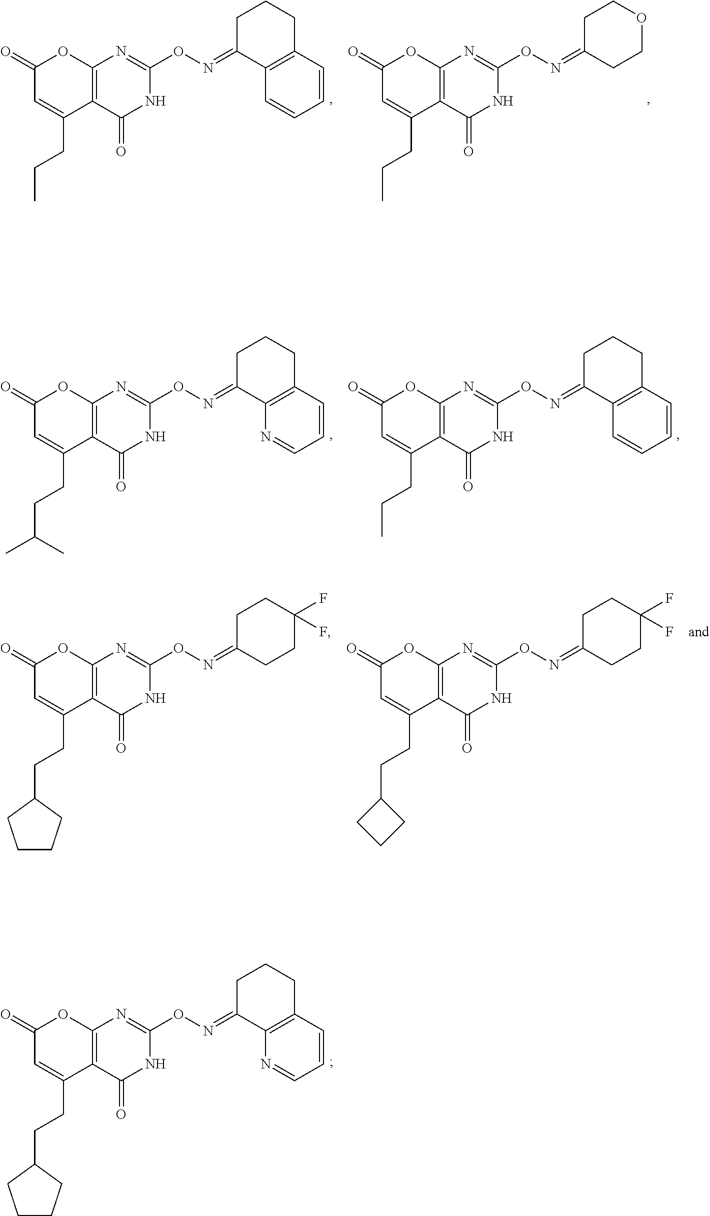

and pharmaceutically acceptable salts, solvates, esters and prodrugs thereof. These compounds are described in U.S. Publication No. 2007/0173495; and International Publication Nos. WO 2008/12759 and WO 2008/06476, herein incorporated by reference.

In some embodiments, the PAR1 antagonist is selected from the group of compounds consisting of the following:

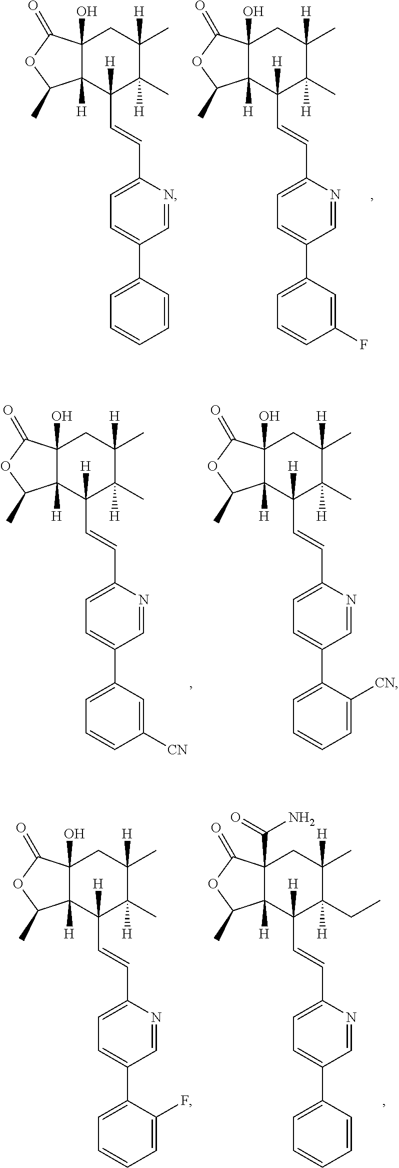

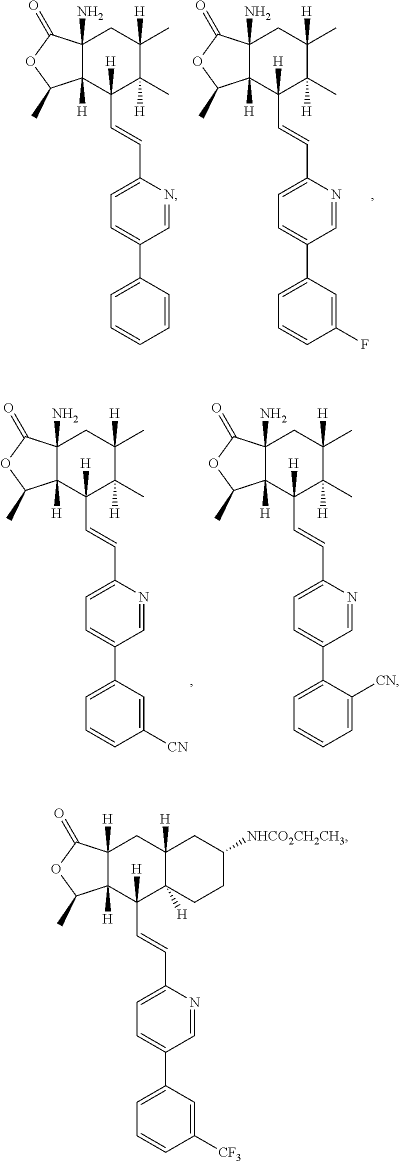

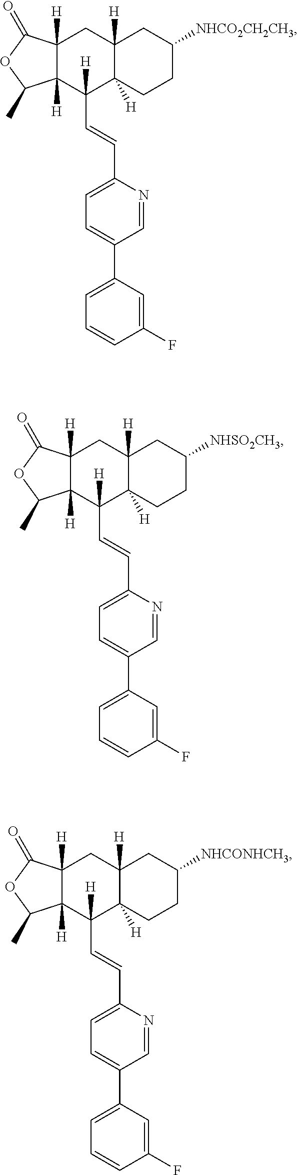

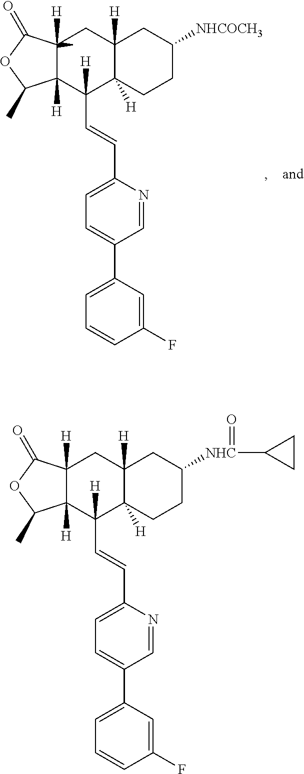

or a pharmaceutically acceptable isomer, salt, solvate or co-crystal form thereof.

In some embodiments, the cardiovascular agent is selected from the group consisting of A1 adenosine receptor agonists, acyl coenzyme A-cholesterol acyl transferase inhibitors, angiotensin antagonists, angiotensin converting enzyme (ACE) inhibitors, anticoagulants beta blockers, apolipoprotein A1 gene expression modulators, apolipoprotein A1 mimetics, apolipoprotein A1 modulators, apolipoprotein A1 synthesis inducers, ATP-dependent potassium channel agonists, bile acid sequestrants, B-type natriuretic peptides, calcium antagonists, calcium channel blockers, CCR2 antagonists, cholesterol absorption inhibitors (e.g., ezetimibe), a cholesteryl ester transfer protein (CETP) inhibitors, cyclooxygenase inhibitors, diuretics, endothelin antagonists, Factor Xa inhibitors, farnesoid X receptor modulators, fibrinolytics, GP IIb/IIIa antagonists neutral endopeptidase inhibitors, HDL elevating agents, HMG-CoA reductase inhibitors, Hsp60 modulators, LDL Cholesterol lowering agents, leptin, leptin analogs, lipoprotein-associated phospholipase A2 inhibitors, 5-lipoxygenase-activating protein inhibitors, liver X receptor agonists, a microsomal triglyceride transport inhibitors, nicotinic receptor agonists, omega-3 fatty acids, p38 MAP kinase Inhibitors, pancreatic lipase inhibitors, peroxisome proliferator-activated receptor (PPAR) modulators, phosphodiesterase inhibitors, phytosterols, platelet aggregation inhibitors, QRX-411, renin-angiotensin system inhibitors, rho A kinase inhibitors, site-1 protease inhibitors, SP-1000, squalene epoxidase inhibitors, squalene synthase inhibitors, stearoyl-CoA desaturase inhibitors, thromboxane A2 biosynthesis inhibitors, thromboxane antagonists, thyroid hormone receptor modulators, and a triglyceride synthesis inhibitors.

In some embodiments, the cardiovascular agent is a PPAR modulator selected from the group consisting of AGX-0104, beclofibrate, benzafibrate, BMS-687453, ciprofibrate, clofibrate, CP-900691, DRF-10945, DRF-11605, DRL-15609, etofibrate, F-16482, F-16618, fenofibrate, FP-16505, gemfibrozil, GFT-505, GW-0742, indeglitazar, KRP-101, KRP-105, MBX-2599, MBX-8025, MP-136, NP-016, NS-220, RWJ-667567, SAR-351034, ZYH-1, ZYH-7.

In some embodiments, the cardiovascular agent is a bile acid sequestrant selected from the group consisting of cholestyramine, colesevelam, colestilan, and colestipol.

In some embodiments, the cardiovascular agent is a cholesterol absorption inhibitor selected from the group consisting of beta-sitosterol, ezetimibe, MD-0727, MK-6213, KT6-971, and tiqueside.

In some embodiments, the cardiovascular agent is a phytosterol selected from the group consisting of FM-VA12, FM-VP24, and FM-VP4.

In some embodiments, the cardiovascular agent is a pancreatic lipase inhibitors selected from the group consisting of ATL-962, carnosic acid, lipstatin, GT-389255, and orlistat.

In one embodiment, the cardiovascular agent is an apolipoprotein A1 modulators selected from the group consisting of AVP-26452, DYB-186, and RVX-208.

In another embodiment, the cardiovascular agent is an apolipoprotein mimetic selected from the group consisting of ARI-1778, hE-18A, KH-01500, and trimerized apolipoprotein A-1.

In yet another embodiment, the cardiovascular agent is a cholesterol synthesis inhibitor selected from the group consisting of BDC-03, and ETC-1002.

In some embodiments, the cardiovascular agent is a CETP inhibitor selected from the group consisting of dalcetrapib, DRL-17822, JTT-302, anacetrapib, TA-8995

In one embodiment, the cardiovascular agent is a microsomal triglyceride protein inhibitors selected from the group consisting of AS-1512680, implitapide, JTT-130, lomitapide, and Slx-4090.

In another embodiment, the cardiovascular agent is a stearoyl-CoA desaturase inhibitors selected from the group consisting of aramchol, CVT-12012, and GRC-9332.

In yet another embodiment, the cardiovascular agent is a lipoprotein-associated phospholipase A2 inhibitors selected from the group consisting of darapladib and rilapladib.

In some embodiments, the cardiovascular agent is a thromboxane antagonist selected from the group consisting of seratrodast, picotamide and ramatroban.

In some embodiments, the cardiovascular agent is a platelet aggregation inhibitor selected from the group consisting of clopidogrel, clopidogrel bisulfate, elinogrel, cilostazol, abciximab, limaprost, ticlopidine and eptifibatide, prasugrel (CS-747), AZD-6140 and CT-50547.

In some embodiments, the cardiovascular agent is an acyl Coenzyme A-cholesterol acyltransferase inhibitor is selected from the group consisting of avasimibe, eflucimibe, K-604, KY-455, KY-505, SMP-797, and VULM-1457.

In some embodiments, the cardiovascular agent is a p38 MAP kinase Inhibitor selected from the group consisting of BMS-582949 and losmapimod.

In some embodiments, the cardiovascular agent is a cyclooxygenase inhibitor selected from the group consisting of aspirin, meloxicam, rofecoxib and celecoxib.

In some embodiments, the cardiovascular agent is an angiotensin antagonist selected from the group consisting of valsartan, telmisartan, candesartan, irbesartan, losartan and eprosartan.

In some embodiments, the cardiovascular agent is tezosentan. In some embodiments, the cardiovascular agent is a phosphodiesterase inhibitor selected from the group consisting of milrinoone and enoximone.

In some embodiments, the cardiovascular agent is an angiotensin converting enzyme (ACE) inhibitor selected from the group consisting of captopril, enalapril, enalapril, spirapril, quinapril, perindopril, ramipril, fosinopril, trandolapril, lisinopril, moexipril and benazapril.

In some embodiments, the cardiovascular agent is a neutral endopeptidase inhibitor selected from the group consisting of candoxatril and ecadotril.

In some embodiments, the cardiovascular agent is an anticoagulant selected from the group consisting of warfarin, ximelagatran, fondaparin, nadroparin, dalteparin and enoxaparin.

In some embodiments, the cardiovascular agent is a diuretic selected from the group consisting of chlorothiazide, hydrochlorothiazide, ethacrynic acid, furosemide and amiloride.

In some embodiments, the cardiovascular agent is a Factor Xa inhibitor selected from the group consisting of apixaban, razaxaban, Arixtra®, Induprux®, KFA-1982, and DX-9065a.

In some embodiments, the cardiovascular agent is a HMG-CoA reductase inhibitor selected from the group consisting of atorvastatin cerivastatin, dalvastatin, fluvastatin, HL-012, lovastatin, pitavastatin, PPD-10558, pravastatin, rosuvastatin, and simvastatin.

In some embodiments, the cardiovascular agent is a calcium antagonist selected from the group consisting of diltiazem, lacidipine, and verapamil.

In some embodiments, the cardiovascular agent is a B-type natriuretic peptide selected from the group consisting of nesiritide and ularitide.

In some embodiments, the cardiovascular agent is a beta blocker selected from the group consisting of nebivolol and betaxolol.

In some embodiments, the cardiovascular agent is amlodipine.

In some embodiments, the cardiovascular agent is milrinoone.

In some embodiments, the cardiovascular agent is a fibrinolytic selected from the group consisting of reteplase, alteplase, and tenecteplase.

In some embodiments, the cardiovascular agent is a GP IIb/IIIa antagonist selected from the group consisting of Integrillin®, abciximab, and tirofiban.

In a preferred embodiment the cardiovascular agent is aspirin, clopidogrel, clopidogrel bisulfate, prasugrel, or elinogrel.

In one embodiment, the cardiovascular agent is a thyroid receptor beta agonist selected from the group consisting of eprotirome, sobetirome, and MB-07344.

In some embodiments, the cardiovascular agent is a liver X receptor agonist selected from the group consisting of CVT-4681, PX-6500, WAY-254011, WYE-672, and XL-652.

A non-limiting example of an apolipoprotein A1 gene expression stimulator is RVX-208.

A non-limiting example of a bile acid readsorption inhibitor is SAR-548304.

A non-limiting example of a triglyceride synthesis inhibitor is WHI-P164.

A non-limiting example of a calcium channel blocker is isradipine.

A non-limiting example of a rho A kinase inhibitor is SLx-2119.

A non-limiting example of a thyroid hormone receptor modulator is 3,5-diiodothyropropionic acid.

A non-limiting example of a farnesoid X receptor modulator is turofexorate isopropyl.

A non-limiting example of a CCR2 antagonist is GSK-1344386B.

A non-limiting example of an A1 adenosine receptor agonist is CVT-2501.

A non-limiting example of a Hsp60 modulator is PTR-709-A.

A non-limiting example of a lipoprotein associated phospholipase A2 inhibitor is rilapladib.

A non-limiting example of a 5-lipooxygenase inhibitor is MK-0633.

A non-limiting example of a site-1 protease inhibitor is PF-429242.

A non-limiting example of a squalene synthase inhibitor is lapaquistat.

A non-limiting example of a ATP-dependent potassium channel agonist is diazoxide.

In one embodiment, the cardiovascular agent is a lipid lowering agent.

In one embodiment, the pharmaceutical composition comprises a PAR1 antagonist, at least one lipid lowering agent, and optionally a pharmaceutically acceptable carrier.

In one embodiment, the lipid lowering agent is selected from the group consisting of acifran, acipimox, AGX-0104, anacetrapib, aramchol, ARI-1778, AS-1512680, ATI-5261, ATL-962, atorvastatin, avasimibe, AVEX-1, AVP-26452, BDC-03, beclofibrate, benzafibrate, beta-sitosterol, BMS-582949, BMS-687453, carnosic acid, carvastatin, cerivastatin, cholestyramine, ciprofibrate, clofibrate, colesevelam, colestilan, colestipol, CP-900691, CRD-5, CRD-510, CVT-2501, CVT-4681, CVT-12012, dalcetrapib, dalvastatin, darapladib, diazoxide, Dihydrocaffeate, 3,5-diiodothyropropionic acid, DRF-10945, DRF-11605, DRL-12424, DRL-15609, DRL-17822, DYB-186, eflucimibe, Eicosapentate, eprotirome, ETC-1002, etofibrate, ETX-6107, ezetimibe, F-16482, fenofibrate, fluvastatin, FM-VA12, FM-VP4, FM-VP24, FP-16505, gemfibrozil, GRC-9332, GSK-256073, GSK-1344386B, GT-389255, GW-0742, hE-18A, HL-012, implitapide, indeglitazar, isradipine, JTT-130, JTT-302, K-604, KH-01500, KT6-971, KY-455, KY-505, lacidipine, lapaquistat, lipstatin, lomitapide, losmapimod, lovastatin, MAHDL-01, MB-07344, MBX-8025, MD-0727, MED-205, MK-1903, MK-0633, MK-6213, MP-136, niacin, NP-016, NS-220, orlistat, PF-429242, pitavastatin, PPD-10558, pravastatin, PTR-709-A, PX-6500, QRX-401, QRX-411, rosuvastatin, rilapladib, RVX-208, SAR-351034, SAR-548304, simvastatin, SLV-341, SLx-2119, Slx-4090, SMP-797, sobetirome, SP-1000, tiqueside, TA-8995, TRIA-662, trimerized apolipoprotein A-1, turofexorate isopropyl, VULM-1457, WAY-254011, WHI-P164, WYE-672, XL-652, ZYH-1, ZYH-7, and ZYT-1.

In one embodiment, the invention is directed to a pharmaceutical composition comprising at least one PAR1 antagonist; at least one lipid lowering agent selected from the group consisting of: acifran, acipimox, AGX-0104, anacetrapib, aramchol, ARI-1778, AS-1512680, ATI-5261, ATL-962, atorvastatin, avasimibe, AVEX-1, AVP-26452, BDC-03, beclofibrate, benzafibrate, beta-sitosterol, BMS-582949, BMS-687453, carnosic acid, carvastatin, cerivastatin, cholestyramine, ciprofibrate, clofibrate, colesevelam, colestilan, colestipol, CP-900691, CRD-5, CRD-510, CVT-2501, CVT-4681, CVT-12012, dalcetrapib, dalvastatin, darapladib, diazoxide, Dihydrocaffeate, 3,5-diiodothyropropionic acid, DRF-10945, DRF-11605, DRL-12424, DRL-15609, DRL-17822, DYB-186, eflucimibe, Eicosapentate, eprotirome, ETC-1002, etofibrate, ETX-6107, ezetimibe, F-16482, fenofibrate, fluvastatin, FM-VA12, FM-VP4, FM-VP24, FP-16505, gemfibrozil, GRC-9332, GSK-256073, GSK-1344386B, GT-389255, GW-0742, hE-18A, HL-012, implitapide, indeglitazar, isradipine, JTT-130, JTT-302, K-604, KH-01500, KT6-971, KY-455, KY-505, lacidipine, lapaquistat, lipstatin, lomitapide, losmapimod, lovastatin, MAHDL-01, MB-07344, MBX-8025, MD-0727, MED-205, MK-1903, MK-0633, MK-6213, MP-136, niacin, NP-016, NS-220, orlistat, PF-429242, pitavastatin, PPD-10558, pravastatin, PTR-709-A, PX-6500, QRX-401, QRX-411, rosuvastatin, rilapladib, RVX-208, SAR-351034, SAR-548304, simvastatin, SLV-341, SLx-2119, Slx-4090, SMP-797, sobetirome, SP-1000, tiqueside, TA-8995, TRIA-662, trimerized apolipoprotein A-1, turofexorate isopropyl, VULM-1457, WAY-254011, WHI-P164, WYE-672, XL-652, ZYH-1, ZYH-7, and ZYT-1; and optionally a pharmaceutically acceptable carrier.

In some embodiments, the invention encompasses a method of treating a mammal in need of said treating comprising administering to said mammal pharmaceutically effective amounts of a PAR1 antagonist and a cardiovascular agent. Various embodiments of this method are in accordance with the pharmaceutical compositions above described.

In some embodiments, the condition is a cardiovascular or circulatory disease or condition, an inflammatory disease or condition, a respiratory tract disease or condition, cancer, or a neurodegenerative disease.

In some embodiments, the condition is a cardiovascular or circulatory disease or condition selected from the group consisting of acute coronary syndrome, secondary prevention, thrombosis, atherosclerosis, restenosis, hypertension, angina pectoris, arrhythmia, heart failure, myocardial infarction, glomerulonephritis, thrombotic stroke, thromboembolic stroke, peripheral vascular diseases, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis, erectile dysfunction, dyslipidemia and plaque rupture in coronary artery disease.

In some embodiments, the condition is an inflammatory disease or condition selected from the group consisting of irritable bowel syndrome, Crohn's disease, nephritis and a radiation- or chemotherapy-induced proliferative or inflammatory disorder of the gastrointestinal tract, lung, urinary bladder, or other organ.

In some embodiments, the condition is a respiratory tract disease or condition selected from the group consisting of reversible airway obstruction, asthma, chronic asthma, bronchitis and chronic airways disease.

In some embodiments, the condition is a cancer selected from the group consisting of renal cell carcinoma and an angiogenesis related disorder.

In some embodiments, the condition is a neurodegenerative disease selected from the group consisting of Parkinson's disease, amyotropic lateral sclerosis, Alzheimer's disease, Huntington's disease and Wilson's disease.

In some embodiments, the condition is selected from the group consisting of acute renal failure, glomerulonephritis, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, radiation fibrosis, endothelial dysfunction, and a wound or a spinal cord injury, or a symptom or result thereof.

In some embodiments, the condition is sepsis.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

As used herein, the term “alkyl” means an aliphatic hydrocarbon group that can be straight or branched and comprises 1 to about 20 carbon atoms in the chain. Preferred alkyl groups comprise 1 to about 12 carbon atoms in the chain. More preferred alkyl groups comprise 1 to about 6 carbon atoms in the chain. “Branched” means that one or more lower alkyl groups such as methyl, ethyl or propyl, are attached to a linear alkyl chain. The alkyl can be substituted by one or more substituents independently selected from the group consisting of halo, aryl, cycloalkyl, cyano, hydroxy, alkoxy, alkylthio, amino, —NH(alkyl), —NH(cycloalkyl), —N(alkyl)₂ (which alkyls can be the same or different), carboxy and —C(O)O—(C₁-C₆)alkyl. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group (straight or branched carbon chain) comprising one or more double bonds in the chain and which can be conjugated or unconjugated. Useful alkenyl groups can comprise 2 to about 15 carbon atoms in the chain, preferably 2 to about 12 carbon atoms in the chain, and more preferably 2 to about 6 carbon atoms in the chain. The alkenyl group can be substituted by one or more substituents independently selected from the group consisting of halo, alkyl, aryl, cycloalkyl, cyano and alkoxy. Non-limiting examples of suitable alkenyl groups include ethenyl, propenyl, n-butenyl, 3-methylbutenyl and n-pentenyl.

Where an alkyl or alkenyl chain joins two other variables and is therefore bivalent, the terms alkylene and alkenylene, respectively, are used.

“Alkoxy” means an alkyl-O— group in which the alkyl group is as previously described. Useful alkoxy groups can comprise 1 to about 12 carbon atoms, preferably 1 to about 6 carbon atoms. Non-limiting examples of suitable alkoxy groups include methoxy, ethoxy and isopropoxy. The alkyl group of the alkoxy is linked to an adjacent moiety through the ether oxygen.

“Alkynyl” means an aliphatic hydrocarbon group comprising at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. Non-limiting examples of suitable alkynyl groups include ethynyl, propynyl, 2-butynyl, 3-methylbutynyl, n-pentynyl, and decynyl. The alkynyl group may be substituted by one or more substituents which may be the same or different, each substituent being independently selected from the group consisting of alkyl, aryl and cycloalkyl.

“Aryl” means an aromatic monocyclic or multicyclic ring system comprising about 5 to about 14 carbon atoms, preferably about 6 to about 10 carbon atoms. The aryl can be substituted with one or more substituents, as defined above, which may be the same or different. Non-limiting examples of suitable aryl groups include phenyl, naphthyl, indenyl, tetrahydronaphthyl and indanyl. “Arylene” means a bivalent phenyl group, including ortho, meta and para-substitution.

Substitution on alkyl, alkenyl and alkynyl chains depends on the length of the chain, and the size and nature of the substituent. Those skilled in the art will appreciate that while longer chains can accommodate multiple substituents, shorter alkyl chains, e.g., methyl or ethyl, can have multiple substitution by halogen, but otherwise are likely to have only one or two substituents other than hydrogen. Shorter unsaturated chains, e.g., ethenyl or ethynyl, are generally unsubstituted or substitution is limited to one or two groups, depending on the number of available carbon bonds.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be substituted with one or more substituents, as defined above, which may be the same or different. Non-limiting examples of suitable monocyclic cycloalkyls include cyclopropyl, cyclobutyl, cyclopentyl, cyclohexyl and the like. Non-limiting examples of suitable multicyclic cycloalkyls include 1-decalinyl, norbornyl, adamantyl and the like. “Cycloalkylene” refers to a corresponding bivalent ring, wherein the points of attachment to other groups include all positional isomers.

“Dihydroxy(C₁-C₆)alkyl” refers to an alkyl chain substituted by two hydroxy groups on two different carbon atoms.

“Fluoroalkyl”, “difluoroalkyl” and “trifluoroalkyl” mean alkyl chains wherein the terminal carbon is substituted by 1, 2 or 3 fluoroatoms, respectively, e.g., —CF₃, —CH₂CF₃, —CH₂CHF₂ or —CH₂CH₂F. “Haloalkyl” means an alkyl chain substituted by 1 to 3 halo atoms.

“Halogen” or “halo” refers to fluorine, chlorine, bromine or iodine radicals. Preferred are fluoro, chloro or bromo, and more preferred are fluoro and chloro.

“Heteroaryl” means an aromatic monocyclic or multicyclic ring system comprising 5 to 14 ring atoms, preferably about 5 to 10 ring atoms, comprised of 1 to 13 carbon atoms and 1 to 4 heteroatoms independently selected from the group consisting of N, O and S, provided that the rings do not include adjacent oxygen and/or sulfur atoms. N-oxides of the ring nitrogens are also included, as well as compounds wherein a ring nitrogen is substituted by a (C₁-C₄)alkyl group to form a quaternary amine. Examples of single-ring heteroaryl groups are pyridyl, oxazolyl, isoxazolyl, oxadiazolyl, furanyl, pyrrolyl, thienyl, imidazolyl, pyrazolyl, tetrazolyl, thiazolyl, isothiazolyl, thiadiazolyl, pyrazinyl, pyrimidyl, pyridazinyl and triazolyl. Examples of bicyclic heteroaryl groups are naphthyridyl (e.g., 1, 5 or 1,7), imidazopyridyl, pyrido[2,3]imidazolyl, pyridopyrimidinyl and 7-azaindolyl. Examples of benzofused heteroaryl groups are indolyl, quinolyl, isoquinolyl, phthalazinyl, benzothienyl (i.e., thionaphthenyl), benzimidazolyl, benzofuranyl, benzoxazolyl and benzofurazanyl. All positional isomers are contemplated, e.g., 2-pyridyl, 3-pyridyl and 4-pyridyl. W-substituted heteroaryl refers to such groups wherein substitutable ring carbon atoms have a substituent as defined above, or where adjacent carbon atoms form a ring with an alkylene group or a methylenedioxy group, or where a nitrogen in the Het ring can be substituted with R²¹-aryl or an optionally substituted alkyl substituent as defined in W.

The term “Het” is exemplified by the single ring, the ring substituted with another ring (which can be the same or different), benzofused heteroaryl groups as defined immediately above, as well as tricyclic groups such as benzoquinolinyl (e.g., 1,4 or 7,8) or phenanthrolinyl (e.g., 1,7; 1,10; or 4,7). Het groups are joined to group B by a carbon ring member, e.g., Het is 2-pyridyl, 3-pyridyl or 2-quinolyl.

Examples of heteroaryl groups wherein adjacent carbon atoms form a ring with an alkylene group are 2,3-cyclopentenopyridine, 2,3-cyclohexenopyridine and 2,3-cycloheptenopyridine.

“Heterocycloalkyl” means a 4 to 6 membered saturated ring containing 3 to 5 carbon atoms and 1 or 2 heteroatoms selected from the group consisting of N, S and O, provided that the heteroatoms are not adjacent. Examples of heterocycloalkyl rings are pyrrolidinyl, piperidinyl, piperazinyl, morpholinyl, thiomorpholinyl, thiazolidinyl, 1,3-dioxolanyl, 1,4-dioxanyl, tetrahydrofuranyl, tetrahydrothiophenyl and tetrahydrothiopyranyl.

The term “heterospirocyclic” refers to a spirocyclic structure containing 3 to 5 carbon atoms and 1 or 2 heteroatoms selected from the group consisting of N, S and O, provided that the heteroatoms are not adjacent.

The term “optional single bond” represented [symbol] by refers to the bond shown by the double dotted line between X and the carbon to which Y and R¹⁵ are attached in the structures of Formulas I and II. “Optional single bond” means that a single bond may be present, or that no bond is present. The “optional double bond” represented by [symbol] refers to the bond shown by the combined solid/single dotted line in the middle ring of the structure shown for Formulas I and II and means that at least a single bond must be present, but that a double bond can be present. When the double bond is present, R¹⁰ is absent.

When R⁴ and R⁵ join to form a ring with the nitrogen to which they are attached, the rings formed are 1-pyrrolidinyl, 1-piperidinyl and 1-piperazinyl, wherein the piperazinyl ring may also be substituted at the 4-position nitrogen by a group R⁷.

The above statements, wherein, for example, R⁴ and R⁵ are said to be independently selected from a group of substituents, means that R⁴ and R⁵ are independently selected when attached to the same nitrogen, but also that where an R⁴ or R⁵ variable occurs more than once in a molecule, those occurrences are independently selected. Similarly, each occurrence of R¹³ or R¹⁴ is independent of any other R¹³ or R¹⁴ in the same Q ring. Those skilled in the art will recognize that the size and nature of the substituent(s) will affect the number of substituents which can be present.

Compounds of the invention have at least one asymmetrical carbon atom and therefore all isomers, including enantiomers, stereoisomers, rotamers, tautomers and racemates of the compounds of Formula I or II (where they exist) are contemplated as being part of this invention. The invention includes d and I isomers in both pure form and in admixture, including racemic mixtures. Isomers can be prepared using conventional techniques, either by reacting optically pure or optically enriched starting materials or by separating isomers of a compound of Formula I or II. Isomers may also include geometric isomers, e.g., when a double bond is present.

“Polymorph” means a crystalline form of a substance that is distinct from another crystalline form but that shares the same chemical formula. Polymorphous forms of the compounds of Formula I or II, whether crystalline or amorphous, also are contemplated as being part of this invention.

It should also be noted that any formula, compound, moiety or chemical illustration with unsatisfied valences in the present specification and/or claims herein is assumed to have sufficient hydrogen atom(s) to satisfy the valences.

“Effective amount” or “therapeutically effective amount” is meant to describe an amount of compound or a composition of the present invention effective in antagonism of a thrombin receptor and thus producing the desired therapeutic, ameliorative, inhibitory or preventative effect.

It should be noted that the literature uses the terms “antagonist” and “inhibitor” interchangeably. According in this application the term “antagonist” is synonymous to the term “inhibitor”.

Compounds of the invention with a basic group can form pharmaceutically acceptable salts with organic and inorganic acids. Examples of suitable acids for salt formation are hydrochloric, sulfuric, phosphoric, acetic, citric, oxalic, malonic, salicylic, malic, fumaric, succinic, ascorbic, maleic, methanesulfonic and other mineral and carboxylic acids well known to those in the art. Preferred embodiments include bisulfate salts. The salt is prepared by contacting the free base form with a sufficient amount of the desired acid to produce a salt. The free base form may be regenerated by treating the salt with a suitable dilute aqueous base solution such as dilute aqueous sodium bicarbonate. The free base form differs from its respective salt form somewhat in certain physical properties, such as solubility in polar solvents, but the salt is otherwise equivalent to its respective free base forms for purposes of the invention. Compounds of the invention can also form pharmaceutically acceptable solvates, including hydrates.

Certain compounds of the invention are acidic (e.g., those compounds which possess a carboxyl group). These compounds form pharmaceutically acceptable salts with inorganic and organic bases. Examples of such salts are the sodium, potassium, calcium, aluminum, lithium, gold and silver salts. Also included are salts formed with pharmaceutically acceptable amines such as ammonia, alkyl amines, hydroxyalkylamines, N-methylglucamine and the like.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or II or a salt and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Co-crystal” means a crystalline structure simultaneously comprising pharmaceutically active molecules and inert molecules. Co-crystals may be formed by combining a weak base with a weak acid selected to match hydrogen bond donors with acceptors. The pKa difference of conjugate pairs may be inconsistent with salt formation in water. The co-crystallizing agents used to form co-crystals are usually bifunctional acids such as fumaric acid, succinic acid, malic acid, and tartaric acid. Co-crystals are discussed in J. F. Remenar et. al., “Crystal Engineering of Novel Cocrystals of a Triazole Drug with 1,4-Dicarboxylic Acids”, Journal of the American Chemical Society, 2003, vol. 125, pp. 8456-8457.

Compounds of the invention with a carboxylic acid group can form pharmaceutically acceptable esters with an alcohol. Examples of suitable alcohols include methanol and ethanol. In the combinations of the present invention of at least one PAR1 antagonist, at least one NAR agonist and optionally one or more other therapeutically effective agents, the two or more active components may each be formulated individually and co-administered simultaneously or sequentially.

Alternatively, the active agents may be formulated in a single pharmaceutical composition comprising a PAR1 antagonist and a NAR agonist in a pharmaceutically acceptable carrier. The components of the combination can be administered individually or together in any conventional dosage form such as capsule, tablet, powder, cachet, suspension, solution, suppository, nasal spray, etc. The dosage of the other therapeutically active agent(s) can be determined from published material, and may range from 1 to 1000 mg per dose.

In this specification, the term “at least one PAR1 antagonist” or “at least one NAR agonist” means that one to three different compounds that are active as PAR1 antagonists or NAR agonists may be used in a pharmaceutical composition or method of treatment. Preferably one PAR1 antagonist and one NAR agonist are used. Similarly, the term “one or more additional cardiovascular agents” means that one to three additional drugs may be administered in combination with a PAR1; preferably, one additional compound is administered in combination with a PAR1 antagonist and NAR agonist combination.

### Formulations and Dosing

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), The Science and Practice of Pharmacy, 20^{th} Edition, Lippincott Williams & Wilkins, Baltimore, Md., (2000).

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The administration of the above-described combinations is carried out by specifying not only the formulations, but also the modes of administration and the dosing regimen.

Preferably, the compound is administered orally. Orally dissolving formulations of PAR1 antagonists are disclosed in U.S. provisional application No. 60/689,207, which is herein incorporated in its entirety by reference.

Preferably, the pharmaceutical preparation is in a unit dosage form. In such form, the preparation is subdivided into suitably sized unit doses containing appropriate quantities of the active component, e.g., an effective amount to achieve the desired purpose.

The dosing regimen for the above-described PAR1 antagonists may comprise a loading dose followed by a series of maintenance doses. The loading dose is about 1 to about 100 mg., preferably about 5 to about 40 mg., more preferably about 10 to about 30 mg., and still more preferably about 15 to about 25 mg. The daily maintenance dose is about 0.1 to about 10 mg., preferably about 0.5 to about 5 mg., more preferably about 0.5 to about 1.5 mg., most preferably about 0.75 to about 1.25 mg.

The daily dose of a PAR1 antagonist for treatment of a disease or condition cited above is about 0.001 to about 100 mg/kg of body weight per day, preferably about 0.001 to about 10 mg/kg. For an average body weight of 70 kg, the dosage level is therefore from about 0.1 to about 700 mg of drug per day, given in a single dose or 2-4 divided doses.

The daily dose of a NAR agonist for treatment of a disease or condition cited above is about 1 to about 1000 mg per day, preferably about 5 to about 300 mg per day for oral administration and 1 to about 100 mg per day, preferably about 2 to about 15 mg per day for intravenous administration, given in a single dose or 2-4 divided doses.

The amount and frequency of administration of the compounds of the invention and/or the pharmaceutically acceptable salts thereof will be regulated according to the judgment of the attending clinician considering such factors as age, condition and size of the patient as well as severity of the symptoms being treated.

### Conditions to be Treated

The present invention also encompasses methods of treating conditions by administration of the above-described combinations. Among the conditions that may be treated (or prevented) with these combinations are the following:

a cardiovascular or circulatory disease or condition, such as acute coronary syndrome, secondary prevention, thrombosis, atherosclerosis, restenosis, hypertension, angina pectoris, arrhythmia, heart failure, myocardial infarction, glomerulonephritis, thrombotic stroke, thromboembolic stroke, peripheral vascular diseases, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis or erectile dysfunction;

an inflammatory disease or condition, such irritable bowel syndrome, Crohn's disease, nephritis or a radiation- or chemotherapy-induced proliferative or inflammatory disorder of the gastrointestinal tract, lung, urinary bladder, or other organ;

a respiratory tract disease or condition, such as reversible airway obstruction, asthma, chronic asthma, bronchitis or chronic airways disease;

cancer, such as renal cell carcinoma or an angiogenesis related disorder; a neurodegenerative disease, such as Parkinson's disease, amyotropic lateral sclerosis, Alzheimer's disease, Huntington's disease or Wilson's disease; and,

other conditions, such as acute renal failure, glomerulonephritis, astrogliosis, a fibrotic disorder of the liver, kidney, lung or intestinal tract, Alzheimer's disease, diabetes, diabetic neuropathy, rheumatoid arthritis, neurodegenerative disease, neurotoxic disease, systemic lupus erythematosus, multiple sclerosis, osteoporosis, glaucoma, macular degeneration, psoriasis, sepsis, radiation fibrosis, endothelial dysfunction, a wound or a spinal cord injury, or a symptom or result thereof.

By way of an example, the effect of NAR and PAR1 compounds on inhibiting atherosclerosis/restenosis following vascular injury or stenting in rabbits on high cholesterol diet can be determined as follows:

Rabbits placed on high cholesterol diet develop atherosclerosis and their lipid levels including triglycerides and low density lipoprotein (LDL) cholesterol are markedly elevated. These rabbits when subjected to either a balloon angioplasty procedure of the carotid artery or stenting procedure develop restenosis or narrowing of the vessel wall due to intimal hyperplasia. These conditions closely mimic atherosclerosis and restenosis in humans with coronary artery disease (CAD).

NAR agonists by their mechanism of action, lower plasma triglycerides and LDL cholesterol and thereby inhibit the development of atherosclerosis. PAR1 antagonists on the other hand, inhibit intimal hyperplasia and restenosis. Thus the combination of NAR agonist and PAR1 antagonist markedly inhibits the development of dyslipidemia, atherosclerosis and restenosis. An additional benefit of the combination in man is that this combination inhibits thrombosis which is widely associated with atherosclerosis and plaque rupture in CAD.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A pharmaceutical composition comprising
i) an effective amount of at least one PAR1 antagonist which is selected from the group consisting of:
a) Formula I-A: 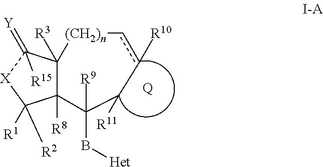
or a pharmaceutically acceptable salt or solvate thereof, wherein:
the single dotted line represents an optional single bond; [symbol]
represents an optional double bond;
n is 0-2;
Q is 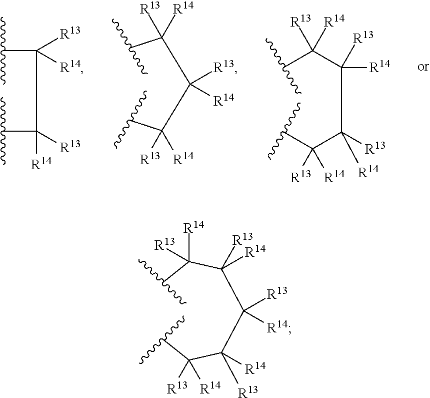
R¹ is independently selected from the group consisting of H, (C₁-C₆)alkyl, fluoro(C₁-C₆)alkyl-, difluoro(C₁-C₆)alkyl-, trifluoro-(C₁-C₆)alkyl-, (C₃-C₁₀)cycloalkyl, (C₂-C₆)alkenyl, hydroxy-(C₁-C₆)alkyl-, and amino(C₁-C₆)alkyl-;
R² is independently selected from the group consisting of H, (C₁-C₆)alkyl, fluoro(C₁-C₆)alkyl-, difluoro(C₁-C₆)alkyl-, trifluoro-(C₁-C₆)alkyl-, (C₃-C₁₀)cycloalkyl, (C₂-C₆)alkenyl, hydroxy-(C₁-C₆)alkyl-, and amino(C₁-C₆)alkyl-;
R³ is H, hydroxy, (C₁-C₆)alkoxy, —SOR¹⁶, —SO₂R¹⁷, —C(O)OR¹⁷, —C(O)NR¹⁸R¹⁹, —(C₁-C₆)alkyl-C(O)NR¹⁸R¹⁹, (C₁-C₆)alkyl, halogen, fluoro(C₁-C₆)alkyl-, difluoro(C₁-C₆)alkyl-, trifluoro(C₁ -C₆)alkyl-, (C₃-C₁₀)cycloalkyl, (C₃-C₁₀-cycloalkyl-(C₁-C₆)alkyl-, (C₂-C₆)alkenyl, aryl(C₁-C₆)alkyl-, aryl(C₂-C₆)alkenyl-, heteroaryl(C₁-C₆)alkyl-, heteroaryl(C₂-C₆)alkenyl-, hydroxy(C₁-C₆)-alkyl-, —NR²²R²³, NR²²R²³—(C₁-C₆)alkyl-, aryl, thio(C₁-C₆)alkyl-, (C₁-C₆)alkyl-thio(C₁-C₆)alkyl-, (C₁-C₆)alkoxy(C₁-C₆)alkyl-, NR¹⁸R¹⁹—C(O)—(C₁-C₆)alkyl- or (C₃-C₁₀)cycloalkyl-(C₁-C₆)alkyl-;
Het is a mono- or bi-cyclic heteroaryl group of 5 to 10 atoms comprised of 1 to 9 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by W;
W is 1 to 4 substituents independently selected from the group consisting of H, (C₁-C₆)alkyl, fluoro(C₁-C₆)alkyl-, difluoro(C₁-C₆)alkyl-, trifluoro(C₁-C₆)alkyl-, (C₃-C₁₀)cycloalkyl, hydroxy(C₁-C₆)alkyl-, dihydroxy(C₁-C₆)alkyl-, NR²⁵R²⁶(C₁-C₆)alkyl-, thio(C₁-C₆)alkyl-, —OH, (C₁-C₆)alkoxy, halogen, —NR⁴R⁵, —C(O)OR₁₇, —C(O)R¹⁶, (C₁-C₆)alkylthio-, R²¹-aryl, R²¹-aryl(C₁-C₆)alkyl-, aryl wherein adjacent carbons form a ring comprising a methylenedioxy group, and R²¹-heteroaryl;
R⁴ and R⁵ are independently selected from the group consisting of H, (C₁-C₆)alkyl, phenyl, benzyl and (C₃-C₁₀)cycloalkyl, or R⁴ and R⁵ taken together are —(CH₂)₄—, —(CH₂)₅— or —(CH₂)₂NR⁷—(CH₂)₂— and form a ring with the nitrogen to which they are attached;
R⁶ is H, (C₁-C₆)alkyl or phenyl;
R⁷ is H, (C₁-C₆)alkyl, —C(O)—R¹⁶, —C(O)OR¹⁷ or —SO₂R¹⁷;
R⁸, R¹⁰ and R¹¹ are independently selected from the group consisting of R¹ and —OR¹, provided that when the optional double bond is present, R¹⁰ is absent;
R⁹ is H, OH or (C₁-C₆)alkoxy;
B is —(CH₂)ₙ₃—, cis or trans —(CH₂)ₙ₄CR¹²═CR^{12a}(CH₂)ₙ₅ or —(CH₂)ₙ₄C≡C(CH₂)ₙ₅—, wherein n₃ is 0-5, n₄ and n₅ are independently 0-2, and R¹² and R^{12a} are independently selected from the group consisting of H, (C₁-C₆)alkyl and halogen;
X is —O— or —NO— when the dotted line represents a single bond, or X is —OH or —NHR²⁰ when the bond is absent;
Y is oxo, thioxo, (H, H), (H, OH) or (H, (C₁-C₆)alkoxy) when the dotted line represents a single bond, or when the bond is absent, Y is oxo, (H, H), (H, OH), (H, SH) or (H, (C₁-C₆)alkoxy);
each R¹³ is independently selected from H, (C₁-C₆)alkyl, (C₃-C₈)cycloalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halo(C₁-C₆)alkyl, and halogen;
each R¹⁴ is independently selected from H, (C₁-C₆)alkyl, —OH, (C₁-C₆)alkoxy, R²⁷-aryl(C₁-C₆)alkyl, heteroaryl, heteroarylalkyl, heterocyclyl, heterocyclylalkyl, —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4, halogen and halo(C₁-C₆)alkyl; or
R¹³ and R¹⁴ taken together form a spirocyclic or a heterospirocyclic ring of 3-6 atoms;
wherein at least one of R¹³ or R¹⁴ is selected from the group consisting of —(CH₂)ₙ₆NHC(O)OR^{16b}, —(CH₂)ₙ₆NHC(O)R^{16b}, —(CH₂)ₙ₆NHC(O)NR⁴R⁵, —(CH₂)ₙ₆NHSO₂R¹⁶, —(CH₂)ₙ₆NHSO₂NR⁴R⁵, and —(CH₂)ₙ₆C(O)NR²⁸R²⁹ where n₆ is 0-4;
R¹⁵ is absent when the dotted line represents a single bond and is H, (C₁-C₆)alkyl, —NR¹⁸R¹⁹, or —OR¹⁷ when the bond is absent;
R¹⁶ is independently selected from the group consisting of (C₁-C₆)alkyl, phenyl and benzyl;
R^{16b} is H, (C₁-C₆)alkyl, (C₁-C₆)alkoxy(C₁-C₆)alkyl-, R²²—O—C(O)—(C₁-C₆)alkyl-, (C₃-C₁₀)cycloalkyl, R²¹-aryl, R²¹-aryl(C₁-C₆)alkyl, halo(C₁-C₆)alkyl, (C₂-C₆)alkenyl, halosubstituted (C₂-C₆)alkenyl, (C₂-C₆)alkynyl, halosubstituted (C₂-C₆)alkynyl, R²¹-heteroaryl, R²¹—(C₁-C₆)alkyl heteroaryl, R²¹—(C₁-C₆)alkyl heterocycloalkyl, R²⁸R²⁹N—(C₁-C₆)alkyl, R²⁸R²⁹N—(CO)—(C₁-C₆)alkyl, R²⁸R²⁹N—(CO)O—(C₁-C₆)alkyl, R²⁸O(CO)N(R²⁹)—(C₁-C₆)alkyl, R²⁸S(O)₂N(R²⁹)—(C₁-C₆)alkyl, R²⁸R²⁹N—(CO)—N(R²⁹)—(C₁-C₆)alkyl, R²⁸R²⁹N—S(O)₂N(R²⁹)—(C₁-C₆)alkyl, R²⁸—(CO)N(R²⁹)—(C₁-C₆)alkyl, R²⁸R²⁹N—S(O)₂—(C₁-C₆)alkyl, HOS(O)₂—(C₁-C₆)alkyl, (OH)₂P(O)₂—(C₁-C₆)alkyl, R²⁸—S—(C₁-C₆)alkyl, R²⁸—S(O)₂—(C₁-C₆)alkyl or hydroxy(C₁-C₆)alkyl;
R¹⁷, R¹⁸ and R¹⁹ are independently selected from the group consisting of H, (C₁-C₆)alkyl, phenyl, and benzyl;
R²⁰ is H, (C₁-C₆)alkyl, phenyl, benzyl, —C(O)R⁶ or —SO₂R⁶;
R²¹ is 1 to 3 substituents independently selected from the group consisting of H, —CN, —CF₃, —OCF₃, halogen, —NO₂, (C₁-C₆)alkyl, —OH, (C₁-C₆)alkoxy, (C₁-C₆)—alkylamino-, di-((C₁-C₆)alkyl)amino-, NR²⁵R²⁶⁻(C₁-C₆)alkyl-, hydroxy-(C₁-C₆)alkyl-, —C(O)OR¹⁷, —COR¹⁷, —NHCOR¹⁶, —NHSO₂R¹⁶, —NHSO₂CH₂CF₃, —C(O)NR²⁵R²⁶, —NR²⁵—C(O)—NR²⁵R²⁶, —S(O)R¹³, —S(O)₂R¹³ and —SR¹³;
R²² is H or (C₁-C₆)alkyl;
R²³ is H, (C₁-C₆)alkyl, —C(O)R²⁴, —SO₂R²⁴, —CONHR²⁴ or —SO₂NHR²⁴;
R²⁴ is (C₁-C₆)alkyl, hydroxy(C₁-C₆)alkyl or NR²⁵R²⁶—((C₁-C₆)alkyl)-;
R²⁵ and R²⁶ are independently selected from the group consisting of H and (C₁-C₆)alkyl;
R²⁷ is 1, 2 or 3 substituents selected from the group consisting of H, (C₁-C₆)alkyl, (C₃-C₁₀)cycloalkyl, (C₁-C₆)alkoxy, halogen and —OH; and
R²⁸ and R²⁹ are independently selected from the group consisting of H, (C₁-C₆)alkyl, (C₁-C₆)alkoxy, R²⁷-aryl(C₁-C₆)alkyl, heteroaryl, heteroaryl(C₁-C₆)alkyl, hydroxy(C₁-C₆)alkyl, (C₁-C₆)alkoxy(C₁-C₆)alkyl, heterocyclyl, heterocyclylalkyl, and halo(C₁-C₆)alkyl; or
R²⁸ and R²⁹ taken together form a spirocyclic or a heterospirocyclic ring of 3-6 atoms;
b) Formula I-B: 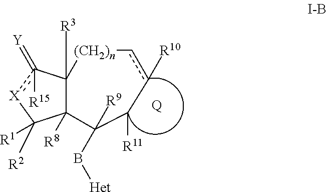
or a pharmaceutically acceptable salt thereof, wherein:
the single dotted line represents an optional double bond;
the double dotted line represents an optional single bond;
n is 0-2;
Q is 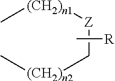
wherein n₁ and n₂ are independently 0-2; or when the double bond is not present, Q is also fused R-substituted aryl or R-substituted heteroaryl;
R is 1 to 3 substituents independently selected from the group consisting of H, (C₁-C₆)alkyl, halogen, hydroxy, amino, (C₁-C₆)alkyl-amino, (C₁-C₆)di-alkylamino, (C₁-C₆)alkoxy, —COR¹⁶, —COOR¹⁷, —SOR¹⁶, —SO₂R¹⁶, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl-(C₂-C₆)alkenyl, heteroaryl(C₁-C₆)-alkyl, heteroaryl(C₂-C₆)alkenyl, hydroxy-(C₁-C₆)alkyl, amino(C₁-C₆)-alkyl, aryl and thio(C₁-C₆)alkyl;
R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)-alkyl, heteroaryl(C₂-C₆)alkenyl, hydroxy-(C₁-C₆)alkyl, amino(C₁-C₆)alkyl, aryl and thio(C₁-C₆)alkyl; or R¹ and R² together form an oxo group;
R³ is H, hydroxy, (C₁-C₆)alkoxy, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl-(C₂-C₆)alkenyl, heteroaryl(C₁-C₆)alkyl, heteroaryl(C₂-C₆)alkenyl, hydroxy-(C₁-C₆)alkyl, amino(C₁-C₆)alkyl, aryl or thio(C₁-C₆)alkyl;
Het is pyridyl, quinolyl or benzoquinolyl, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl; (C₃-C₁₀)cycloalkyl; heterocycloalkyl; heterocycloalkyl substituted by (C₁-C₆)alkyl or (C₂-C₆)alkenyl; (C₂-C₆)alkenyl; R²¹-aryl(C₁-C₆)alkyl; R²¹-aryl-(C₂-C₆)-alkenyl; heteroaryl(C₁-C₆)alkyl; heteroaryl(C₂-C₆)alkenyl; hydroxy-(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl; thio(C₁-C₆)alkyl; (C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; (C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, aryl wherein adjacent carbons form a ring with a methylenedioxy group, —C(O)NR⁴R⁵ and heteroaryl; R²¹-aryl; aryl wherein adjacent carbons form a ring with a methylenedioxy group; heteroaryl; heteroaryl substituted by 1 to 4 substituents selected from the group consisting of halogen, (C₁-C₆)alkoxy, (C₁-C₆)alkyl, (C₁-C₆)alkyl-amino, di-((C₁-C₆)alkyl)amino, —OCF₃, —NO₂, hydroxy(C₁-C₆)alkyl, —CHO and phenyl; and heteroaryl wherein adjacent carbon atoms form a ring with a C₃-C₅ alkylene group or a methylenedioxy group;
R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₄—, —(CH₂)₅— or —(CH₂)₂NR⁷—(CH₂)₂— and form a ring with the nitrogen to which they are attached;
R⁶ is independently selected from the group consisting of H, C₁-C₆ alkyl or phenyl;
R⁷ is H or (C₁-C₆)alkyl;
R⁸, R¹⁰ and R¹¹ are independently selected from the group consisting of R¹ and —OR¹, provided that when the optional double bond is present, R¹⁰ is absent, and when ring Q is aromatic, R¹⁰ and R¹¹ are absent;
R⁹ is H, OH, (C₁-C₆)alkoxy, halogen or halo(C₁-C₆)alkyl;
B is —(CH₂)ₙ₃—, 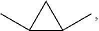
cis or trans —(C₂)ₙ₄CR¹²═CR^{12a}(CH₂)ₙ₅ or, —(CH₂)ₙ₄C═CR(CH₂)ₙ₅—, wherein n₃ is 0-5, n₄ and n₅ are independently 0-2, and R¹² and R^{12a} are independently selected from the group consisting of H, (C₁-C₆)alkyl and halogen;
X is —O—;
Y is oxo, thioxo, (H, H), (H, OH) or (H, (C₁-C₆)alkoxy);
R¹⁵ is absent;
R¹⁶ and R^{16a} are independently selected from the group consisting of (C₁-C₆)lower alkyl, phenyl or benzyl;
R¹⁷, R¹⁸ and R¹⁹ are independently selected from the group consisting of H, (C₁-C₆)alkyl, phenyl, benzyl;
R²⁰ is H, (C₁-C₆)alkyl, phenyl, benzyl, —C(O)R⁶ or —SO₂R⁶;
R²¹ is 1 to 3 substituents independently selected from the group consisting of —CF₃, —OCF₃, halogen, —NO₂, (C₁-C₆)alkyl, (C₁-C₆)alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶ and —NHSO₂CH₂CF₃; and
Z is —CH₂—, —C(O)—, —C(═NOR¹⁷)— or —C(R¹³R¹⁴)—, wherein R¹³ and R¹⁴, together with the carbon to which they are attached, form a spirocycloalkyl group of 3 to 6 carbons, or a spiroheterocycloalkyl group of 3 to 6 members, comprised of 2 to 5 carbon atoms and 1 or 2 heteroatoms selected from the group consisting of O, S and N,
c) Formula I-C: 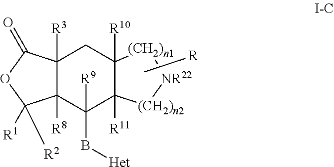
or a pharmaceutically acceptable salt thereof, wherein:
R is 1 to 3 substituents independently selected from the group consisting of H, (C₁-C₆)alkyl, halogen, hydroxy, amino, (C₁-C₆)alkyl-amino, (C₁-C₆)-dialkylamino, (C₁-C₆)alkoxy, —COR¹⁶, —COOR¹⁷, —SOR¹⁶, —SO₂R¹⁶, —SO₂NR¹⁷R¹⁸, —NR¹⁷SO₂R¹⁸, —NR¹⁶COR^{16a}, —NR¹⁶COOR^{16a}, —NR¹⁶CONR⁴R⁵, fluoro-(C₁-C₆)alkyl, difluoro(C₁-C₆)alkyl, trifluoro(C₁-C₆)alkyl, (C₃-C₁₀)cycloalkyl, aryl(C₁-C₆)alkyl, hydroxy(C₁-C₆)alkyl, amino-(C₁-C₆)-alkyl, aryl and thio(C₁-C₆)alkyl;
R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, hydroxy-(C₁-C₆)alkyl, amino(C₁-C₆)alkyl, aryl and thio(C₁-C₆)alkyl; or R¹ and R² together form an oxo group;
R³ is H, hydroxy, (C₁-C₆)alkoxy, aryloxy, aryl(C₁-C₆)alkyloxy, heteroaryloxy, heteroaryl(C₁-C₆)alkyloxy, (C₃-C₁₀)cycloalkyloxy, —SOR¹⁶, —SO₂R¹⁷, —SO₂NR¹⁸R¹⁹, —SR¹⁸, —SO₃H, —C(O)OR¹⁷, —C(O)NR¹⁸R¹⁹, —OC(O)R³², —OC(O)NR³³R³⁴, —(CR³³R³⁴)ₙOR³², —NR⁴R⁵, —NR³³COOR³², —NR³³COR³², —NR³³S(O)₂R³², —NR³³CONR³³R³⁴, —NR³³S(O)₂NR³³R³⁴, —(CR³³R³⁴)ₙNR⁴R⁵, —(CR³³R³⁴)ₙNR³³COOR³², —(CR³³R³⁴)ₙNR³³COR³², —(CR³³R³⁴)ₙNR³³S(O)₂R³², —(CR³³R³⁴)ₙNR³³CONR³³R³⁴, —(CR³³R³⁴)ₙNR³³S(O)₂NR³³R³⁴, (C₁-C₆)alkyl, halogen, (C₃-C₁₀)cycloalkyl, (C₂-C₆)alkenyl, —CN, aryl, heteroaryl, heterocycloalkyl, —P(O)(OR⁷)₂ or (C₁-C₆)alkyl substituted by 1 to 3 substituents independently selected from the group consisting of halogen, —OH, —NH₂, aryl, —COON, —SO₃H, thio and (C₁-C₆)alkylthio;
n is 1, 2, 3 or 4;
n1 and n2 are independently 0-3, provided both are not 0;
Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of (C₁-C₆)alkyl; —NR⁴R⁵; —NHCOR²⁶; —NHSO₂R¹⁶; R²¹-aryl; aryl wherein adjacent carbons form a ring with a methylenedioxy group; and
R²¹-heteroaryl;
R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₃—, —(CH₂)₄—, —(CH₂)₅— or —(CH₂)₂NR⁷—(CH₂)₂— and form a ring with the nitrogen to which they are attached;
R⁷ is H or (C₁-C₆)alkyl;
R⁸, R¹⁰ and R¹¹ independently independently selected from the group consisting of R¹ and —OR¹;
R⁹ is H, OH, —NR⁴R⁵, (C₁-C₆)alkoxy, halogen or halo(C₁-C₆)alkyl;
B is —(CH₂)n₃— or cis or trans —(CH₂)n₄CR¹²═CR^{12a}(CH₂)n₅, wherein n₃ is 0-5, n₄ and n₅ are independently 0-2, and R¹² and R^{12a} are independently selected from the group consisting of H, (C₁-C₆)alkyl and halogen;
R¹⁶ and R^{16a} are independently selected from the group consisting of (C₁-C₆)alkyl, phenyl and benzyl;
R¹⁷, R¹⁸ and R¹⁹ are independently selected from the group consisting of H, (C₁-C₆)alkyl, phenyl and benzyl;
R²¹ is 1 to 3 substituents independently selected from the group consisting of H, —CF₃, —OCF₃, halogen, —NO₂, —CN, (C₁-C₆)alkyl, (C₁-C₆)alkoxy, —NH₂, (C₁-C₆)-alkyl-amino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —CONR²⁴R²⁵, —NHCOR¹⁶, —NHSO₂R¹⁶, —NHSO₂CH₂CF₃, —SO₂NR²⁴R²⁵, —NR²⁹C(O)NR²⁴R²⁵, —SO₂R³⁰, —P(O)(OR²⁹)₂, aryl, aryl(C₁-C₆)alkyl, heteroaryl, heterocycloalkyl, and —CR²⁹(═NOR²⁸);
R²² is —COR²³, —S(O)R³¹, —S(O)₂R³¹, —SO₂NR²⁴R²⁵ or —COOR²⁷;
R²³ is halo(C₁-C₆)alkyl; (C₂-C₆)alkenyl; halo(C₂-C₆)alkenyl; (C₂-C₆)alkynyl; (C₃-C₁₀)-cycloalkyl; (C₃-C₁₀)cycloalkyl(C₁-C₆)alkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₂-C₆)alkyl; heteroaryl; heterocycloalkyl; (C₁-C_{C}₆)alkyl substituted by 1-3 substituents independently selected from —COOH and —SO₃H; or 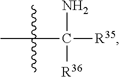
wherein R³⁵ and R³⁶ are independently selected from the group consisting of H, (C₁-C₆)alkyl, or R³⁷-substituted (C₁-C₆)alkyl, wherein R³⁷ is selected from the group consisting of HO—, HS—, CH₂S—, —NH₂, phenyl, p-hydroxyphenyl and indolyl;
R²⁴ and R²⁵ are independently selected form the group consisting of H, (C₁-C₆)alkyl, halo(C₁-C₆)alkyl, (C₂-C₆)alkenyl, halo(C₂-C₆)alkyl, (C₂-C₆)alkynyl, aryl, aryl-(C₁-C₆)alkyl, (C₃-C₁₀)cycloalkyl, halo(C₃-C₁₀)cycloalkyl, (C₁-C₃)alkoxy(C₁-C₃)-alkyl, hydroxy and (C₁-C₆)alkoxy;
R²⁶ is (C₃-C₁₀)cycloalkyl, aryl, aryl-(C₁-C₆)alkyl, heteroaryl, heteroaryl-(C₁-C₆)alkyl or (C₁-C₆)alkylamino;
R²⁷ is (C₁-C₆)alkyl, phenyl, benzyl, (C₁-C₃)alkoxy(C₁-C₃)-alkyl, (C₃-C₁₀)cycloalkyl, carboxy(C₁-C₆)alkyl, sulfo(C₁-C₆)alkyl, or (C₁-C₆)alkyl substituted by NR¹⁸R¹⁹ and carboxy;
R²⁸ is H, (C₁-C₆)alkyl, phenyl, benzyl or (C₁-C₃)alkoxy(C₁-C₃)alkyl;
R²⁹ and R³⁰ are independently selected from the group consisting of H and (C₁-C₆)alkyl;
R³¹ is (C₁-C₆)alkyl; halo(C₁-C₆)alkyl; (C₂-C₆)alkenyl; halo(C₂-C₆)alkyl; (C₂-C₆)alkynyl; (C₃-C₁₀)cycloalkyl; (C₃-C₁₀)cycloalkyl substituted by 1 to 3 substituents selected from the group consisting of halo, (C₁-C₃)alkoxy(C₁-C₃)alkyl, hydroxy and (C₁-C₆)alkoxy; aryl; aryl(C₁-C₆)alkyl; heteroaryl; heterocycloalkyl; (C₁-C₆)alkyl substituted by 1-3 substituents independently selected from —COOH and —SO₃H; or (C₁-C₆)alkoxy;
R³² is R³⁵—(C₁-C₆)alkyl, R³⁵—(C₃-C₁₀)cycloalkyl, R³⁵—(C₂-C₆)alkenyl, R³⁵—(C₂-C₆)-alkynyl or R³⁵-aryl, wherein R³⁵ is 1 or 2 substituents independently selected from the group consisting of H, —COOH, —NH₂, —SO₃H, oxo and ═NOR²⁸; and
R³³ and R³⁴ are independently selected from the group consisting of H, (C₁-C₆)alkyl and (C₃-C₁₀)cycloalkyl;
d) Formula I-D: 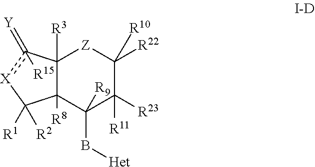
or a pharmaceutically acceptable salt thereof, wherein:
Z is —(CH₂)ₙ—; 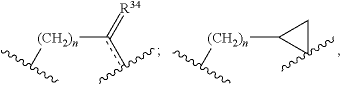
wherein R¹⁰ is absent; or 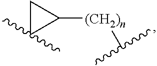
wherein R³ is absent;
the single dotted line represents an optional double bond;
the double dotted line represents an optional single bond;
n is 0-2;
R¹ and R² are independently selected from the group consisting of H, (C₁-C₆)alkyl, fluoro(C₁-C₆)alkyl, difluoro(C₁-C₆)alkyl, trifluoro-(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy-(C₁-C₆)alkyl, (C₁-C₆)alkoxy(C₁-C₆)alkyl, amino-(C₁-C₆)alkyl, aryl and thio(C₁-C₆)alkyl; or R¹ and R² together form an oxo group;
R³ is H, hydroxy, (C₁-C₆)alkoxy, —NR¹⁸R¹⁹, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl;
R³⁴ is (H, R³), (H, R⁴³), oxo or ═NOR¹⁷ when the optional double bond is absent; R³⁴ is R⁴⁴ when the double bond is present;
Het is a mono-, bi- or tricyclic heteroaromatic group of 5 to 14 atoms comprised of 1 to 13 carbon atoms and 1 to 4 heteroatoms independently selected from the group consisting of N, O and S, wherein a ring nitrogen can form an N-oxide or a quaternary group with a C₁-C₄ alkyl group, wherein Het is attached to B by a carbon atom ring member, and wherein the Het group is substituted by 1 to 4 substituents, W, independently selected from the group consisting of H; (C₁-C₆)alkyl; fluoro(C₁-C₆)alkyl; difluoro(C₁-C₆)alkyl; trifluoro-(C₁-C₆)-alkyl; (C₃-C₁₀)cycloalkyl; heterocycloalkyl; heterocycloalkyl substituted by (C₁-C₆)alkyl, (C₂-C₆)alkenyl, OH—(C₁-C₆)alkyl, or oxo; (C₂-C₆)alkenyl; R²¹-aryl(C₁-C₆)alkyl; R²¹-aryl-(C₂-C₆)-alkenyl; R₂₁-aryloxy; R²¹-aryl-NH—; heteroaryl(C₁-C₆)alkyl; heteroaryl(C₂-C₆)-alkenyl; heteroaryloxy; heteroaryl-NH—; hydroxy(C₁-C₆)alkyl; dihydroxy(C₁-C₆)alkyl; amino(C₁-C₆)alkyl; (C₁-C₆)alkylamino-(C₁-C₆)alkyl; di-((C₁-C₆)alkyl)-amino(C₁-C₆)alkyl, thio(C₁-C₆)alkyl; (C₁-C₆)alkoxy; (C₂-C₆)alkenyloxy; halogen; —NR⁴R⁵; —CN; —OH; —COOR¹⁷; —COR¹⁶; —OSO₂CF₃; —CH₂OCH₂CF₃; (C₁-C₆)alkylthio; —C(O)NR⁴R⁵; —OCHR⁶-phenyl; phenoxy-(C₁-C₆)alkyl; —NHCOR¹⁶; —NHSO₂R¹⁶; biphenyl; —OC(R⁶)₂COOR⁷; —OC(R⁶)₂C(O)NR⁴R⁵; (C₁-C₆)alkoxy; —C(═NOR¹⁷)R¹⁸; (C₁-C₆)alkoxy substituted by (C₁-C₆)alkyl, amino, —OH, COOR¹⁷, —NHCOOR¹⁷, —CONR⁴R⁵, aryl, aryl substituted by 1 to 3 substituents independently selected from the group consisting of halogen, —CF₃, (C₁-C₆)alkyl, (C₁-C₆)alkoxy and —COOR¹⁷, aryl wherein adjacent carbons form a ring with a methylenedioxy group, —C(O)NR⁴R⁵ or heteroaryl; R²¹-aryl; aryl wherein adjacent carbons form a ring with a methylenedioxy group; R⁴¹-heteroaryl; and heteroaryl wherein adjacent carbon atoms form a ring with a C₃-C₅ alkylene group or a methylenedioxy group;
R⁴ and R⁵ are independently selected from the group consisting of H, (C₁-C₆)alkyl, phenyl, benzyl and (C₃-C₁₀)cycloalkyl, or R⁴ and R⁵ together are —(CH₂)₄—, —(CH₂)₅— or —(CH₂)₂NR⁷—(CH₂)₂— and form a ring with the nitrogen to which they are attached;
R⁶ is independently selected from the group consisting of H, (C₁-C₆)alkyl, phenyl, (C₃-C₁₀)cycloalkyl, (C₃-C₁₀)cycloalkyl(C₁-C₆)alkyl, (C₁-C₆)alkoxy(C₁-C₆)alkyl, hydroxy(C₁-C₆)alkyl and amino(C₁-C₆)alkyl;
R⁷ is H or (C₁-C₆)alkyl;
R⁸, R¹⁰ and R¹¹ are independently selected from the group consisting of R¹ and —OR¹, provided that when the optional double bond is present, R¹⁰ is absent;
R⁹ is H, OH, (C₁-C₆)alkoxy, halogen or halo(C₁-C₆)alkyl;
B is —(CH₂)ₙ₃—, —CH₂—O—, —CH₂S—, —CH₂—NR⁶—, —C(O)NR⁶—, —NR⁶C(O)—, 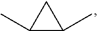
cis or trans —(CH₂)ₙ₄CR¹²═CR^{12a}(CH₂)ₙ₅ or —(CH₂)ₙ₄C═CR(CH₂)ₙ₅—, wherein n₃ is 0-5, n₄ and n₅ are independently 0-2, and R¹² and R^{12a} are independently selected from the group consisting of H, (C₁-C₆)alkyl and halogen;
X is —O— or —NR⁶— when the double dotted line represents a single bond, or X is H, —OH or —NHR²⁰ when the bond is absent;
Y is oxo, thioxo, (H, H), (H, OH) or (H, (C₁-C₆)alkoxy) when the double dotted line represents a single bond, or when the bond is absent, Y is oxo, ═NOR¹⁷, (H, H), (H, OH), (H, SH), (H, (C₁-C₆)alkoxy) or (H, —NHR⁴⁵);
R¹⁵ is absent when the double dotted line represents a single bond; R¹⁵ is H, (C₁-C₆)alkyl, —NR¹⁸R¹⁹ or —OR¹⁷ when the bond is absent; or Y is 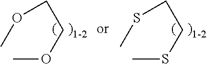
and R¹⁵ is H or (C₁-C₆)alkyl;
R¹⁶ is (C₁-C₆)lower alkyl, phenyl or benzyl;
R¹⁷, R¹⁸ and R¹⁹ are independently selected from the group consisting of H, (C₁-C₆)alkyl, phenyl, benzyl;
R²⁰ is H, (C₁-C₆)alkyl, phenyl, benzyl, —C(O)R⁶ or —SO₂R⁶;
R²¹ is 1 to 3 substituents independently selected from the group consisting of hydrogen, —CF₃, —OCF₃, halogen, —NO₂, (C₁-C₆)alkyl, (C₁-C₆)alkoxy, (C₁-C₆)alkylamino, di-((C₁-C₆)alkyl)amino, amino(C₁-C₆)alkyl, (C₁-C₆)-alkylamino(C₁-C₆)alkyl, di-((C₁-C₆)alkyl)-amino(C₁-C₆)alkyl, hydroxy-(C₁-C₆)alkyl, —COOR¹⁷, —COR¹⁷, —NHCOR¹⁶, —NHSO₂R¹⁶, —NHSO₂CH₂CF₃, heteroaryl or —C(═NOR¹⁷)R¹⁸;
R²² and R²³ are independently selected from the group consisting of hydrogen, R²⁴—(C₁-C₆)alkyl, R²⁴⁻(C₂-C₆)alkenyl, R²⁴—(C₂-C₆)alkynyl, R²⁷-hetero-cycloalkyl, R²⁵-aryl, R²⁵-aryl(C₁-C₆)alkyl, R²⁹—(C₃-C₁₀)cycloalkyl, R²⁹(C₃-C₁₀)cycloalkenyl, —OH, —OC(O)R³⁰, —C(O)OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₁-C₆)alkoxy, R²⁴—(C₂-C₆)-alkenyloxy, R²⁴—(C₂-C₆)alkynyloxy, R²⁷-heterocycloalkyloxy, R²⁹—(C₃-C₁₀)cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);
or R²² and R¹⁰ together with the carbon to which they are attached, or R²³ and R¹¹ together with the carbon to which they are attached, independently form a R⁴²-substituted carbocyclic ring of 3-10 atoms, or a R⁴²-substituted heterocyclic ring of 4-10 atoms wherein 1-3 ring members are independently selected from the group consisting of —O—, —NH— and —SO₀₋₂—, provided that when R²² and R¹⁰ form a ring, the optional double bond is absent;
R²⁴ is 1, 2 or 3 substituents independently selected from the group consisting of hydrogen, halogen, —OH, (C₁-C₆)alkoxy, R³⁵-aryl, (C₁-C₆)alkyl-C(O)—, (C₂-C₆)-alkenyl-C(O)—, (C₂-C₆)alkynyl-C(O)—, heterocycloalkyl, R²⁶—(C₃-C₁₀)cycloalkyl, R²⁶—(C₃-C₁₀)cycloalkenyl, —OC(O)R³⁰, —C(O)OR³⁰, —C(O)R³⁰, —C(O)NR³⁰R³¹, —NR³⁰R³¹, —NR³⁰C(O)R³¹, —NR³⁰C(O)NR³¹R³², —NHSO₂R³⁰, —OC(O)NR³⁰R³¹, R²⁴—(C₂-C₆)-alkenyloxy, R²⁴—(C₂-C₆)alkynyloxy, R²⁷-heterocycloalkyloxy, R²⁹—(C₃-C₁₀)cycloalkyloxy, R²⁹—(C₃-C₁₀)cyclo-alkenyloxy, R²⁹—(C₃-C₁₀)cycloalkyl-NH—, —NHSO₂NHR¹⁶ and —CH(═NOR¹⁷);
R²⁵ is 1, 2 or 3 substituents independently selected from the group consisting of hydrogen, heterocycloalkyl, halogen, —COOR³⁶, —CN, —C(O)NR³⁷R³⁸, —NR³⁹C(O)R⁴⁰, —OR³⁶, (C₃-C₁₀)cycloalkyl, (C₃-C₁₀)cycloalkyl-(C₁-C₆)alkyl, (C₁-C₆)alkyl(C₃-C₁₀)cycloalkyl-(C₁-C₆)alkyl, halo(C₁-C₆)alkyl(C₃-C₁₀)cycloalkyl(C₁-C₆)alkyl, hydroxy(C₁-C₆)alkyl, (C₁-C₆)alkoxy(C₁-C₆)alkyl, and R⁴¹-heteroaryl; or two R²⁵ groups on adjacent ring carbons form a fused methylenedioxy group
R²⁶ is 1, 2, or 3 substituents independently selected from the group consisting of hydrogen, halogen and (C₁-C₆)alkoxy;
R²⁷ is 1, 2 or 3 substituents independently selected from the group consisting of hydrogen, R²⁸—(C₁-C₆)alkyl, R²⁸—(C₂-C₆)alkenyl, R²⁸—(C₂-C₆)alkynyl,
R²⁸ is hydrogen, —OH or (C₁-C₆)alkoxy;
R²⁹ is 1, 2 or 3 substituents independently selected from the group consisting of hydrogen, (C₁-C₆)alkyl, —OH, (C₁-C₆)alkoxy and halogen;
R³⁰, R³¹ and R³² are independently selected from the group consisting of hydrogen, (C₁-C₆)-alkyl, (C₁-C₆)alkoxy(C₁-C₆)-alkyl, R²⁵-aryl(C₁-C₆)-alkyl, R³³—(C₃-C₁₀)cycloalkyl, R³⁴⁻(C₃-C₁₀)cycloalkyl(C₁-C₆)alkyl, R²⁵-aryl, heterocycloalkyl, heteroaryl, heterocycloalkyl(C₁-C₆)alkyl and heteroaryl(C₁-C₆)alkyl;
R³³ is hydrogen, (C₁-C₆)alkyl, OH—(C₁-C₆)alkyl or (C₁-C₆)alkoxy;
R³⁵ is 1 to 4 substituents independently selected from the group consisting of hydrogen, (C₁-C₆)alkyl, —OH, halogen, —CN, (C₁-C₆)alkoxy, trihalo(C₁-C₆)alkoxy, (C₁-C₆)alkylamino, di((C₁-C₆)alkyl)amino, —OCF₃, OH—(C₁-C₆)alkyl, —CHO, —C(O)(C₁-C₆)-alkylamino, —C(O)di((C₁-C₆)alkyl)amino, —NH₂, —NHC(O)(C₁-C₆)alkyl and —N((C₁-C₆)alkyl)C(O)(C₁-C₆)alkyl;
R³⁶ is hydrogen, (C₁-C₆)alkyl, halo(C₁-C₆)alkyl, dihalo(C₁-C₆)alkyl or trifluoro(C₁-C₆)alkyl,
R³⁷ and R³⁸ are independently selected from the group consisting of hydrogen, (C₁-C₆)alkyl, aryl(C₁-C₆)alkyl, phenyl and (C₃-C₁₅)cycloalkyl, or R³⁷ and R³⁸ together are —(CH₂)₄—, —(CH₂)₅— or —(CH₂)₂—NR³⁹—(CH₂)₂— and form a ring with the nitrogen to which they are attached;
R³⁹ and R⁴⁰ are independently selected from the group consisting of hydrogen, (C₁-C₆)alkyl, aryl(C₁-C₆)alkyl, phenyl and (C₃-C₁₅)-cycloalkyl, or R³⁹ and R⁴⁰ in the group —NR³⁹C(O)R⁴⁰, together with the carbon and nitrogen atoms to which they are attached, form a cyclic lactam having 5-8 ring members;
R⁴¹ is 1 to 4 substituents independently selected from the group consisting of hydrogen, (C₁-C₆)alkyl, (C₁-C₆)alkoxy, (C₁-C₆)alkylamino, di((C₁-C₆)alkyl)amino, —OCF₃, OH—(C₁-C₆)alkyl, —CHO and phenyl;
R⁴² is 1 to 3 substituents independently selected from the group consisting of hydrogen, —OH, (C₁-C₆)alkyl and (C₁-C₆)alkoxy;
R⁴³ is —NR³⁰R³¹, —NR³⁰C(O)R³¹, —NR³⁰C(O)NR³¹R³², —NHSO₂R³⁰ or —NHCOOR¹⁷;
R⁴⁴ is H, (C₁-C₆)alkoxy, —SOR¹⁶, —SO₂R¹⁷, —C(O)OR¹⁷, —C(O)NR¹⁸R¹⁹, (C₁-C₆)alkyl, halogen, fluoro(C₁-C₆)alkyl, difluoro(C₁-C₆)alkyl, trifluoro(C₁-C₆)alkyl, (C₃-C₁₀)cycloalkyl, (C₂-C₆)alkenyl, aryl(C₁-C₆)alkyl, aryl(C₂-C₆)alkenyl, heteroaryl(C₁-C₆)alkyl, heteroaryl(C₂-C₆)alkenyl, hydroxy(C₁-C₆)alkyl, amino(C₁-C₆)alkyl, aryl, thio(C₁-C₆)alkyl, (C₁-C₆)alkoxy(C₁-C₆)alkyl or (C₁-C₆)alkylamino(C₁-C₆)alkyl; and
R⁴⁵ is H, (C₁-C₆)alkyl, —COOR¹⁶ or —SO₂; and
e) Formula I-E-1: 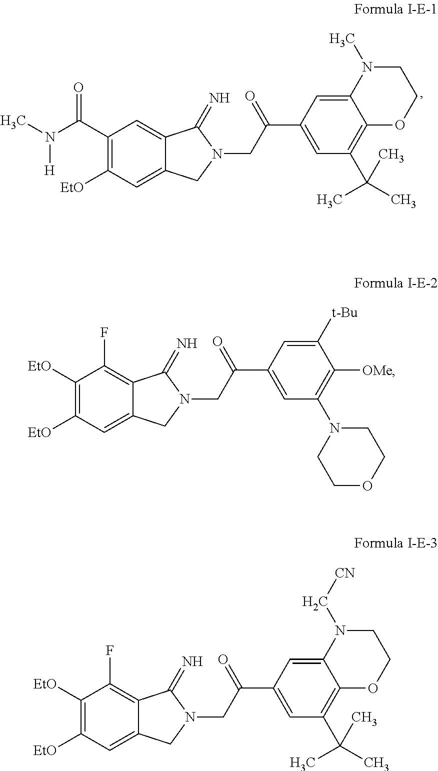
or a pharmaceutically acceptable salt thereof; and
ii) an effective amount of at least one NAR agonist which is selected from the group consisting of:
a) Formula II: 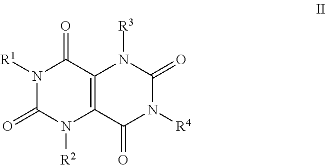
or a pharmaceutically acceptable salt, solvate, ester or prodrug thereof, wherein:
R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, heteroaryl, —OR⁵, —SR⁵, —N(R⁶)₂, —CN, —C(O)OR⁵ and —C(O)N(R⁶)₂;
R² is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵, —NHC(O)—R⁶ and —C(O)N(R⁶)₂;
R³ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R⁴ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
each occurrence of R⁵ is independently H, (C₁-C₆)alkyl, aryl or (C₃-C₁₀)cycloalkyl;
each occurrence of R⁶ is independently H, (C₁-C₆)alkyl, -(alkylene)ₙ-aryl or (C₃-C₁₀)cycloalkyl; and
each occurrence of n is independently 0 or 1;
b) Formula III: 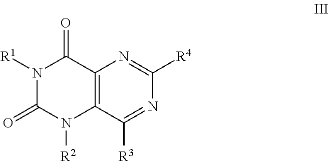
and pharmaceutically acceptable salts, solvates, esters and prodrugs thereof, wherein:
R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R² is H, (C₁-C₆)alkyl, -(alkylene)ₙ-aryl, -(alkylene)ₙ-(C₃-C₁₀)cycloalkyl, -(alkylene)ₙ—(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R³ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R⁴ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
each occurrence of R⁵ is independently H, (C₁-C₆)alkyl, aryl or (C₃-C₁₀)cycloalkyl;
each occurrence of R⁶ is independently H, (C₁-C₆)alkyl, -(alkylene)ₙ-aryl or (C₃-C₁₀)cycloalkyl; and
each occurrence of n is independently 0 or 1;
c) Formula IV: 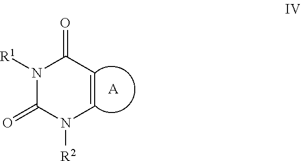
and pharmaceutically acceptable salts and solvates thereof, wherein:
A is selected from the group consisting of: 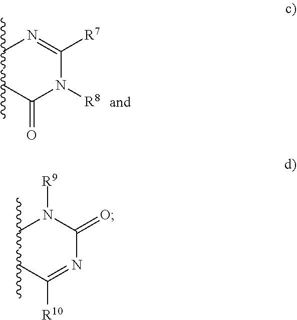
R¹ is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R² is H, (C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl or -(alkylene)ₙ-heteroaryl, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
each occurrence of R⁵ is independently H, (C₁-C₆)alkyl, aryl or (C₃-C₁₀)cycloalkyl;
each occurrence of R⁶ is independently H, (C₁-C₆)alkyl, -(alkylene)ₙ-aryl or (C₃-C₁₀)cycloalkyl;
R⁷ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R⁸ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R⁹ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, —OR⁵, —SR⁵, —N(R⁶)₂, —CN, —C(O)OR⁵ and —C(O)N(R⁶)₂;
R¹⁰ is H, (C₁-C₆)alkyl, halo(C₁-C₆)alkyl, -(alkylene)ₙ-aryl, -(alkylene)ₙ-(C₃-C₁₀)cycloalkyl, -(alkylene)ₙ-(C₃-C₁₀)cycloalkenyl, -(alkylene)ₙ-heterocycloalkyl, -(alkylene)ₙ-heterocycloalkenyl, -(alkylene)ₙ-heteroaryl, —OR⁵, —SR⁵, —N(R⁶)₂, —CN, —C(O)OR⁵ or —C(O)N(R⁶)₂, wherein any aryl, (C₃-C₁₀)cycloalkyl, (C₃-C₁₀)cycloalkylene, heterocycloalkyl, heterocycloalkenyl or heteroaryl group can be unsubstituted or substituted with up to 4 substituents, which can be the same or different, and are selected from: (C₁-C₆)alkyl, aryl, halo, halo(C₁-C₆)alkyl, OR⁵, —SR⁵, —N(R⁶)₂, —CN, —C(O)OR⁵ and —C(O)N(R⁶)₂; and
each occurrence of n is independently 0 or 1;
d) Formula V: 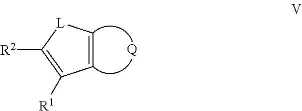
or a pharmaceutically acceptable salt, solvate, ester, or tautomer thereof, wherein:
Q is selected from the group consisting of: 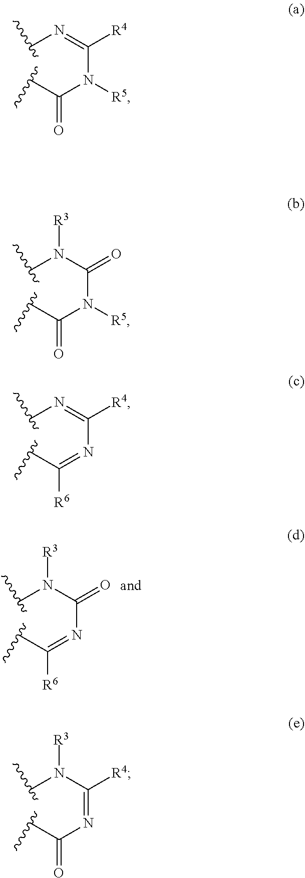
L is selected from the group consisting of: 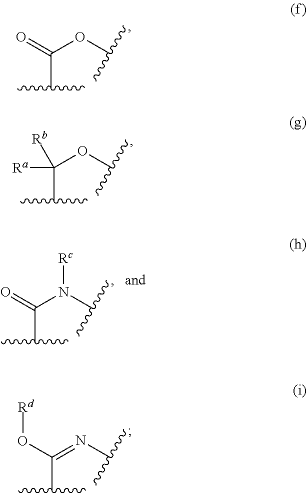
R¹ is selected from the group consisting of H, (C₁-C₆)alkyl, (C₂-C₆)alkenyl, (C₂-C₆)alkynyl, halo(C₁-C₆)alkyl, (C₁-C₆)alkyl substituted with one or more hydroxyl groups, (C₃-C₁₀)cycloalkyl, —C(O)—(C₁-C₆)alkyl, —(C₂-C₆)alkylene-C(O)—O—(C₁-C₆)alkyl, —O—R¹⁰, -alkylene-O—(C₁-C₆)alkyl, aryl, -alkylene-aryl, heteroaryl, -alkylene-heteroaryl, halogen, —(CH₂)ₙ—N(R⁷)₂, -alkylene-(C₃-C₁₀)cycloalkyl, and -alkylene-(C₃-C₁₀)cycloalkenyl,
wherein said (C₃-C₁₀)cycloalkyl or the (C₃-C₁₀)cycloalkyl portion of said -alkylene-(C₃-C₁₀)cycloalkyl of R¹ is unsubstituted or substituted with one or more X groups, said aryl or the aryl portion of said -alkylene-aryl of R¹ is unsubstituted or substituted with one or more Y groups, and said heteroaryl or the heteroaryl portion of said -alkylene-heteroaryl of R¹ is unsubstituted or substituted with one or more Y groups;
R² is selected from the group consisting of H, halogen, (C₁-C₆)alkyl, halo(C₁-C₆)alkyl, (C₁-C₆)alkyl substituted with one or more —OH, —C(O)—(C₁-C₆)alkyl, —C(O)—O—(C₁-C₆)alkyl, —C(O)—OH, —O—R¹⁰, -alkylene-O—(C₁-C₆)alkyl, unsubstituted aryl, aryl substituted with one or more Y groups, unsubstituted heteroaryl, heteroaryl substituted with one or more Y groups, and halogen; or
R¹ and R² together with the ring carbon atoms to which they are shown attached, form a 5- or 6-membered cycloalkenyl ring or a 5- or 6-membered heterocyclic ring having 1 or 2 heteroatoms;
R³ is selected from the group consisting of H, (C₁-C₆)alkyl, (C₁-C₆)alkyl substituted with one or more hydroxyl groups, -alkylene-O—(C₁-C₆)alkyl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, (C₂-C₆)alkenyl, aryl, and heteroaryl,
wherein said (C₃-C₁₀)cycloalkyl or the (C₃-C₁₀)cycloalkyl portion of said -alkylene-(C₃-C₁₀)cycloalkyl of R³ is unsubstituted or substituted with one or more X groups, said aryl of R³ is unsubstituted or substituted with one or more Y groups, and said heteroaryl of R³ is unsubstituted or substituted with one or more Y groups;
R⁴ is selected from the group consisting of H, halogen, (C₁-C₆)alkyl, —O—R¹⁰, —C(O)—O—(C₁-C₆)alkyl, —S(O)ₘ—R⁹, —N(R⁷)₂, —N(R⁷)—NH—C(O)—(C₁-C₆)alkyl, —N(R⁷)—NH—C(O)—O—(C₁-C₆)alkyl, —O—N═C(R¹²)₂, —N(R⁷)—N═C(R¹²)₂, —C(O)—(C₁-C_{C}₆)alkyl, unsubstituted heterocyclyl, heterocyclyl substituted with one or more X groups, —O—N(R⁷)—C(O)—O—(C₁-C₆)alkyl, —C(O)—N(R⁷)₂, —CN, —N₃, and —O—C(O)—(C₁-C₆)alkyl;
R⁵ is selected from the group consisting of H, (C₁-C₆)alkyl, -alkylene-C(O)—R⁸, -alkylene-C(O)—N(R¹¹)₂, -alkylene-C(═N—O—(C₁-C₆)alkyl)-aryl, (C₃-C₁₀)cycloalkyl, -alkylene-(C₃-C₁₀)cycloalkyl, -alkylene-C(O)—O—(C₁-C₆)alkyl, -alkylene-O—C(O)—(C₁-C₆)alkyl, -alkylene-C(O)-heterocyclyl, and (C₂-C₆)alkenyl,
wherein said (C₃-C₁₀)cycloalkyl or the (C₃-C₁₀)cycloalkyl portion of said -alkylene-(C₃-C₁₀)cycloalkyl of R⁵ is unsubstituted or substituted with one or more X groups, and the aryl portion of said -alkylene-C(═N—O—(C₁-C₆)alkyl)-aryl of R⁵ is unsubstituted or substituted with one or more Y groups;
R⁶ is selected from the group consisting of H, (C₁-C₆)alkyl, (C₂-C₆)alkenyl, (C₁-C₆)alkyl substituted with one or more hydroxyl groups, -alkylene-O—(C₁-C₆)alkyl, —O—R¹⁰, halogen, aryl, heteroaryl, and —N(R⁷)₂,
wherein said aryl of R⁶ is unsubstituted or substituted with one or more Y groups, and said heteroaryl of R⁶ is unsubstituted or substituted with one or more Z groups;
each R⁷ is independently selected from the group consisting of H, (C₁-C₆)alkyl, (C₃-C₁₀)cycloalkyl, aryl, —C(O)—(C₁-C₆)alkyl, and —C(O)-aryl,
wherein said (C₃-C₁₀)cycloalkyl of R⁷ is unsubstituted or substituted with one or more X groups, and the aryl portion of said —C(O)-aryl or said aryl of R⁷ is unsubstituted or substituted with one or more Y groups; or
two R⁷ groups, together with the N atom to which they are bonded form a heterocyclyl;
R⁸ is selected from the group consisting of aryl, —OH, and heterocyclyl,
wherein said heterocyclyl of R⁸ is unsubstituted or substituted with one or more X groups, and said aryl of R⁸ is unsubstituted or substituted with one or more Y groups;
R⁹ is selected from the group consisting of (C₁-C₆)alkyl, -alkylene-(C₃-C₁₀)cycloalkyl, (C₂-C₆)alkenyl, —N(R¹¹)₂, and -alkylene-aryl,
wherein the (C₃-C₁₀)cycloalkyl portion of said -alkylene-(C₃-C₁₀)cycloalkyl of R⁹ is unsubstituted or substituted with one or more X groups, and the aryl portion of said -alkylene-aryl of R⁹ is unsubstituted or substituted with one or more Y groups, and
with the proviso that when R⁹ is —N(R¹¹)₂, m is 1 or 2;
R¹⁰ is selected from the group consisting of H, (C₁-C₆)alkyl, -alkylene-aryl, -alkenylene-aryl, -alkylene-heteroaryl, (C₂-C₆)alkenyl, —C(O)—(C₁-C₆)alkyl, (C₂-C₆)alkynyl, and -alkylene-(C₃-C₁₀)cycloalkyl,
wherein the (C₃-C₁₀)cycloalkyl portion of said -alkylene-(C₃-C₁₀)cycloalkyl of R¹⁰ is unsubstituted or substituted with one or more X groups, the aryl portion of said -alkylene-aryl or -alkenylene-aryl of R¹⁰ is unsubstituted or substituted with one or more Y groups, and the heteroaryl portion of said -alkylene-heteroaryl of R¹⁰ is unsubstituted or substituted with one or more Z groups;
each R¹¹ is independently selected from the group consisting of H, (C₁-C₆)alkyl, and aryl,
wherein said aryl of R¹¹ is unsubstituted or substituted with one or more Y groups; or
two R¹¹ groups, together with the N atom to which they are attached, form a heterocyclyl;
each R¹² is independently selected from the group consisting of (C₁-C₆)alkyl, aryl, and heteroaryl,
wherein said aryl of R¹² is unsubstituted or substituted with one or more Y groups and said heteroaryl of R¹² is unsubstituted or substituted with one or more Z groups;
R^{a} and R^{b} are each independently selected from the group consisting of H, (C₁-C₆)alkyl, aryl, and heteroaryl,
wherein said aryl of R^{a} and R^{b} is unsubstituted or substituted with one or more Y groups, and said heteroaryl of R^{a} and R^{b} is unsubstituted or substituted with one or more Z groups;
R^{c} is selected from the group consisting of H, (C₁-C₆)alkyl, alkylene-aryl, and —C(O)—(C₁-C₆)alkyl,
wherein the aryl portion of said alkylene-aryl of R^{c} is unsubstituted or substituted with one or more Y groups;
R^{d} is selected from the group consisting of H, (C₁-C₆)alkyl, and alkylene-aryl,
wherein the aryl portion of said alkylene-aryl of R^{d} is unsubstituted or substituted with one or more Y groups;
each X is independently selected from the group consisting of halogen, (C₁-C₆)alkyl, halo(C₁-C₆)alkyl, —O—(C₁-C₆)alkyl, —O-halo(C₁-C₆)alkyl, and —OH;
each Y is independently selected from the group consisting of halogen, (C₁-C₆)alkyl, halo(C₁-C₆)alkyl, —O—(C₁-C₆)alkyl, —O-halo(C₁-C₆)alkyl, —CN, —NO₂, —OH, —S(O₂)—(C₁-C₆)alkyl, —S(O₂)-aryl, —S(O₂)—NH₂, —S(O₂)—NH—(C₁-C₆)alkyl, —S(O₂)—NH-aryl, —S(O₂)—N((C₁-C₆)alkyl)₂, —S(O₂)—N(aryl)₂, —S(O₂)—N((C₁-C₆)alkyl)(aryl), and aryl;
each Z is independently selected from the group consisting of (C₁-C₆)alkyl, halo(C₁-C₆)alkyl, halogen, —O—(C₁-C₆)alkyl, —O-halo(C₁-C₆)alkyl, —CN, —OH, aryl, and N-oxide;
n is 0, 1, 2, or 3;
m is 0, 1, or 2; and
with the proviso that when L is (f), and R², R³ and R⁵ are each H, then R¹ is not —CH₃; and
e) Formula VI: 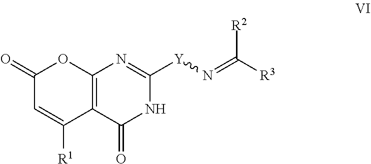
or a pharmaceutically acceptable salt, solvate, ester, or tautomer thereof, wherein:
R¹ is selected from the group consisting of H, R⁴, halo(C₁-C₆)alkyl, -alkylene-R⁴, -alkylene-R⁵, -alkylene-R⁶, (C₂-C₆)alkenyl, (C₂-C₆)alkynyl, and -alkylene-O—(C₁-C₆)alkyl;
R² is selected from the group consisting of (C₁-C₆)alkyl, halo(C₁-C₆)alkyl, -alkylene-R⁵, R⁴, R⁵, R⁶, R⁷ and -alkylene-O—R⁸;
R³ is selected from the group consisting of (C₁-C₆)alkyl, halo(C₁-C₆)alkyl, -alkylene-R⁵, R⁴, R⁵, R⁶, and R⁷; or
R² and R³ together with the carbon atom to which they are both attached form a (C₃-C₁₀)cycloalkyl or heterocycloalkyl ring, wherein said (C₃-C₁₀)cycloalkyl or heterocycloalkyl ring is unsubstituted or independently substituted with one or more X⁵ groups, and wherein said (C₃-C₁₀)cycloalkyl ring can form a spirocyclic compound with a second (C₃-C₁₀)cycloalkyl ring or with a heterocycloalkyl ring, wherein the second (C₃-C₁₀)cycloalkyl ring or the heterocycloalkyl ring is unsubstituted or independently substituted with one or more X⁵ groups;
R⁴ is unsubstituted (C₃-C₁₀)cycloalkyl or (C₃-C₁₀)cycloalkyl substituted with one or more X¹ groups;
R⁵ is unsubstituted aryl and aryl substituted with one or more X² groups;
R⁶ is selected from the group consisting of unsubstituted heteroaryl and heteroaryl substituted with one or more X³ groups;
R⁷ is unsubstituted heterocycloalkyl and heterocycloalkyl substituted with one or more X⁴ groups;
R⁸ is selected from the group consisting of H, (C₁-C₆)alkyl, R⁴, R⁵, R⁶, R⁷, —C(O)—(C₁-C₆)alkyl, —C(O)—R⁵;
each R⁹ is independently selected from the group consisting of H, (C₁-C₆)alkyl, R⁴, R⁵, R⁶, and R⁷;
R¹⁰ is selected from the group consisting of R⁹, —C(O)—(C₁-C₆)alkyl, and —C(O)—R⁵;
Y is —O— or —N(R¹⁰)—;
each X¹ is independently selected from the group consisting of halogen, (C₁-C₆)alkyl, —O—(C₁-C₆)alkyl, —OH, halo(C₁-C₆)alkyl, aryl, and (C₂-C₆)alkyne;
each X² is independently selected from the group consisting of halogen, (C₁-C₆)alkyl, —O—(C₁-C₆)alkyl, —OH, halo(C₁-C₆)alkyl, aryl, and (C₂-C₆)alkyne;
each X³ is independently selected from the group consisting of halogen, (C₁-C₆)alkyl, and N-oxide;
each X⁴ is independently selected form the group consisting of (C₁-C₆)alkyl, R⁵, —C(O)—(C₁-C₆)alkyl, —C(O)—R⁵, —C(O)—O—(C₁-C₆)alkyl, -alkylene-R⁵, R⁴, and —S(O₂)—(C₁-C₆)alkyl; and
each X⁵ is independently selected from the group consisting of (C₁-C₆)alkyl, a fused aryl ring, —C(O)—(C₁-C₆)alkyl, a fused heteroaryl ring, —C(O)—O—(C₁-C₆)alkyl, —C(O)—R⁵, —S(O₂)—(C₁-C₆)alkyl, —C(O)—N(R⁹)₂, R⁵, R⁶, —C(O)—R⁴, —C(O)—O—R⁴, —S(O₂)—R⁴, —S(O₂)-alkylene-R⁴, —S(O₂)-alkylene-R⁵, —N(R⁹)—C(O)—O—(C₁-C₆)alkyl, —N(R⁹)—C(O)—O—R⁴, —N(R⁹)—C(O)—N(R⁹)₂ and —N(R⁹)₂;
wherein said fused aryl ring of X⁵ is unsubstituted or independently substituted with one or more substituent selected from -alkylene-R⁷ or X², and said fused heteroaryl ring of X⁵ is unsubstituted or substituted with one or more X³ groups;
f) Formula VII, VIII-A, VIII-B, IX or X: 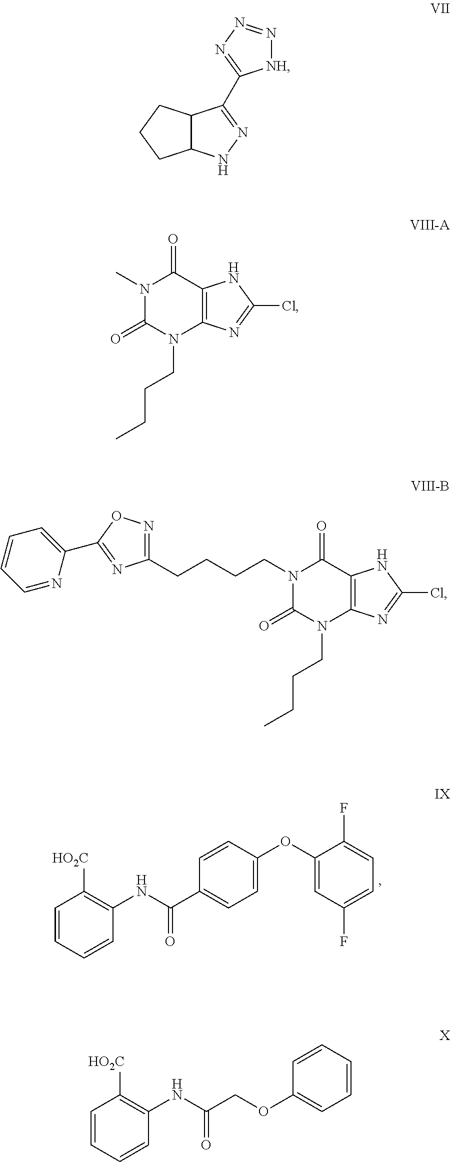
iii) optionally, an effective amount of at least one cardiovascular agent and
iv) optionally a pharmaceutically acceptable carrier.

2. The pharmaceutical composition according to claim 1 wherein at least one cardiovascular agent is present.

3. The pharmaceutical composition according to claim 2, wherein the cardiovascular agent is selected from the group consisting of thromboxane A2 biosynthesis inhibitors; thromboxane antagonists; adenosine diphosphate (ADP) inhibitors; cyclooxygenase inhibitors; angiotensin antagonists; endothelin antagonists; phosphodiesterase inhibitors; angiotensin converting enzyme (ACE) inhibitors; neutral endopeptidase inhibitors; anticoagulants; diuretics; platelet aggregation inhibitors; GP IIb/IIIa antagonists; and GP1b antagonists.

4. The pharmaceutical composition according to claim 1 wherein the NAR agonist is a compound selected from the group consisting of: 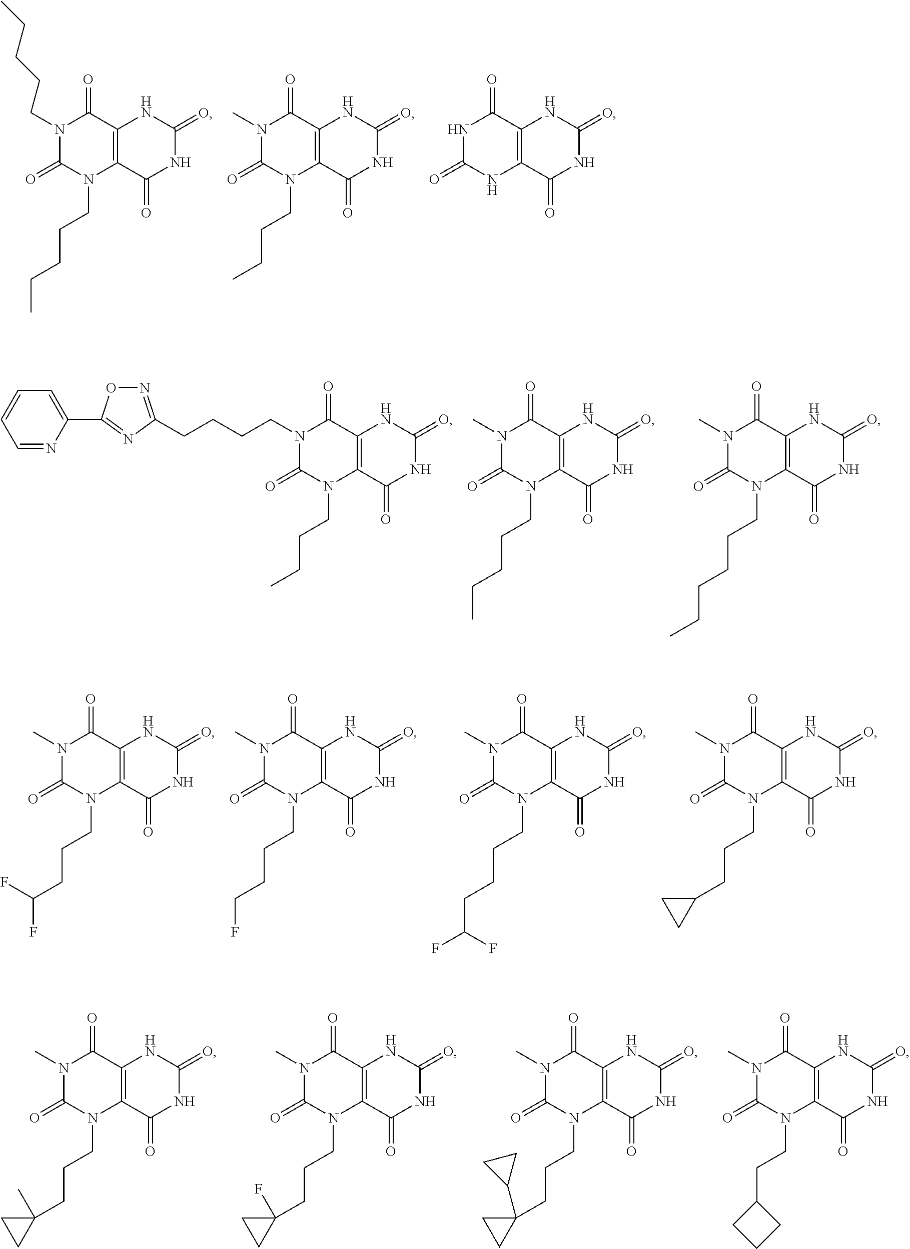 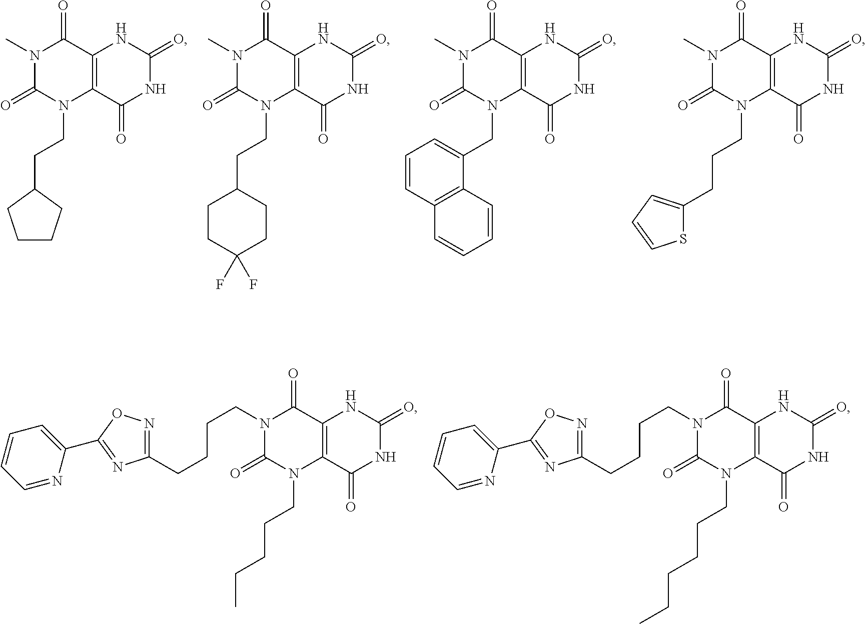 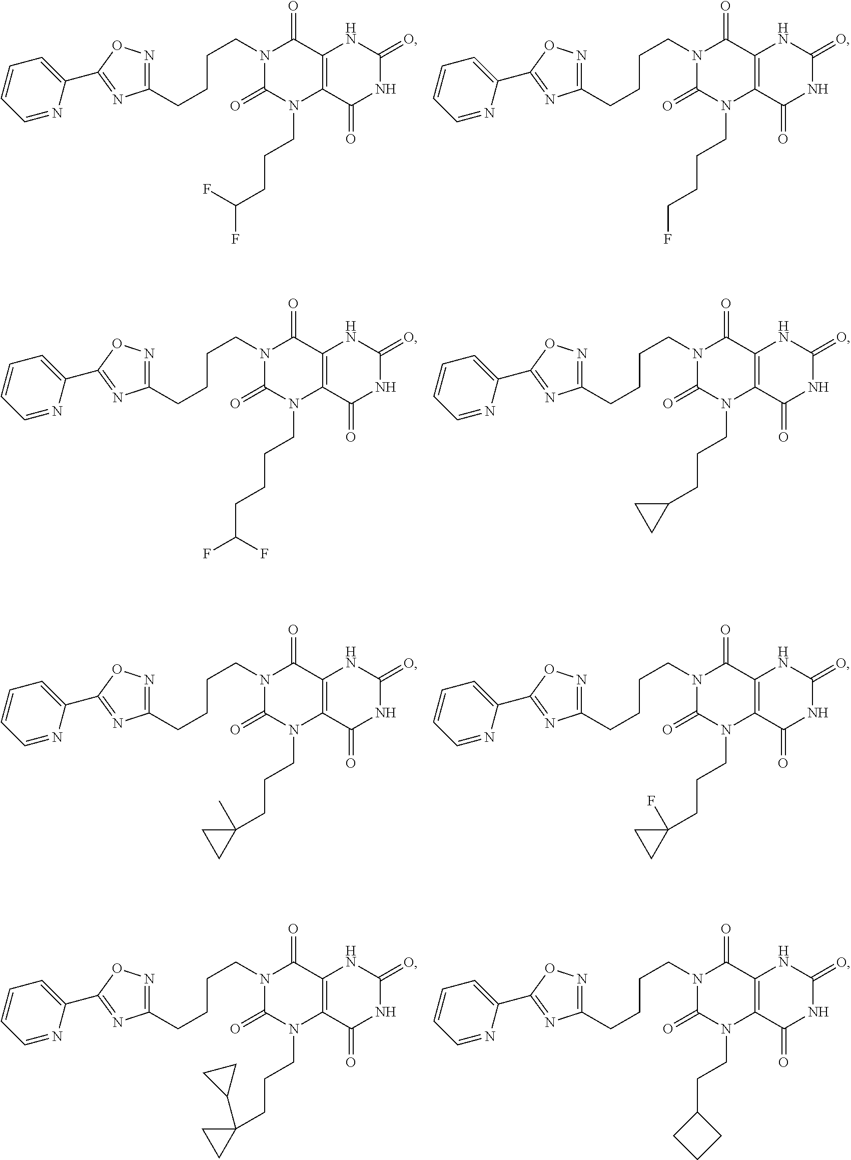 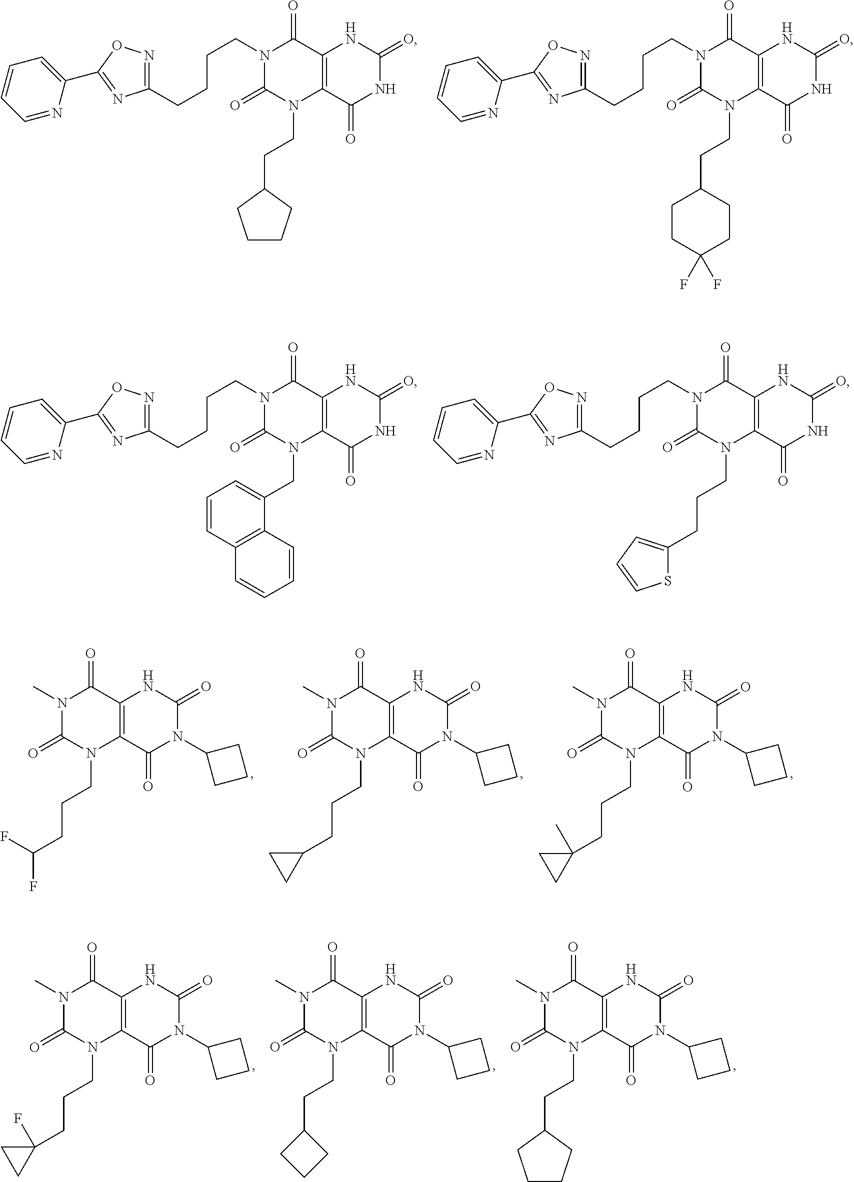 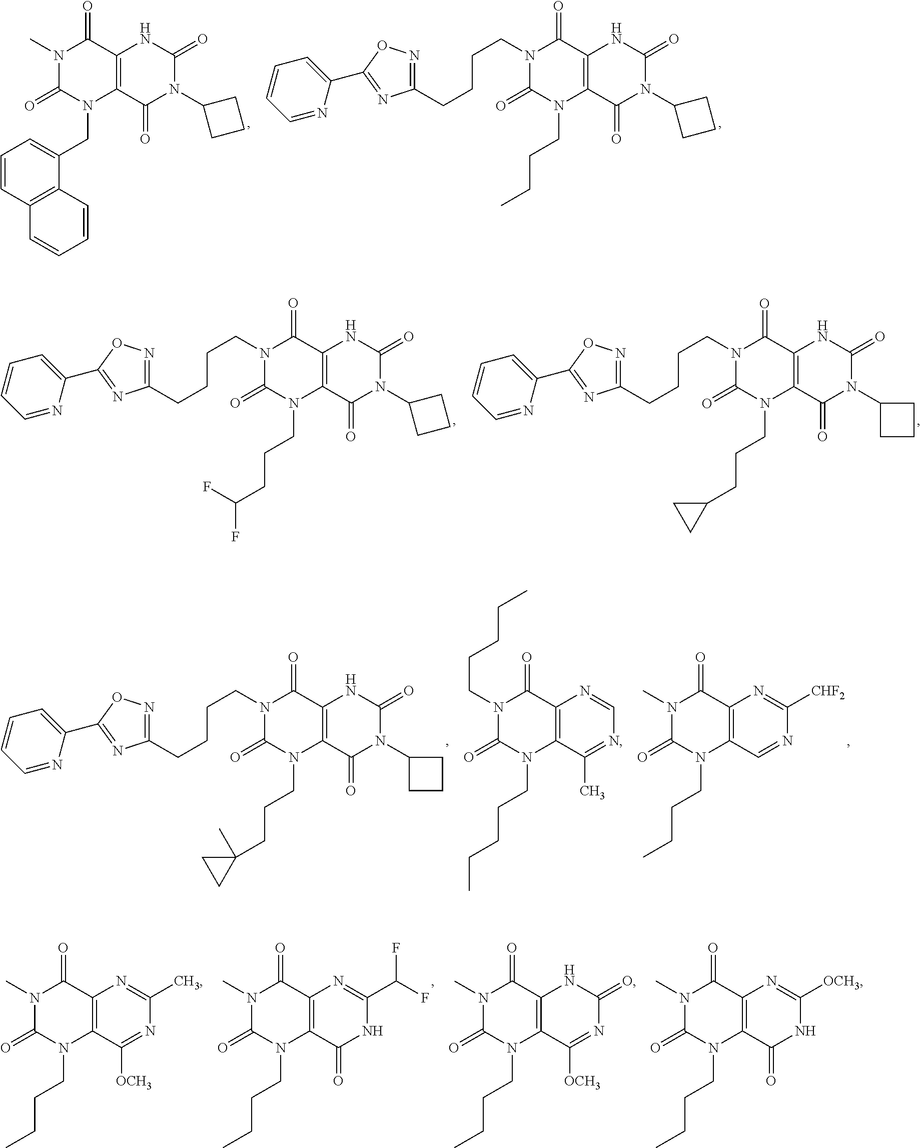 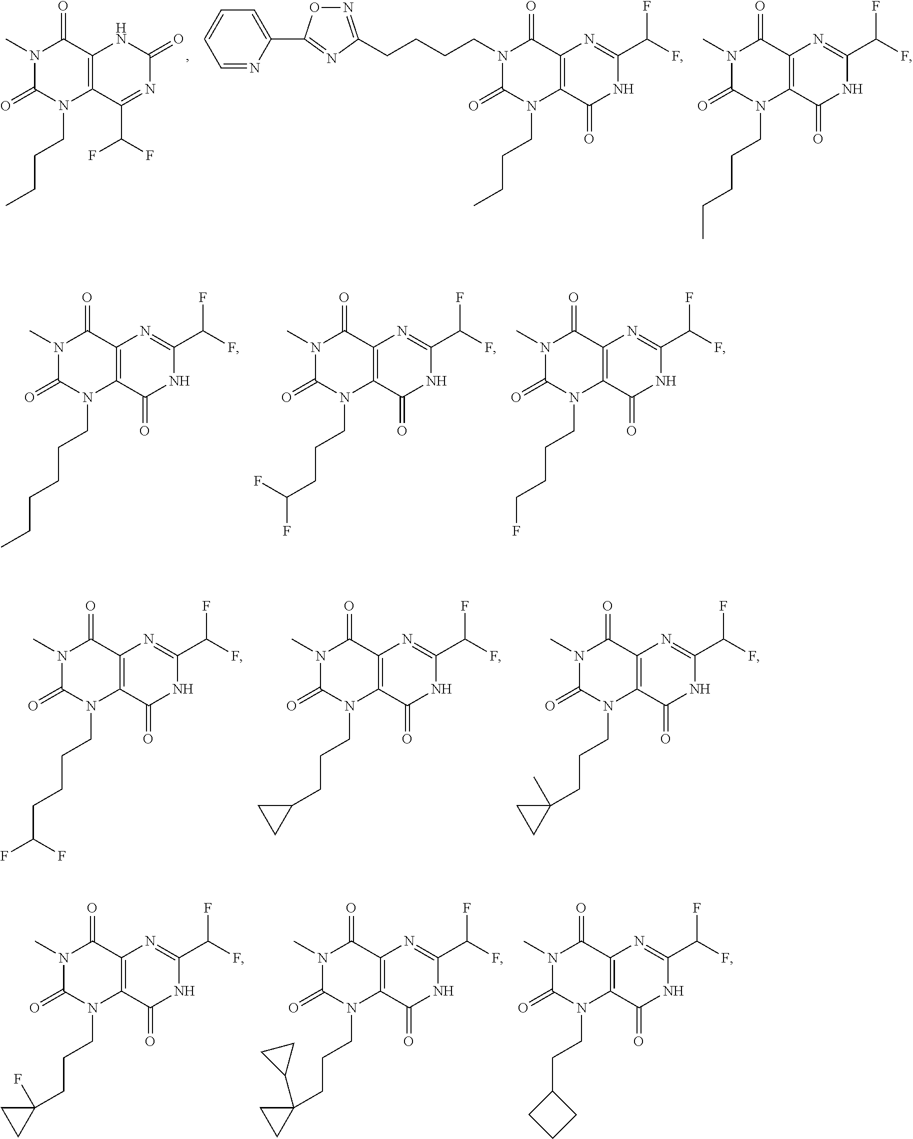 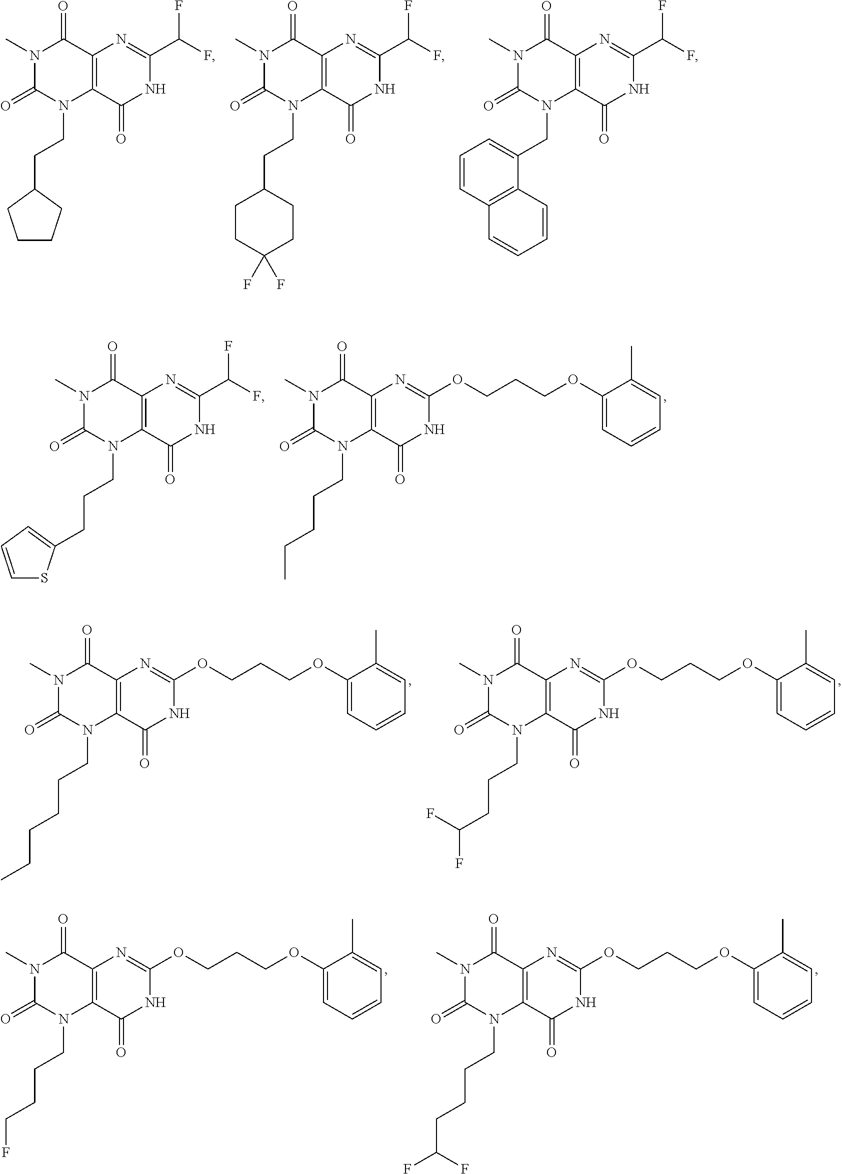 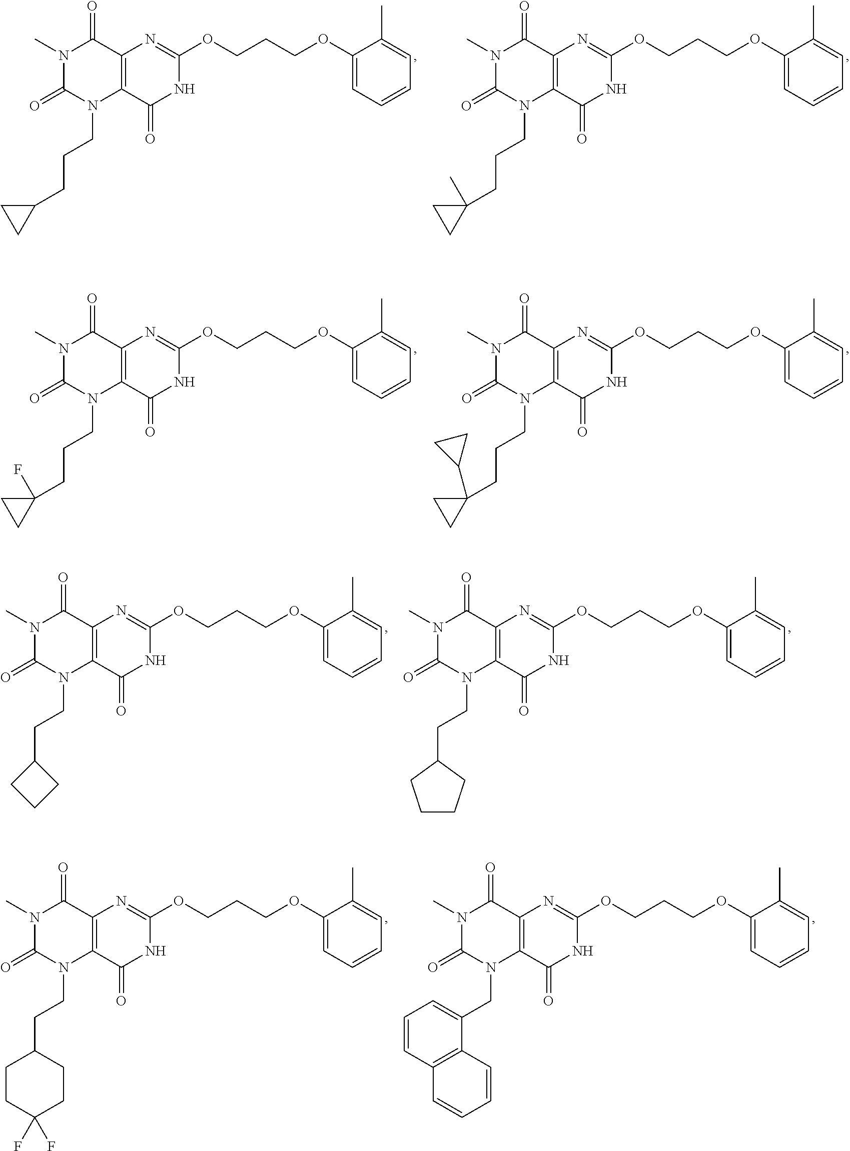 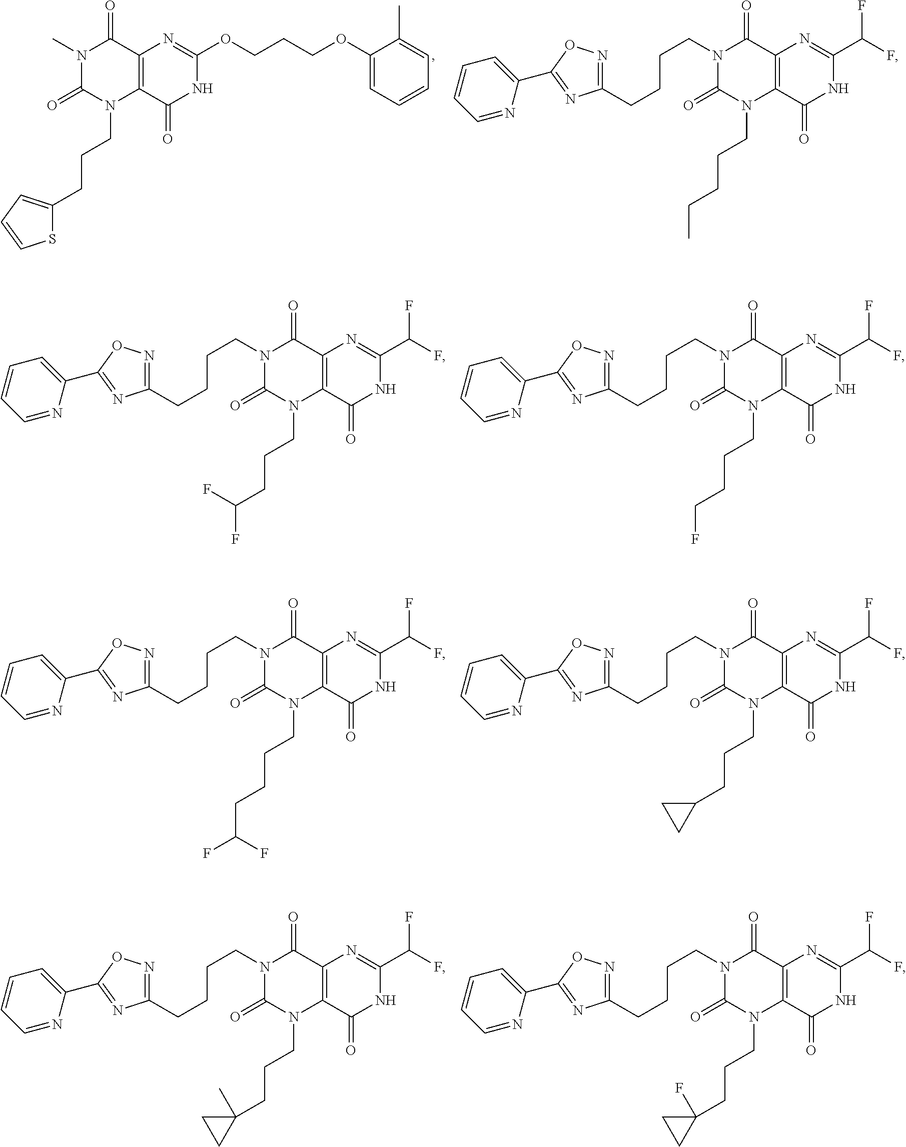 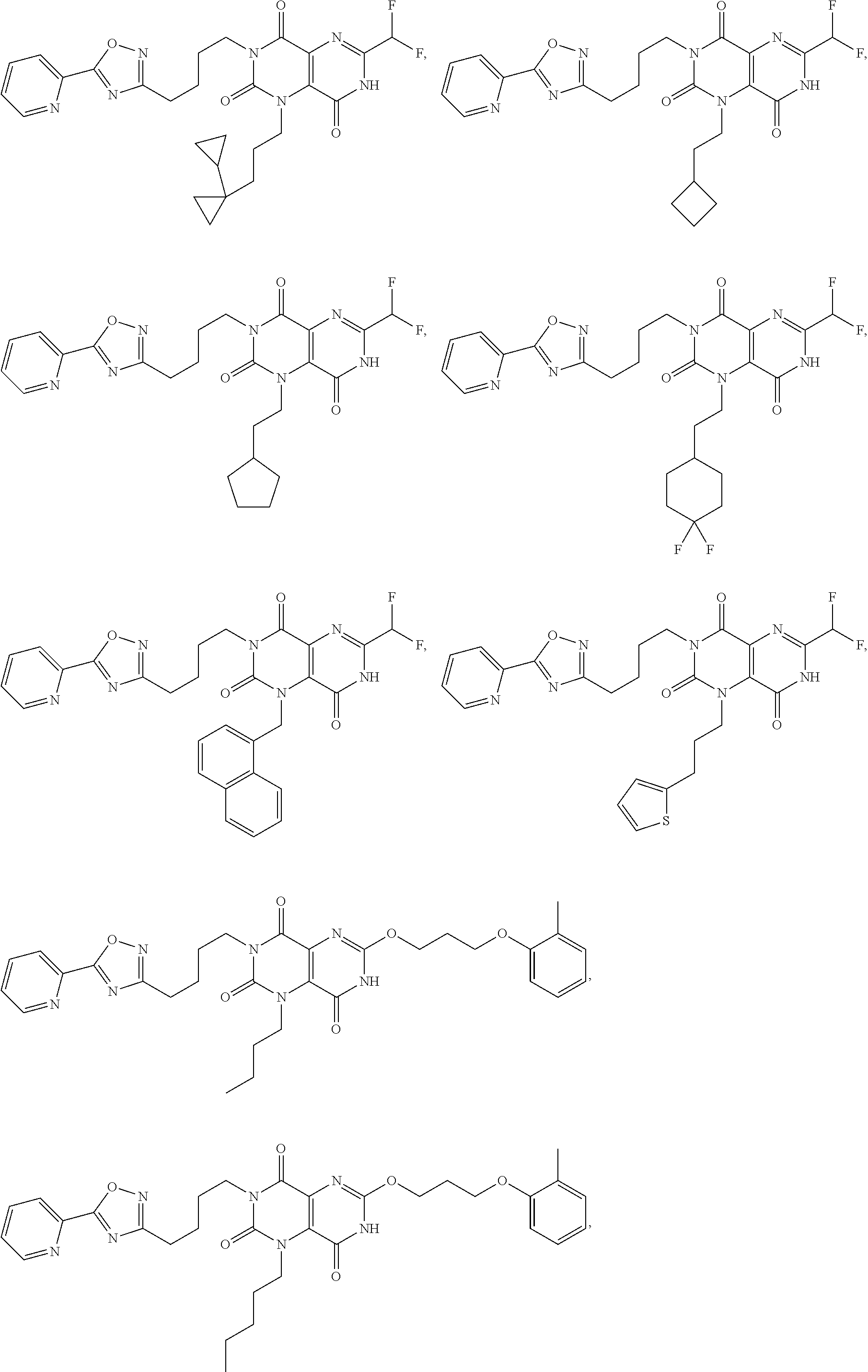 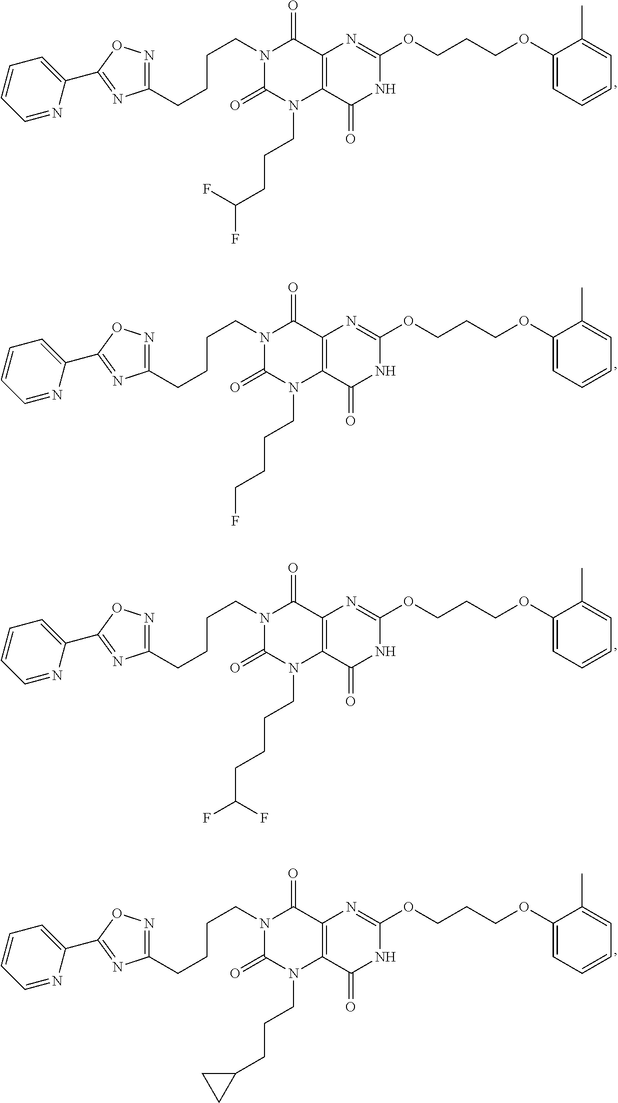 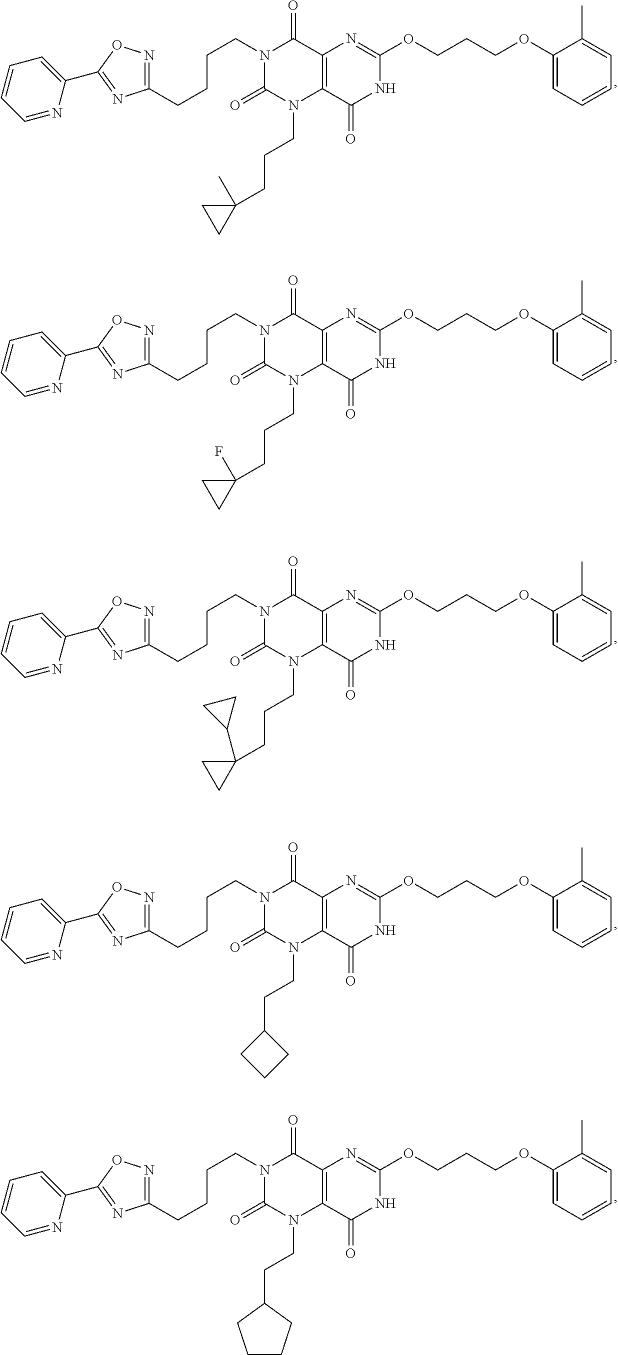 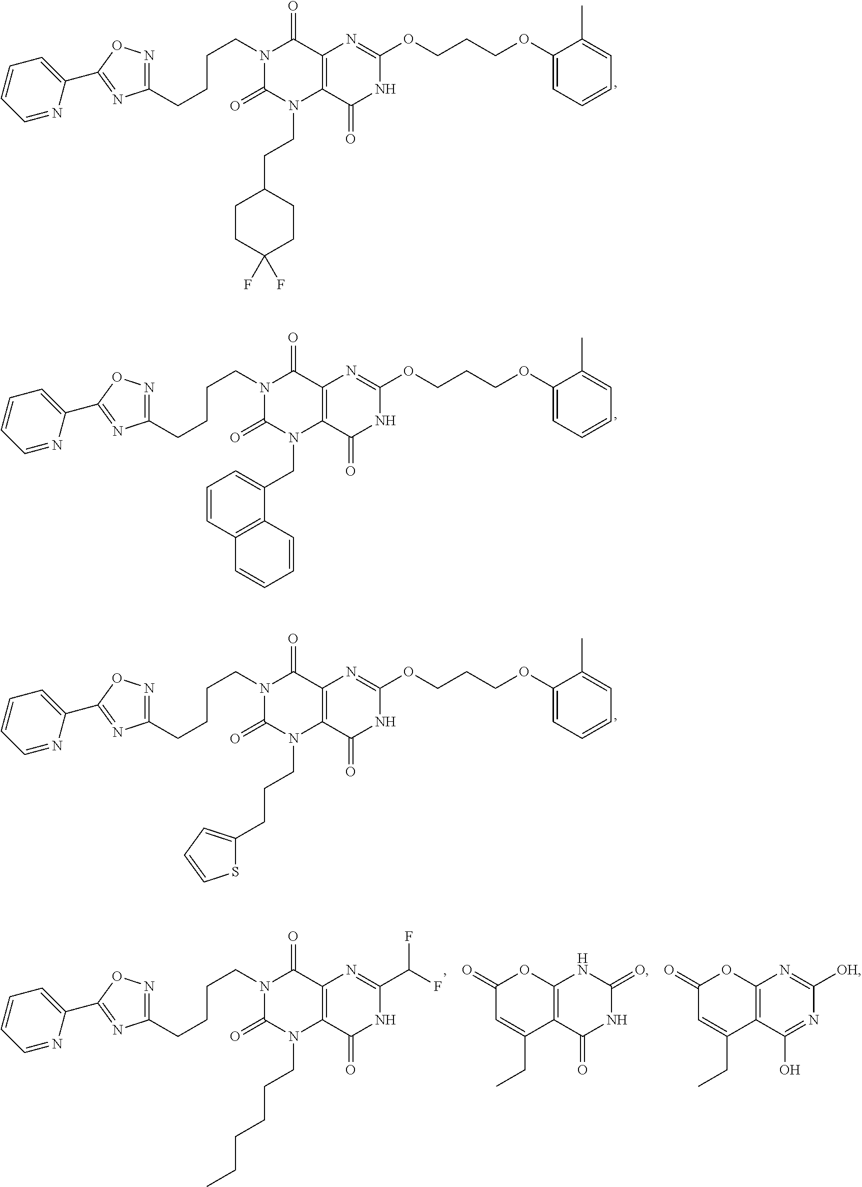 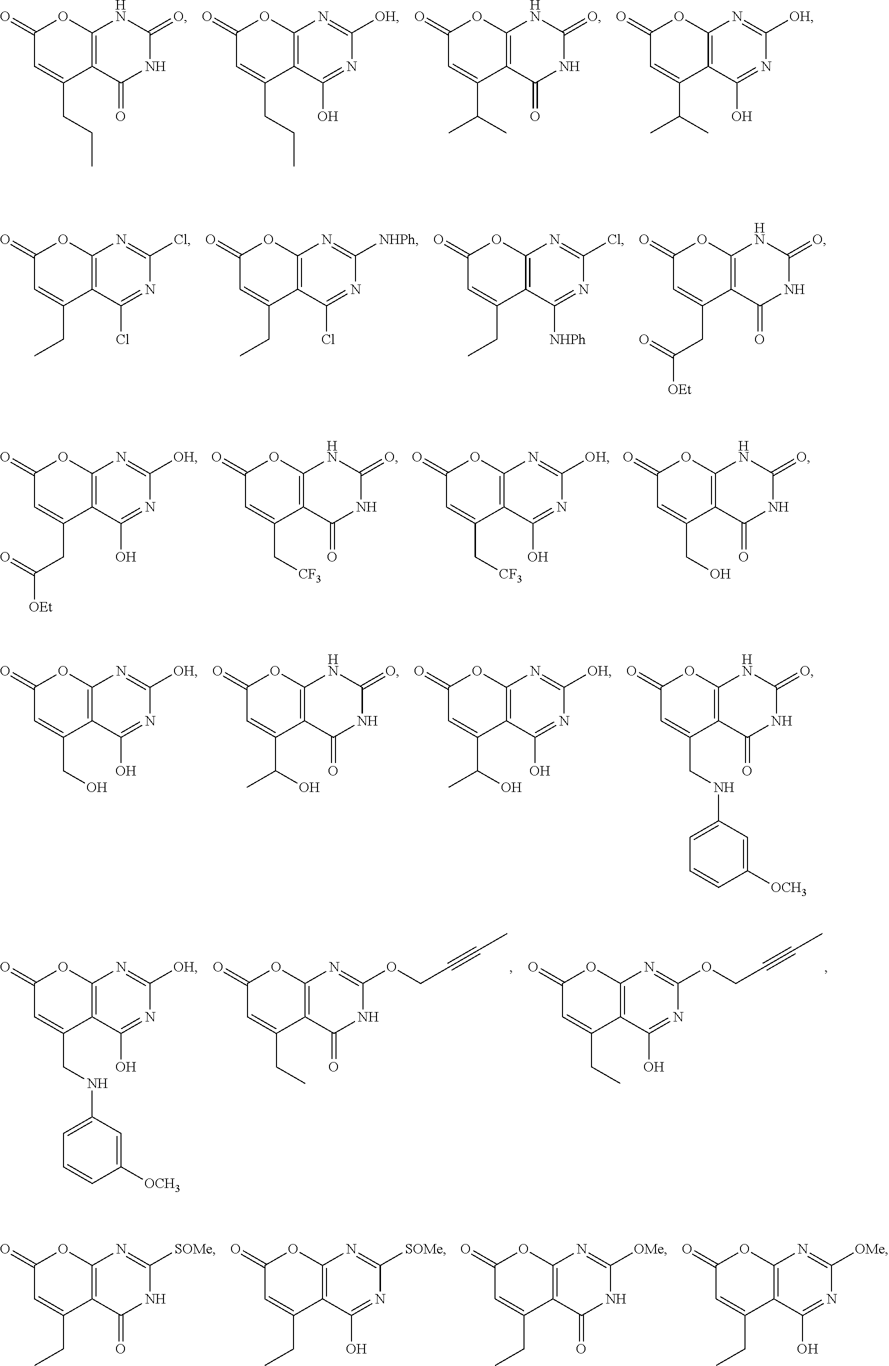 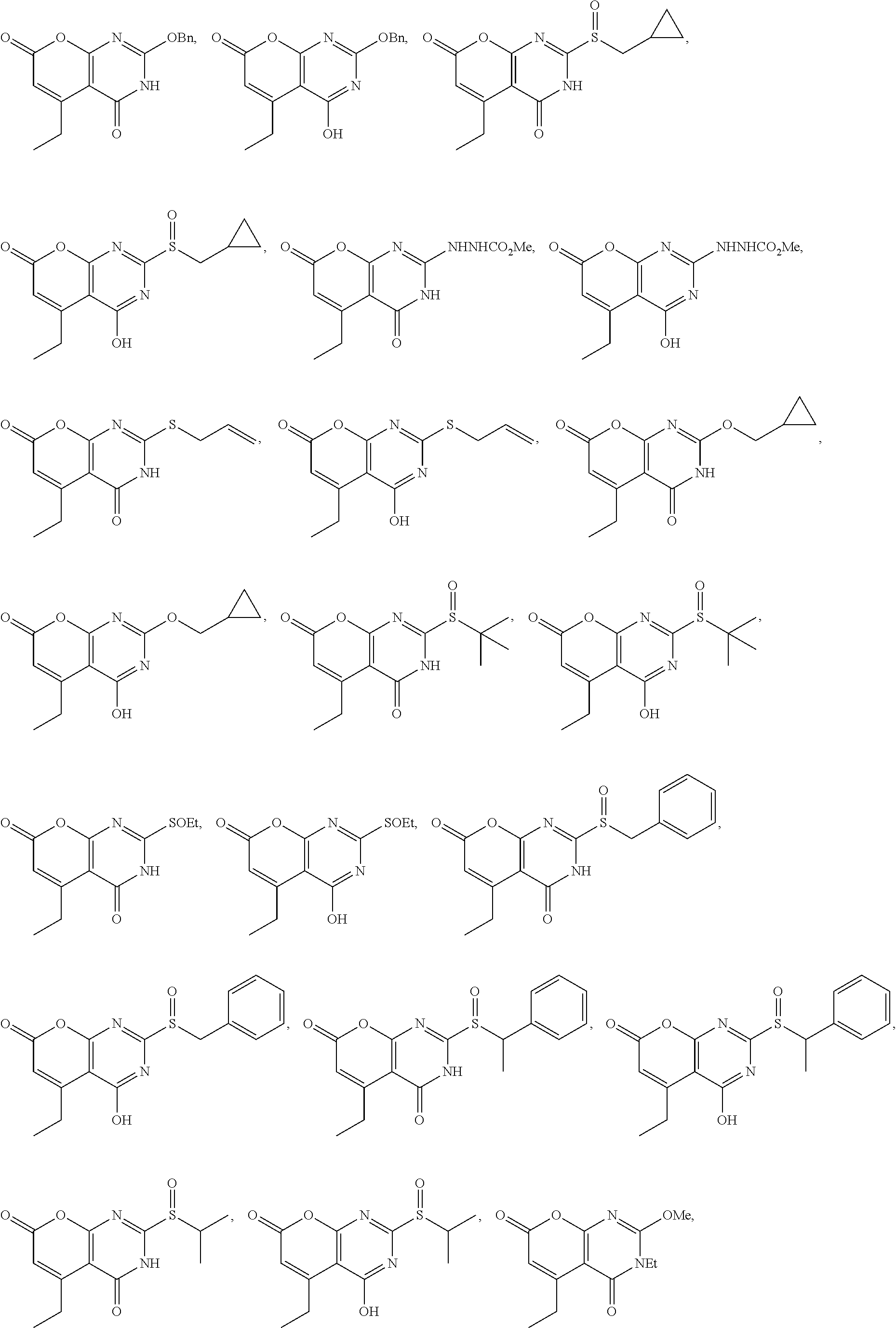 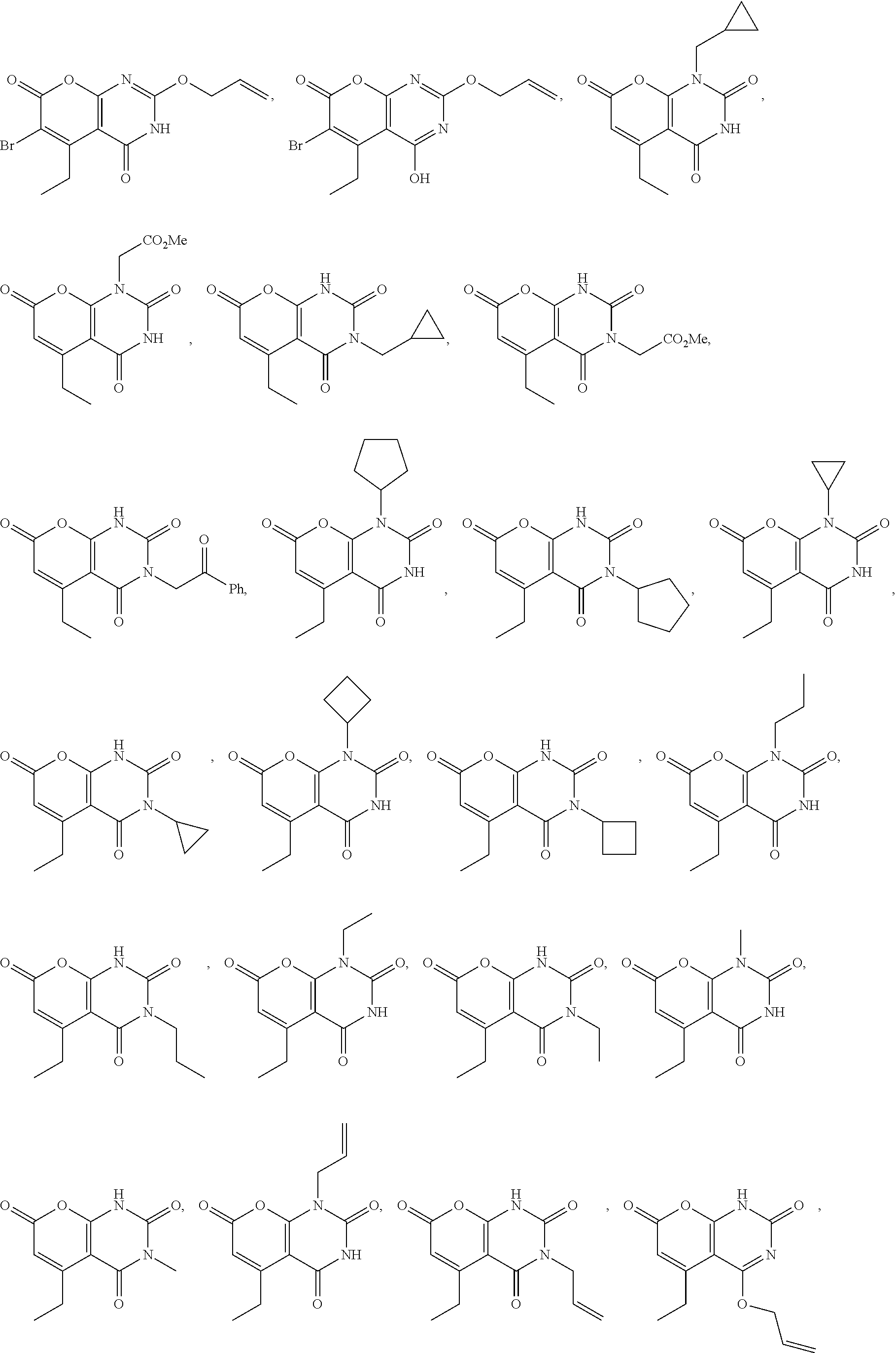 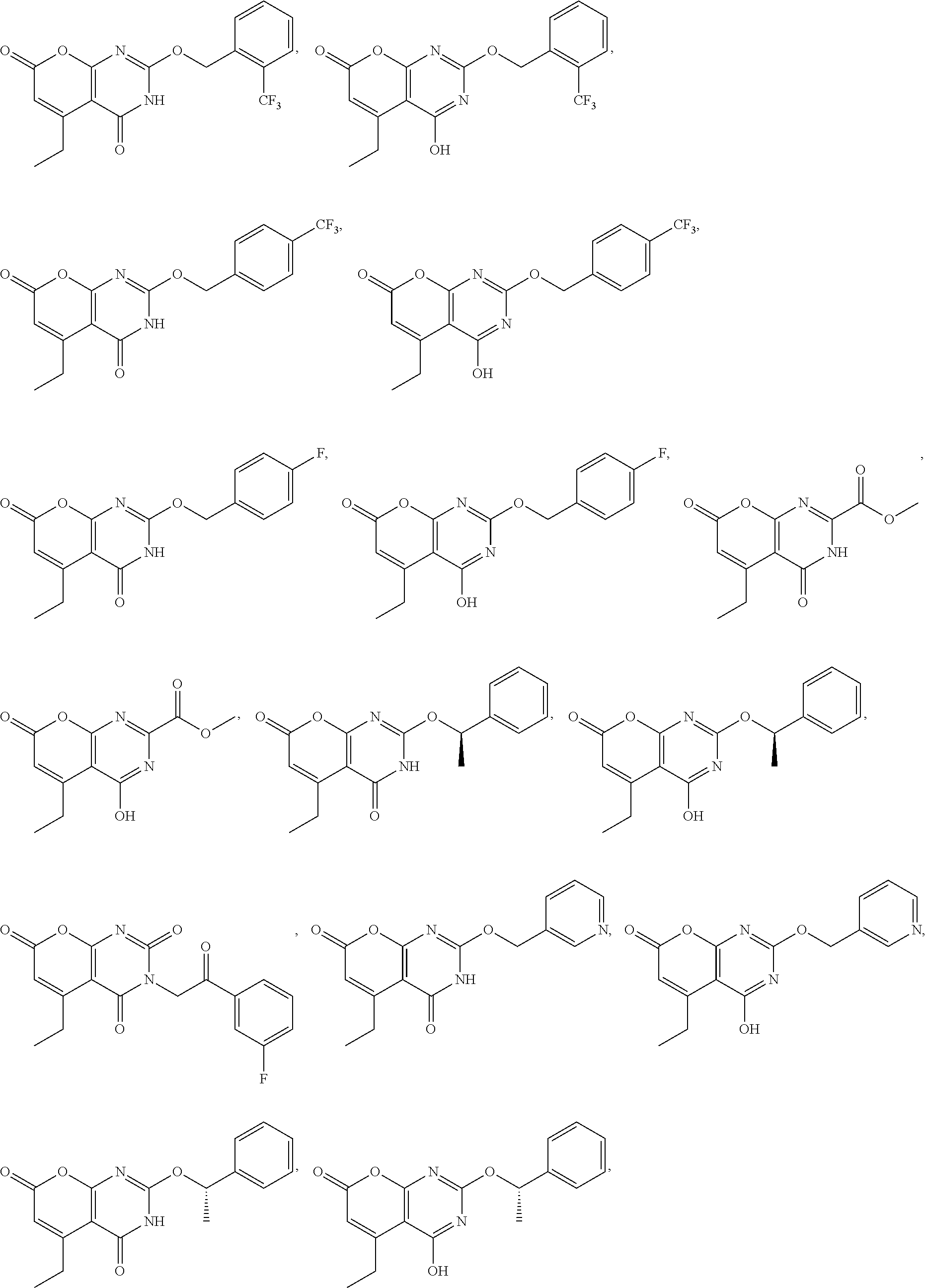 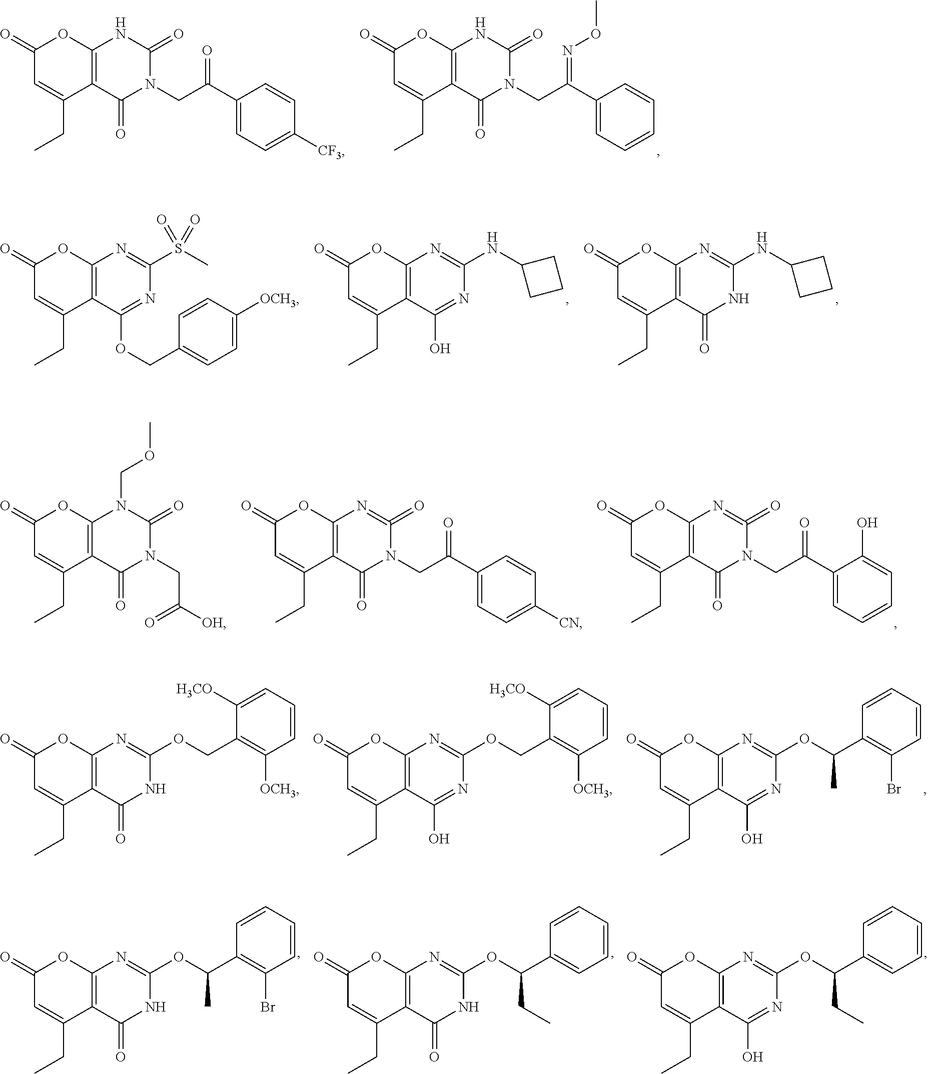 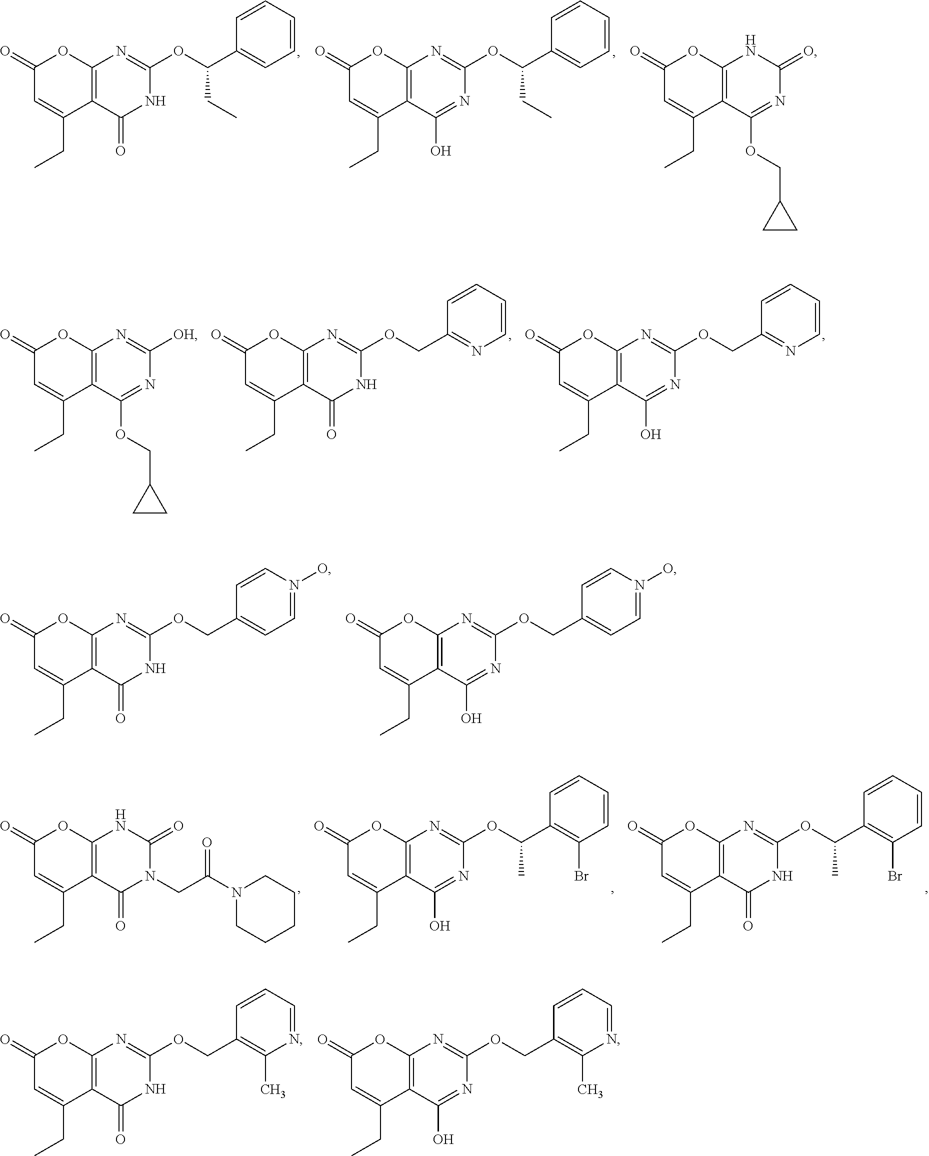 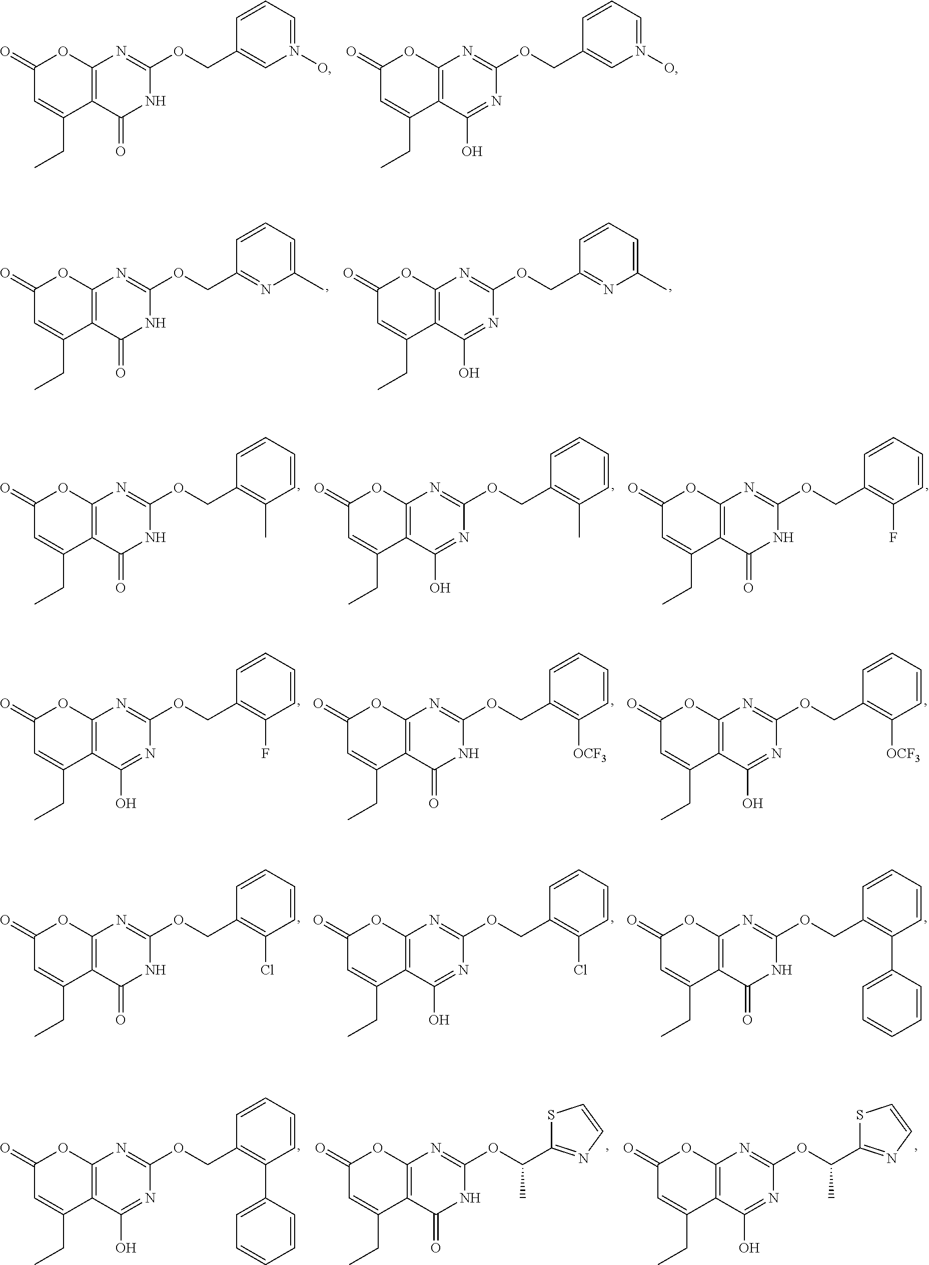 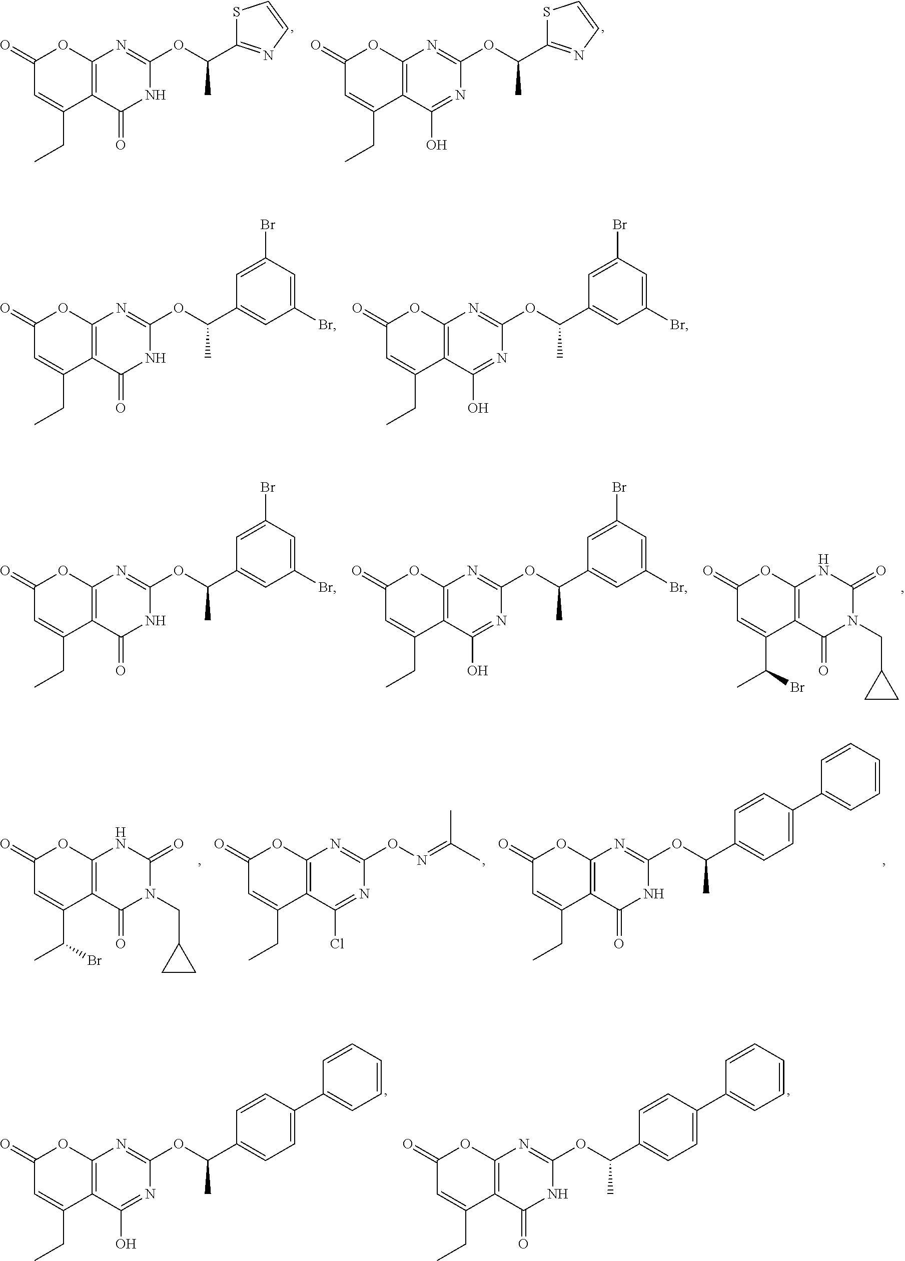 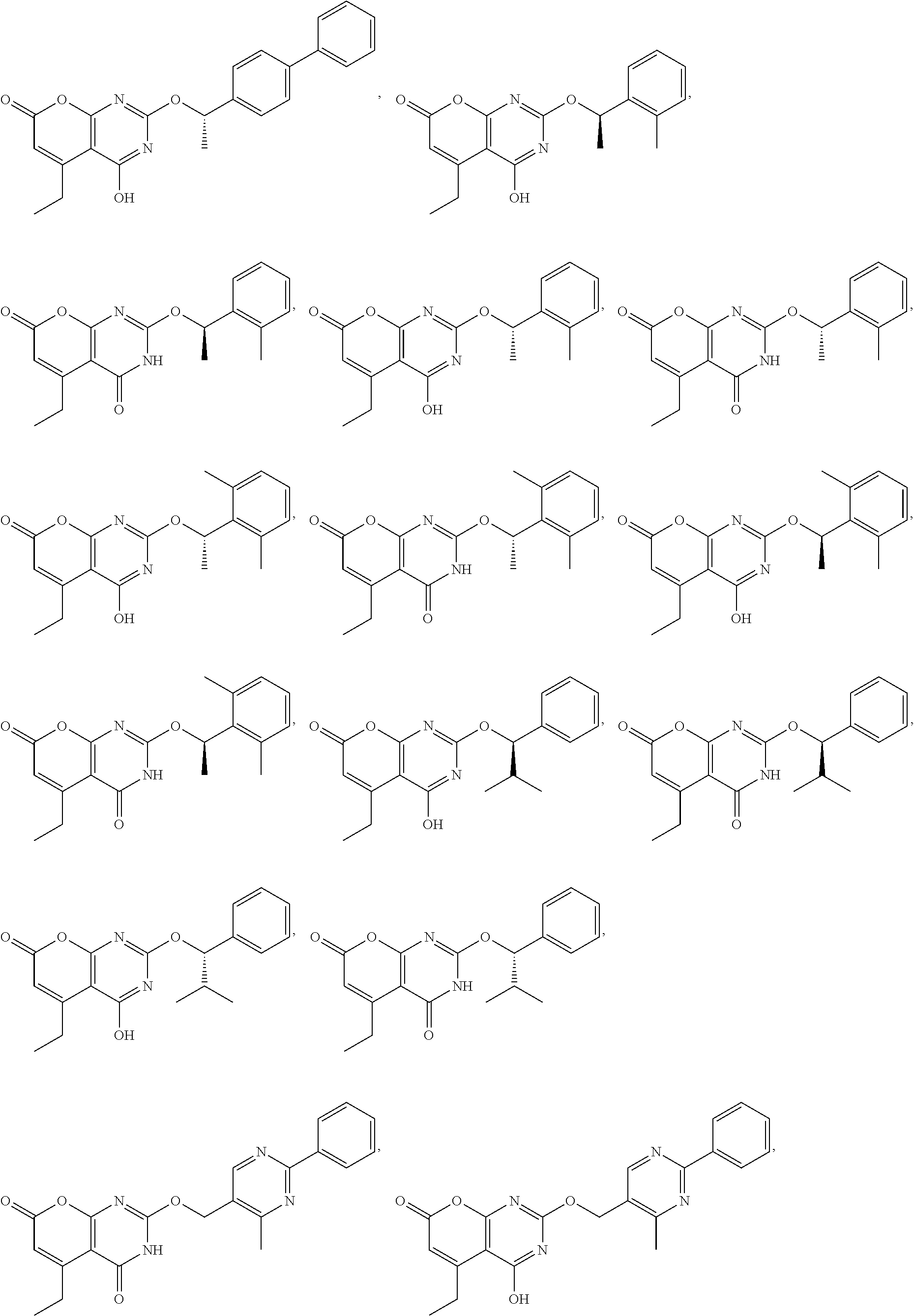 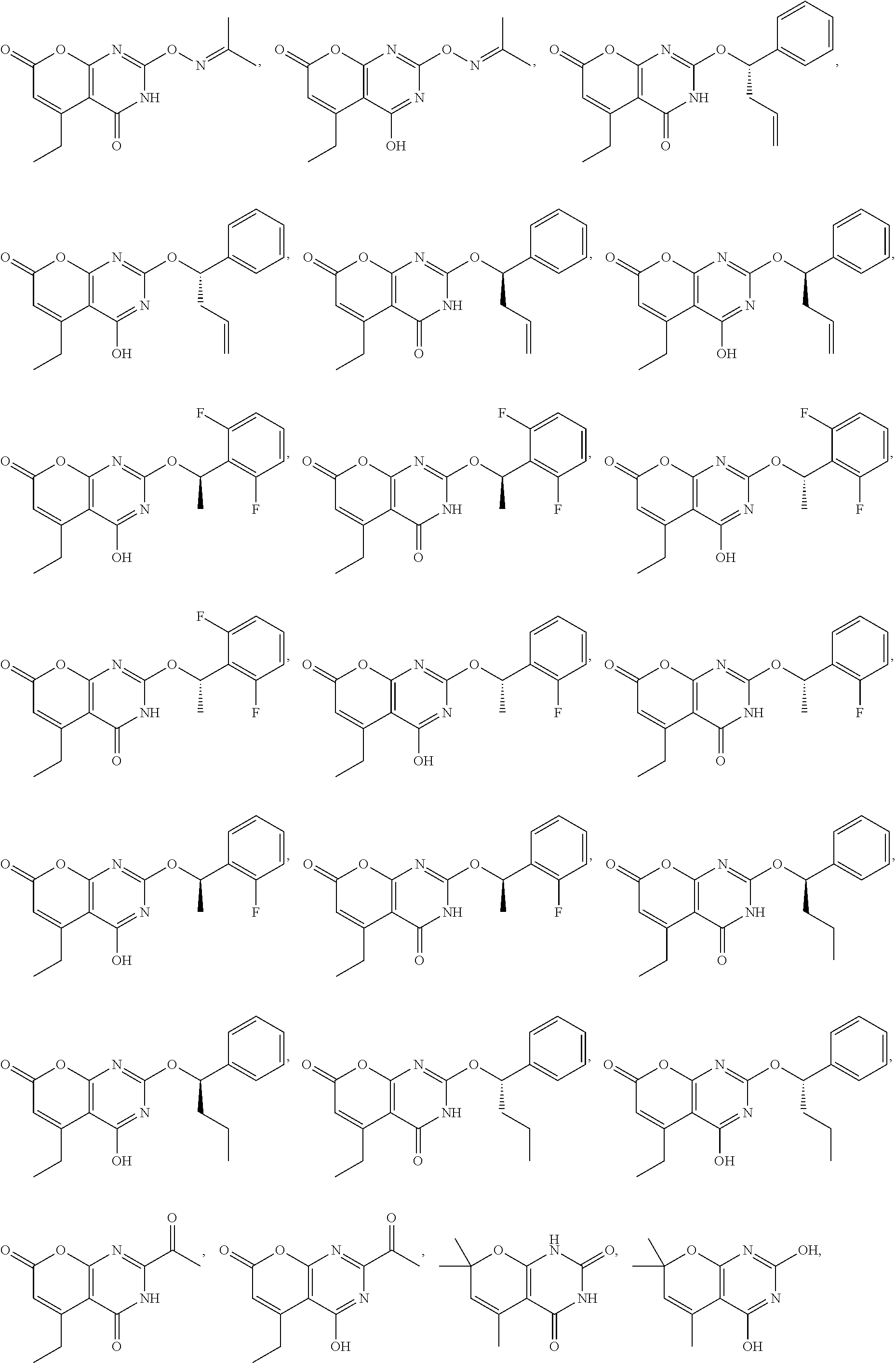 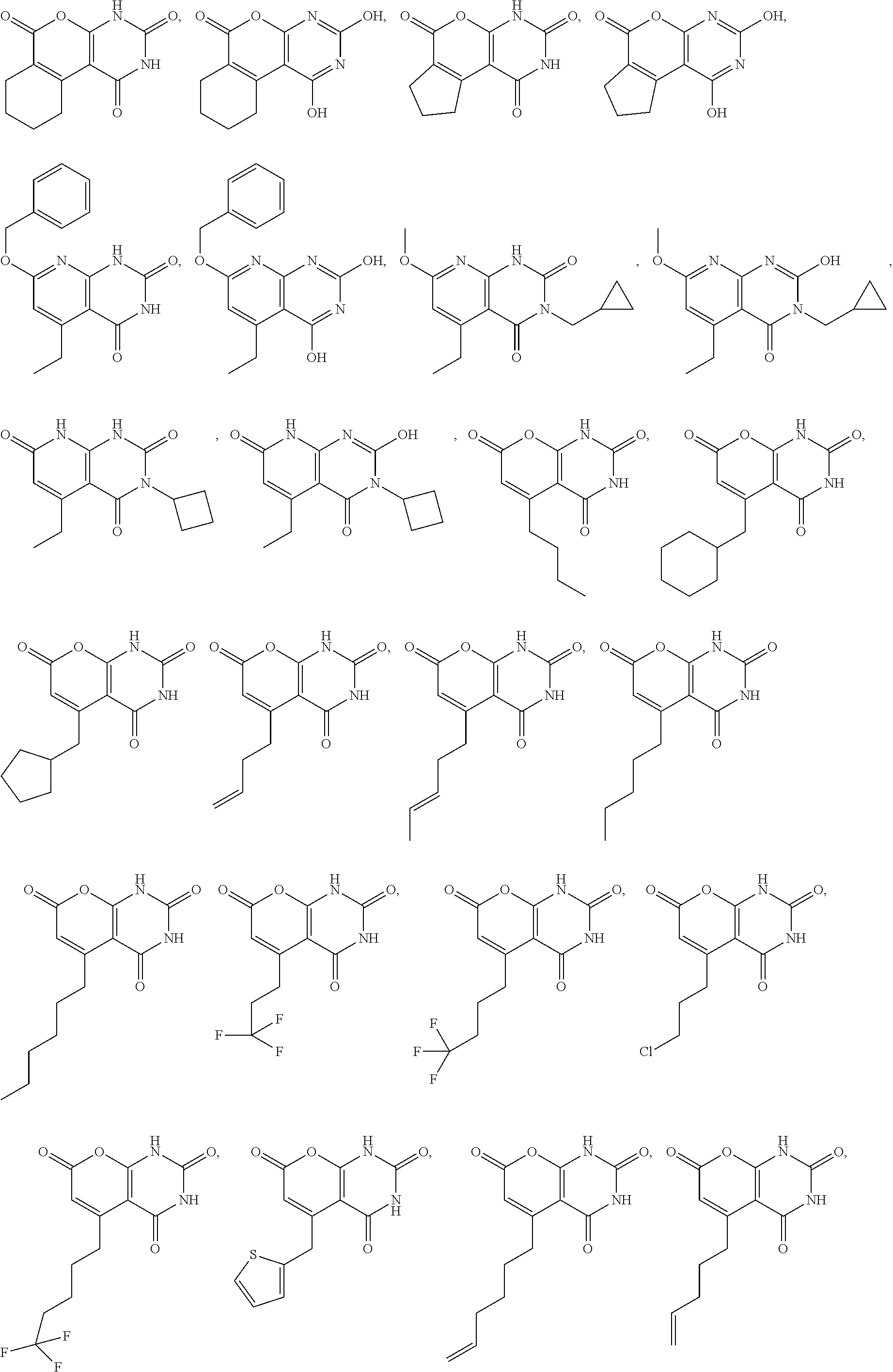 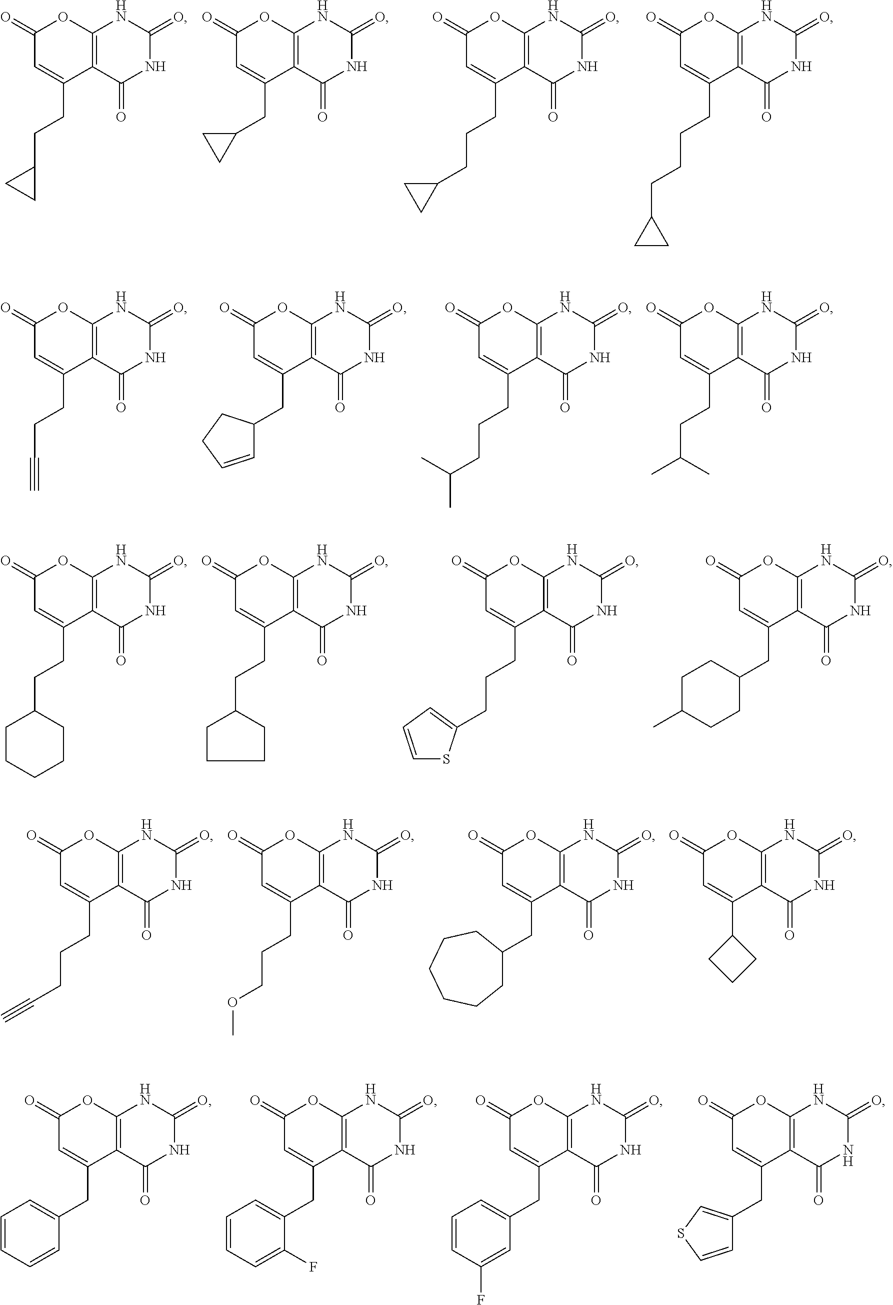 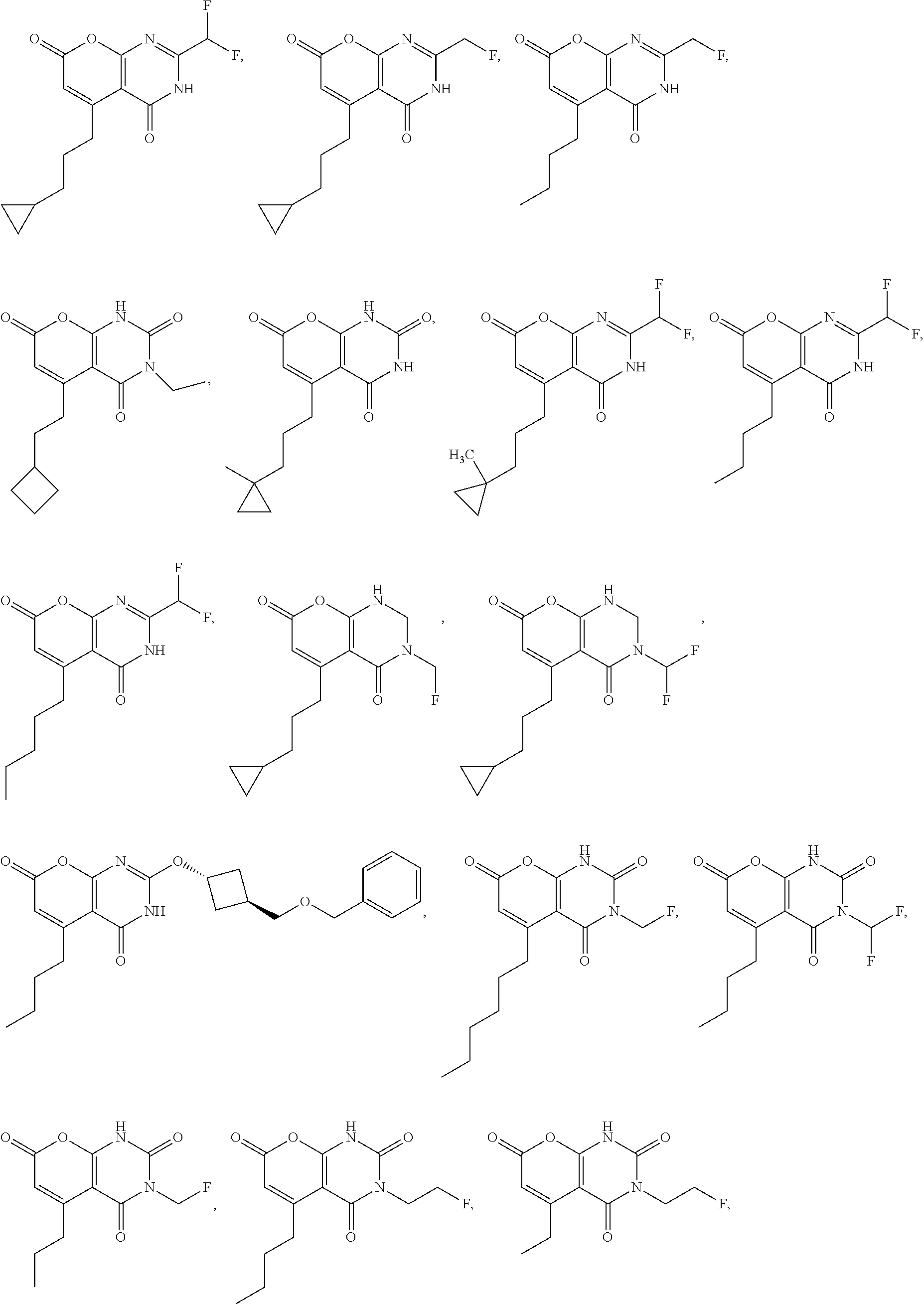 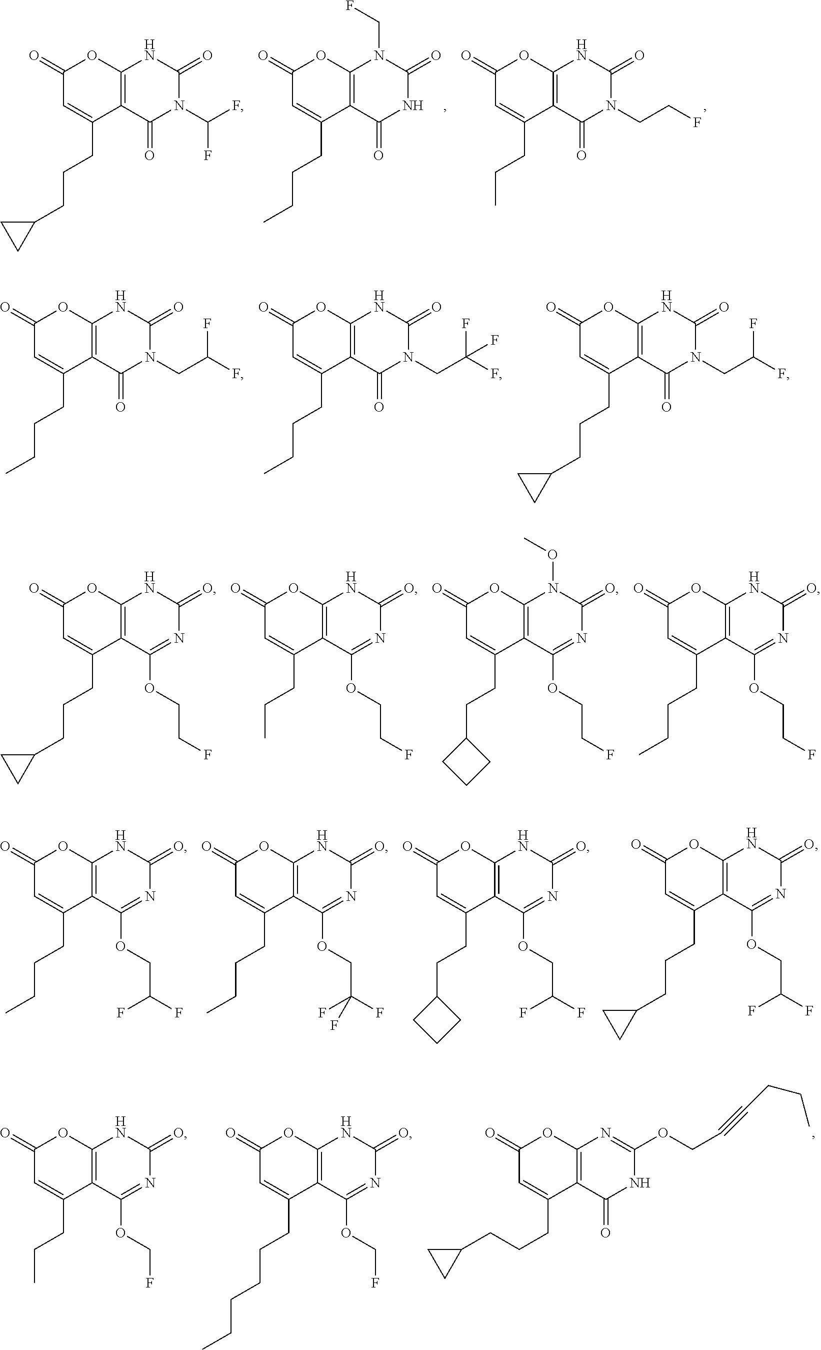 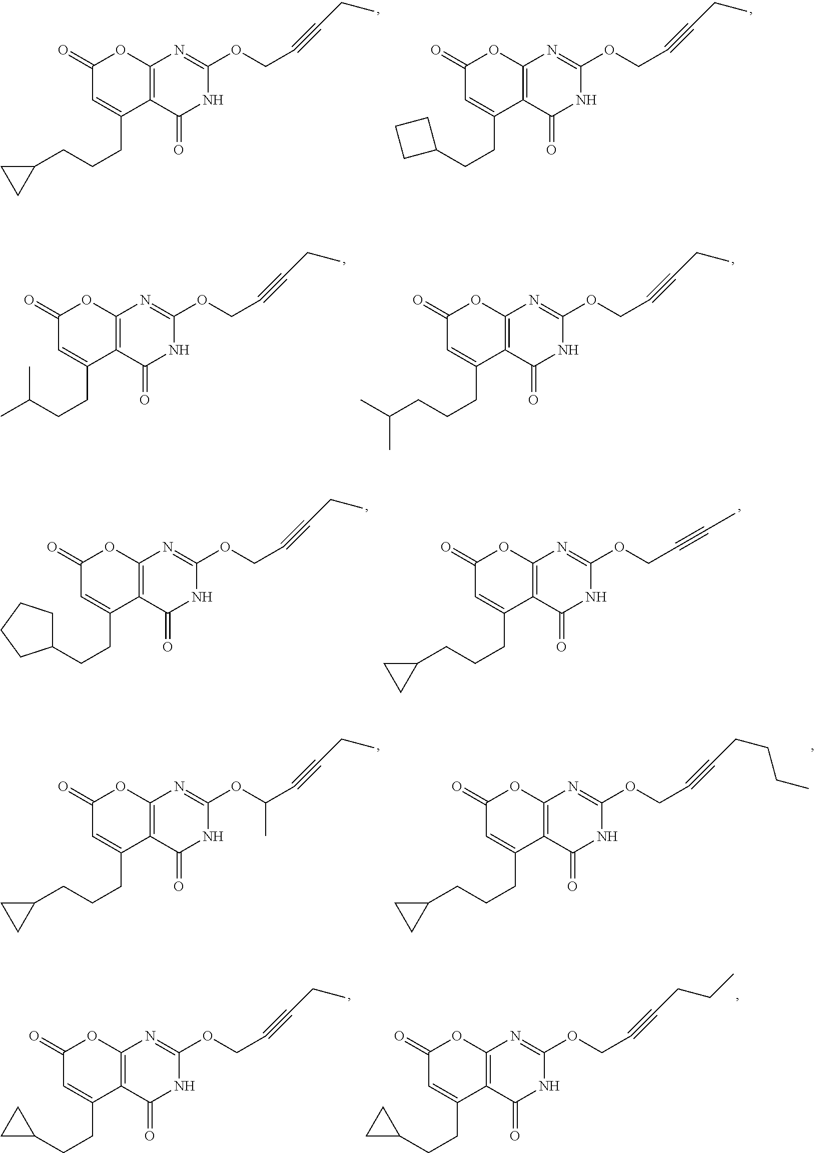 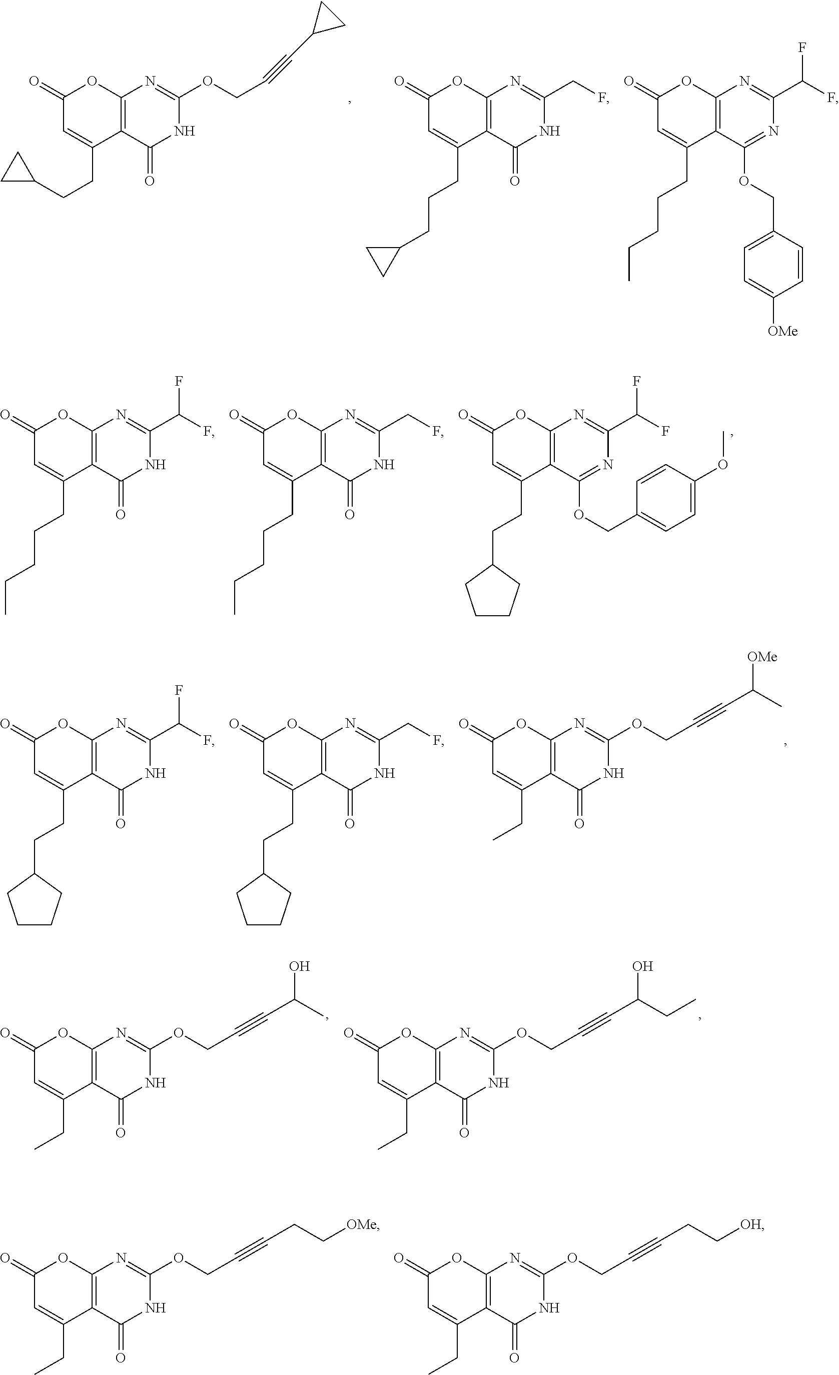 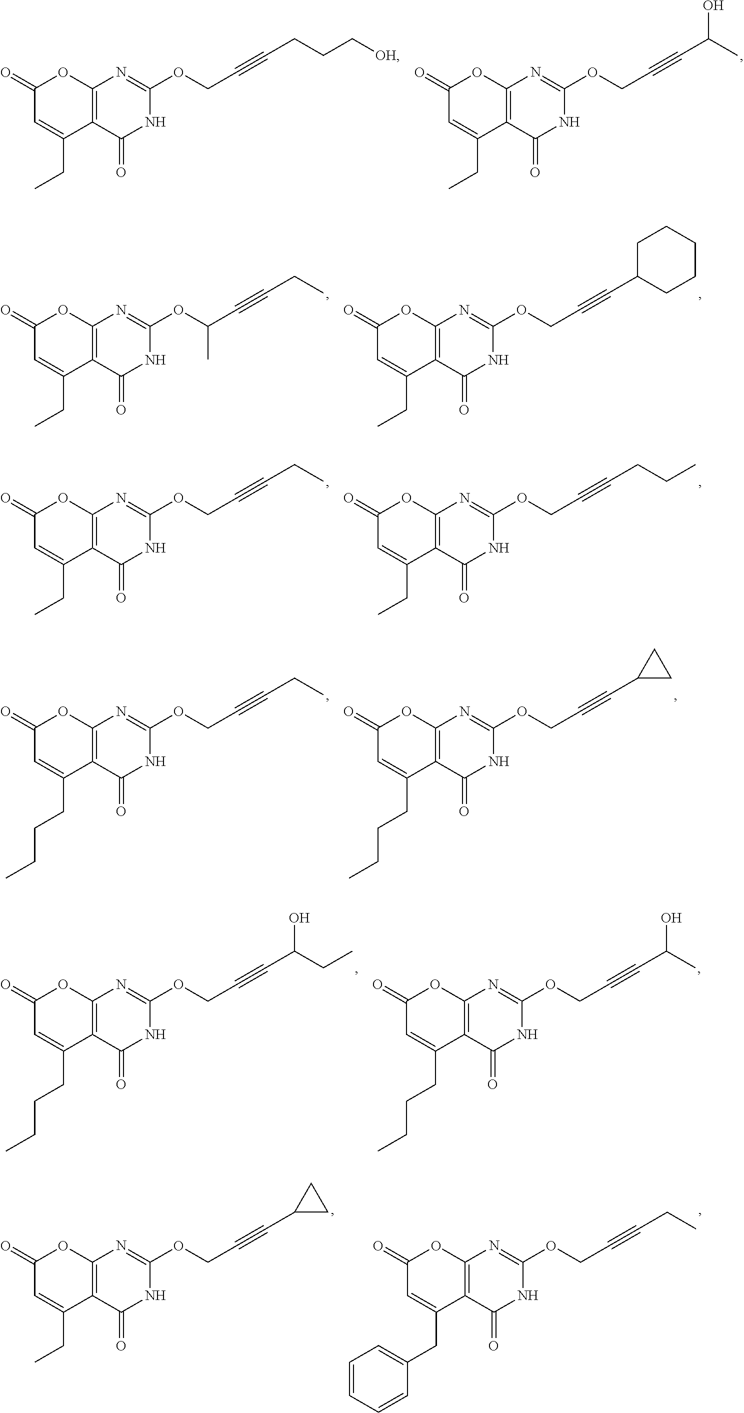 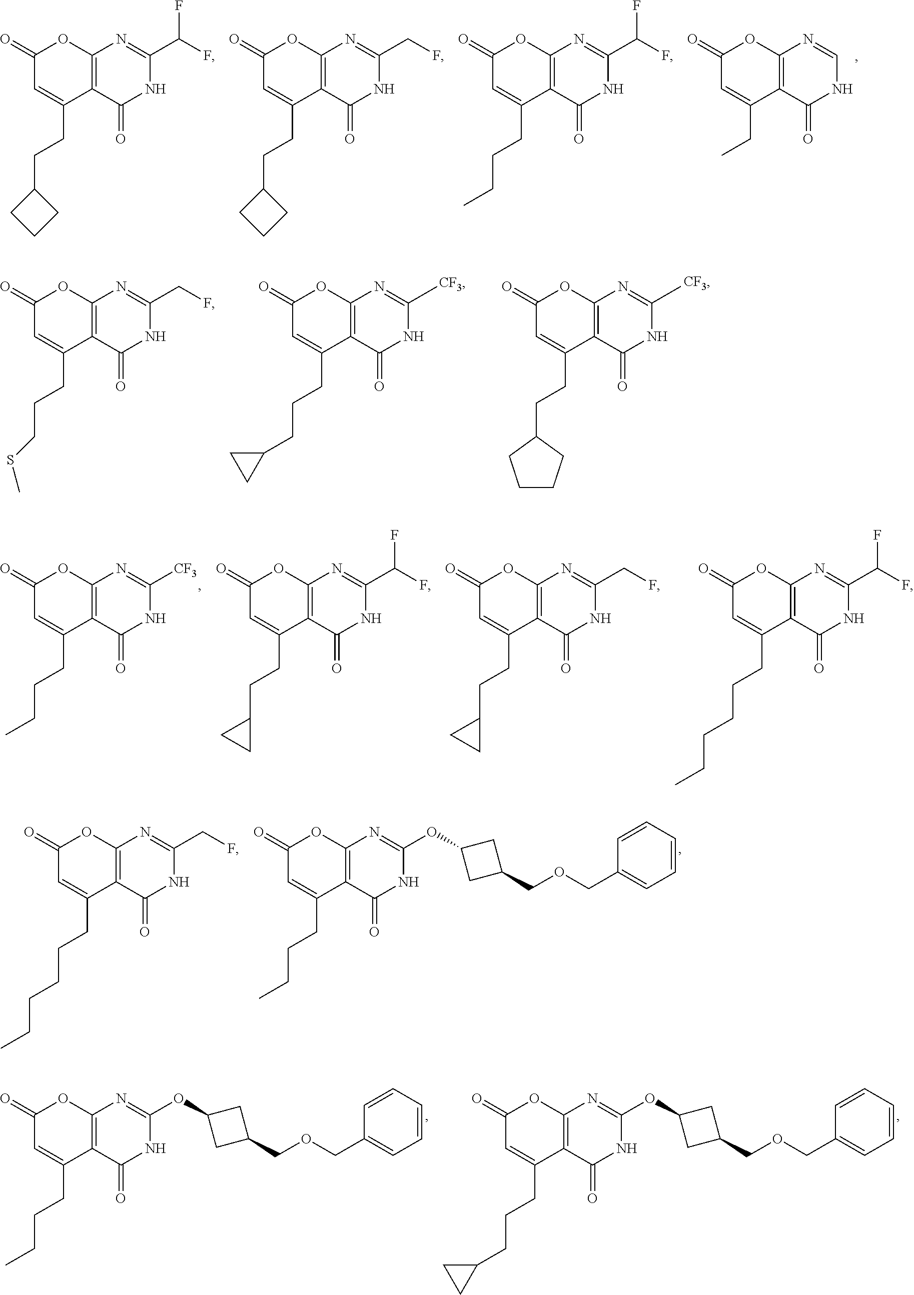 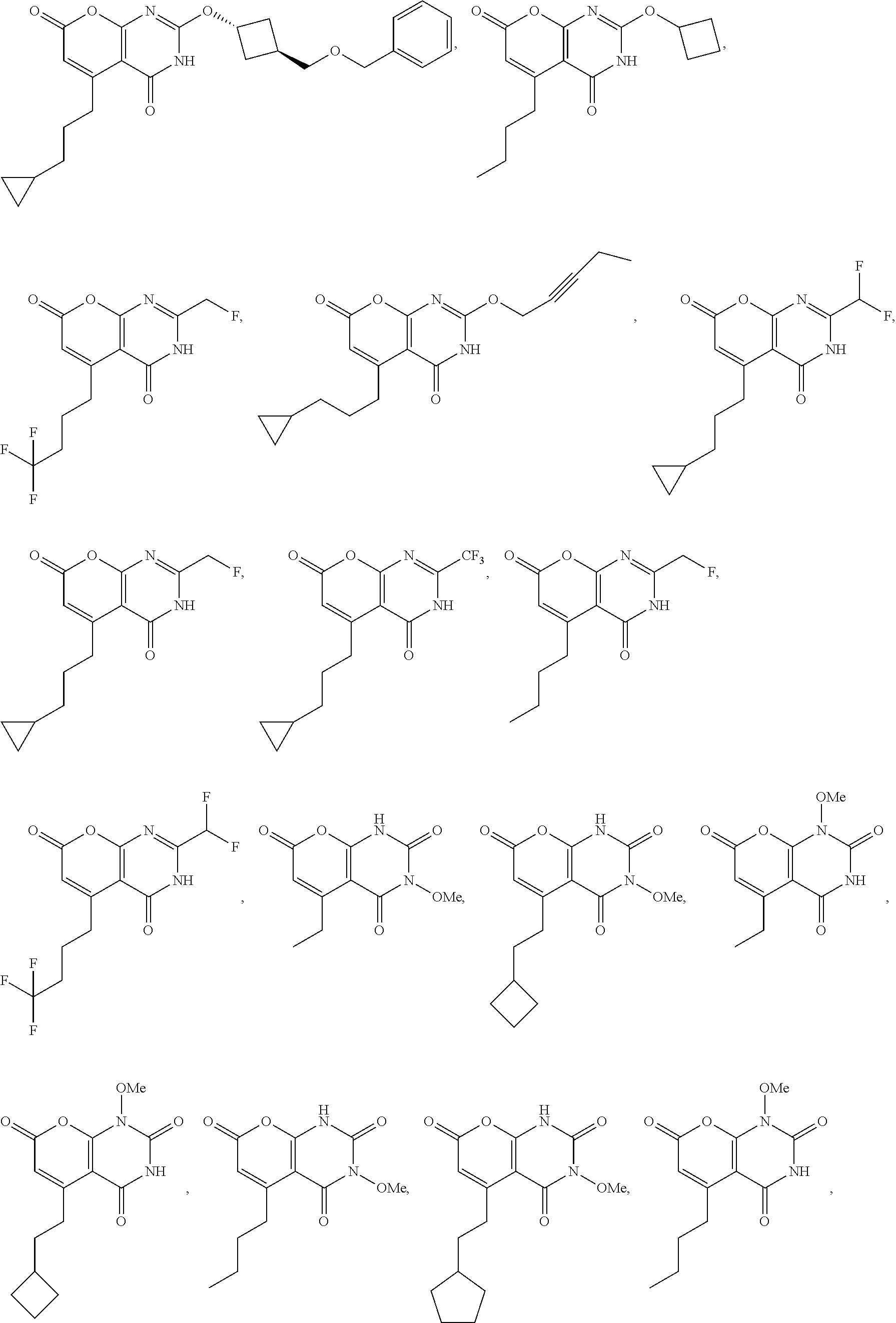 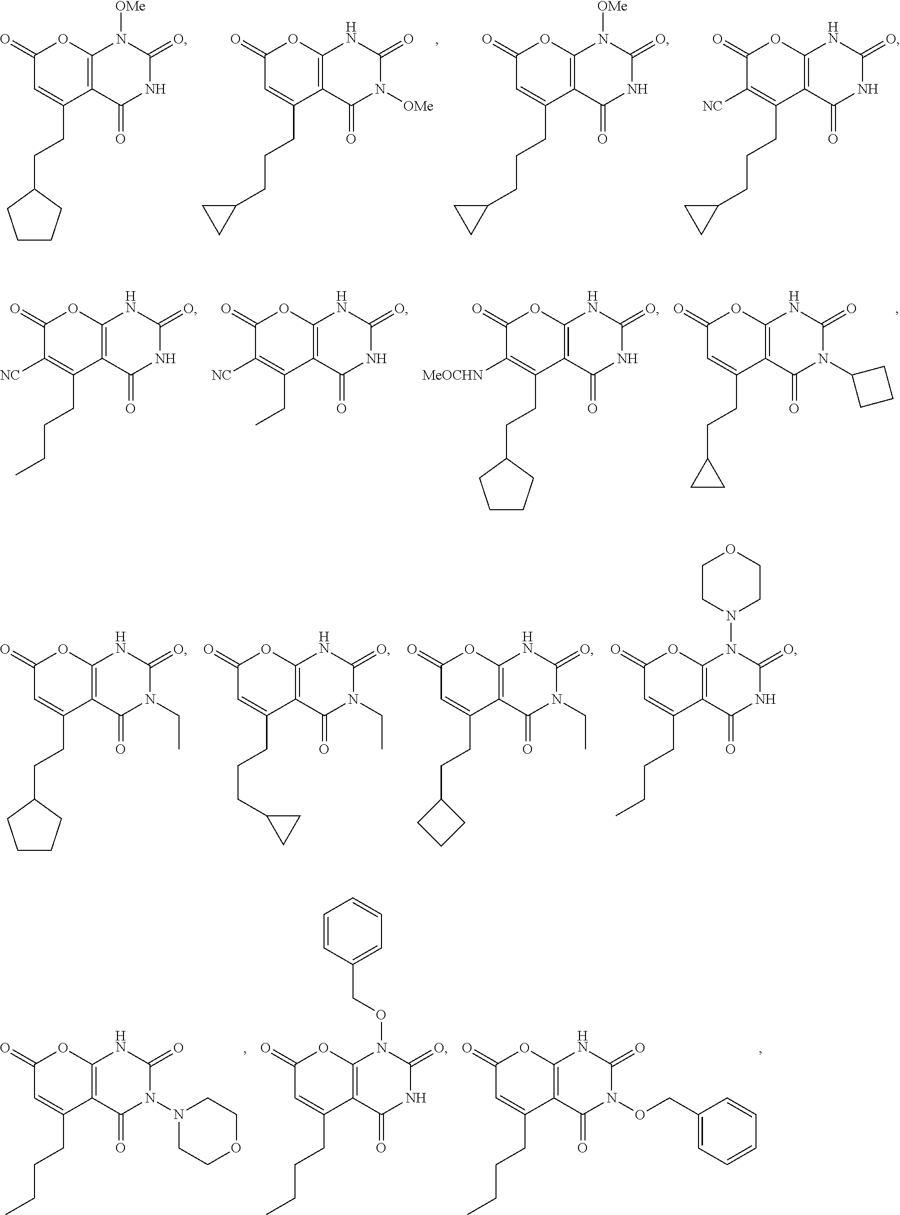 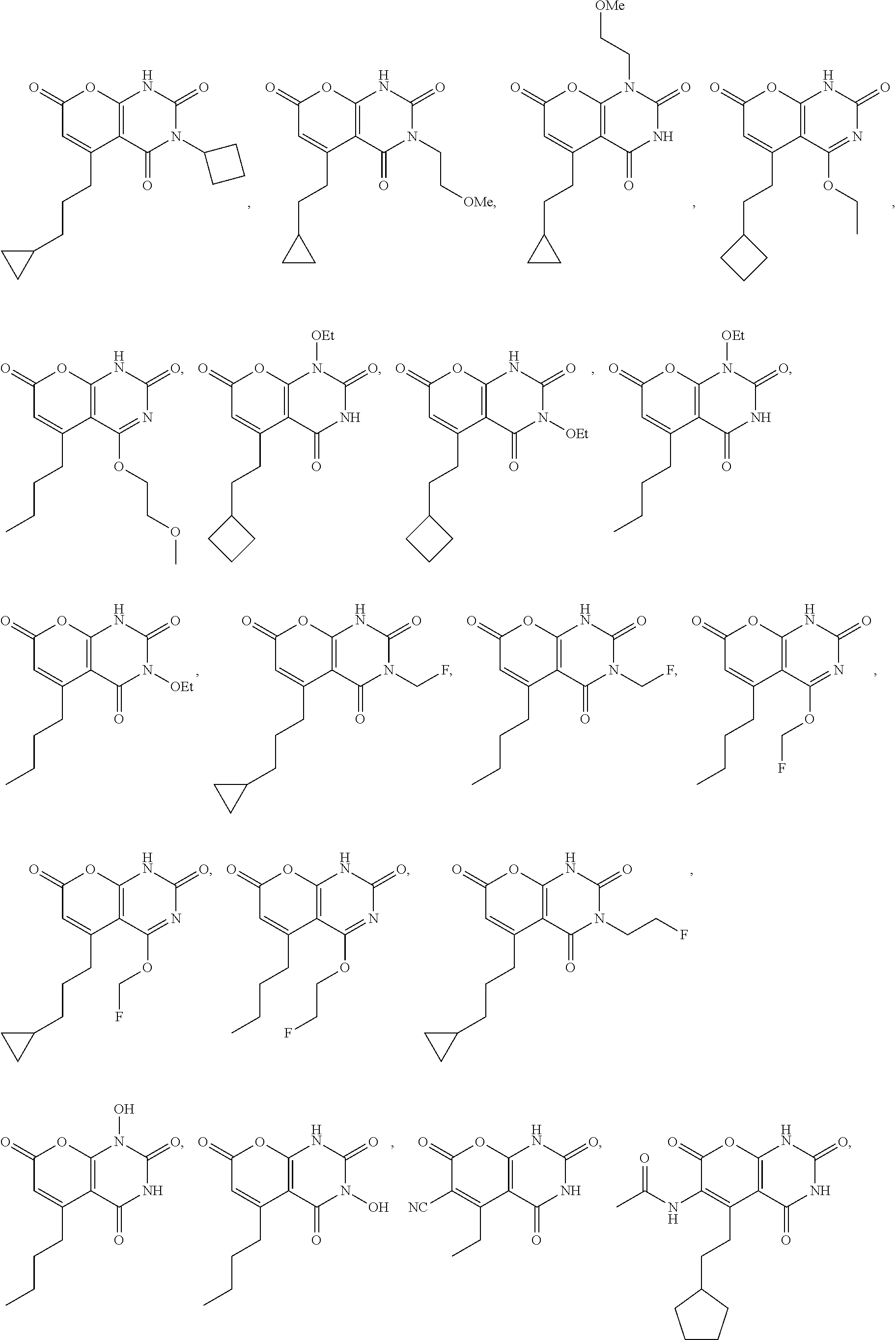 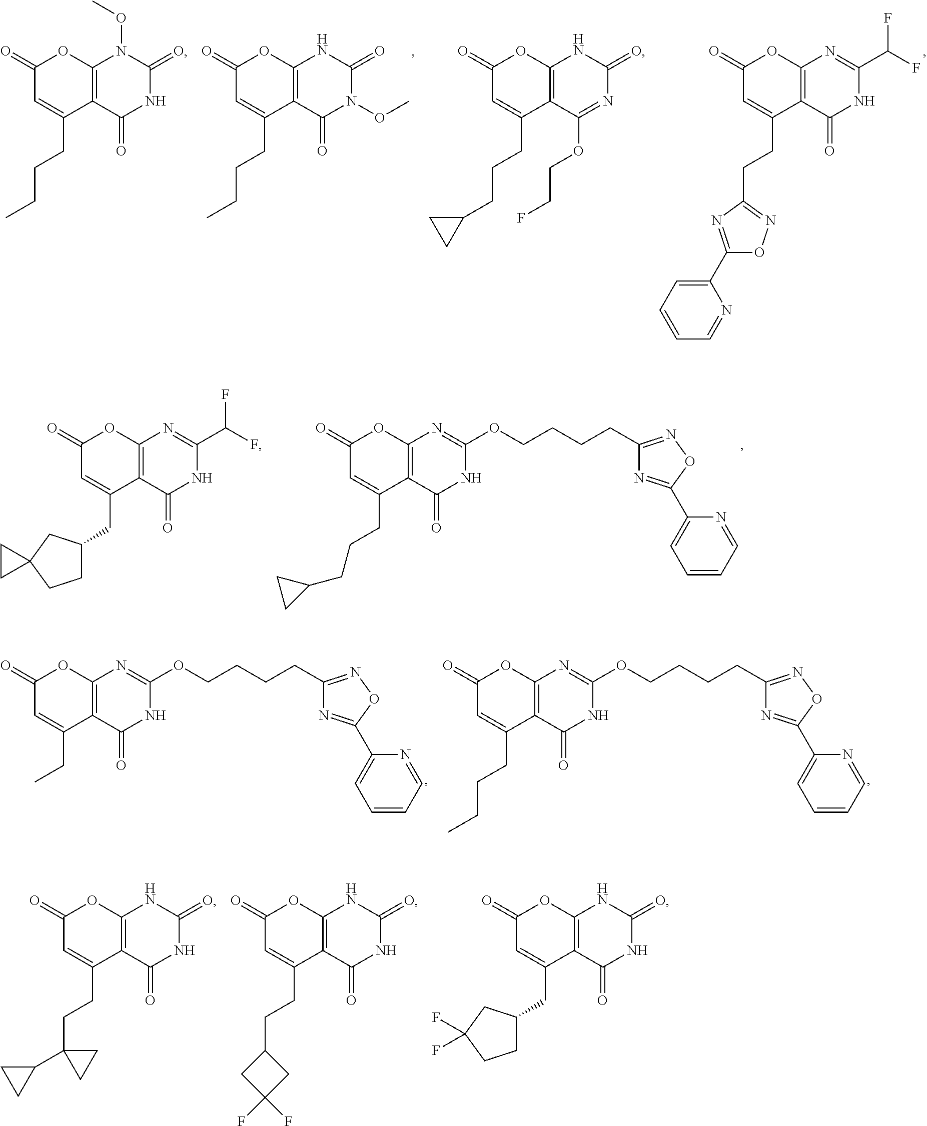 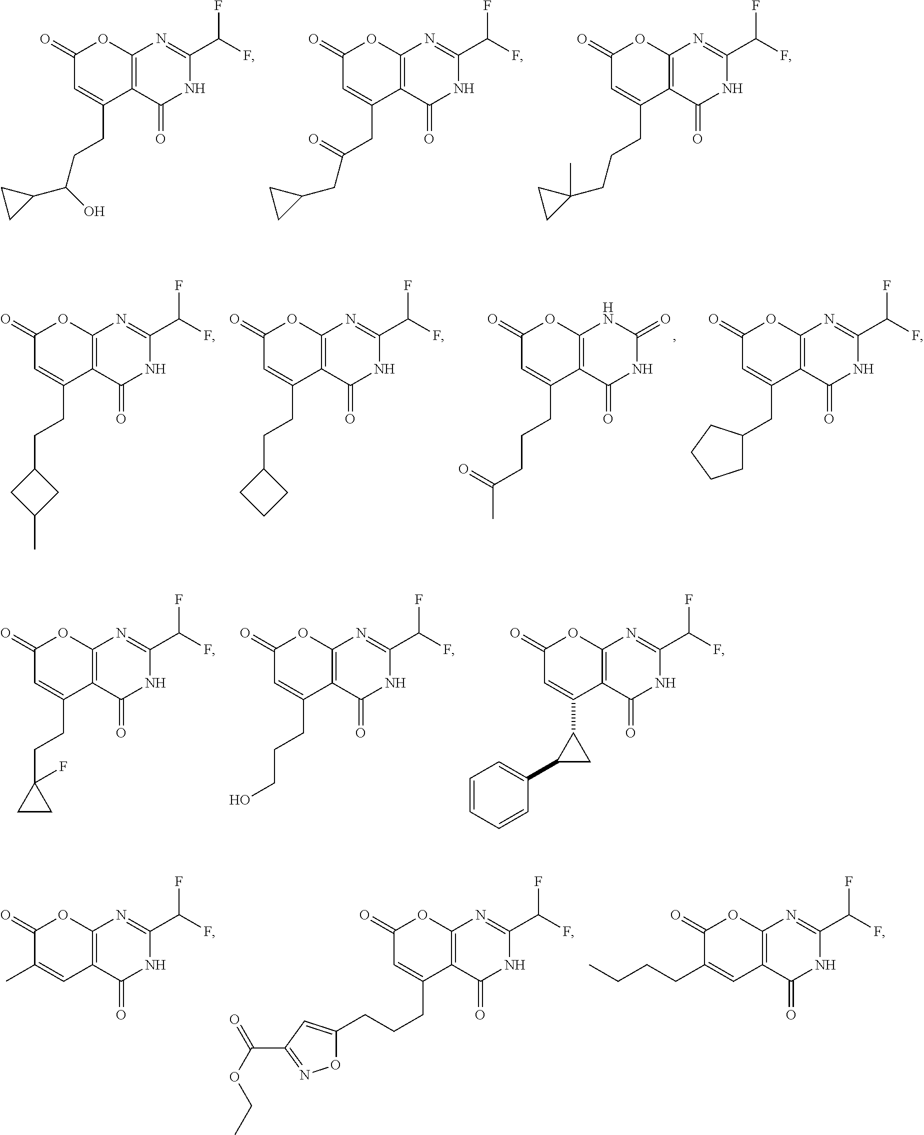 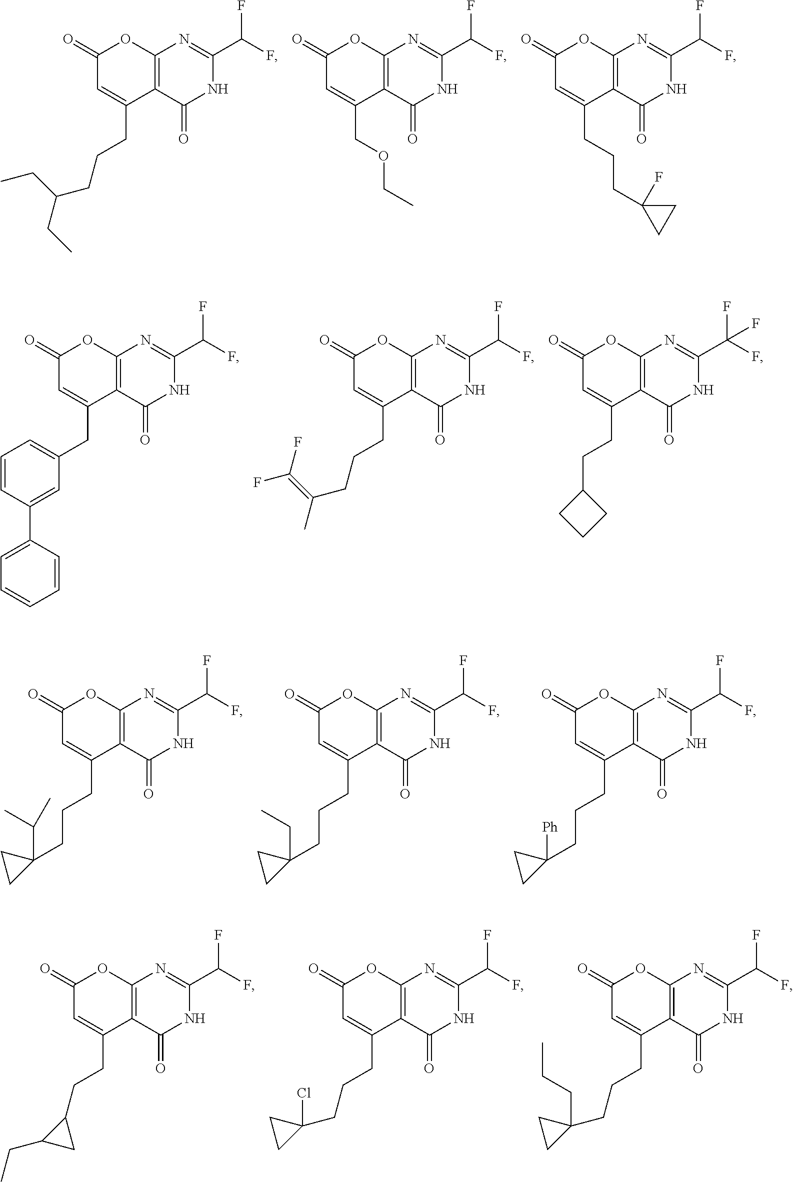 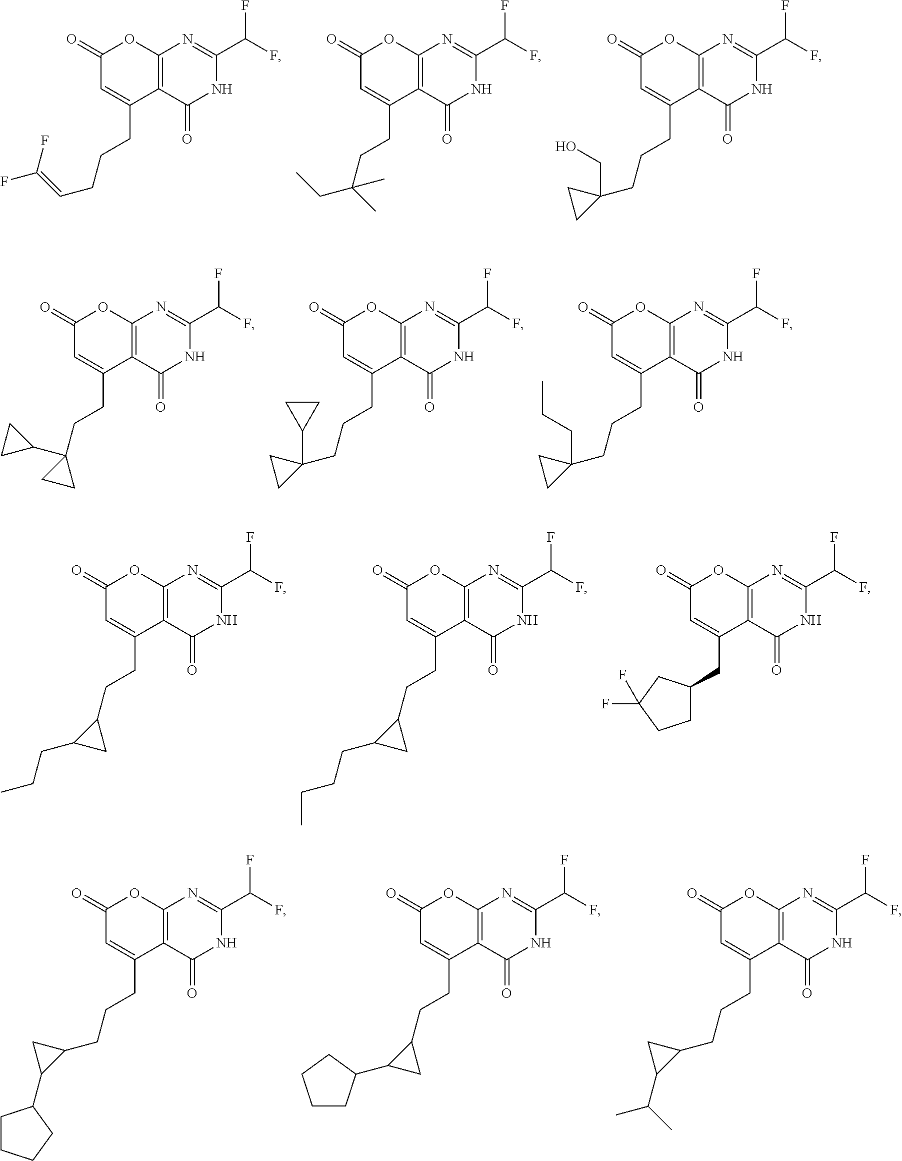 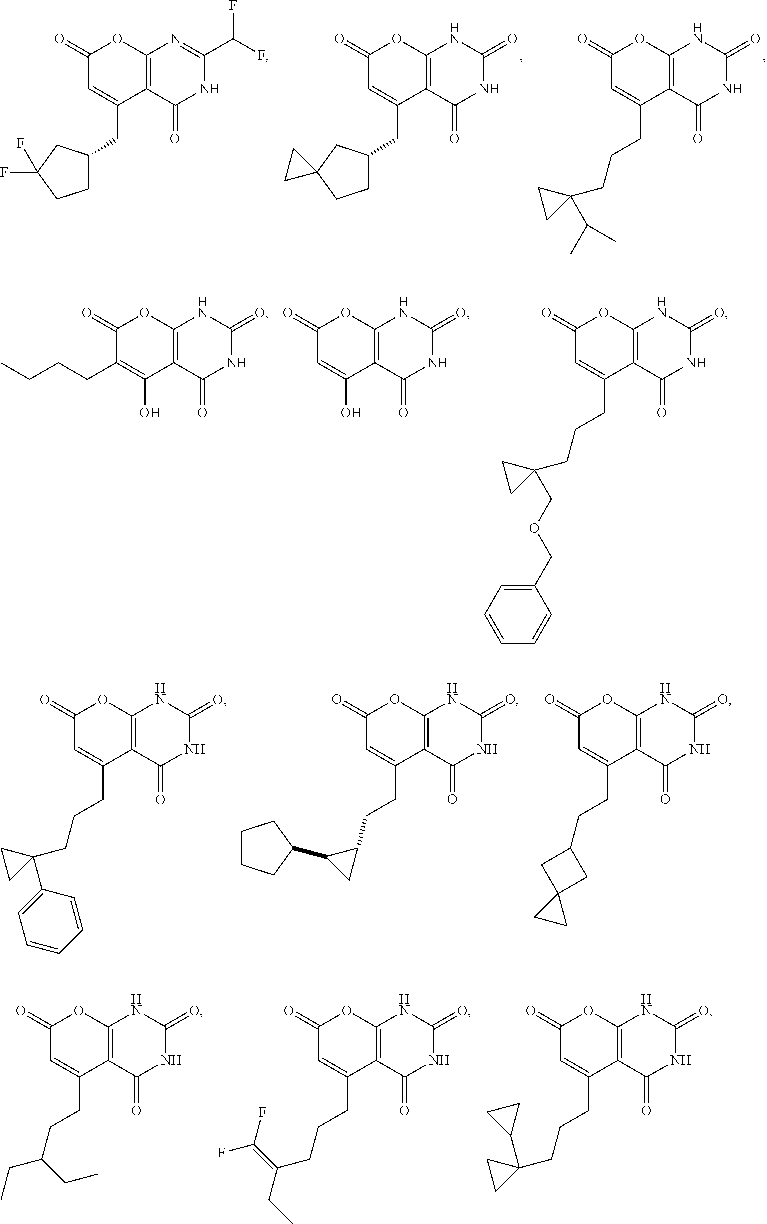 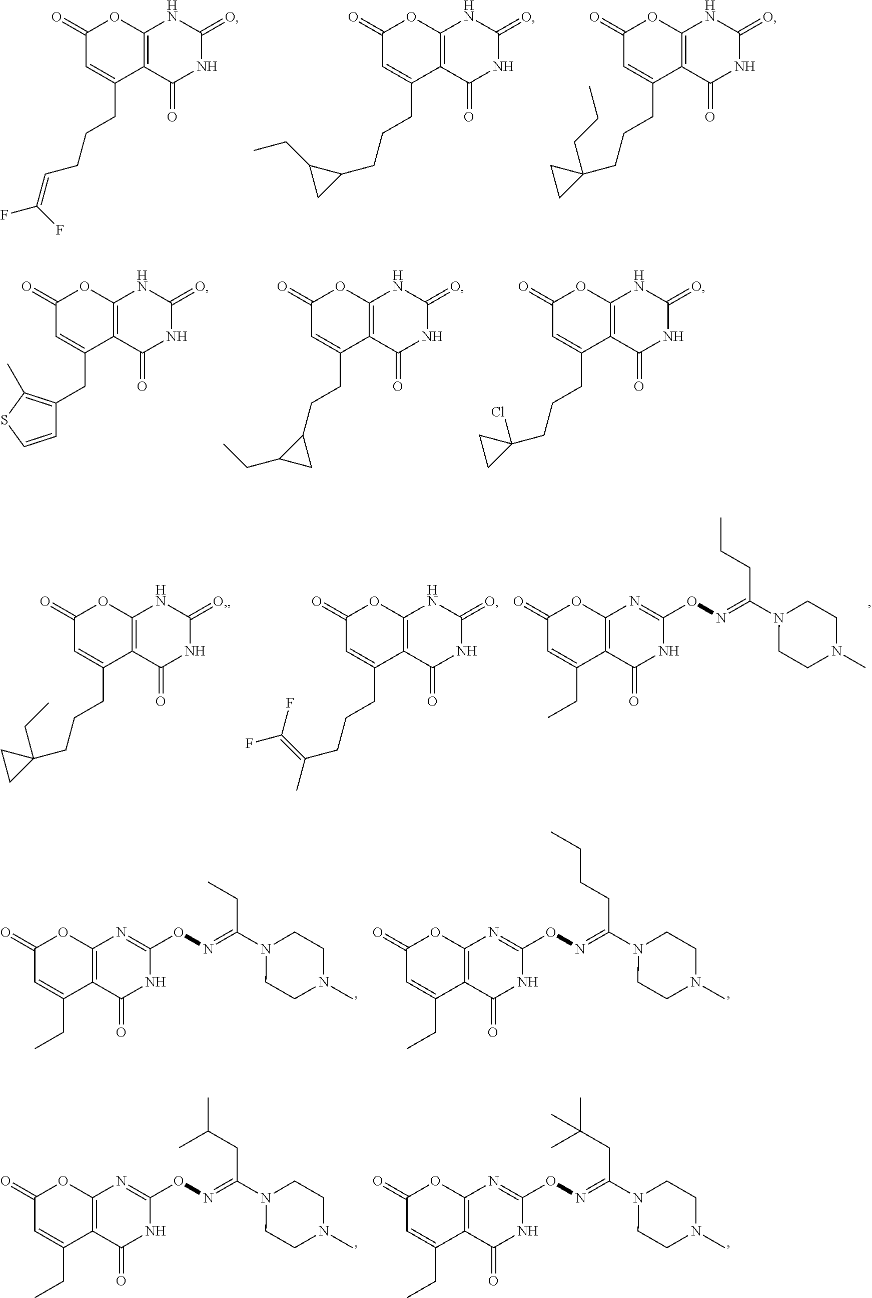 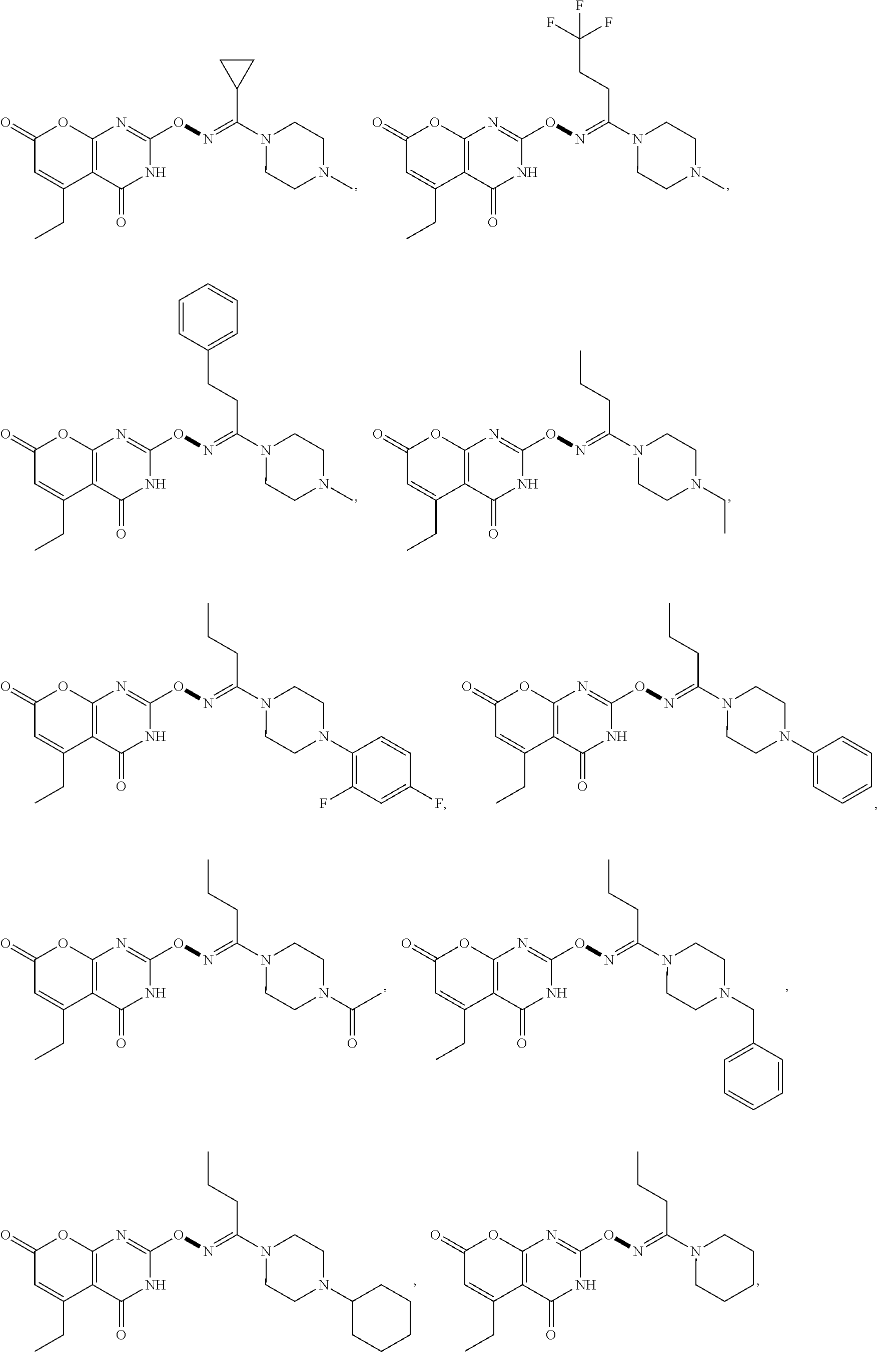 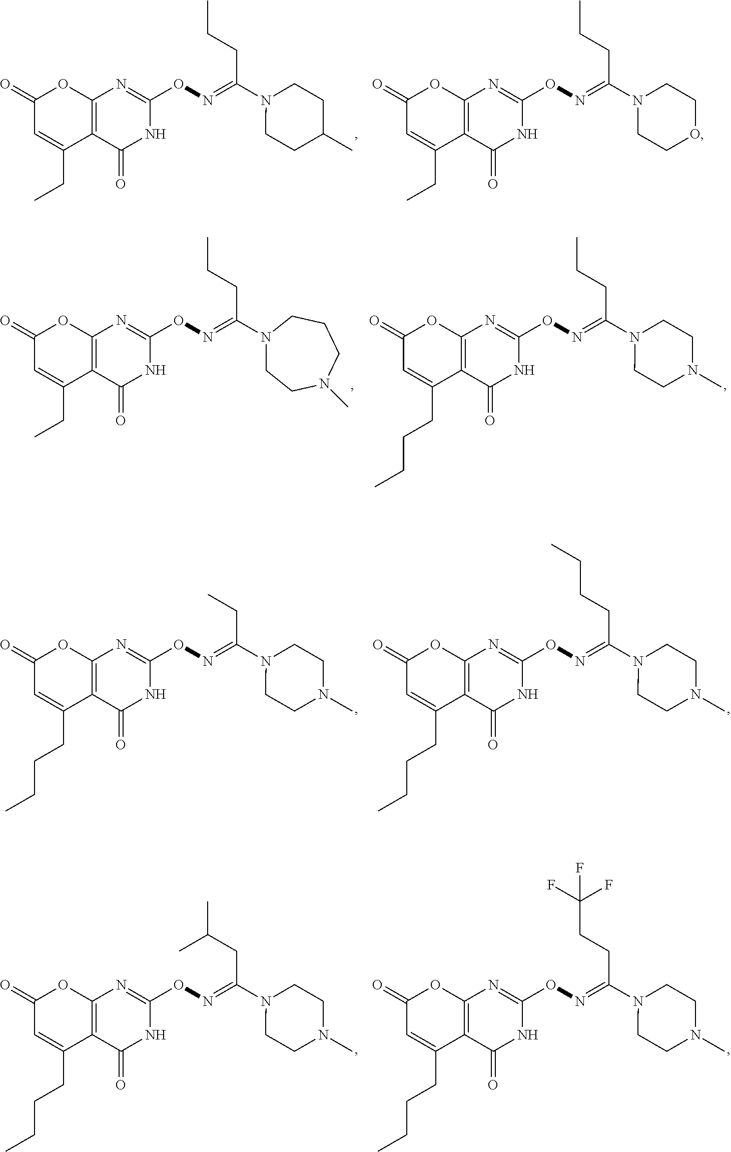 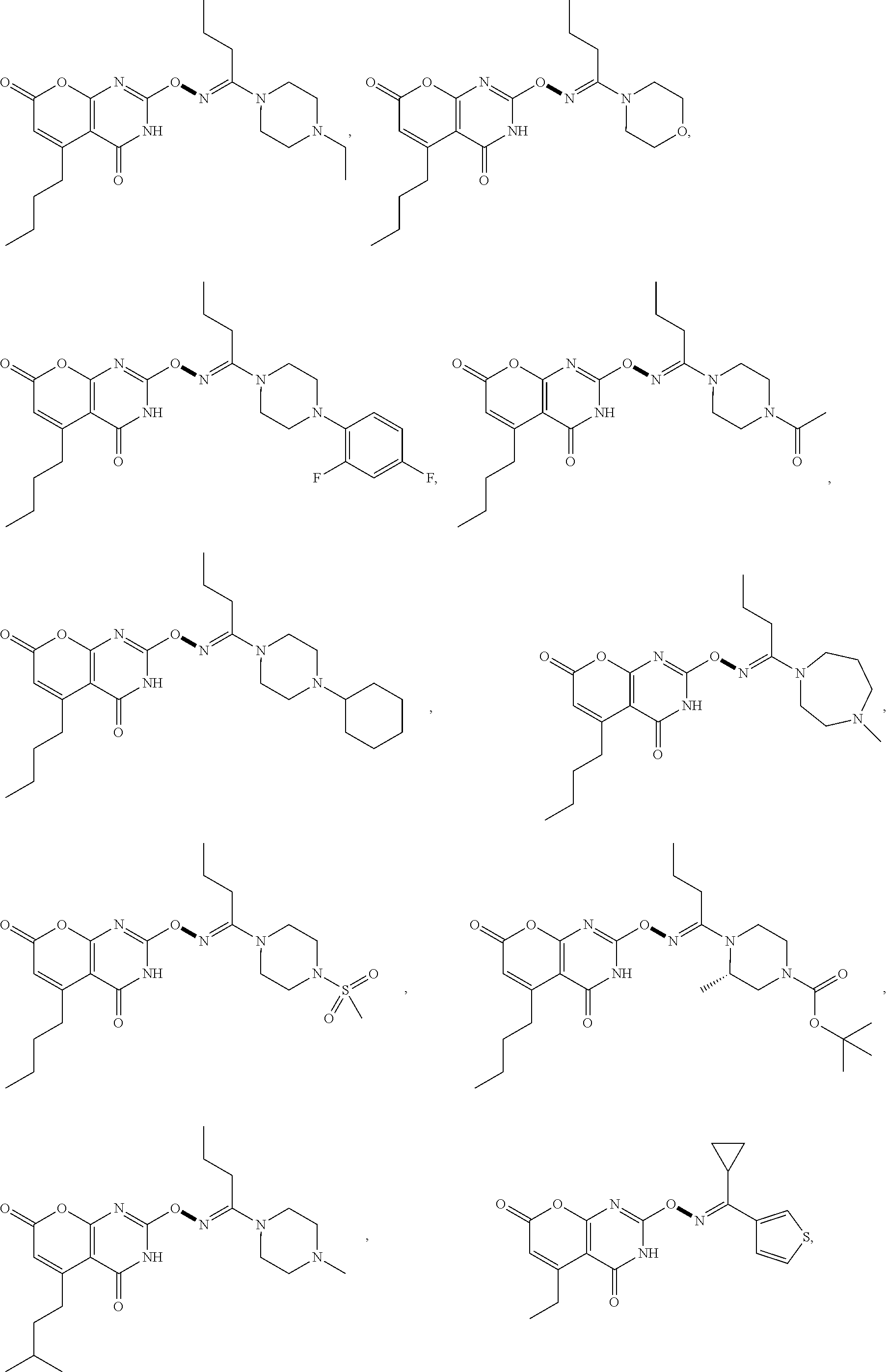 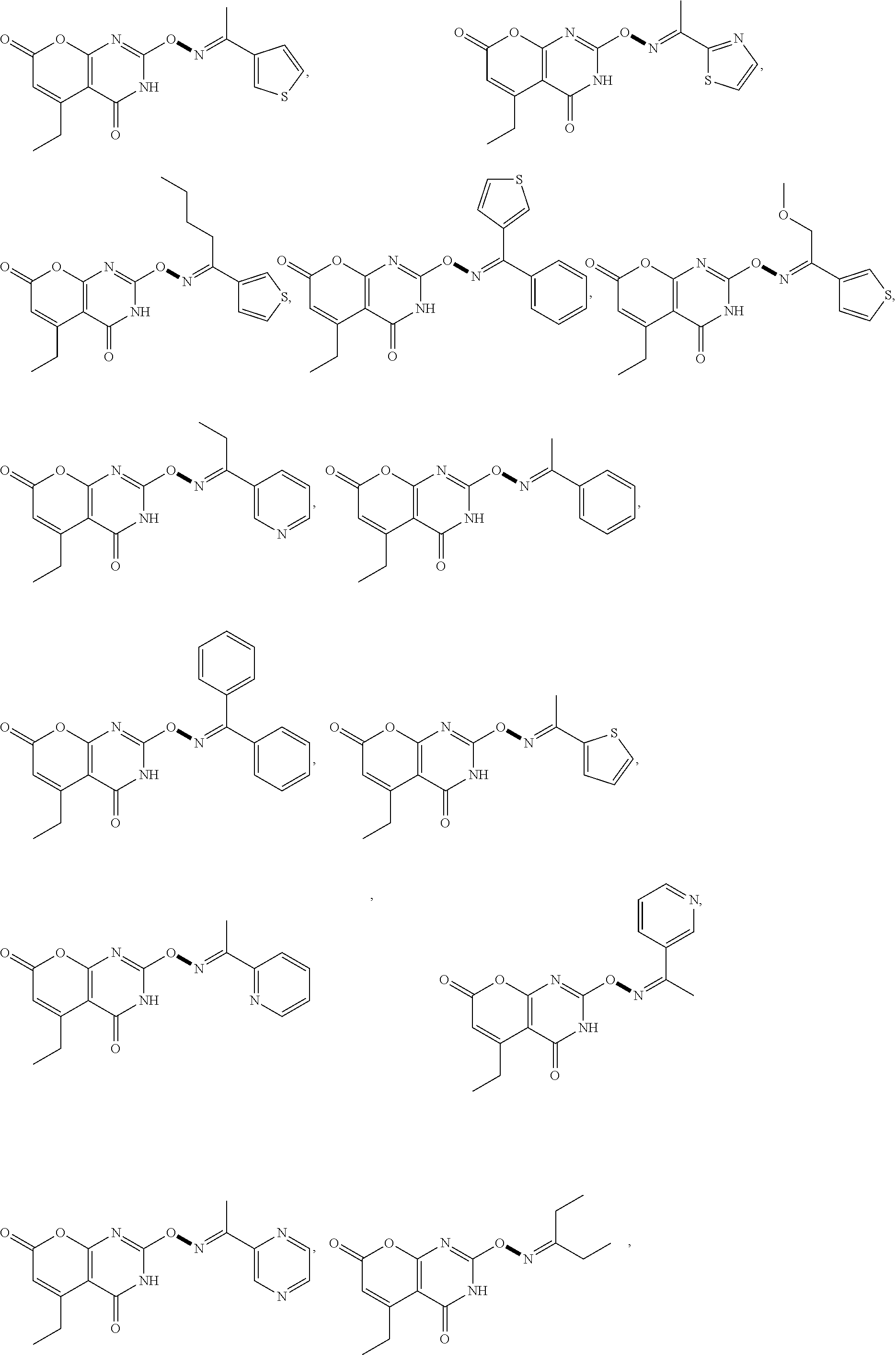 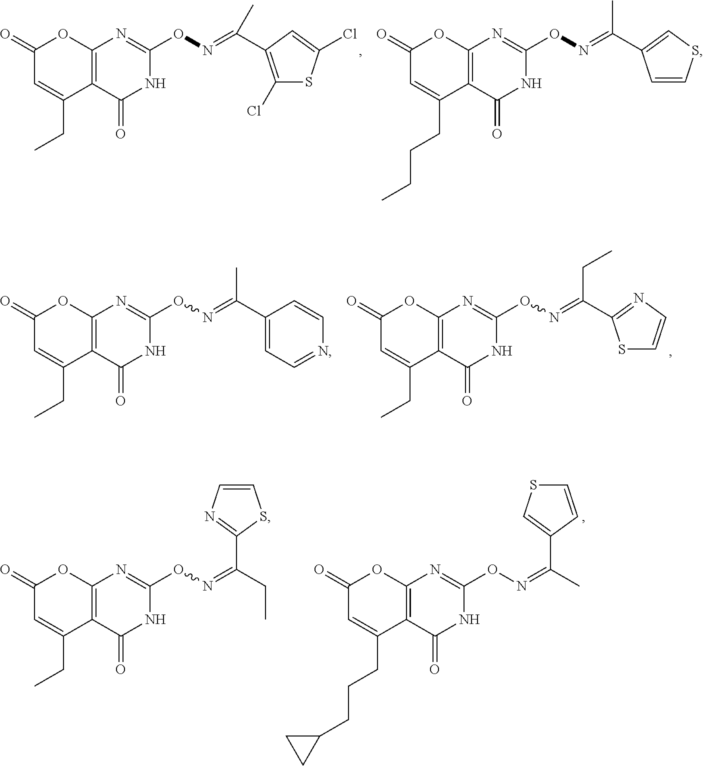 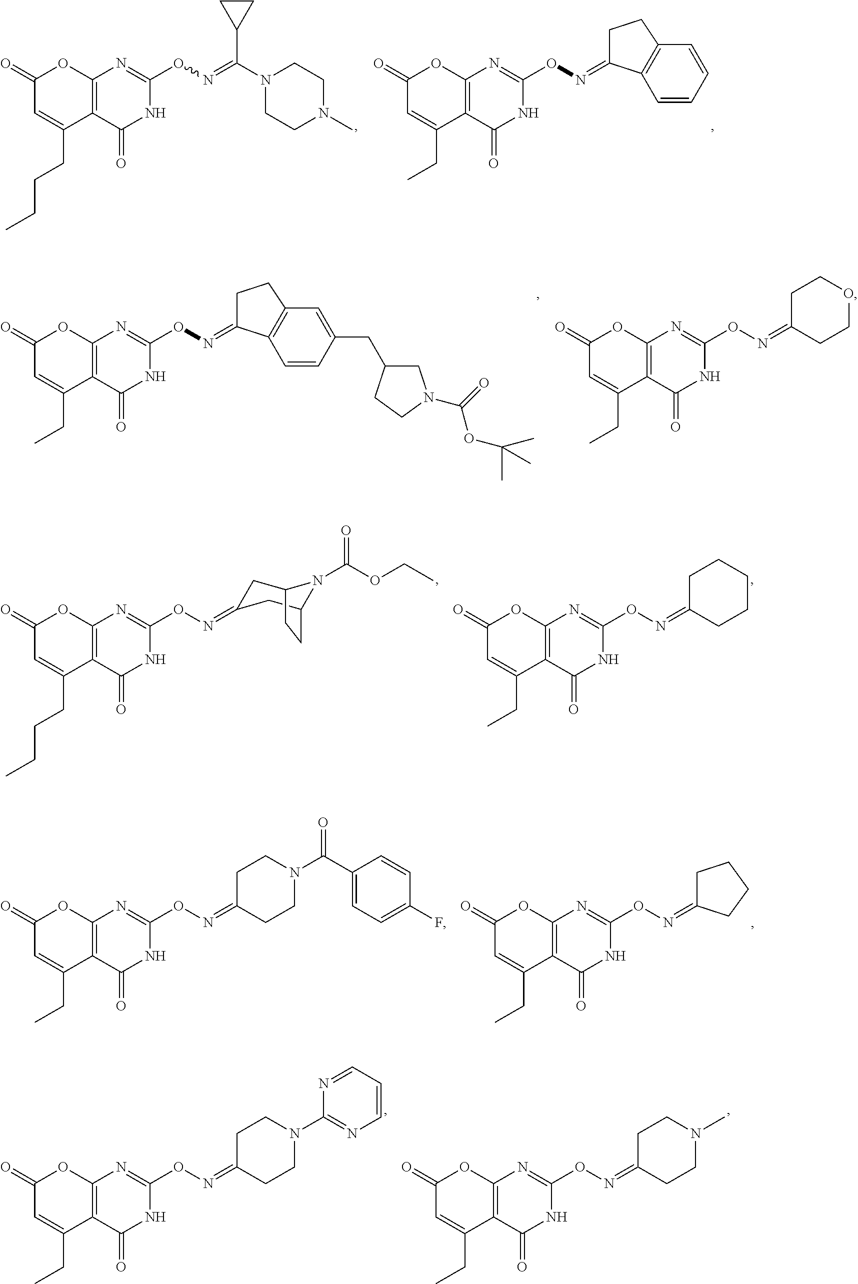 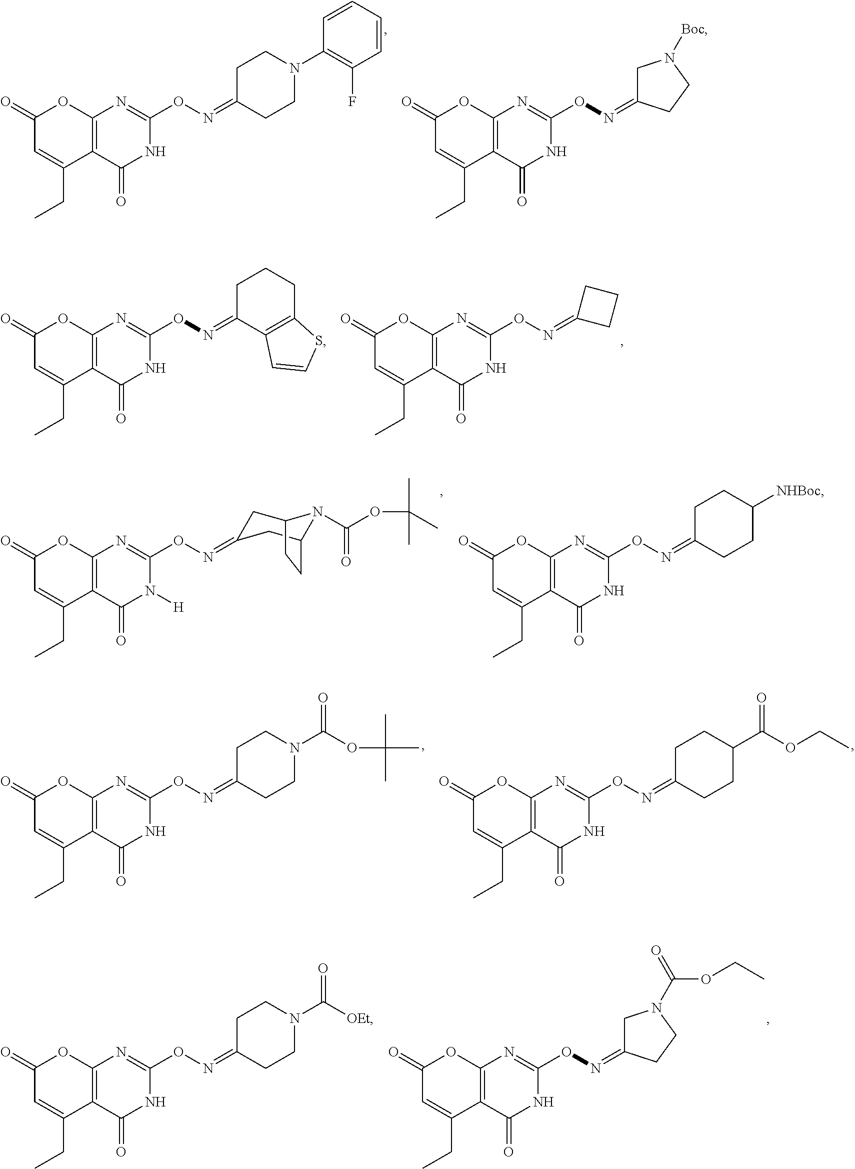 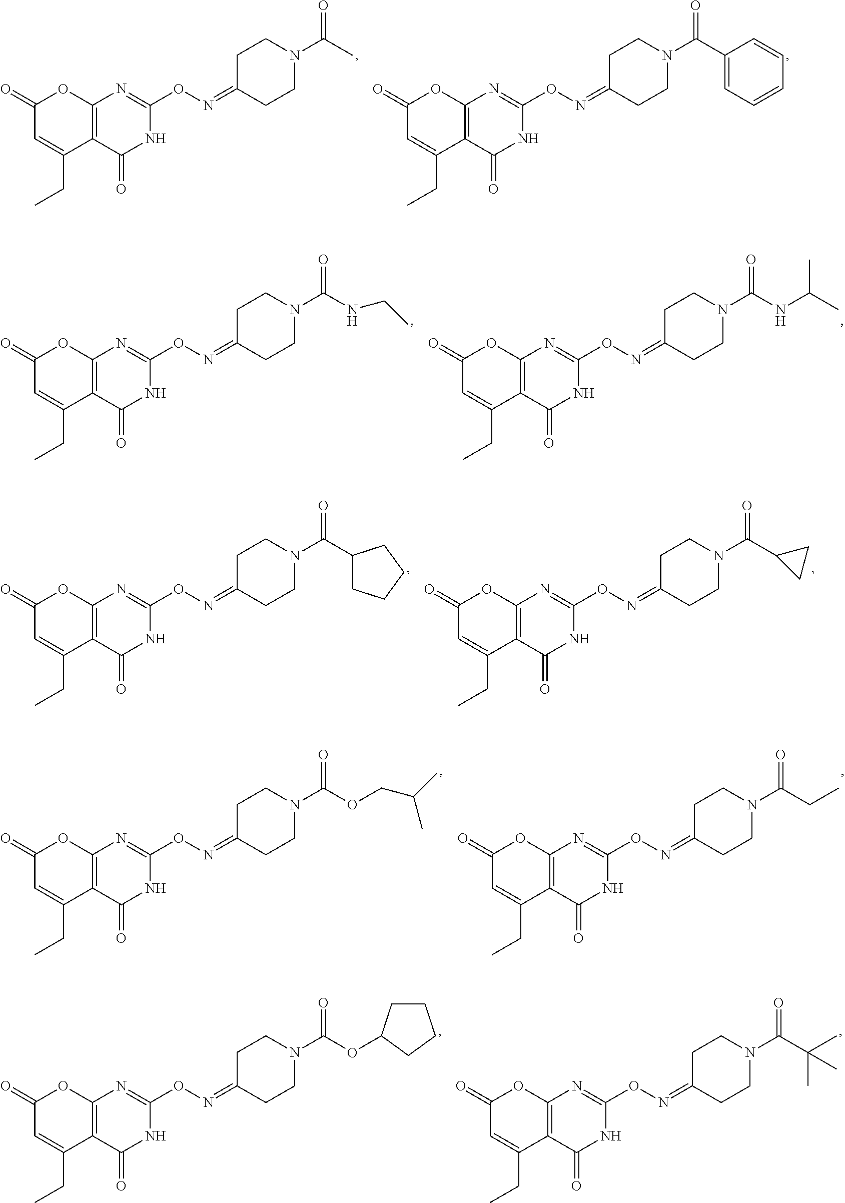 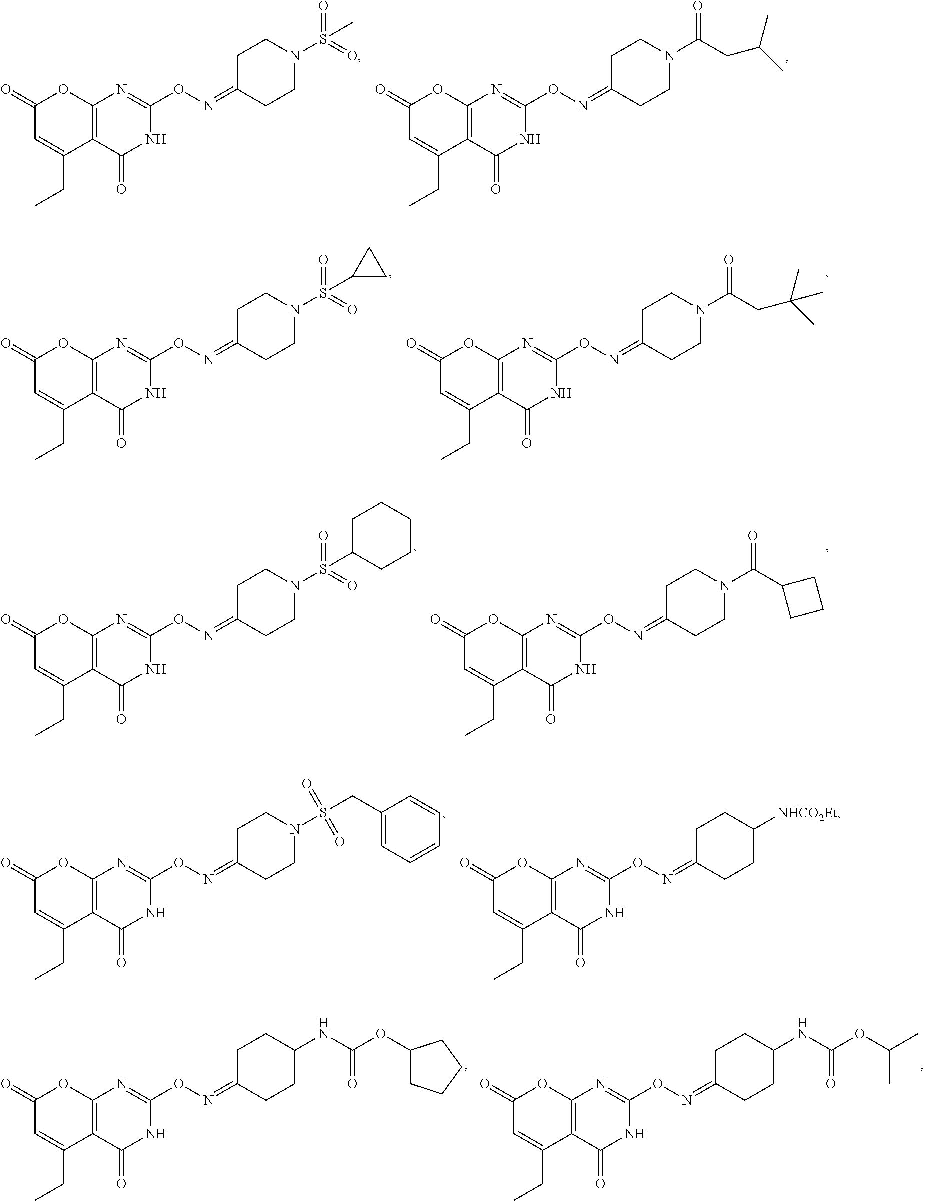 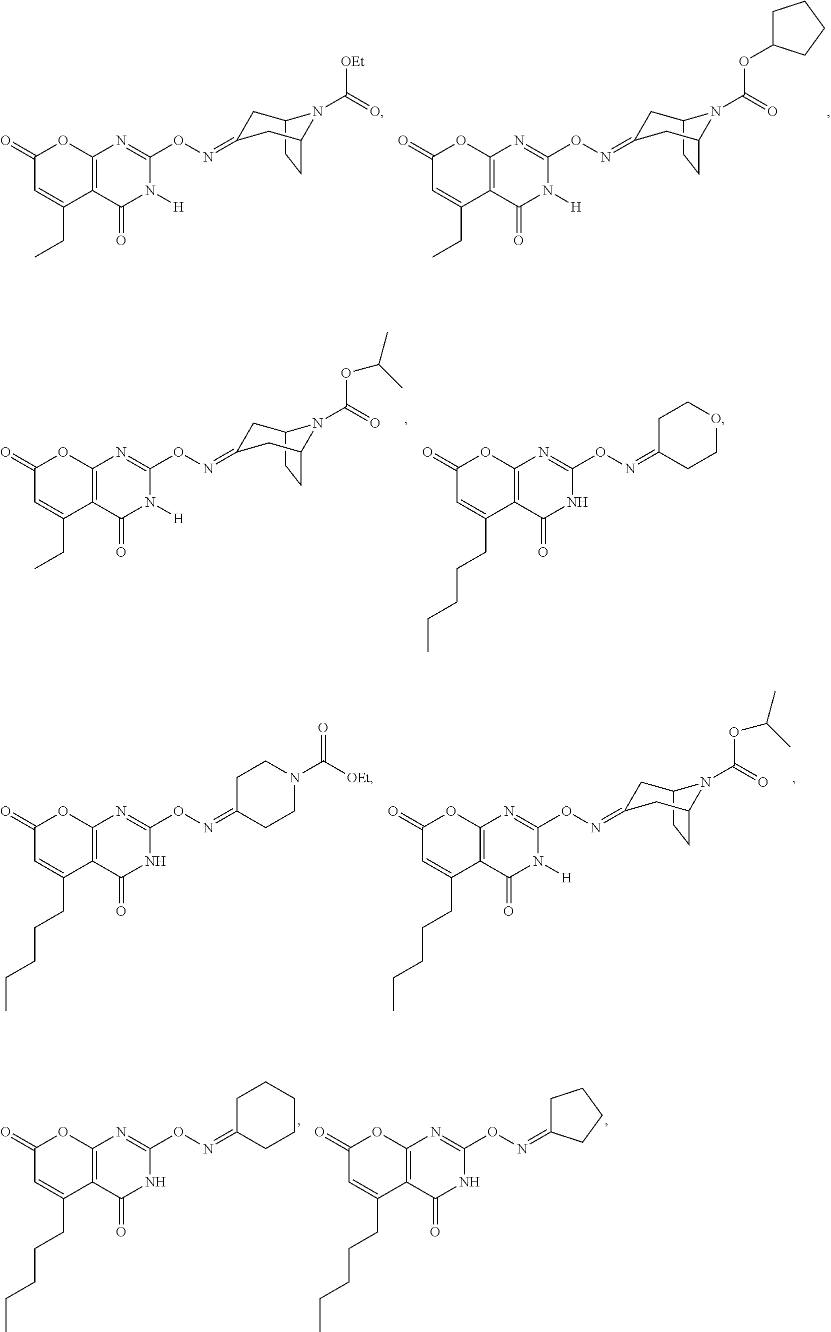 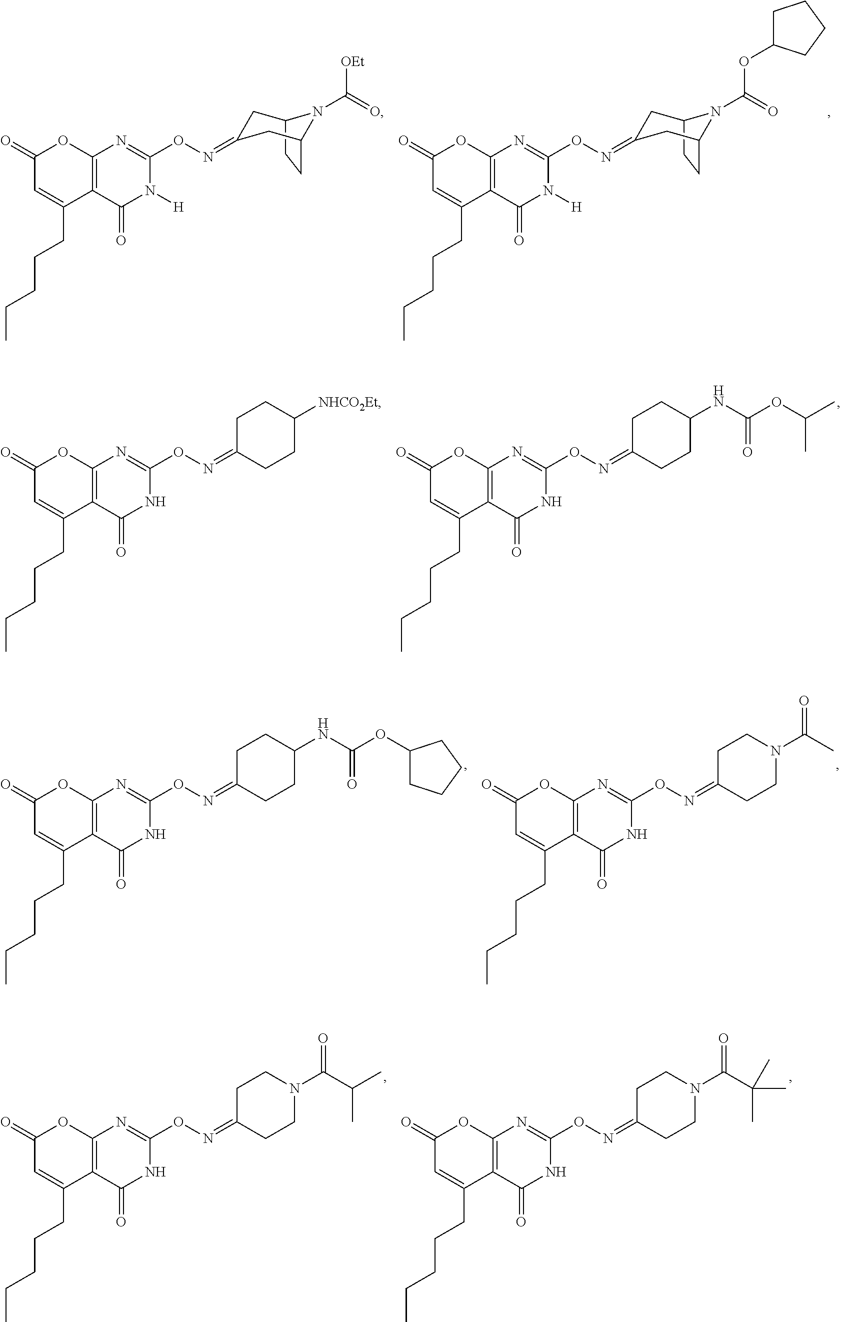 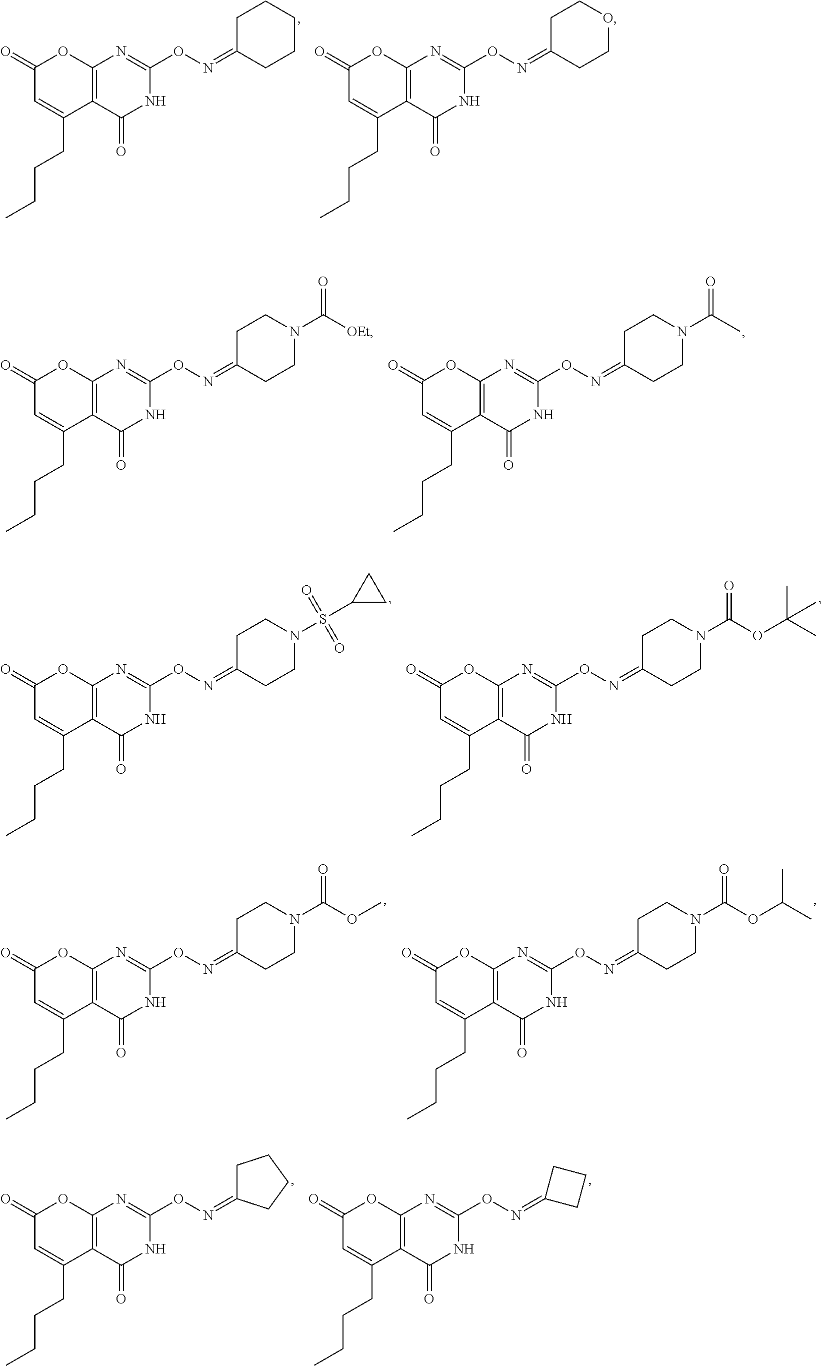 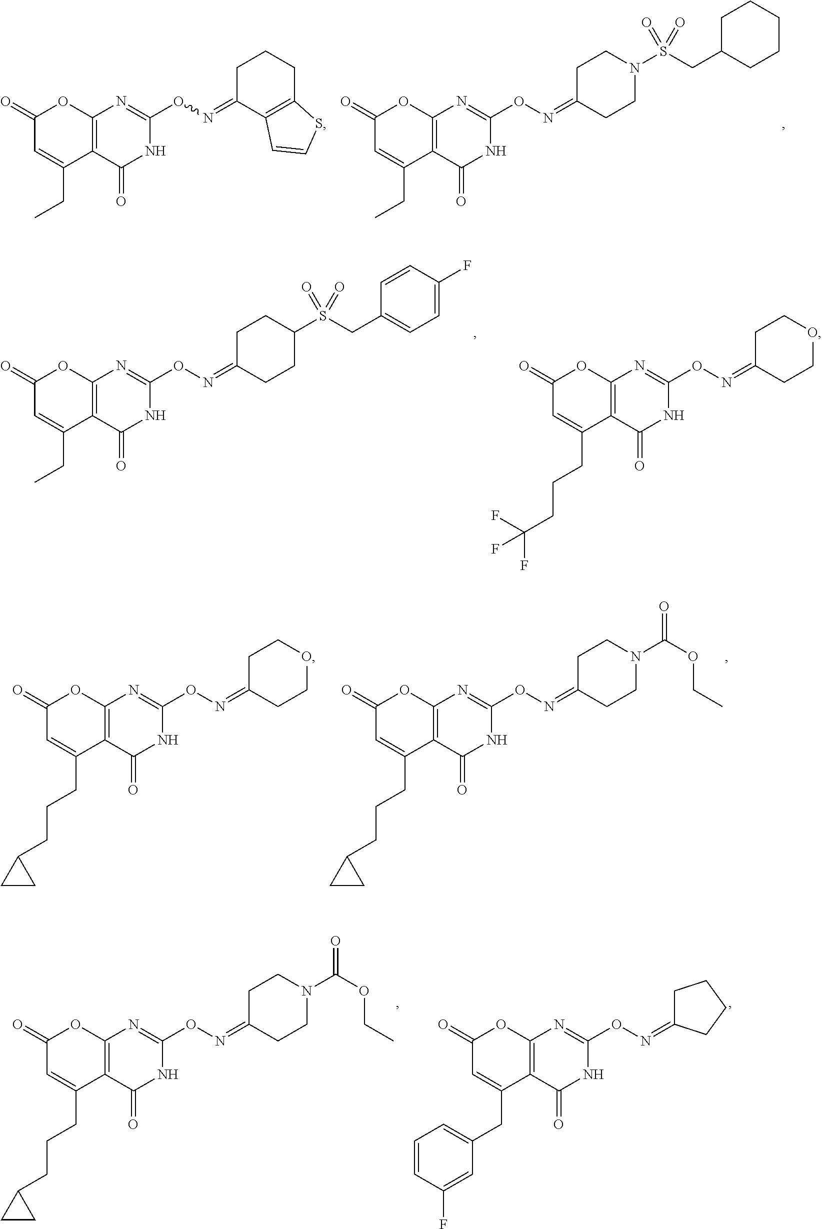 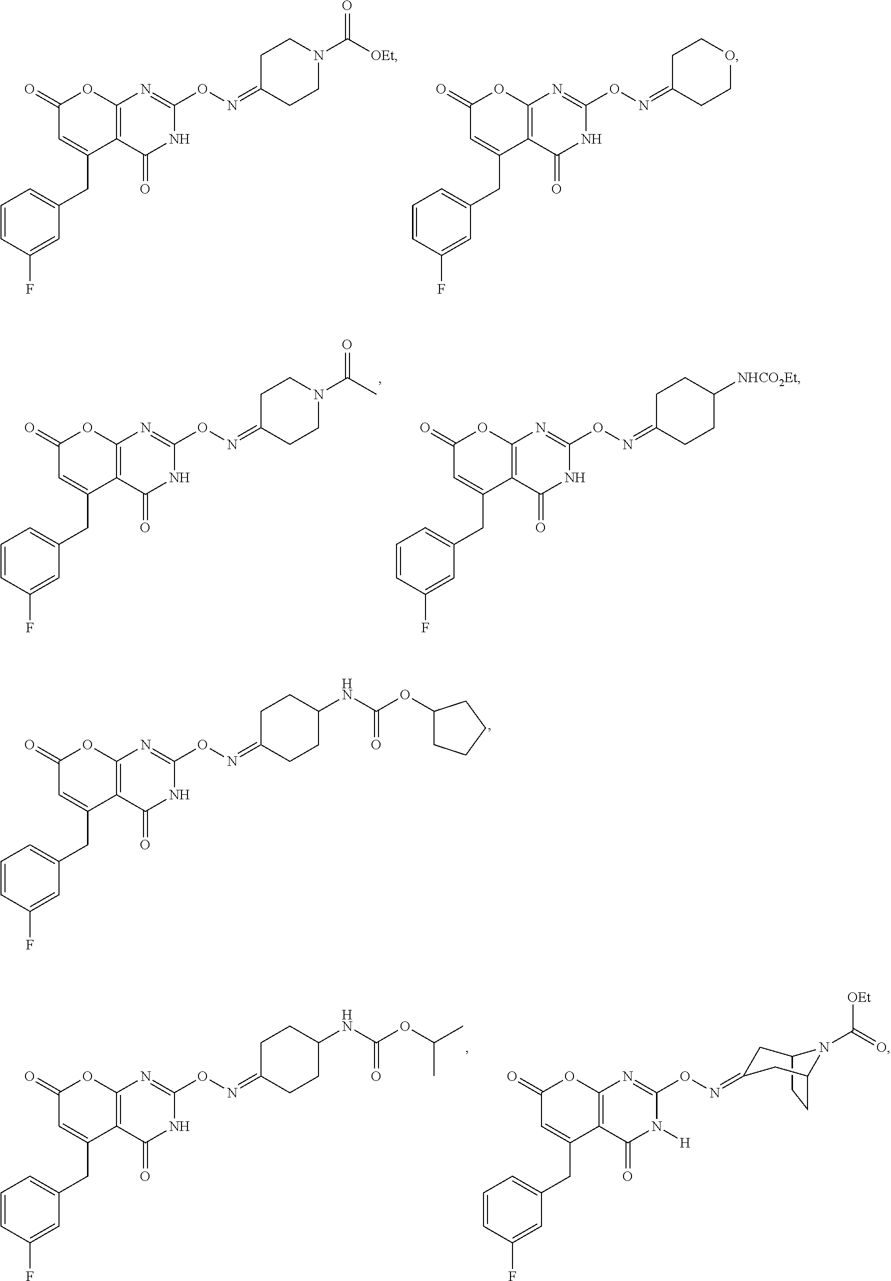 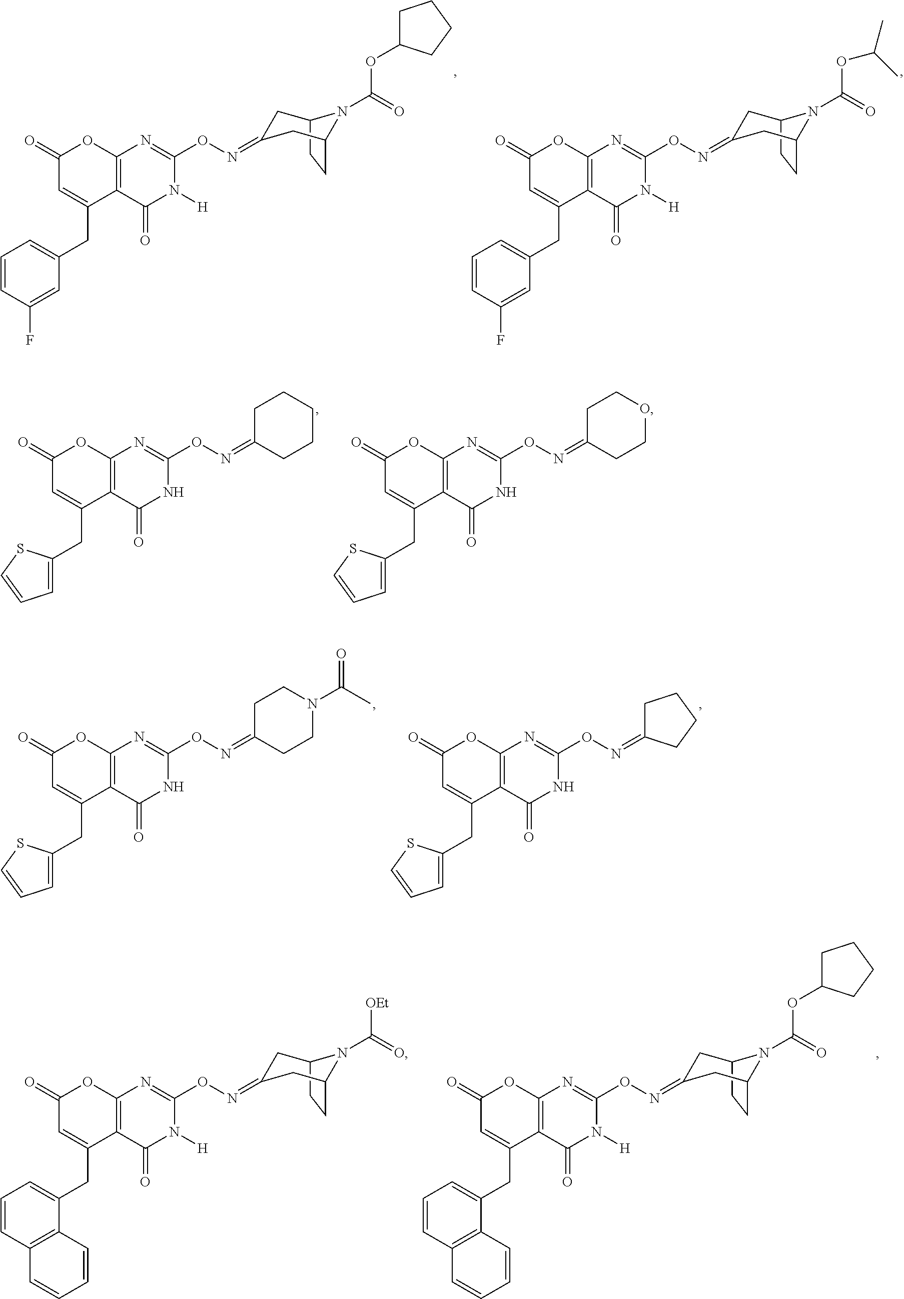 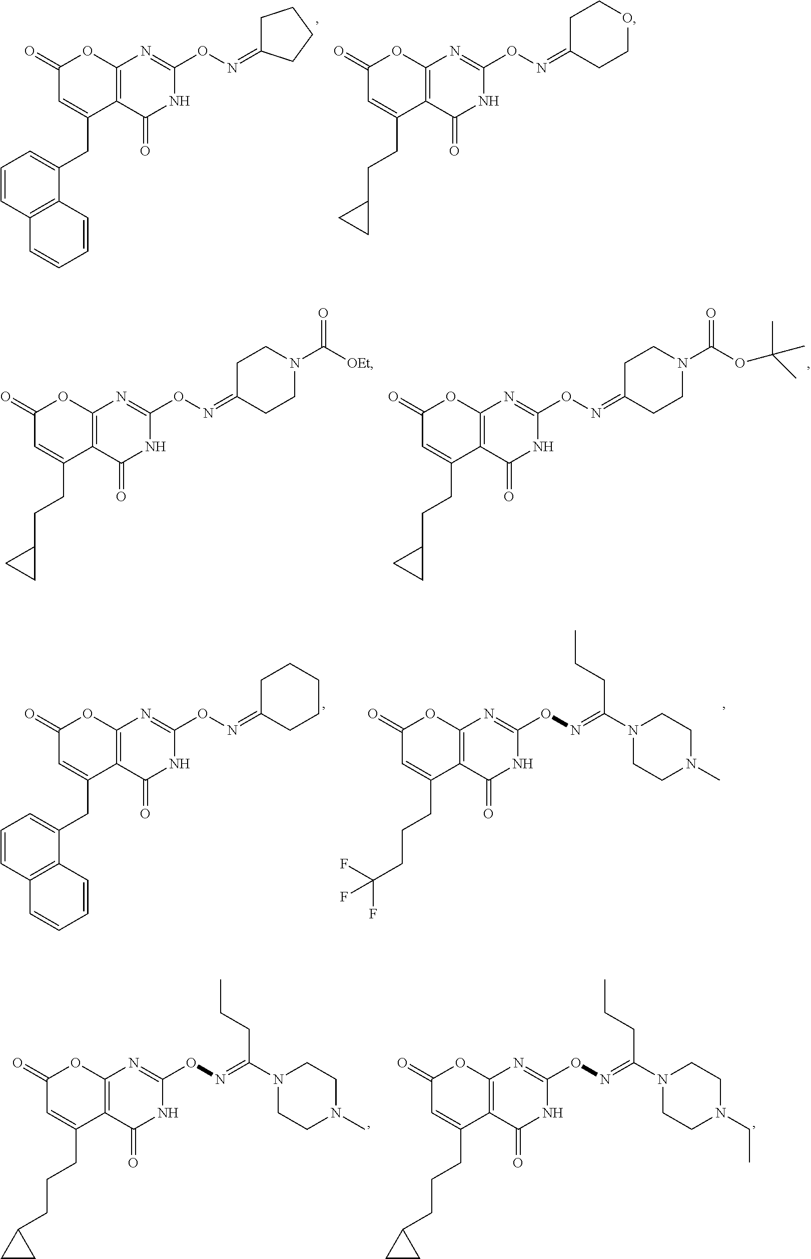 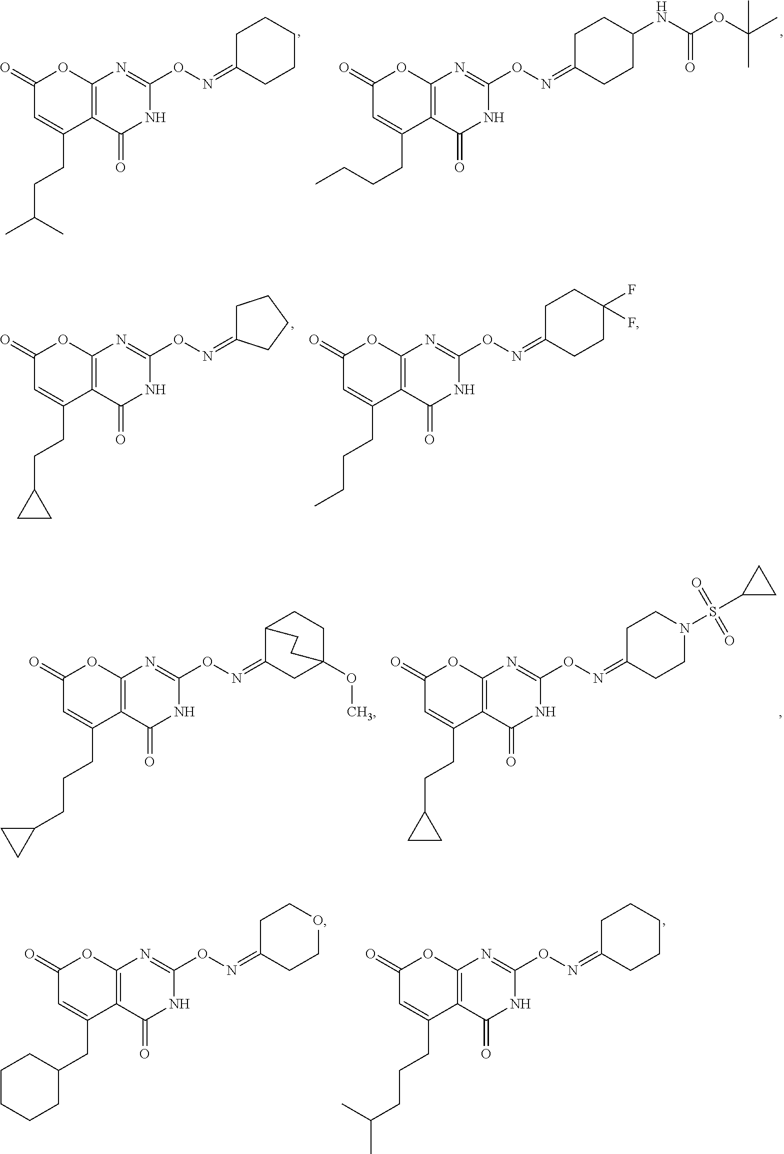 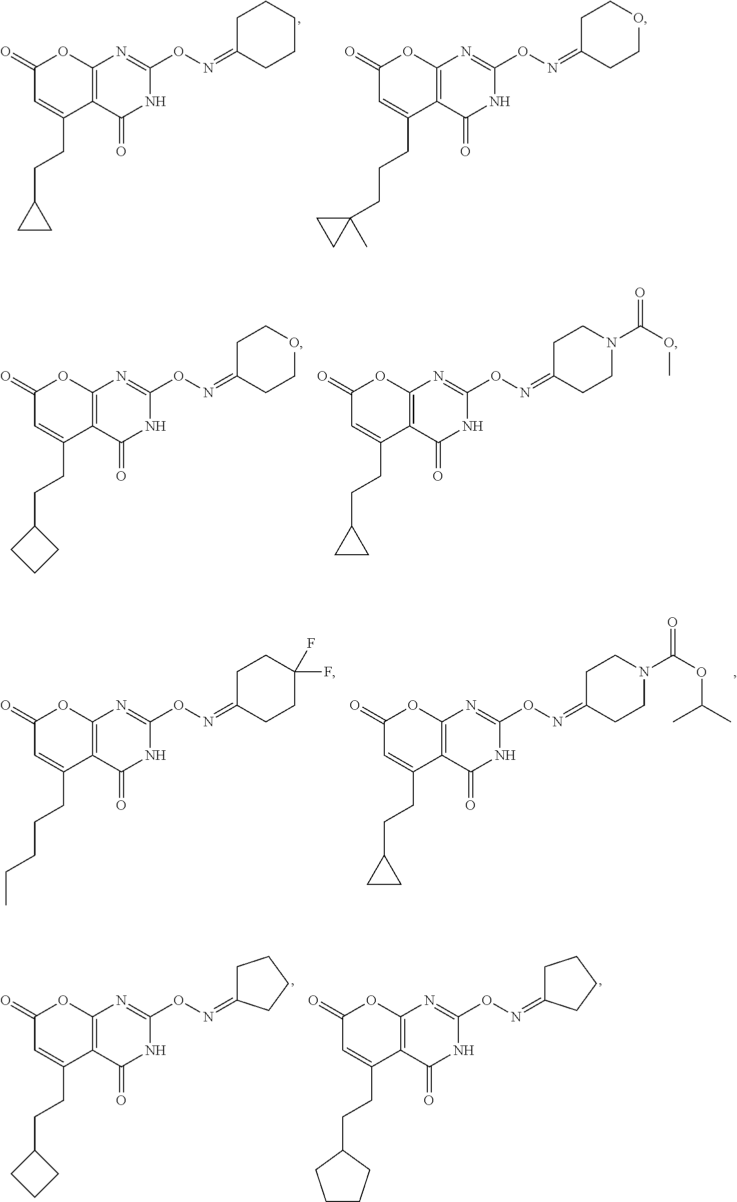 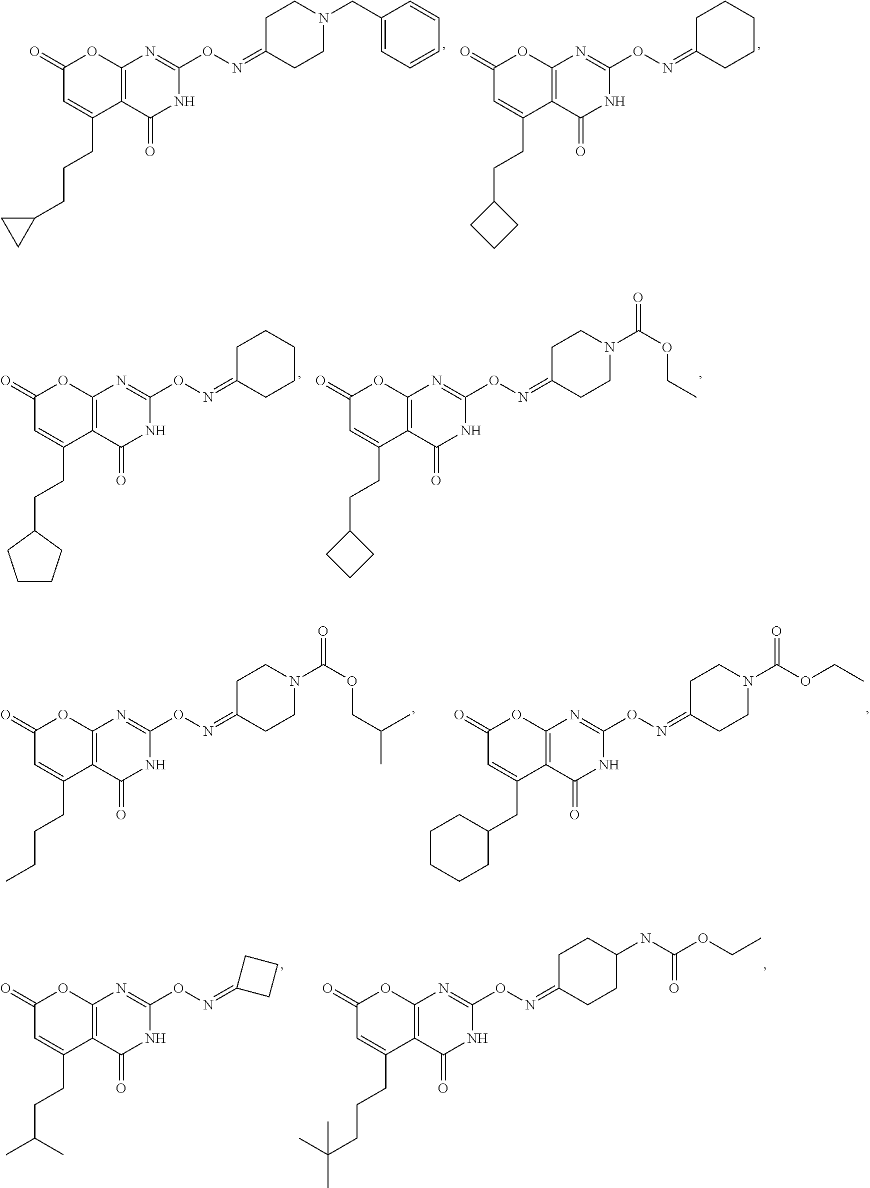 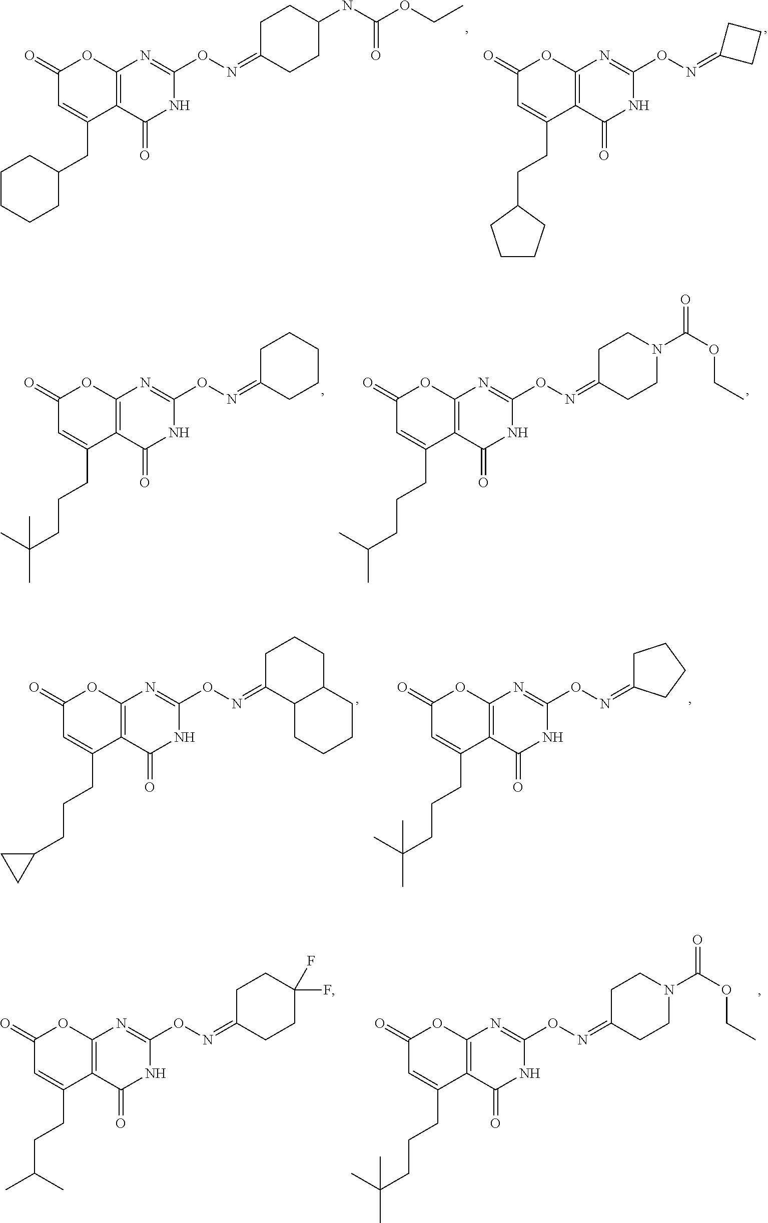 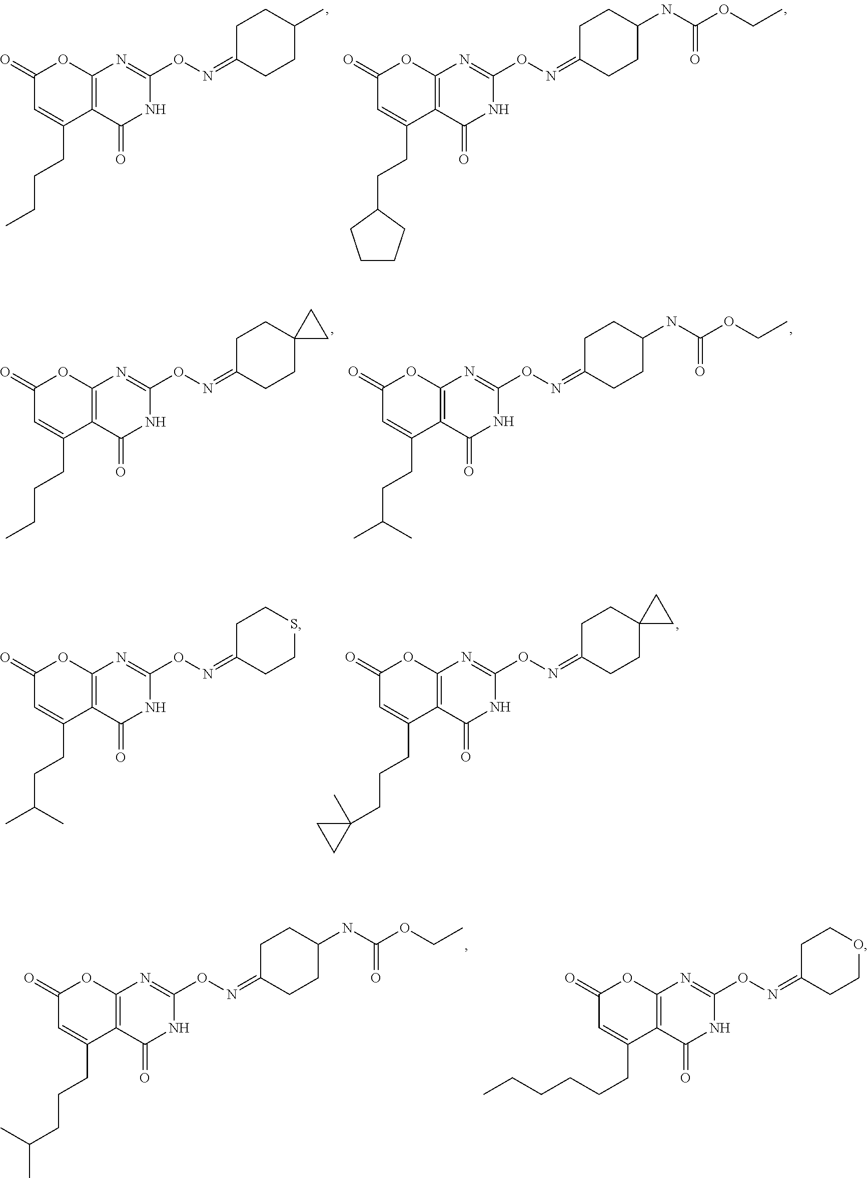 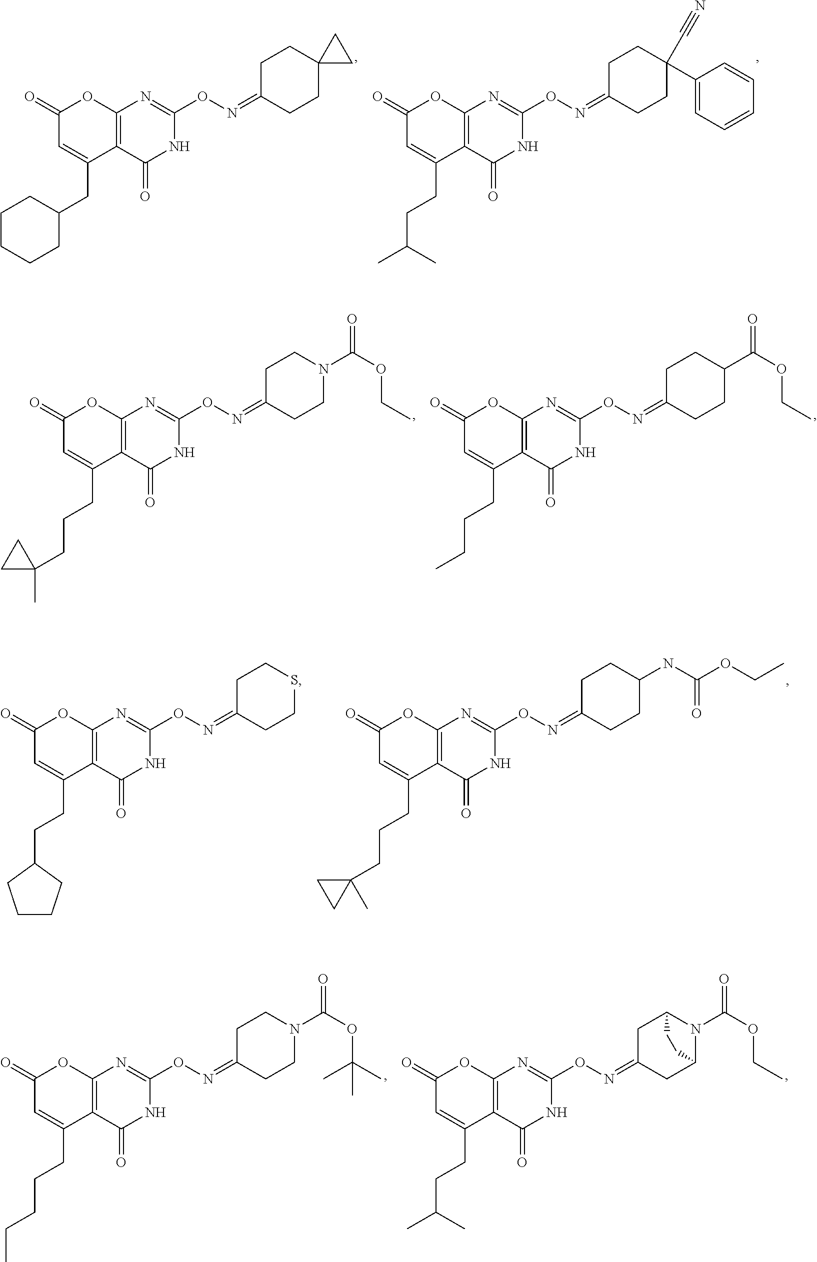 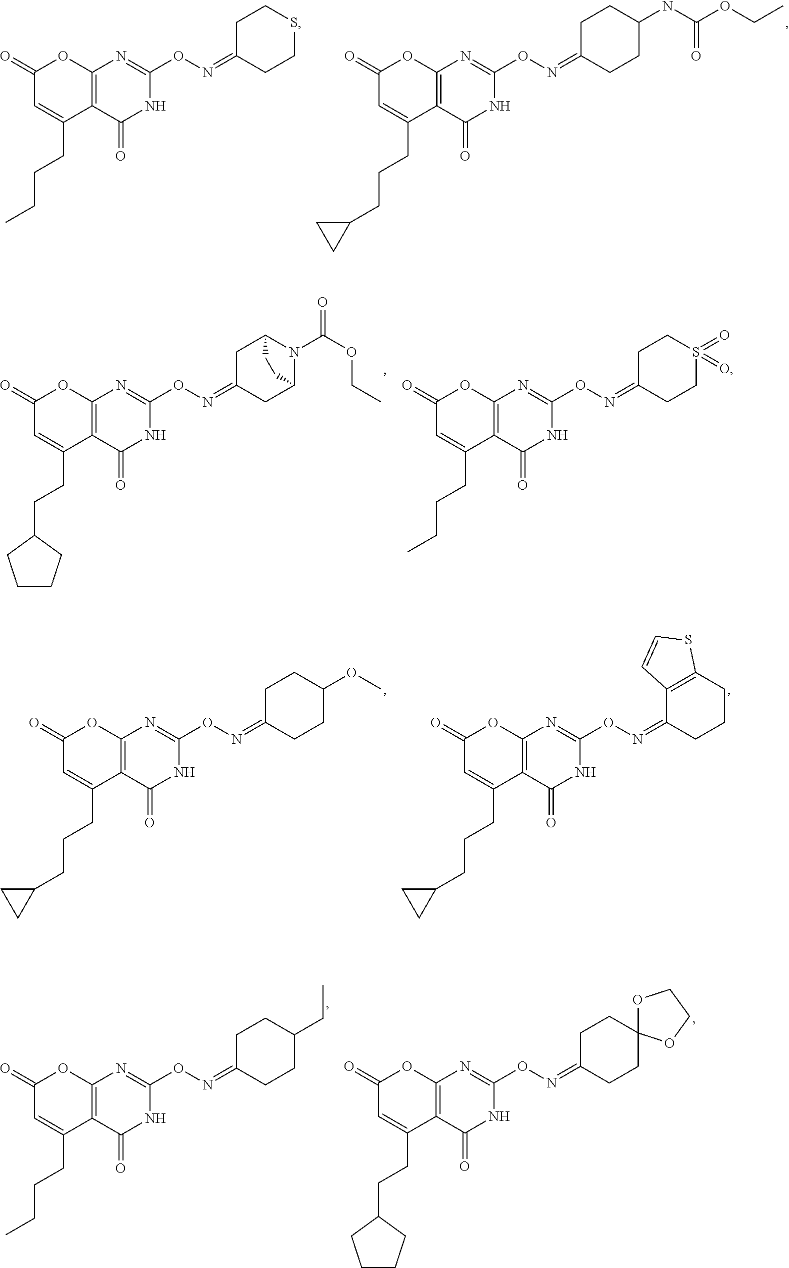 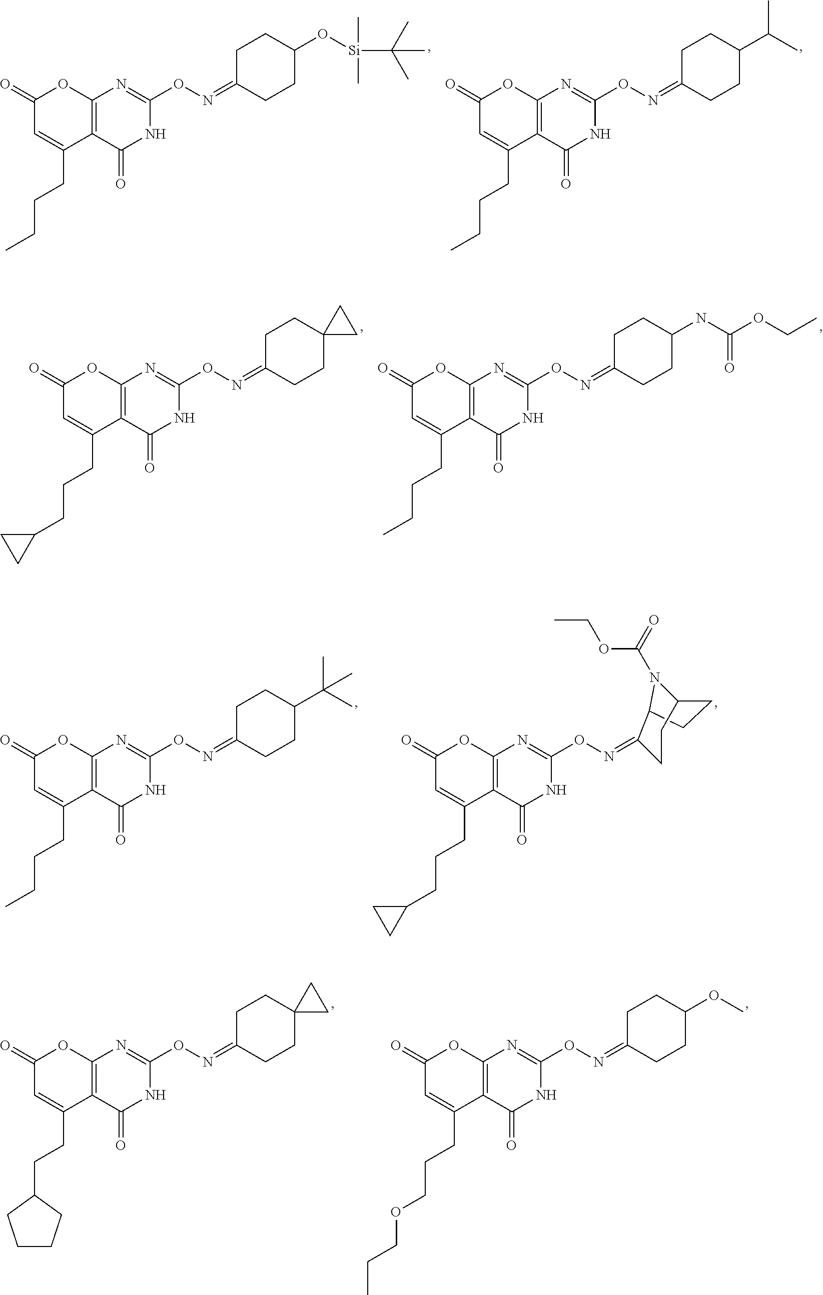 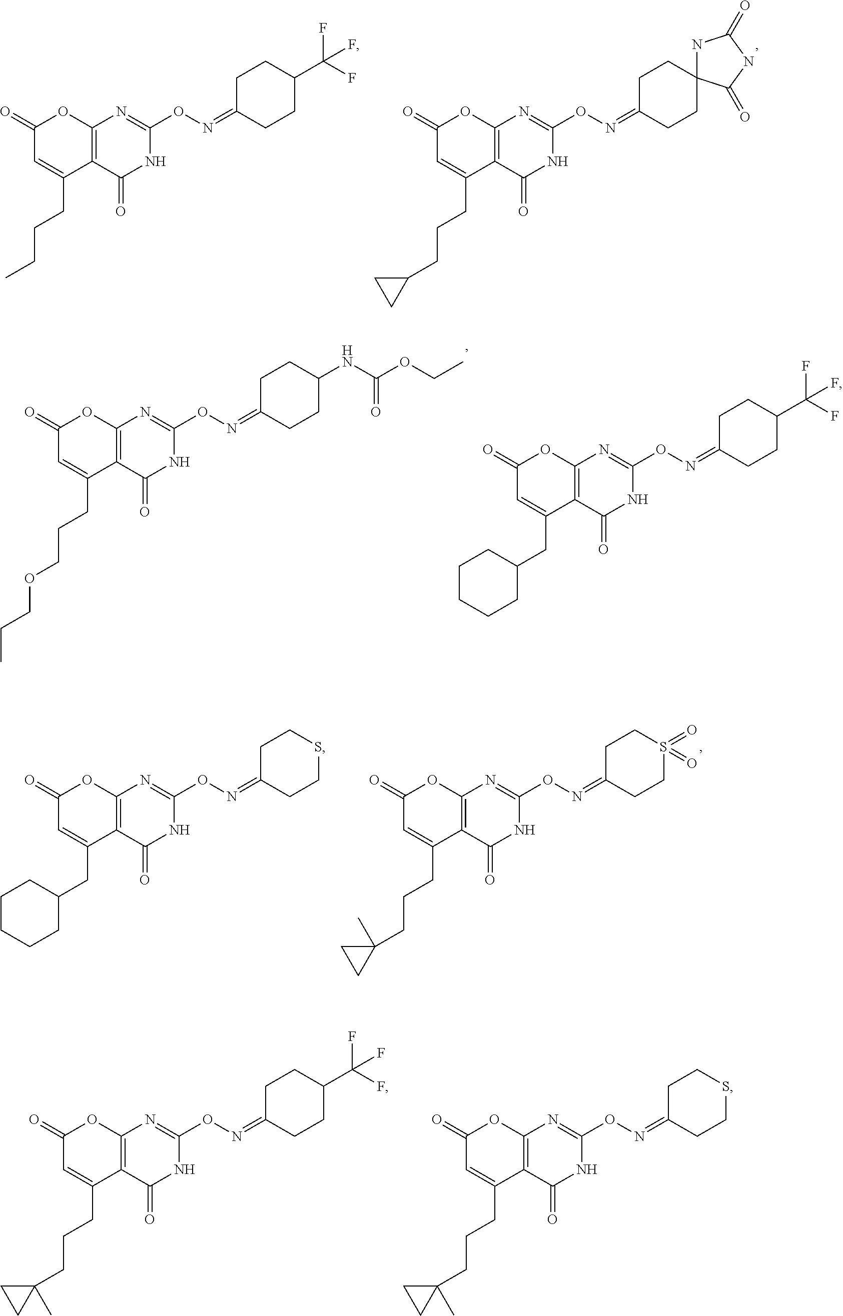 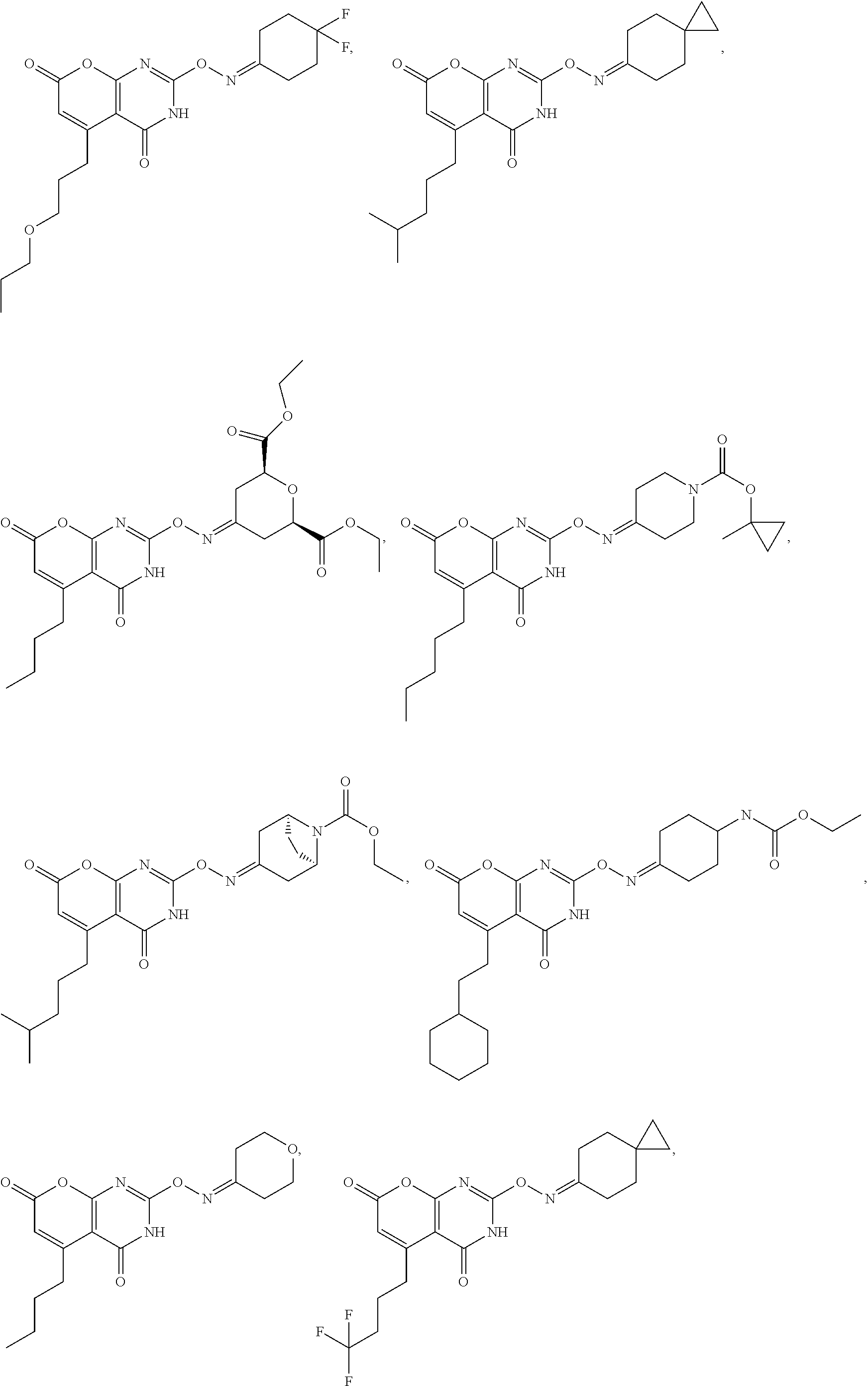 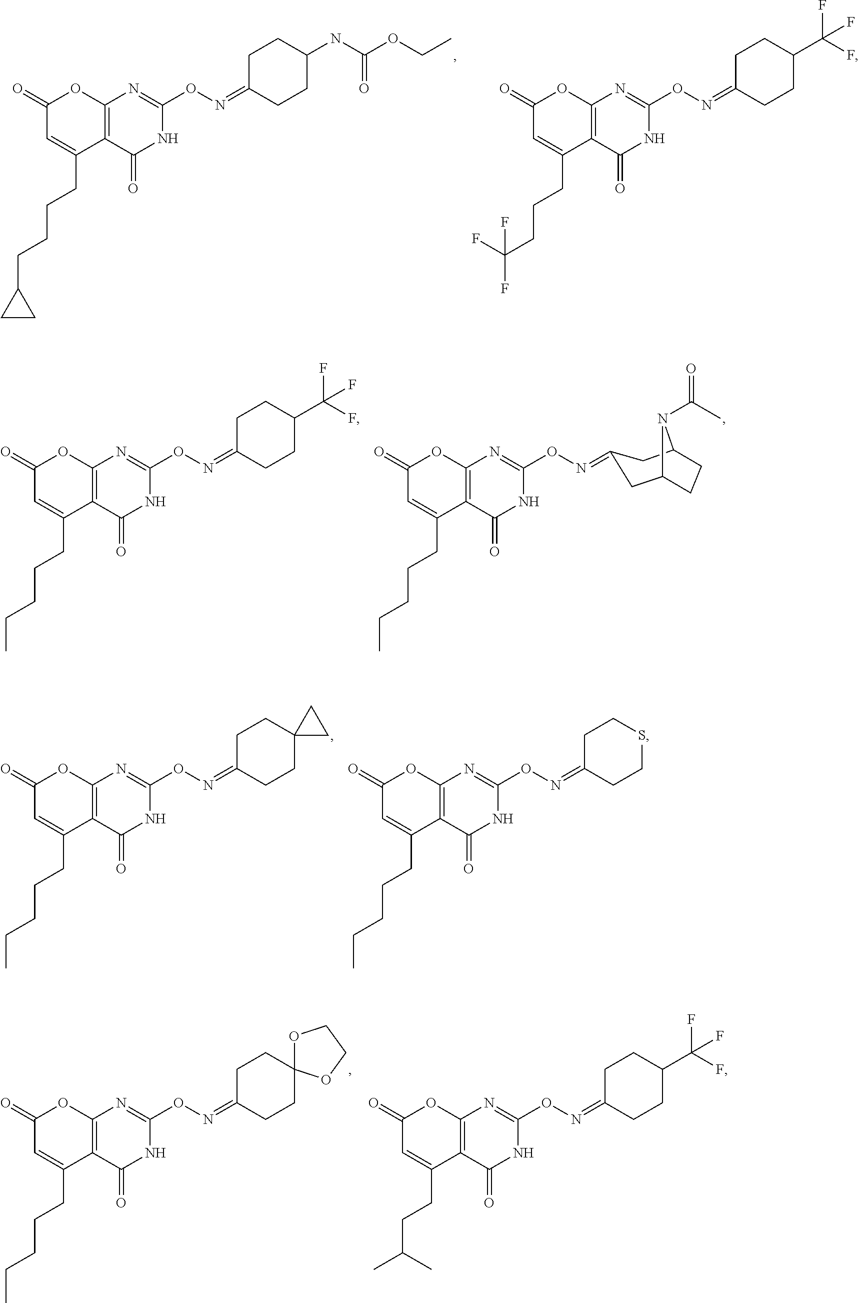 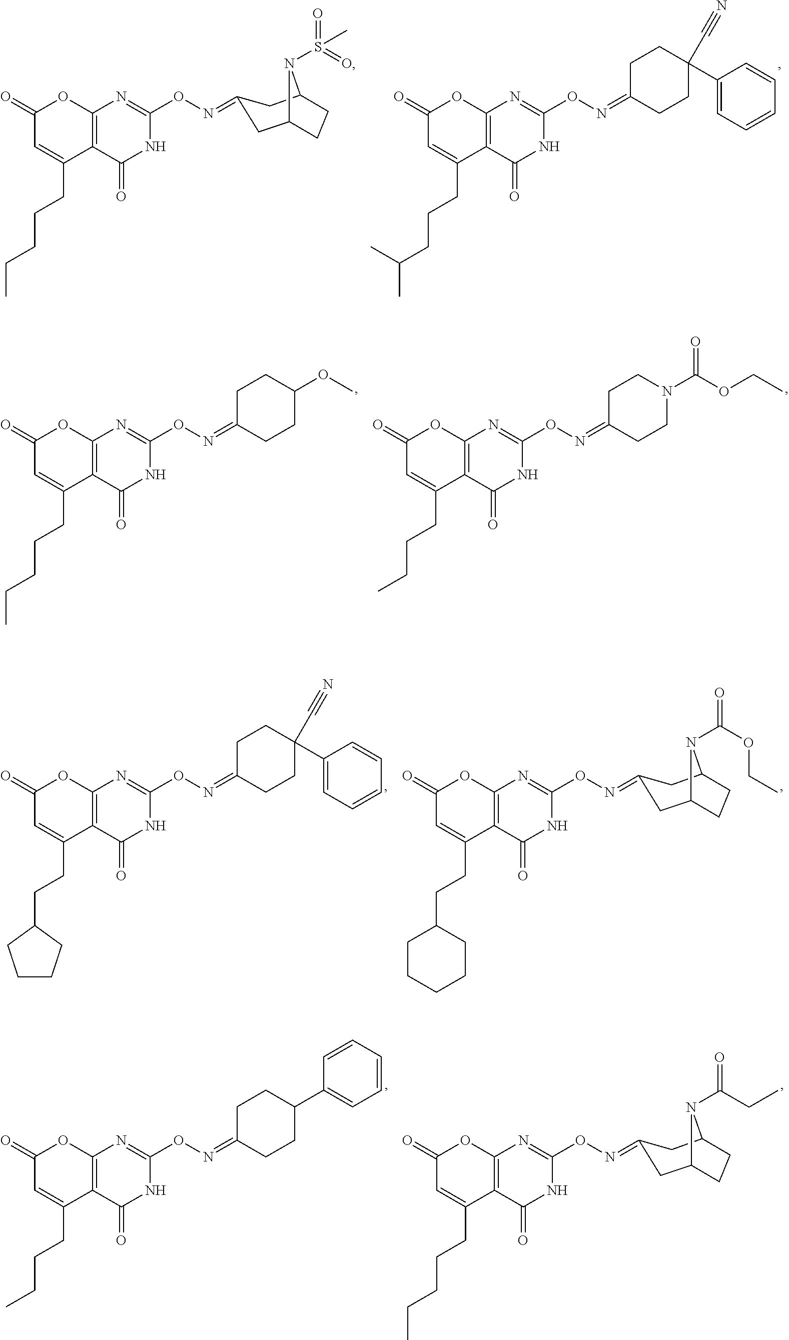 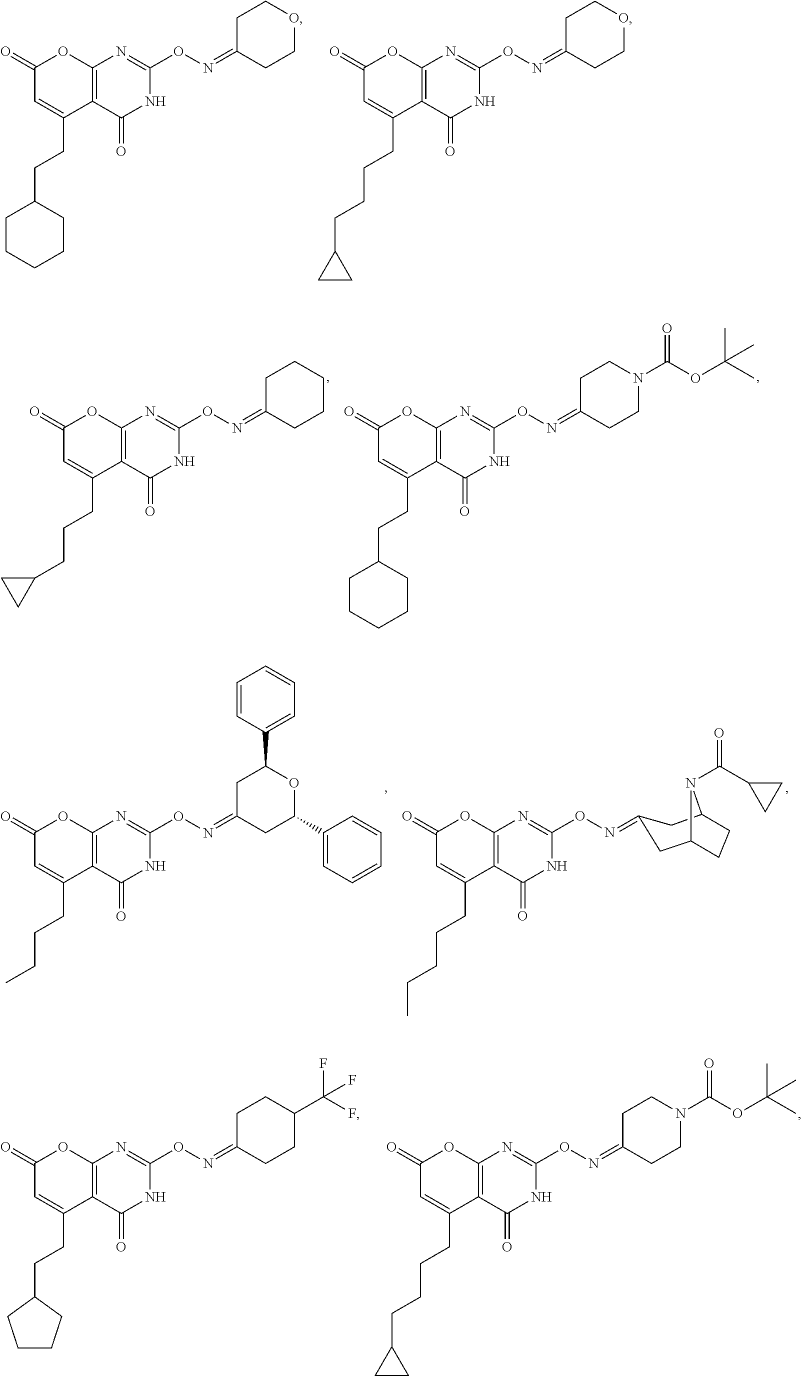 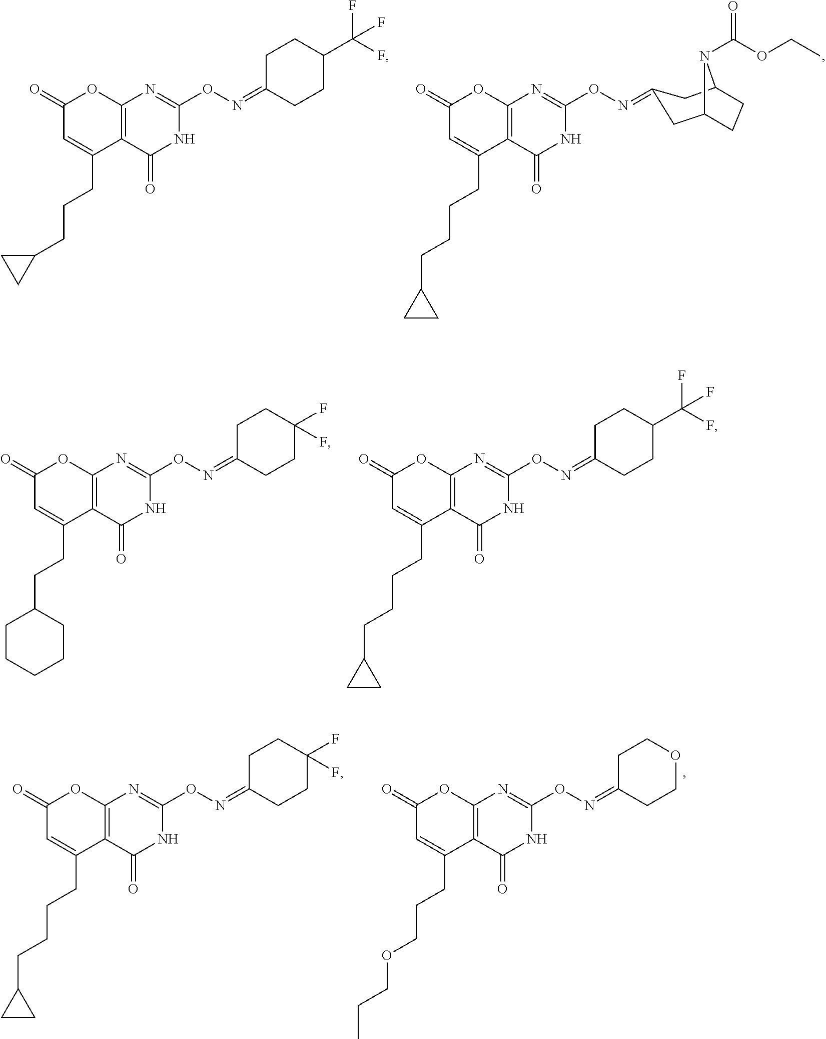 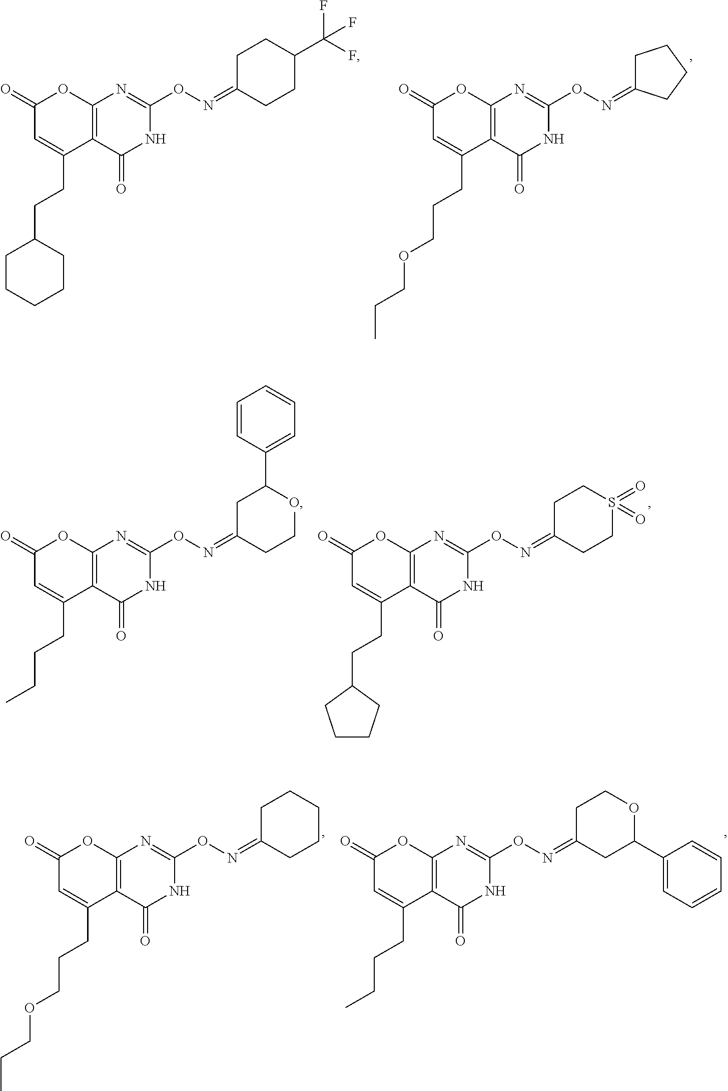 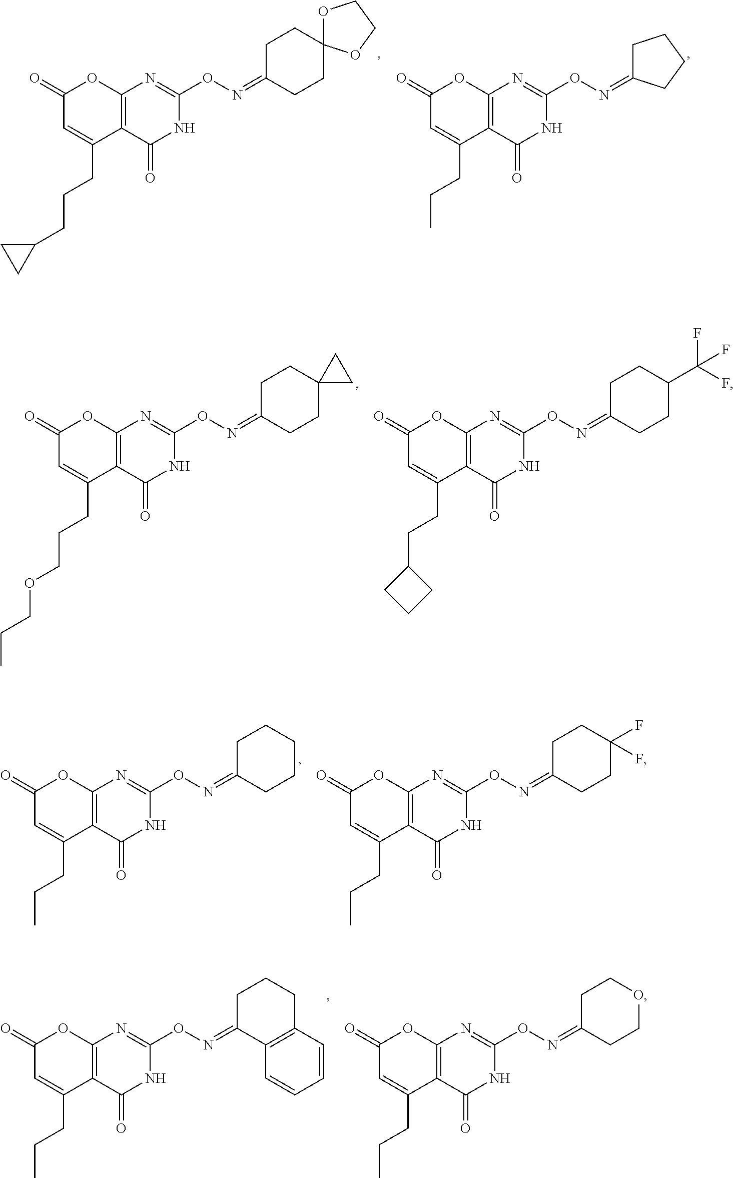 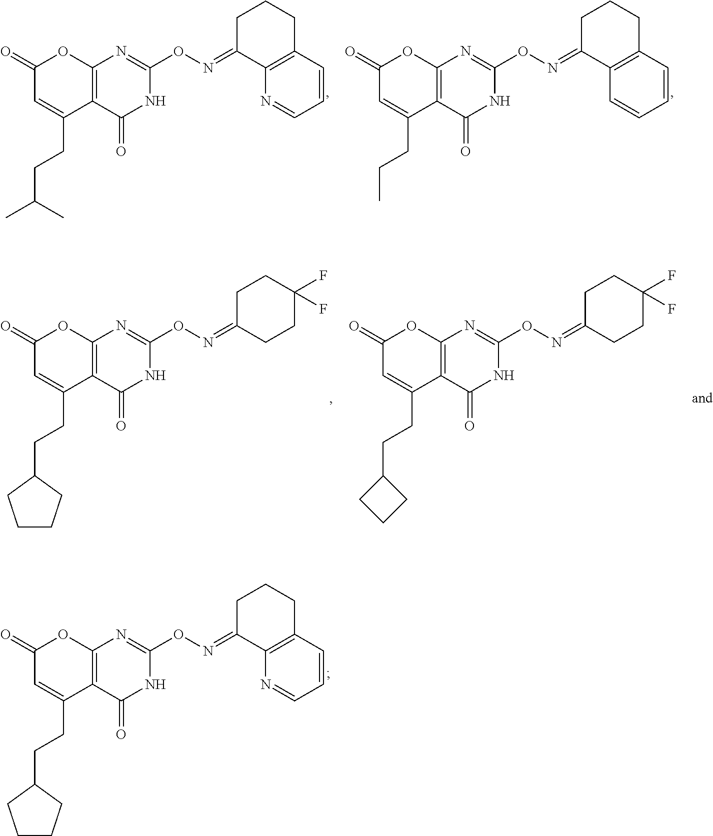
or pharmaceutically acceptable salts, solvates, esters and prodrugs thereof.

5. The pharmaceutical composition according to claim 4, wherein the PAR-1 antagonist is a compound of Formula I-A or a pharmaceutically acceptable salt thereof.

6. The pharmaceutical composition according to claim 5, wherein at least one cardiovascular agent is present.

7. The pharmaceutical composition according to claim 4, wherein the PAR-1 antagonist is a compound of Formula I-B or a pharmaceutically acceptable salt thereof.

8. The pharmaceutical composition according to claim 7, wherein at least one cardiovascular agent is present.

9. The pharmaceutical composition according to claim 4, wherein the PAR-1 antagonist is a compound of Formula I-C or a pharmaceutically acceptable salt thereof.

10. The pharmaceutical composition according to claim 9, wherein at least one cardiovascular agent is present.

11. The pharmaceutical composition according to claim 4, wherein the PAR-1 antagonist is a compound of Formula I-D or a pharmaceutically acceptable salt thereof.

12. The pharmaceutical composition according to claim 11, wherein at least one cardiovascular agent is present.

13. The pharmaceutical composition according to claim 4, wherein the PAR-1 antagonist is a compound of Formula I-1E-1, I-1E-2 or a pharmaceutically acceptable salt thereof.

14. The pharmaceutical composition according to claim 13, wherein at least one cardiovascular agent is present.

15. The pharmaceutical composition according to claim 4, wherein the PAR-1 antagonist is a compound selected from the group consisting of: 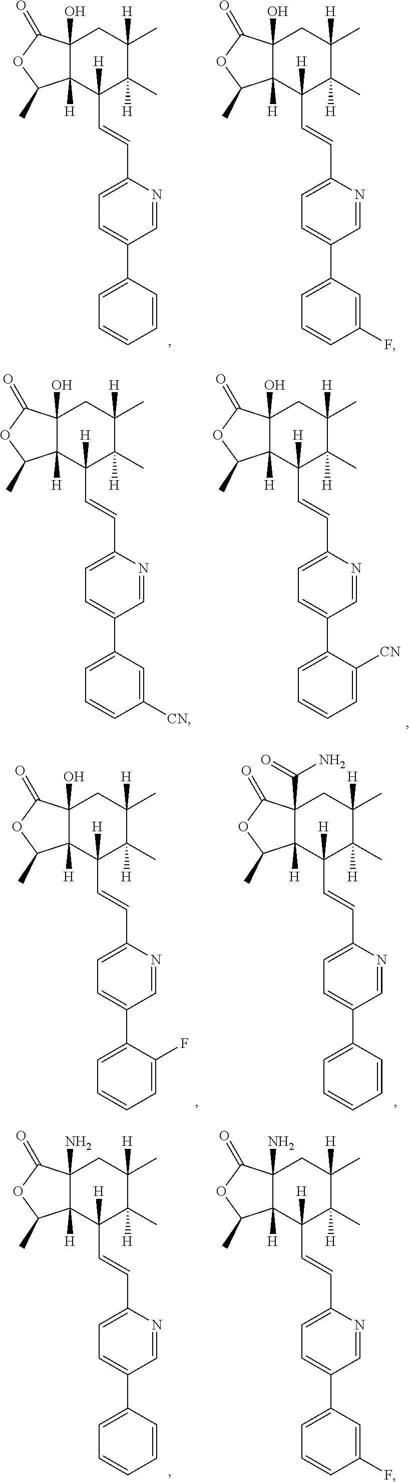 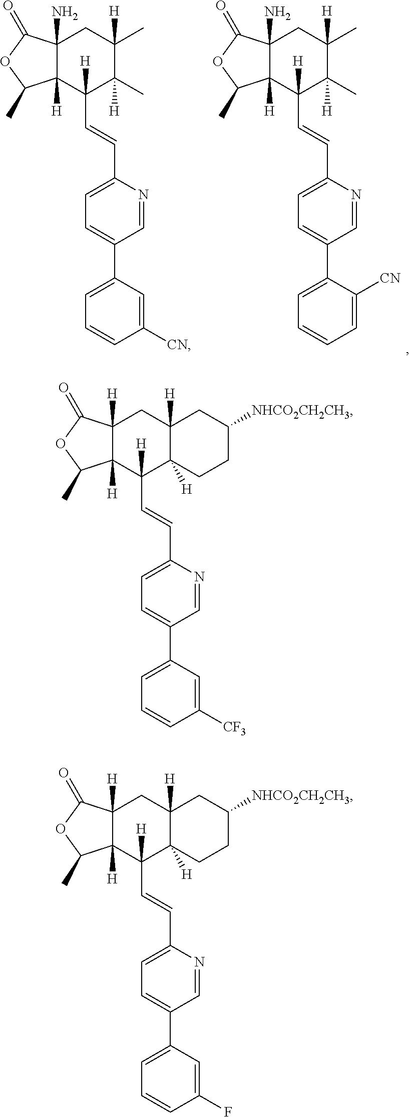 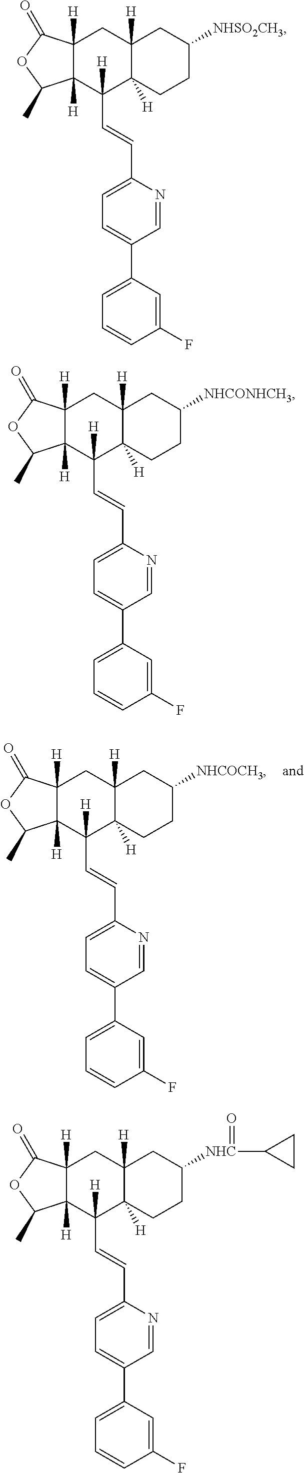
or a pharmaceutically acceptable salt or solvate thereof.

16. The pharmaceutical composition according to claim 15, wherein at least one cardiovascular agent is present.

17. The pharmaceutical composition according to claim 16, wherein the cardiovascular agent is aspirin, clopidogrel, clopidogrel bisulfate, prasugrel, or elinogrel.

18. The pharmaceutical composition according to claim 4, wherein the PAR-1 antagonist is the bisulfate salt of

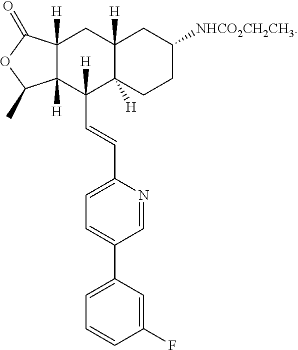

19. The pharmaceutical composition according to claim 18, wherein at least one cardiovascular agent is present.

20. The pharmaceutical composition according to claim 19, wherein the cardiovascular agent is aspirin, clopidogrel, clopidogrel bisulfate, prasugrel or elinogrel.

21. The pharmaceutical composition according to claim 4, wherein the PAR-1 antagonist is a compound selected from the group consisting of: 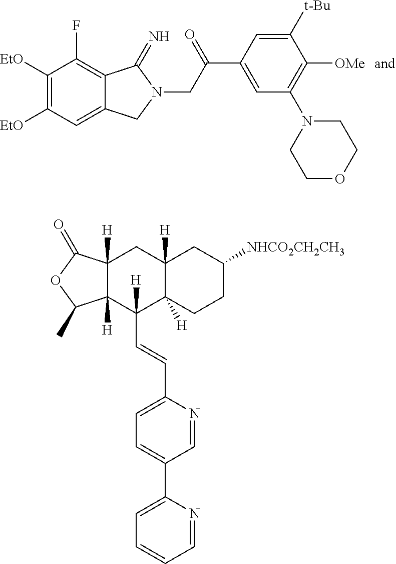
or a pharmaceutically salt thereof.

22. The pharmaceutical composition according to claim 21, wherein the cardiovascular agent is aspirin, clopidogrel bisulfate or elinogrel.

23. A method of treating diseases associated with acute coronary syndrome, secondary prevention, thrombosis, atherosclerosis, restenosis, hypertension, angina pectoris, arrhythmia, heart failure, myocardial infarction, glomerulonephritis, thrombotic stroke, thromboembolic stroke, peripheral vascular diseases, deep vein thrombosis, venous thromboembolism, a cardiovascular disease associated with hormone replacement therapy, disseminated intravascular coagulation syndrome, renal ischemia, cerebral stroke, cerebral ischemia, cerebral infarction, migraine, renal vascular homeostasis, erectile dysfunction, dyslipidemia, plaque rupture in coronary artery disease, inflammatory disorders, fibrotic disorders of the liver, kidney, lung and intestinal tract, astrogliosis, disseminated intracascular coagulation syndrome, neurodegenerative diseases, and cancer in a mammal which comprises administering to a mammal in need thereof, either together or concomitantly, an effective amount of at least one PAR1 antagonist and at least one NAR agonist.

24. A method according to claim 23, wherein the disease is selected from the group consisting of thrombosis, dyslipidemia, atherosclerosis, restenosis, and plaque rupture in coronary artery disease.
